(19) United States
(12) Patent Application Publication
(10) Pub. No.: US 2023/0312753 A1
(43) Pub. Date: Oct. 5, 2023

(54) PRECURSOR TRI-SPECIFIC ANTIBODY CONSTRUCTS AND METHODS OF USE THEREOF

(71) Applicant: Immunorizon Ltd., Rehovot (IL)

(72) Inventors: Oren BOGIN, Tel-Aviv (IL); Liat DASSA, Tel Aviv (IL)

(73) Assignee: Immunorizon Ltd., Rehovot (IL)

(21) Appl. No.: 17/922,366

(22) PCT Filed: May 4, 2021
(86) PCT No.: PCT/IL2021/050506

§ 371 Date: Oct. 30, 2022

Related U.S. Application Data
(60) Provisional application No. 63/026,143, filed on May 18, 2020.
(60) Provisional application No. 63/019,443, filed on May 4, 2020.

(51) Int. Cl. C12N 15/63; C07K 16/46; A61P 35/00

(52) U.S. Cl. CPC C07K 16/468; A61P 35/00; C12N 15/63; C07K 2317/31; C07K 2317/51; C07K 2317/515; C07K 2317/55; C07K 2317/565; C07K 2317/622

(57) ABSTRACT

Disclosed herein are precursor tri- specific antibody constructs comprising (i) a first binding domain that binds to a tumor associated antigen, (ii) a second binding domain that binds to a first natural killer (NK) cell surface antigen, and (iii) a third binding domain that binds to a T cell surface antigen or a second NK cell surface antigen. The antibody constructs further comprises regulatory domains that regulate binding to the T cell or NK cell surface antigen. Pharmaceutical compositions comprising the precursor constructs and their uses for treating tumors are also disclosed.

### FIELD OF THE INVENTION

The present disclosure relates generally to the field of antibody construct. In one embodiment, disclosed herein are precursor tri-specific antibody constructs and methods of using the same (e.g. treatment of cancer).

### SEQUENCE LISTING STATEMENT

The instant application contains a Sequence Listing which has been submitted electronically in ASCII format and is hereby incorporated by reference in its entirety. Said ASCII copy, created on Apr. 29, 2021, is named P-594180-PC_SL.txt and is 1,730,884 bytes in size.

### BACKGROUND OF THE INVENTION

The functionality of monoclonal antibodies (non conjugated or naked antibody) currently approved by drug regulatory agencies worldwide for clinical use in oncology setting are known to use one or a combination of the following mechanisms: 1) blocking cell growth signaling, 2) blocking blood supply to cancer cells, 3) directly mediating cell apoptosis, 4) eliciting immunological effector functions such as antibody dependent cellular cytoxicity (ADCC), antibody dependent cellular phagocytosis (ADCP) and complement dependent cytotoxicity (CDC), and 5) promoting adaptive immunity towards tumors.

Monoclonal antibody therapies have demonstrated survival benefits in the clinic. However, the overall response rates in cancer patients are low, and the survival benefits are marginal (several months) compared to chemotherapy. Although the underlying reasons for the lack of robust clinical anti-cancer activities are not fully understood, research has suggested that cancer cells often quickly develop compensating signaling pathways to escape cell death. Also, cancer stem cells (CSC), which are considered as potent cancer initiating cells, are less active at cell proliferation therefore they tend to sustain the lack of growth signal better.

In an attempt to improve anti-tumor activity of monoclonal antibodies, multi-specific antibodies are being developed. In contrast to classical monoclonal antibodies, which are the standard first-line therapy in several tumor entities, these multi-specific antibodies may bring together a tumor cell and the means to destroy the tumor cell, thereby increasing the efficiency of treatment. These multi-specific antibodies provide for new treatment options for cancer patients.

Another anti-cancer therapeutic approach is to utilize T cells. T cells provide defense against cancer throughout life by patrolling the body in search for newly arisen cancer cells and eliminating them effectively and promptly. Therapeutic approaches utilizing T cells have proved successful in cancer treatment of at least metastatic melanoma, metastatic kidney cancer, asymptomatic metastatic hormone refractory prostate cancer, and advanced melanoma.

While the use of targeting activated T cells provides one pathway for destruction of tumor cells, another pathway of cellular cytotoxicity is through the recruitment and targeting of nature killer (NK) cells. NK cells are white blood cells, part of the innate immune system that plays a major role in the host-rejection of tumors. NK cells are cytotoxic, wherein small granules in their cytoplasm contain special proteins such as perforin and proteases known as granzymes. Upon release in close proximity to a tumor cell slated for killing, perforin forms pores in the cell membrane of the targeted cell through which the granzymes and associated molecules can enter, inducing apoptosis. Therapeutic antibodies, such as RITUXAN and HERCEPTIN, can drive killing of bound tumors through NK-cell-mediated antibody-dependent cell-mediated cytotoxicity (ADCC).

Tumor escape from NK cell immune surveillance predominantly occurs via two mechanisms: reduction of activating signals or increases in inhibitory signals delivered to NK cells. Thus, another potential target of cancer immunotherapy is the removal/blocking of molecules that suppress NK activation, and removal/blocking of molecules that result in NK cell hypo-responsiveness. Restoring NK cell antitumor activity is critical for establishing host immunity against cancer, which is a primary objective of cancer immunotherapy.

Another consideration in tumor cytotoxicity is the tumor microenvironment (TME). The TME includes novel targets that can help direct and improve the actions of antibody therapies by potentiating host antitumor immune responses. For example, T cells play an unexpectedly critical role in anti-tumor antigen antibody therapy, although their importance is often not observed due to studies being performed in immunodeficient mice. IL-2 treatment was shown to amplify monoclonal antibody therapy not simply via the previously assumed NK-mediated ADCC, but also by boosting the CD8+ T cell adaptive response, since IL-2 exerts significant pleiotropic effects on regulatory, helper, and cytolytic memory T-cells (Liao et al., Immunity. 2013, 38:13) (Zhu EF et.al Cancer Cell. 2015 27:489).

A pitfall of antibody therapeutics used in cancer treatment is the “off-target” binding of the antibody to non-cancer tumor-associated-antigen-expressing cells, especially if such binding leads to cytotoxicity. Thus, “off-target” binding by multi-specific and bispecific antibodies presents a potential challenge to controlling their “off-target” activity against normal tissues that also express the antigen, even at extremely low levels. These “off-target” effects are a serious limitation to multi-specific and bispecific antibody therapeutics. Another drawback of many bispecific or multi-specific antibodies is their short half-life.

There remains a need to provide multi-specific trivalent antibodies with qualities that specifically target cytotoxicity to tumor cells while reducing the toxic side effects and preserving the antibodies effectiveness.

### SUMMARY OF THE INVENTION

Reducing the non-specific toxic side effects of multi-specific antibodies and concurrently enhancing the effectiveness of these antibodies require an antibody having a precursor form that (1) engages a target associated with a tumor cell, a tumor-associated cell, or a tumor cell environment, and (2) activates cytotoxic cells, for example T cells or NK cells, once localized within or adjacent to the tumor microenvironment (TME). Further, it is essential that such multi-specific antibodies do not reduce significantly the immunogenicity to a tumor or tumor-associated target. In one embodiment, the precursor tri-specific antibody constructs described herein addresses these needs by attaching a regulatable half-life enhancing component and a blocking component that inhibits the antibody from engaging a toxicity-providing cell prior to binding to a tumor or tumor-associated target or at the TME. Further, the tri-specific antibodies presented herein may concurrently engage two different types of cytotoxic cells (e.g. T cells and NK cells).

In one embodiment, after the precursor tri-specific antibody constructs described herein are administered to a cancer patient, the precursor antibody constructs eventually reach the tumor-associated targets or the TME. At the vicinity of the tumor-associated targets or the TME, the precursor antibody constructs would bind to a tumor-associated antigen, and the blocking component(s) that inhibits the antibody from engaging T cells and/or NK cells would be removed by proteases present at the TME. Subsequently, the antibody constructs (now in “active” form) would bind to T cells and/or NK cells, thereby recruiting T cells and/or NK cells from the circulation to the tumor-associated targets or the TME.

In one embodiment, disclosed herein is a precursor tri-specific antibody construct, comprising: a) a first binding domain that binds to a tumor associated antigen (TAA); b) a second binding domain comprising a cytokine receptor engager or a second binding domain that binds to a first natural killer (NK) cell surface antigen; c) a third binding domain that binds to a T cell surface antigen or a second NK cell surface antigen; and d) a regulatory domain comprising either (i) a first and a second sub-regulatory domain, the first sub-regulatory domain comprising a first protease cleavage domain and a half-life prolonging (HLP) domain, and the second sub-regulatory domain comprising a second protease cleavage domain and a CAP component that reduces the ability of the third binding domain to bind to its target antigen; or (ii) a single regulatory domain comprising a protease cleavage domain, a half-life prolonging (HLP) domain, and a CAP component that reduces the ability of the third binding domain to bind to its target antigen.

In one embodiment, when the second binding domain binds to a NK cell surface antigen, the second binding domain further comprises a third regulatory domain comprising a third protease cleavage domain and a CAP component that reduces the ability of the second binding domain to bind to the NK cell surface antigen.

In one embodiment, the first binding domain, the second binding domain each comprises a single chain variable fragment (scFv). In one embodiment, the third binding domain comprises a Fab antigen binding fragment.

In one embodiment, the T cell surface antigen is CD3. In another embodiment, one or both of the NK cell surface antigens bound by the antibody construct disclosed herein can be an activating NK cell receptor or an inhibitory NK cell receptor. It is known in the art that activation of the NK cells is mediated by a network of activating and inhibitory receptors; it is the integration of the activating and inhibitory signals that determines if the NK cells become cytotoxic (see e.g. Chester et al., Frontiers in Immunology, 2015, 6:Article 601). Activating receptors for NK cells include, but are not limited to, CD16, TRAIL, NKG2D, 2B4, DNAM-1, NKp30, NKp44, NKp46 and NKp80. Inhibitory receptors for NK cells include, but are not limited to, KIR (killer cell immunoglobulin-like receptor) and CD94/NKG2A heterodimer. Moreover, there are co-stimulatory proteins with key roles in regulating the activation of NK cells, for example, CD137, OX40 and CD27. In another embodiment, one or both of the NK cell surface antigens bound by the antibody construct disclosed herein can be, but is not limited to, CD16 (FcγRIII), CD16a (FcγRIIIa), CD56, sMICA/B, ILT, SLAMF7, NKp44, NKp30, DNAM-1, NKG2A, NKG2D, NKG2C/CD94, NKp46, KIR2/DL3, KIR2DL1, NKRP1, NKG2E/CD94, NKG2F/CD94, CD69, LLT1, ILT2, AICL, CD26, NKp80, KIR family receptors, or CD122/IL-2Rbeta.

In one embodiment, the second binding domain and the third binding domain bind to different NK cell surface antigens. In another embodiment, the second binding domain and the third binding domain bind to the same NK cell surface antigen. In one embodiment, the second binding domain binds to NKG2A and the third binding domain binds to NKG2D. In another embodiment, the second binding domain binds to NKG2D and the third binding domain binds to NKG2A.

In one embodiment of the precursor tri-specific antibody construct, the first binding domain binds to a TAA, the second binding domain binds to a NK cell surface antigen, and the third binding domain binds to a T cell surface antigen.

In one embodiment of the precursor tri-specific antibody construct, the first binding domain binds to a TAA, the second binding domain binds to a NK cell surface antigen, and the third binding domain binds to a T cell surface antigen, wherein the second binding domain further comprises a third regulatory domain comprising a third protease cleavage domain and a CAP component that reduces the ability of the second binding domain to bind to the NK cell surface antigen.

In one embodiment of the precursor tri-specific antibody construct, the first binding domain binds to a TAA, the second binding domain binds to a first NK cell surface antigen, and the third binding domain binds to a second NK cell surface antigen.

In one embodiment of the precursor tri-specific antibody construct, the first binding domain binds to a TAA, the second binding domain binds to a first NK cell surface antigen, and the third binding domain binds to a second NK cell surface antigen, wherein the second binding domain further comprises a third regulatory domain comprising a third protease cleavage domain and a CAP component that reduces the ability of the second binding domain to bind to the NK cell surface antigen.

In one embodiment of the precursor tri-specific antibody construct, the first binding domain binds to a TAA, the cytokine receptor engager of the second binding domain comprises a cytokine that binds to a cytokine receptor, and the third binding domain binds to a NK cell surface antigen. In one embodiment, the cytokine is a pro-inflammatory cytokine. In another embodiment, the cytokine is an anti-inflammatory cytokine. In one embodiment, the cytokine can be IL-15, IL-2, IL-12, TNF-alpha, IL-6, TGF-beta, IL-10, IL-8, IL-17, IL-21, INF, and VEGF. In one embodiment, the cytokine is IL-15 that binds to an IL-15 receptor.

In one embodiment, the HLP molecule comprises a human serum albumin (HSA) polypeptide.

In one embodiment, the CAP component that reduces binding to the T cell surface antigen comprises an amino acid sequence of an extracellular epitope of human CD3ε. In one embodiment, the CAP component comprises the amino acid sequence of SEQ ID NO:5, or a homolog thereof.

In one embodiment, the CAP component that reduces binding to the NK cell surface antigen comprises an amino acid sequence of an extracellular epitope of the NK surface antigen.

In one embodiment, the protease cleavage domains of the antibody construct disclosed herein are cleaved by the same protease. In another embodiment, the protease cleavage domains of the antibody construct disclosed herein are cleaved by different proteases. In one embodiment, one or more of the protease cleavage domains comprise an amino acid sequence cleavable by a serine protease, a cysteine protease, an aspartate protease, or a matrix metalloprotease (MMP). In another embodiment, one or more of the protease cleavage domains comprise amino acid sequence that is a combination substrate cleaved by one or more of MMP2/9, uPA, matriptase, and legumain.

In one embodiment, the MMP can be, but is not limited to, matrix metalloprotease 1 (MMP-1), matrix metalloprotease 2 (MMP-2), matrix metalloprotease 9 (MMP-9), or matrix metalloprotease 14 (MMP-14). In one embodiment, the serine protease is an urokinase-type plasminogen activator (uPA) protease or a membrane-type serine protease (MT-SP1). In one embodiment, the combination substrate has the amino acid sequence of SEQ ID NO:35. In another embodiment, one or more of the protease cleavage domains comprise an amino acid sequence having the sequence of one of SEQ ID NOs:9-14 and SEQ ID NO:35.

In one embodiment, the tumor associated antigen (TAA) can be, but is not limited to, a tumor cell surface antigen, a tumor micro-environment antigen, a stromal antigen in the tumor micro-environment (TME), an angiogenic antigen in the TME, or an antigen on a blood vessel in a TME.

In one embodiment, the TAA can be, but is not limited to, EGFR, FcγRI, FcγRIIa FcγRIIb FcγRIIIa FcγRIIIb, CD28, CD137, CTLA-4, FAS, fibroblast growth factor receptor 1 (FGFR1), FGFR2, FGFR3, FGFR4, glucocorticoid-induced TNFR-related (GITR) protein, lymphotoxin-beta receptor (LTβR), toll-like receptors (TLR), tumor necrosis factor-related apoptosis-inducing ligand-receptor 1 (TRAIL receptor 1), TRAIL receptor 2, prostate-specific membrane antigen (PSMA) protein, prostate stem cell antigen (PSCA) protein, tumor-associated protein carbonic anhydrase IX (CAIX), epidermal growth factor receptor 1 (EGFR1), EGFRvIII, human epidermal growth factor receptor 2 (Her2/neu; Erb2), ErbB3 (HER3), Folate receptor, ephrin receptors, PDGFRa, ErbB-2, CD20, CD22, CD30, CD33, CD40, CD37, CD38, CD70, CD74, CD56, CD40), CD80, CD86, CD2, p53, cMet (tyrosine-protein kinase Met) (hepatocyte growth factor receptor) (HGFR), MAGE-A1, MAGE-A2, MAGE-A3, MAGE-A4, MAGE-A6, MAGE-A10, MAGE-A12, BAGE, DAM-6, DAM -10, GAGE-1, GAGE-2, GAGE-8, GAGE-3, GAGE -4, GAGE-5, GAGE-6, GAGE-7B, NA88-A, NY-ESO-1, BRCA1, BRCA2, MART-1, MC1R, Gp100, PSA, PSM, Tyrosinase, Wilms’ tumor antigen (WT1), TRP-1, TRP-2, ART-4, CAMEL, Cyp-B, hTERT, hTRT, iCE, MUC1, MUC2, P-cadherin, Myostatin (GDF8), Cripto (TDGF1), MUC5AC, PRAME, P15, RU1, RU2, SART-1, SART-3, WT1, AFP, β-catenin/m, Caspase-8/m, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, ETV6/AML, LDLR/FUT, Pml/RARα, TEL/AML1, CD28, CD137, CanAg, Mesothelin, DR5, PD-1, PD1L, IGF-1R, CXCR4, Neuropilin 1, Glypicans, EphA2, CD138, B7-H3, B7-H4, gpA33, GPC3, SSTR2, ROR1, 5T4, or VEGF-R2.

In one embodiment, the TAA is EGFR, ROR1, PSMA, or 5T4. In one embodiment, when the antigen is EGFR, the first binding domain comprises the amino acid sequence of SEQ ID NO:34 or SEQ ID NO:37; when the antigen is ROR1, the first binding domain comprises the amino acid sequence of SEQ ID NO:156 or SEQ ID NO:166; when the antigen is PSMA, the first binding domain comprises the amino acid sequence of SEQ ID NO:168 or SEQ ID NO:170; and when the antigen is 5T4, the first binding domain comprises the amino acid sequence of SEQ ID NO:172 or SEQ ID NO:174.

In one embodiment, the tumor micro-environment antigen can be, but is not limited to, KIR, NKG2A, ILT, LILR, or TIGIT.

In one embodiment, the stromal antigen in the tumor micro-environment can be, but is not limited to, fibroblast activation protein (FAP), alpha smooth muscle actin (αSMA), PDGFRα, Integrin α11β1(ITGA11)VEGF, Tenascin-C, periostin, fibroblast specific protein 1 (S10A4, FSP1), desmin, vimentin, paladin, urokinase-type plasminogen activator receptor associated protein (UPARAP), galectin-3, podoplanin, platelet, CCL2, or CXCL12.

In one embodiment, the angiogenic antigen in the tumor micro-environment can be, but is not limited to, bFGF, INF, or VEGF.

In one embodiment, the antigen on a blood vessel in the tumor micro-environment comprises an endothelial cell surface antigen such as CD31, CD105, CD146, and CD144 etc.

In one embodiment, the third binding domain comprises a Fab region having a heavy chain (VH-CH) region and a light chain (VL-CL) region, and the first binding domain is located C-terminally to the VL-CL or VH-CH region of the third binding domain. In one embodiment, when the first binding domain is located C-terminally to the VL-CL region, the second binding domain is located C-terminally to the VH-CH region. Alternatively, when the first binding domain is located C-terminally to the VH-CH region, the second binding domain is located C-terminally to the VL-CL region.

In another embodiment, the third binding domain comprises a heavy chain variable region (VH) and a light chain variable region (VL), wherein the first regulatory domain, which comprises a HLP domain located N-terminally to the protease cleavage domain, is located N-terminally to the VH or VL region of the third binding domain. In one embodiment, when the first regulatory domain is located N-terminally to the VL region, the second regulatory domain, which comprises a CAP component located N-terminally to the protease cleavage domain, is located N-terminally to the VH region. Alternatively, when the first regulatory domain is located N-terminally to the VH region, the second regulatory domain, which comprises a CAP component located N-terminally to the protease cleavage domain, is located N-terminally to the VL region.

In another embodiment, the third binding domain comprises a heavy chain variable region (VH) and a light chain variable region (VL), wherein the single regulatory domain comprising a protease cleavage domain, a half-life prolonging (HLP) domain, and a CAP component is located N-terminally to the VH region or to the VL region of the third binding domain.

In one embodiment, the precursor tri-specific antibody construct comprises two polypeptides, polypeptide A and polypeptide B, each of which comprising one or more heavy chain variable region (VH) and one or more light chain variable region (VL), for example,
(a) polypeptide A comprises components having an order N-terminal to C-terminal: HLP domain, protease cleavage domain, VH of the third binding domain, first binding domain comprising first VH-first VL; and polypeptide B comprises components having an order N-terminal to C-terminal: CAP component, protease cleavage domain, VL of the third binding domain, second binding domain comprising second VH-second VL; or
(b) polypeptide A comprises components having an order N-terminal to C-terminal: HLP domain, protease cleavage domain, VH of the third binding domain, first binding domain comprising first VL-first VH; and polypeptide B comprises components having an order N-terminal to C-terminal: CAP component, protease cleavage domain, VL of the third binding domain, second binding domain comprising second VH-second VL; or
(c) polypeptide A comprises components having an order N-terminal to C-terminal: HLP domain, protease cleavage domain, VH of the third binding domain, first binding domain comprising first VH-first VL; and polypeptide B comprises components having an order N-terminal to C-terminal: CAP component, protease cleavage domain, VL of the third binding domain, second binding domain comprising second VL-second VH; or
(d) polypeptide A comprises components having an order N-terminal to C-terminal: HLP domain, protease cleavage domain, VH of the third binding domain, first binding domain comprising first VL-first VH; and polypeptide B comprises components having an order N-terminal to C-terminal: CAP component, protease cleavage domain, VL of the third binding domain, second binding domain comprising second VL-second VH; or
(e) polypeptide A comprises components having an order N-terminal to C-terminal: CAP component, protease cleavage domain, VH of the third binding domain, first binding domain comprising first VH-first VL; and polypeptide B comprises components having an order N-terminal to C-terminal: HLP domain, protease cleavage domain, VL of the third binding domain, second binding domain comprising second VH-second VL; or
(f) polypeptide A comprises components having an order N-terminal to C-terminal: CAP component, protease cleavage domain, VH of the third binding domain, first binding domain comprising first VL-first VH; and polypeptide B comprises components having an order N-terminal to C-terminal: HLP domain, protease cleavage domain, VL of the third binding domain, second binding domain comprising second VH-second VL; or
(g) polypeptide A comprises components having an order N-terminal to C-terminal: CAP component, protease cleavage domain, VH of the third binding domain, first binding domain comprising first VH-first VL; and polypeptide B comprises components having an order N-terminal to C-terminal: HLP domain, protease cleavage domain, VL of the third binding domain, second binding domain comprising second VL-second VH; or
(h) polypeptide A comprises components having an order N-terminal to C-terminal: CAP component, protease cleavage domain, VH of the third binding domain, first binding domain comprising first VL-first VH; and polypeptide B comprises components having an order N-terminal to C-terminal: HLP domain, protease cleavage domain, VL of the third binding domain, second binding domain comprising second VL-second VH.

In one embodiment, the precursor tri-specific antibody construct comprises two polypeptides, polypeptide A and polypeptide B, each of which comprising one or more heavy chain variable region (VH) and one or more light chain variable region (VL), for example,
(a) polypeptide A comprises components having an order N-terminal to C-terminal: VH of the third binding domain, first binding domain comprising first VH-first VL; and polypeptide B comprises components having an order N-terminal to C-terminal: VL of the third binding domain, second binding domain comprising second VH-second VL, wherein a regulatory domain is located N-terminal to either polypeptide A or B, the regulatory domain comprises components having an order N-terminal to C-terminal: a CAP component, a half-life prolonging (HLP) domain, and a protease cleavage domain; or
(b) polypeptide A comprises components having an order N-terminal to C-terminal: VH of the third binding domain, first binding domain comprising first VL-first VH; and polypeptide B comprises components having an order N-terminal to C-terminal: VL of the third binding domain, second binding domain comprising second VH-second VL, wherein a regulatory domain is located N-terminal to either polypeptide A or B, the regulatory domain comprises components having an order N-terminal to C-terminal: a CAP component, a half-life prolonging (HLP) domain, and a protease cleavage domain; or
(c) polypeptide A comprises components having an order N-terminal to C-terminal: VH of the third binding domain, first binding domain comprising first VH-first VL; and polypeptide B comprises components having an order N-terminal to C-terminal: VL of the third binding domain, second binding domain comprising second VL-second VH, wherein a regulatory domain is located N-terminal to either polypeptide A or B, the regulatory domain comprises components having an order N-terminal to C-terminal: a CAP component, a half-life prolonging (HLP) domain, and a protease cleavage domain; or
(d) polypeptide A comprises components having an order N-terminal to C-terminal: VH of the third binding domain, first binding domain comprising first VL-first VH; and polypeptide B comprises components having an order N-terminal to C-terminal: VL of the third binding domain, second binding domain comprising second VL-second VH, wherein a regulatory domain is located N-terminal to either polypeptide A or B, the regulatory domain comprises components having an order N-terminal to C-terminal: a CAP component, a half-life prolonging (HLP) domain, and a protease cleavage domain.

In another embodiment, the precursor tri-specific antibody construct comprises two polypeptides, polypeptide A and polypeptide B, each of which comprising one or more heavy chain variable region (VH) and one or more light chain variable region (VL), for example,
(a) polypeptide A comprises components having an order N-terminal to C-terminal: HLP domain, protease cleavage domain, VH of the third binding domain, first binding domain comprising first VH-first VL; and polypeptide B comprises components having an order N-terminal to C-terminal: CAP component, protease cleavage domain, VL of the third binding domain, second binding domain comprising second VH-second VL, protease cleavage domain, and a second CAP component; or
(b) polypeptide A comprises components having an order N-terminal to C-terminal: HLP domain, protease cleavage domain, VH of the third binding domain, first binding domain comprising first VL-first VH; and polypeptide B comprises components having an order N-terminal to C-terminal: CAP component, protease cleavage domain, VL of the third binding domain, second binding domain comprising second VH-second VL, protease cleavage domain, and a second CAP component; or
(c) polypeptide A comprises components having an order N-terminal to C-terminal: HLP domain, protease cleavage domain, VH of the third binding domain, first binding domain comprising first VH-first VL; and polypeptide B comprises components having an order N-terminal to C-terminal: CAP component, protease cleavage domain, VL of the third binding domain, second binding domain comprising second VL-second VH, protease cleavage domain, and a second CAP component; or
(d) polypeptide A comprises components having an order N-terminal to C-terminal: HLP domain, protease cleavage domain, VH of the third binding domain, first binding domain comprising first VL-first VH; and polypeptide B comprises components having an order N-terminal to C-terminal: CAP component, protease cleavage domain, VL of the third binding domain, second binding domain comprising second VL-second VH, protease cleavage domain, and a second CAP component; or
(e) polypeptide A comprises components having an order N-terminal to C-terminal: CAP component, protease cleavage domain, VH of the third binding domain, first binding domain comprising first VH-first VL; and polypeptide B comprises components having an order N-terminal to C-terminal: HLP domain, protease cleavage domain, VL of the third binding domain, second binding domain comprising second VH-second VL, protease cleavage domain, and a second CAP component; or
(f) polypeptide A comprises components having an order N-terminal to C-terminal: CAP component, protease cleavage domain, VH of the third binding domain, first binding domain comprising first VL-first VH; and polypeptide B comprises components having an order N-terminal to C-terminal: HLP domain, protease cleavage domain, VL of the third binding domain, second binding domain comprising second VH-second VL, protease cleavage domain, and a second CAP component; or
(g) polypeptide A comprises components having an order N-terminal to C-terminal: CAP component, protease cleavage domain, VH of the third binding domain, first binding domain comprising first VH-first VL; and polypeptide B comprises components having an order N-terminal to C-terminal: HLP domain, protease cleavage domain, VL of the third binding domain, second binding domain comprising second VL-second VH, protease cleavage domain, and a second CAP component; or
(h) polypeptide A comprises components having an order N-terminal to C-terminal: CAP component, protease cleavage domain, VH of the third binding domain, first binding domain comprising first VL-first VH; and polypeptide B comprises components having an order N-terminal to C-terminal: HLP domain, protease cleavage domain, VL of the third binding domain, second binding domain comprising second VL-second VH, protease cleavage domain, and a second CAP component.

In another embodiment, the precursor tri-specific antibody construct comprises two polypeptides, polypeptide A and polypeptide B, each of which comprising one or more heavy chain variable region (VH) and one or more light chain variable region (VL), for example,
(a) polypeptide A comprises components having an order N-terminal to C-terminal: VH of the third binding domain, first binding domain comprising first VH-first VL; and polypeptide B comprises components having an order N-terminal to C-terminal: VL of the third binding domain, second binding domain comprising second VH-second VL, protease cleavage domain, and a CAP component, wherein a regulatory domain is located N-terminal to either polypeptide A or B, the regulatory domain comprises components having an order N-terminal to C-terminal: a second CAP component, a half-life prolonging (HLP) domain, and a protease cleavage domain; or
(b) polypeptide A comprises components having an order N-terminal to C-terminal: VH of the third binding domain, first binding domain comprising first VL-first VH; and polypeptide B comprises components having an order N-terminal to C-terminal: VL of the third binding domain, second binding domain comprising second VH-second VL, protease cleavage domain, and a CAP component, wherein a regulatory domain is located N-terminal to either polypeptide A or B, the regulatory domain comprises components having an order N-terminal to C-terminal: a second CAP component, a half-life prolonging (HLP) domain, and a protease cleavage domain; or
(c) polypeptide A comprises components having an order N-terminal to C-terminal: VH of the third binding domain, first binding domain comprising first VH-first VL; and polypeptide B comprises components having an order N-terminal to C-terminal: VL of the third binding domain, second binding domain comprising second VL-second VH, protease cleavage domain, and a CAP component, wherein a regulatory domain is located N-terminal to either polypeptide A or B, the regulatory domain comprises components having an order N-terminal to C-terminal: a second CAP component, a half-life prolonging (HLP) domain, and a protease cleavage domain; or
(d) polypeptide A comprises components having an order N-terminal to C-terminal: VH of the third binding domain, first binding domain comprising first VL-first VH; and polypeptide B comprises components having an order N-terminal to C-terminal: VL of the third binding domain, second binding domain comprising second VL-second VH, protease cleavage domain, and a CAP component, wherein a regulatory domain is located N-terminal to either polypeptide A or B, the regulatory domain comprises components having an order N-terminal to C-terminal: a second CAP component, a half-life prolonging (HLP) domain, and a protease cleavage domain.

In another embodiment, the precursor tri-specific antibody construct comprises two polypeptides, polypeptide A and polypeptide B, comprising heavy chain variable region (VH) or light chain variable region (VL), wherein
(a) polypeptide A comprises components having an order N-terminal to C-terminal: HLP domain, protease cleavage domain, VH of the third binding domain, first binding domain comprising first VH-first VL; and polypeptide B comprises components having an order N-terminal to C-terminal: CAP component, protease cleavage domain, VL of the third binding domain, and a second binding domain comprising a cytokine receptor engager; or
(b) polypeptide A comprises components having an order N-terminal to C-terminal: HLP domain, protease cleavage domain, VH of the third binding domain, first binding domain comprising first VL-first VH; and polypeptide B comprises components having an order N-terminal to C-terminal: CAP component, protease cleavage domain, VL of the third binding domain, and a second binding domain comprising a cytokine receptor engager; or
(c) polypeptide A comprises components having an order N-terminal to C-terminal: CAP component, protease cleavage domain, VH of the third binding domain, first binding domain comprising first VH-first VL; and polypeptide B comprises components having an order N-terminal to C-terminal: HLP domain, protease cleavage domain, VL of the third binding domain, and a second binding domain comprising a cytokine receptor engager; or
(d) polypeptide A comprises components having an order N-terminal to C-terminal: CAP component, protease cleavage domain, VH of the third binding domain, first binding domain comprising first VL-first VH; and polypeptide B comprises components having an order N-terminal to C-terminal: HLP domain, protease cleavage domain, VL of the third binding domain, and a second binding domain comprising a cytokine receptor engager.

In another embodiment, the precursor tri-specific antibody construct comprises two polypeptides, polypeptide A and polypeptide B, comprising heavy chain variable region (VH) or light chain variable region (VL), wherein
(a) polypeptide A comprises components having an order N-terminal to C-terminal: VH of the third binding domain, first binding domain comprising first VH-first VL; and polypeptide B comprises components having an order N-terminal to C-terminal: VL of the third binding domain, and a second binding domain comprising a cytokine receptor engager, wherein a regulatory domain is located N-terminal to either polypeptide A or B, the regulatory domain comprises components having an order N-terminal to C-terminal: a CAP component, a half-life prolonging (HLP) domain, and a protease cleavage domain; or
(b) polypeptide A comprises components having an order N-terminal to C-terminal: VH of the third binding domain, first binding domain comprising first VL-first VH; and polypeptide B comprises components having an order N-terminal to C-terminal: VL of the third binding domain, and a second binding domain comprising a cytokine receptor engager, wherein a regulatory domain is located N-terminal to either polypeptide A or B, the regulatory domain comprises components having an order N-terminal to C-terminal: a CAP component, a half-life prolonging (HLP) domain, and a protease cleavage domain; or
(c) polypeptide A comprises components having an order N-terminal to C-terminal: VH of the third binding domain, first binding domain comprising first VH-first VL; and polypeptide B comprises components having an order N-terminal to C-terminal: VL of the third binding domain, and a second binding domain comprising a cytokine receptor engager, wherein a regulatory domain is located N-terminal to either polypeptide A or B, the regulatory domain comprises components having an order N-terminal to C-terminal: a CAP component, a half-life prolonging (HLP) domain, and a protease cleavage domain; or
(d) polypeptide A comprises components having an order N-terminal to C-terminal: VH of the third binding domain, first binding domain comprising first VL-first VH; and polypeptide B comprises components having an order N-terminal to C-terminal: VL of the third binding domain, and a second binding domain comprising a cytokine receptor engager, wherein a regulatory domain is located N-terminal to either polypeptide A or B, the regulatory domain comprises components having an order N-terminal to C-terminal: a CAP component, a half-life prolonging (HLP) domain, and a protease cleavage domain.

In some embodiments of the above-described precursor tri-specific antibody constructs, a second binding domain comprises two scFv, each binding to the same or different target antigen. The VH and VL domains of the two scFv can be arranged, from the N-terminal to C-terminal, as VH-VL-VH-VL, VH-VL-VL-VH, VL-VH-VH-VL, or VL-VH-VL-VH,

In one embodiment, the third binding domain comprises a light chain variable region (VL) and a heavy chain variable region (VH), wherein the VL comprises a light chain CDR1 having the sequence of one of SEQ ID NOs:107-109, a light chain CDR2 (SEQ ID NO:110), and a light chain CDR3 having the sequence of one of SEQ ID NOs:111-112, and the VH comprises a heavy chain CDR1 (SEQ ID NO:104), a heavy chain CDR2 (SEQ ID NO:105), and a heavy chain CDR3 (SEQ ID NO:106). In one embodiment, the VL comprises an amino acid sequence having the sequence of one of SEQ ID NOs:75-103 and 116, or an amino acid sequence having at least 80% homology thereof. In one embodiment, the VH comprises the amino acid sequence having the sequence of one of SEQ ID NOs:46-72 and 114, or an amino acid sequence having at least 80% homology thereto.

In one embodiment, disclosed herein is a pharmaceutical composition comprising a pharmaceutically acceptable carrier and the precursor tri-specific antibody construct disclosed herein.

In one embodiment, disclosed herein is a nucleic acid construct comprising one or more nucleic acid sequences, wherein the nucleic acid construct encodes a precursor tri-specific antibody disclosed herein. In one embodiment, there is provided an expression vector comprising such nucleic acid construct. In another embodiment, there is provided an isolated host cell comprising such expression vector.

In one embodiment, there is provided a method of treating, preventing, inhibiting the growth of, delaying disease progression, reducing tumor load, or reducing the incidence of a cancer or a tumor, or any combination thereof, in a subject in need of such treatment, comprising a step of administering to the subject a pharmaceutical composition comprising a precursor tri-specific antibody construct disclosed herein. In one embodiment, this method reduces the minimal residual disease, increases remission, increases remission duration, reduces tumor relapse rate, prevents metastasis of the tumor or the cancer, or reduces the rate of metastasis of the tumor or the cancer, or any combination thereof, compared with a subject not administered with the pharmaceutical composition. In one embodiment, the cancer or tumor can be a solid tumor or non-solid tumor, or the cancer or tumor can be a metastasis of a cancer or tumor.

In one embodiment, examples of non-solid tumor include, but are not limited to, a hematopoietic malignancy, a blood cell cancer, a leukemia, a myelodysplastic syndrome, a lymphoma, a multiple myeloma (a plasma cell myeloma), an acute lymphoblastic leukemia, an acute myelogenous leukemia, a chronic myelogenous leukemia, a Hodgkin lymphoma, a non-Hodgkin lymphoma, and plasma cell leukemia; or wherein the solid tumor is selected from the group consisting of a sarcoma, a carcinoma, a fibrosarcoma, a myxosarcoma, a liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, an Ewing’s tumor, a leiomyosarcoma, a rhabdomyosarcoma, a colon carcinoma, a pancreatic cancer or tumor, a breast cancer or tumor, an ovarian cancer or tumor, a prostate cancer or tumor, a squamous cell carcinoma, a basal cell carcinoma, an adenocarcinoma, a sweat gland carcinoma, a sebaceous gland carcinoma, a papillary carcinoma, a papillary adenocarcinomas, a cystadenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilm’s tumor, a cervical cancer or tumor, a uterine cancer or tumor, a testicular cancer or tumor, a lung carcinoma, a small cell lung carcinoma, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodenroglioma, a schwannoma, a meningioma, a melanoma, a neuroblastoma, or a retinoblastoma.

In another embodiment, there is provided a method of treating, preventing, inhibiting the growth of, delaying disease progression, reducing tumor load, or reducing the incidence of a cancer or a tumor, or any combination thereof, in a subject in need of such treatment, comprising a step of administering to the subject a pharmaceutical composition comprising nucleic acid construct (which may one or more nucleic acid sequences) that encodes a precursor tri-specific antibody construct disclosed herein. In one embodiment, this method reduces the minimal residual disease, increases remission, increases remission duration, reduces tumor relapse rate, prevents metastasis of the tumor or the cancer, or reduces the rate of metastasis of the tumor or the cancer, or any combination thereof, compared with a subject not administered with the pharmaceutical composition.

In another embodiment, there is provided a method of producing a precursor tri-specific antibody construct disclosed herein, the method comprising the steps of: (i) culturing a host cell comprising nucleic acid sequences that encodes precursor tri-specific antibody construct polypeptides A and B, (ii) expressing the polypeptides A and B, (iii) isolating the expressed polypeptides A and B, and (iv) dimerizing the polypeptides A and B. In one embodiment, expressing these two polypeptides comprises expression from a single type of host cells, or expression from two types of host cells each expressing a different polypeptide, polypeptide A and polypeptide B, respectively.

### BRIEF DESCRIPTION OF THE DRAWINGS

The subject matter regarded as the precursor tri-specific (tri-body) antibody constructs that bind to at least an NK cell surface antigen disclosed herein is particularly pointed out and distinctly claimed in the concluding portion of the specification. The precursor tri-specific (tri-body) antibody constructs, however, both as to organization and method of use, together with objects, features, and advantages thereof, may best be understood by reference to the following detailed description when read with the accompanying drawings in which:

FIG. 1 shows flow diagrams of protease specific activation within tumor tissue or within a tumor environment of precursor tri-specific antibody constructs, wherein T cell and/or NK cell engagement and activation is limited to tumor sites.

FIG. 2A presents one embodiment of a precursor tri-specific antibody (tri-body) construct having antibody binding domains and regulatory domains as described herein, the Fab portion recognizes a CD3 surface antigen, one scFv recognizes a tumor associated antigen (TAA), and one scFv recognizes a NK cell surface antigen (e.g. NKG2A or NKG2D). FIG. 2B presents another embodiment of the tri-body construct of FIG. 2A. FIG. 2C presents one embodiment of a precursor tri-specific antibody construct as described herein but lacking the regulatory domain comprising the half-life extending component (HSA). FIG. 2D presents one embodiment of a precursor tri-specific antibody construct as described herein but lacking the regulatory domain comprising the CD3 CAP domain. FIG. 2E presents one embodiment of an active tri-specific (tribody) antibody construct, lacking the regulatory domains with the HSA and CAP domains. FIG. 2F presents another embodiment of an active tri-specific (tribody) antibody construct, lacking the regulatory domains with the HSA and CAP domains. FIG. 2G presents one embodiment of a precursor tri-specific antibody construct as described herein, wherein the regulatory domain N-terminal to the Fab comprises a single regulatory domain comprising a CAP domain, a HSA sequence, and a protease cleavable linker on the same polypeptide.

FIG. 3A presents one embodiment of a precursor tri-specific antibody (tri-body) construct having antibody binding domains and regulatory domains as described herein, the Fab portion recognizes a CD3 surface antigen, one scFv recognizes a tumor associated antigen (TAA), and one scFv recognizes a NK cell surface antigen (e.g. NKG2A or NKG2D). The anti-NK scFv further comprises a regulatory domain comprising a CAP domain. FIG. 3B presents one embodiment of a precursor tri-specific antibody construct as described herein, wherein the regulatory domain N-terminal to the Fab comprises a single regulatory domain comprising a CAP domain, a HSA sequence, and a protease cleavable linker on the same polypeptide. FIG. 3C presents another embodiment of a precursor tri-specific antibody construct as described in FIG. 3B. FIG. 3D presents one embodiment of a precursor tri-specific antibody construct as described herein but lacking the regulatory domain comprising the half-life extending component (HSA). FIG. 3E presents one embodiment of a precursor tri-specific antibody construct as described herein but lacking the regulatory domain comprising the CD3 CAP domain. FIG. 3F presents one embodiment of an active tri-specific (tribody) antibody construct, lacking the regulatory domains with the HSA and CAP domains.

FIG. 4A presents one embodiment of a precursor tri-specific antibody (tri-body) construct having antibody binding domains as described herein, the Fab portion recognizes a NK cell surface antigen (e.g. NKG2A or NKG2D), one scFv recognizes a tumor associated antigen (TAA). The regulatory domain N-terminal to the Fab comprises a single regulatory domain comprising a CAP domain, a HSA sequence, and a protease cleavable linker on the same polypeptide. The tri-body construct also has a cytokine receptor engager comprising a cytokine that binds to a cytokine receptor, for example, an IL-15 that binds to IL-15 receptor. FIG. 4B presents another embodiment of the tri-body construct of FIG. 4A, wherein the regulatory domain N-terminal to the Fab is the one shown in FIG. 2A. FIG. 4C presents one embodiment of an active tri-specific (tribody) antibody construct derived from the precursor construct of FIGS. 4A or 4B, wherein the Fab portion recognizes a NK cell surface antigen (e.g. NKG2A or NKG2D), one scFv recognizes a tumor associated antigen (TAA), and there is a cytokine receptor engager such as IL-15.

FIG. 5A presents one embodiment of a precursor tri-specific antibody (tri-body) construct having three antibody binding domains and regulatory domains as described herein, the Fab portion recognizes a NK cell surface antigen (e.g. NKG2D), one scFv recognizes a tumor associated antigen (TAA), and one scFv recognizes another NK cell surface antigen (e.g. NKG2A). FIG. 5B presents another embodiment of a precursor tri-specific antibody construct of FIG. 5A, wherein the Fab binds to NKG2A and one scFv binds to NKG2D. In another embodiment, the anti-NK Fab binds to NKG2A and the anti-NK-scFv binds to NKG2D. FIG. 5C presents one embodiment of a precursor tri-specific antibody (tri-body) construct having the antibody binding domains and regulatory domains as described herein, the Fab portion recognizes a NK cell surface antigen (e.g. NKG2D), one scFv recognizes a tumor associated antigen (TAA), and one scFv recognizes another NK cell surface antigen (e.g. NKG2A). The regulatory domain N-terminal to the Fab comprises a single polypeptide chain. FIG. 5D presents another embodiment of a precursor tri-specific antibody construct of FIG. 5C, wherein the Fab binds to NKG2A and one scFv binds to NKG2D. FIG. 5E presents one embodiment of an active tri-specific (tribody) antibody construct derived from the precursor constructs of FIGS. 5A-5D, wherein the anti-NK Fab binds to NKG2D and the anti-NK-scFv binds to NKG2A. FIG. 5F presents another embodiment of an active tri-specific (tribody) antibody construct derived from the precursor constructs of FIGS. 5A-5D, wherein the anti-NK Fab binds to NKG2A and the anti-NK-scFv binds to NKG2D.

FIGS. 6A and 6B present embodiments of an amino acid sequence of a Heavy Chain (HC) polypeptide of an activated tri-specific (tri-body) antibody construct (Construct 1; VLVH) and an optimized nucleotide sequences encoding the Heavy Chain (HC) activated construct, Amino acid sequences are presented N-terminal to C-terminal, and nucleic acid sequences are presented 5′ to 3′. FIG. 6A presents one embodiment of an amino acid sequence of a Heavy Chain (HC) polypeptide of an activated construct, having the N-terminal to C-terminal order and components as follows: h1F3.5-G1Fd anti-EGFR VL-linker-VH (SEQ ID NO: 138). The amino acid sequences of the component parts of the HC polypeptide shown in FIG. 6A include: Linker (SEQ ID NO: 158), anti-CD3epsilon variable heavy chain and constant heavy chain region 1 (SEQ ID NO: 113), followed by two marked cysteine residues (marked bold and underline), which may participate in disulfide double bond, followed by an anti-EGFR scFv VL (SEQ ID NO: 34)-linker (SEQ ID NO: 39)-VH (SEQ ID NO: 37) chain. FIG. 6B presents one embodiment of an optimized nucleic acid sequence (DNA) encoding a Heavy Chain (HC) polypeptide of an activated tri-specific (tri-body) construct, having the 5′ to 3′ order and components as follows (SEQ ID NO: 150). The nucleic acid sequences encoding the component parts of the HC polypeptide shown in FIG. 6A include: Linker (SEQ ID NO: 154), anti-CD3epsilon variable heavy chain and constant heavy chain region 1 (SEQ ID NO: 155), followed by two marked cysteine residues (marked bold and underline), which may participate in disulfide double bond, followed by an anti-EGFR scFv VL (SEQ ID NO: 36)-linker (SEQ ID NO: 40)-VH (SEQ ID NO: 38) chain.

FIGS. 7A and 7B present embodiments of an amino acid sequence of a Light Chain (LC) polypeptide of an activated tri-specific (tri-body) antibody construct (Construct 1; VLVH) and an optimized nucleotide sequences encoding the Light Chain (LC) activated construct, Amino acid sequences are presented N-terminal to C-terminal, and nucleic acid sequences are presented 5′ to 3′. FIG. 7A presents one embodiment of an amino acid sequence of a Light Chain (LC) polypeptide of an activated construct, having the N-terminal to C-terminal order and components as follows: h1F3.1-λLC anti-EGFR VL-linker-VH (SEQ ID NO: 139). The amino acid sequences of the component parts of the LC polypeptide shown in FIG. 7A include: Linker (SEQ ID NO: 158), anti-CD3epsilon variable light chain and lambda light chain (SEQ ID NO: 74), followed by marked cysteine residues (marked bold and underline), which may participate in a disulfide double bond, followed by an anti-EGFR scFv VL (SEQ ID NO: 34)-linker (SEQ ID NO: 39)-VH (SEQ ID NO: 37) chain. FIG. 7B presents one embodiment of an optimized nucleic acid sequence (DNA) encoding a Light Chain (LC) polypeptide of an activated tri-specific (tri-body) construct, having the 5′ to 3′ order and components as follows (SEQ ID NO: 151). The nucleic acid sequences encoding the component parts of the LC polypeptide shown in FIG. 7A include: Linker (SEQ ID NO: 154), anti-CD3epsilon variable light chain and lambda light chain region (SEQ ID NO: 159), followed by marked cysteine residues (marked bold and underline), which may participate in a disulfide double bond, followed by an anti-EGFR scFv VL (SEQ ID NO: 36)-linker (SEQ ID NO: 40)-VH (SEQ ID NO: 38) chain.

FIGS. 8A and 8B present embodiments of an amino acid sequence of a Heavy Chain (HC) polypeptide of an activated tri-specific (tri-body) antibody construct (Construct 2; VHVL) and an optimized nucleotide sequences encoding the Heavy Chain (HC) activated construct. Amino acid sequences are presented N-terminal to C-terminal, and nucleic acid sequences are presented 5′ to 3′. FIG. 8A presents one embodiment of an amino acid sequence of a Heavy Chain (HC) polypeptide of an activated construct, having the N-terminal to C-terminal order and components as follows: h1F3.5-G1Fd-(VH-linker-VL) (SEQ ID NO: 140). The amino acid sequences of the component parts of the HC polypeptide shown in FIG. 8A include: Linker (SEQ ID NO: 158), anti-CD3epsilon variable heavy chain and constant heavy chain region 1 (SEQ ID NO: 113), followed by two marked cysteine residues (marked bold and underline), which may participate in disulfide double bond, followed by an anti-EGFR scFv VH (SEQ ID NO: 37)-linker (SEQ ID NO: 39)-VL (SEQ ID NO: 34) chain. FIG. 8B presents one embodiment of an optimized nucleic acid sequence (DNA) encoding a Heavy Chain (HC) polypeptide of an activated tri-specific (tri-body) construct, having the 5′ to 3′ order and components as follows (SEQ ID NO: 152). The nucleic acid sequences encoding the component parts of the HC polypeptide shown in FIG. 8A include: Linker (SEQ ID NO: 154), anti-CD3epsilon variable heavy chain and constant heavy chain region 1 (SEQ ID NO: 155), followed by two marked cysteine residues (marked bold and underline), which may participate in disulfide double bonds, followed by an anti-EGFR scFv VH (SEQ ID NO: 38)-linker (SEQ ID NO: 40)-VH (SEQ ID NO: 36) chain.

FIGS. 9A and 9B present embodiments of an amino acid sequence of a Light Chain (LC) polypeptide of an activated tri-specific (tri-body) antibody construct (Construct 2; VHVL) and an optimized nucleotide sequences encoding the Light Chain (LC) activated construct. Amino acid sequences are presented N-terminal to C-terminal, and nucleic acid sequences are presented 5′ to 3′. FIG. 9A presents one embodiment of an amino acid sequence of a Light Chain (LC) polypeptide of an activated construct, having the N-terminal to C-terminal order and components as follows: h1F3.1-λLC- Anti-EGFR (VH-linker-VL) (SEQ ID NO: 141). The amino acid sequences of the component parts of the LC polypeptide shown in FIG. 9A include: Linker (SEQ ID NO: 158), anti-CD3epsilon variable light chain and lambda light chain (SEQ ID NO: 74), followed by marked cysteine residues (marked bold and underline), which may participate in a disulfide double bond, followed by an anti-EGFR scFv VH (SEQ ID NO: 37)-linker (SEQ ID NO: 39)-VL (SEQ ID NO: 34) chain. FIG. 9B presents one embodiment of an optimized nucleic acid sequence (DNA) encoding a Light Chain (LC) polypeptide of an activated tri-specific (tri-body) construct, having the 5′ to 3′ order and components as follows (SEQ ID NO: 153). The nucleic acid sequences encoding the component parts of the LC polypeptide shown in FIG. 9A include: Linker (SEQ ID NO: 154), anti-CD3epsilon variable light chain and lambda light chain region (SEQ ID NO: 159), followed by marked cysteine residues (marked bold and underline), which may participate in a disulfide double bond, followed by an anti-EGFR scFv VH (SEQ ID NO: 38)-linker (SEQ ID NO: 40)-VL (SEQ ID NO: 36) chain.

FIGS. 10A and 10B present embodiments of an amino acid sequence of a Heavy Chain (HC) polypeptide of precursor tri-specific (tri-body) antibody construct (Construct 3; VLVH) and an optimized nucleotide sequences encoding the Heavy Chain (HC) precursor construct, Amino acid sequences are presented N-terminal to C-terminal, and nucleic acid sequences are presented 5′ to 3′. FIG. 10A presents one embodiment of an amino acid sequence of a Heavy Chain (HC) polypeptide of a precursor construct, having the N-terminal to C-terminal order and components as follows: hHSA-G-PLGLAG (MMP2/9)- (cloning)-h1F3.5-G1Fd anti-EGFR VL-linker-VH (SEQ ID NO: 130). The amino acid sequences of the component parts of the HC polypeptide shown in FIG. 10A include: human serum albumin (HSA) (SEQ ID NO: 7), MMP2/9 protease cleavable Linker (SEQ ID NO: 160 (linker with cleavable sequence and SEQ ID NO: 9 (cleavable sequence)), anti-CD3epsilon variable heavy chain and constant heavy chain region 1 (SEQ ID NO: 113), followed by two marked cysteine residues (marked bold and underline), which may participate in disulfide double bonds, followed by an anti-EGFR scFv VL (SEQ ID NO: 34)-linker (SEQ ID NO: 39)-VH (SEQ ID NO: 37) chain. FIG. 10B presents one embodiment of an optimized nucleic acid sequence (DNA) encoding a Heavy Chain (HC) polypeptide of the precursor tri-specific (tri-body) antibody construct, having the 5′ to 3′ order and components as follows (SEQ ID NO: 142). The nucleic acid sequences encoding the component parts of the HC polypeptide shown in FIG. 10A include: human serum albumin (HSA) (SEQ ID NO: 8), MMP2/9 protease cleavable Linker (SEQ ID NO: 161 (linker with cleavable sequence and SEQ ID NO: 33 (cleavable sequence)), anti-CD3epsilon variable heavy chain and constant heavy chain region 1 (SEQ ID NO: 155), followed by two marked cysteine residues (marked bold and underline), which may participate in disulfide double bond, followed by an anti-EGFR scFv VL (SEQ ID NO: 36)-linker (SEQ ID NO: 40)-VH (SEQ ID NO: 38) chain.

FIGS. 11A and 11B present embodiments of an amino acid sequence of a Light Chain (LC) polypeptide of a precursor tri-specific (tri-body) antibody construct (Construct 3; VLVH) and an optimized nucleotide sequences encoding the Light Chain (LC) precursor construct, Amino acid sequences are presented N-terminal to C-terminal, and nucleic acid sequences are presented 5′ to 3′. FIG. 11A presents one embodiment of an amino acid sequence of a Light Chain (LC) polypeptide of a precursor construct, having the N-terminal to C-terminal order and components as follows: Cap-h1F3.1-λLC anti-EGFR VL-linker-VH MM2/9 cleavage; SEQ ID NO: 131). The amino acid sequences of the component parts of the LC polypeptide shown in FIG. 11A include: CAP (SEQ ID NO: 5), MMP2/9 protease cleavable Linker (SEQ ID NO: 160 and SEQ ID NO: 9 (cleavable sequence)), anti-CD3epsilon variable light chain and lambda light chain (SEQ ID NO: 74), followed by marked cysteine residues (marked bold and underline), which may participate in a disulfide double bond, followed by an anti-EGFR scFv VL (SEQ ID NO: 34)-linker (SEQ ID NO: 39)-VH (SEQ ID NO: 37) chain. FIG. 11B presents one embodiment of an optimized nucleic acid sequence (DNA) encoding the Light Chain (LC) polypeptide of the precursor tri-specific (tri-body) antibody construct, having the 5′ to 3′ order and components as follows (SEQ ID NO: 143). The nucleic acid sequences encoding the component parts of the LC polypeptide shown in FIG. 11A include: CAP (SEQ ID NO: 164), MMP2/9 protease cleavable Linker (SEQ ID NO: 161 and SEQ ID NO: 33 (cleavable sequence)), anti-CD3epsilon variable light chain and lambda light chain region (SEQ ID NO: 159), followed by marked cysteine residues (marked bold and underline), which may participate in a disulfide double bond, followed by an anti-EGFR scFv VL (SEQ ID NO: 36)-linker (SEQ ID NO: 40)-VH (SEQ ID NO: 38) chain.

FIGS. 12A and 12B present embodiments of an amino acid sequence of a Heavy Chain (HC) polypeptide of precursor tri-specific (tri-body) antibody construct (Construct 4; VHVL) and an optimized nucleotide sequences encoding the Heavy Chain (HC) precursor construct. Amino acid sequences are presented N-terminal to C-terminal, and nucleic acid sequences are presented 5′ to 3′. FIG. 12A presents one embodiment of an amino acid sequence of a Heavy Chain (HC) polypeptide of a precursor construct, having the N-terminal to C-terminal order and components as follows: hHSA-G-PLGLAG (MMP2/9)- (cloning)-h1F3.5-G1Fd Anti-EGFR (VH-linker-VL) (SEQ ID NO: 132). The amino acid sequences of the component parts of the HC polypeptide shown in FIG. 12A include: human serum albumin (HSA) (SEQ ID NO: 7), MMP2/9 protease cleavable Linker (SEQ ID NO: 160 and SEQ ID NO: 9 (cleavable portion)), anti-CD3epsilon variable heavy chain and constant heavy chain region 1 (SEQ ID NO: 113), followed by two marked cysteine residues (marked bold and underline), which may participate in disulfide double bonds, followed by an anti-EGFR scFv VH (SEQ ID NO: 37)-linker (SEQ ID NO: 40)-VL (SEQ ID NO: 34) chain. FIG. 12B presents one embodiment of an optimized nucleic acid sequence (DNA) encoding the Heavy Chain (HC) polypeptide of the precursor tri-specific (tri-body) antibody construct, having the 5′ to 3′ order and components as follows (SEQ ID NO: 144). The nucleic acid sequences encoding the component parts of the HC polypeptide shown in FIG. 12A include: human serum albumin (HSA) (SEQ ID NO: 8), MMP2/9 protease cleavable Linker (SEQ ID NO: 161 and SEQ ID NO: 33 (cleavable linker)), anti-CD3epsilon variable heavy chain and constant heavy chain region 1 (SEQ ID NO: 155), followed by two marked cysteine residues (marked bold and underline), which may participate in disulfide double bond, followed by an anti-EGFR scFv VH (SEQ ID NO: 38)-linker (SEQ ID NO: 40)-VL (SEQ ID NO: 36) chain.

FIGS. 13A and 13B present embodiments of an amino acid sequence of a Light Chain (LC) polypeptide of a precursor tri-specific (tri-body) antibody construct (Construct 4; VLVH) and an optimized nucleotide sequences encoding the Light Chain (LC) precursor construct. Amino acid sequences are presented N-terminal to C-terminal, and nucleic acid sequences are presented 5′ to 3′. FIG. 13A presents one embodiment of an amino acid sequence of a Light Chain (LC) polypeptide of a precursor construct, having the N-terminal to C-terminal order and components as follows: Cap-MMP2/9 cleavage -h1F3.1-λLC-Anti-EGFR(VH-linker-VL) (SEQ ID NO: 133; plasmid 7). The amino acid sequences of the component parts of the LC polypeptide shown in FIG. 13A include: CAP (SEQ ID NO: 5), MMP2/9 protease cleavable Linker (SEQ ID NO: 160 and SEQ ID NO: 9 (cleavable linker), anti-CD3epsilon variable light chain and lambda light chain (SEQ ID NO: 74), followed by marked cysteine residues (marked bold and underline), which may participate in a disulfide double bond, followed by an anti-EGFR scFv VH (SEQ ID NO: 37)-linker (SEQ ID NO: 40)-VL (SEQ ID NO: 34) chain. FIG. 13B presents one embodiment of an optimized nucleic acid sequence (DNA) encoding the Light Chain (LC) polypeptide of the precursor tri-specific (tri-body) antibody construct, having the 5′ to 3′ order and components as follows (SEQ ID NO: 145). The nucleic acid sequences encoding the component parts of the LC polypeptide shown in FIG. 13A include: CAP (SEQ ID NO: 164), MMP2/9 protease cleavable Linker (SEQ ID NO: 161 and SEQ ID NO: 33 (cleavable sequence)), anti-CD3epsilon variable light chain and lambda light chain region (SEQ ID NO: 159), followed by marked cysteine residues (marked bold and underline), which may participate in a disulfide double bond, followed by an anti-EGFR scFv VH (SEQ ID NO: 38)-linker (SEQ ID NO: 40)-VL (SEQ ID NO: 36) chain.

FIGS. 14A and 14B present embodiments of an amino acid sequence of a Heavy Chain (HC) polypeptide of non-cleavable (non-activatable) precursor tri-specific (tri-body) antibody construct (Construct 5; VLVH) and an optimized nucleotide sequences encoding the Heavy Chain (HC) non-cleavable precursor construct. Amino acid sequences are presented N-terminal to C-terminal, and nucleic acid sequences are presented 5′ to 3′. FIG. 14A presents one embodiment of an amino acid sequence of a Heavy Chain (HC) polypeptide of a non-cleavable precursor construct, having the N-terminal to C-terminal order and components as follows: hHSA-G-PLGLAG NC-h1F3.5-G1Fd anti-EGFR VL-linker-VH (SEQ ID NO: 134). The amino acid sequences of the component parts of the HC polypeptide shown in FIG. 14A include: human serum albumin (HSA) (SEQ ID NO: 7), non-cleavable Linker (SEQ ID NO: 162), anti-CD3epsilon variable heavy chain and constant heavy chain region 1 (SEQ ID NO: 113), followed by two marked cysteine residues (marked bold and underline), which may participate in disulfide double bonds, followed by an anti-EGFR scFv VL (SEQ ID NO: 34)-linker (SEQ ID NO: 40)-VH (SEQ ID NO: 37) chain. FIG. 14B presents one embodiment of an optimized nucleic acid sequence (DNA) encoding a Heavy Chain (HC) polypeptide of the non-cleavable precursor tri-specific (tri-body) antibody construct, having the 5′ to 3′ order and components as follows (SEQ ID NO: 146). The nucleic acid sequences encoding the component parts of the HC polypeptide shown in FIG. 14A include: human serum albumin (HSA) (SEQ ID NO: 8), non-cleavable Linker (SEQ ID NO: 163), anti-CD3epsilon variable heavy chain and constant heavy chain region 1 (SEQ ID NO: 155), followed by two marked cysteine residues (marked bold and underline), which may participate in disulfide double bond, followed by an anti-EGFR scFv VL (SEQ ID NO: 36)-linker (SEQ ID NO: 40)-VH (SEQ ID NO: 38) chain.

FIGS. 15A and 15B present embodiments of an amino acid sequence of a Light Chain (LC) polypeptide of a non-cleavable precursor tri-specific (tri-body) antibody construct (Construct 5; VLVH) and an optimized nucleotide sequences encoding the Light Chain (LC) non-cleavable precursor construct. Amino acid sequences are presented N-terminal to C-terminal, and nucleic acid sequences are presented 5′ to 3′. FIG. 15A presents one embodiment of an amino acid sequence of a Light Chain (LC) polypeptide of a non-cleavable precursor construct, having the N-terminal to C-terminal order and components as follows: Cap-(h1F3.1-λLC anti-EGFR VL-linker-VH Non-Cleavable (SEQ ID NO: 135). The amino acid sequences of the component parts of the LC polypeptide shown in FIG. 15A include: CAP (SEQ ID NO: 5), non-cleavable Linker (SEQ ID NO: 162), anti-CD3epsilon variable light chain and lambda light chain (SEQ ID NO: 74), followed by marked cysteine residues (marked bold and underline), which may participate in a disulfide double bond, followed by an anti-EGFR scFv VL (SEQ ID NO: 34)-linker (SEQ ID NO: 40)-VH (SEQ ID NO: 37) chain. FIG. 15B presents one embodiment of an optimized nucleic acid sequence (DNA) encoding the Light Chain (LC) polypeptide of the non-cleavable precursor tri-specific (tri-body) antibody construct, having the 5′ to 3′ order and components as follows (SEQ ID NO: 147). The nucleic acid sequences encoding the component parts of the LC polypeptide shown in FIG. 13A include: CAP (SEQ ID NO: 164), non-cleavable Linker (SEQ ID NO: 163),anti-CD3epsilon variable light chain and lambda light chain region (SEQ ID NO: 159), followed by marked cysteine residues (marked bold and underline), which may participate in a disulfide double bond, followed by an anti-EGFR scFv VL (SEQ ID NO: 36)-linker (SEQ ID NO: 40)-VH (SEQ ID NO: 38) chain.

FIGS. 16A and 16B present embodiments of an amino acid sequence of a Heavy Chain (HC) polypeptide of a non-cleavable precursor tri-specific (tri-body) antibody construct (Construct 6; VHVL) and an optimized nucleotide sequences encoding the Heavy Chain (HC) non-cleavable precursor construct. Amino acid sequences are presented N-terminal to C-terminal, and nucleic acid sequences are presented 5′ to 3′. FIG. 16A presents one embodiment of an amino acid sequence of a Heavy Chain (HC) polypeptide of a non-cleavable precursor construct, having the N-terminal to C-terminal order and components as follows: hHSA-G-PLGLAG (NC)- (cloning)-h1F3.5-G1Fd-(VH-linker-VL) (SEQ ID NO: 136). The amino acid sequences of the component parts of the HC polypeptide shown in FIG. 16A include: human serum albumin (HSA) (SEQ ID NO: 7), non-cleavable Linker (SEQ ID NO: 162), anti-CD3epsilon variable heavy chain and constant heavy chain region 1 (SEQ ID NO: 113), followed by two marked cysteine residues (marked bold and underline), which may participate in disulfide double bonds, followed by an anti-EGFR scFv VH (SEQ ID NO: 37)-linker (SEQ ID NO: 40)-VL (SEQ ID NO: 34) chain. FIG. 16B presents one embodiment of an optimized nucleic acid sequence (DNA) encoding the Heavy Chain (HC) polypeptide of the non-cleavable precursor tri-specific (tri-body) antibody construct, having the 5′ to 3′ order and components as follows (SEQ ID NO: 148). The nucleic acid sequences encoding the component parts of the HC polypeptide shown in FIG. 16A include: human serum albumin (HSA) (SEQ ID NO: 8), non-cleavable Linker (SEQ ID NO: 163), anti-CD3epsilon variable heavy chain and constant heavy chain region 1 (SEQ ID NO: 155), followed by two marked cysteine residues (marked bold and underline), which may participate in disulfide double bond, followed by an anti-EGFR scFv VH (SEQ ID NO: 38)-linker (SEQ ID NO: 40)-VL (SEQ ID NO: 36) chain.

FIGS. 17A and 17B present embodiments of an amino acid sequence of a Light Chain (LC) polypeptide of a non-cleavable precursor tri-specific (tri-body) antibody construct (Construct 6; VLVH) and an optimized nucleotide sequences encoding the Light Chain (LC) non-cleavable precursor construct. Amino acid sequences are presented N-terminal to C-terminal, and nucleic acid sequences are presented 5′ to 3′. FIG. 17A presents one embodiment of an amino acid sequence of a Light Chain (LC) polypeptide of a precursor construct, having the N-terminal to C-terminal order and components as follows: Cap (NC)-h1F3.1-λLC- Anti-EGFR (VH-linker-V) (SEQ ID NO: 137). The amino acid sequences of the component parts of the LC polypeptide shown in FIG. 17A include: CAP (SEQ ID NO: 5), non-cleavable Linker (SEQ ID NO: 162), anti-CD3epsilon variable light chain and lambda light chain (SEQ ID NO: 74), followed by marked cysteine residues (marked bold and underline), which may participate in a disulfide double bond, followed by an anti-EGFR scFv VH (SEQ ID NO: 37)-linker (SEQ ID NO: 40)-VL (SEQ ID NO: 34) chain. FIG. 17B presents one embodiment of an optimized nucleic acid sequence (DNA) encoding the Light Chain (LC) polypeptide of the non-cleavable precursor tri-specific (tri-body) antibody construct, having the 5′ to 3′ order and components as follows (SEQ ID NO: 149). The nucleic acid sequences encoding the component parts of the LC polypeptide shown in FIG. 17A include: CAP (SEQ ID NO: 164), non-cleavable Linker (SEQ ID NO: 163), anti-CD3epsilon variable light chain and lambda light chain region (SEQ ID NO: 159), followed by marked cysteine residues (marked bold and underline), which may participate in a disulfide double bond, followed by an anti-EGFR scFv VH (SEQ ID NO: 38)-linker (SEQ ID NO: 40)-VL (SEQ ID NO: 36) chain.

FIGS. 18A-18B present embodiments of amino acid and nucleic acid sequences of scFv anti-ROR1 VL-VH. FIG. 18A presents one embodiment of an amino acid sequence of an scFv anti-ROR1 having the N-terminal to C-terminal order: VL-VH, and components as follows: anti-ROR1 VL, linker, and anti-ROR1 VH (SEQ ID NO: 156). FIG. 18B presents one embodiment of an optimized nucleic acid sequence encoding an scFv anti-ROR1 having the N-terminal to C-terminal order: VL-VH (SEQ ID NO: 157).

FIGS. 19A-19B present embodiments of amino acid and nucleic acid sequences of scFv anti-ROR1 VH-VL. FIG. 19A presents one embodiment of an amino acid sequence of an scFv anti-ROR1 having the N-terminal to C-terminal order: VH-VL, and components as follows: anti-ROR1 VH, linker, and anti-ROR1 VL (SEQ ID NO: 166). FIG. 19B presents one embodiment of an optimized nucleic acid sequence encoding an scFv anti-ROR1 having the N-terminal to C-terminal order: VH-VL (SEQ ID NO: 167).

FIGS. 20A-20B present embodiments of amino acid and nucleic acid sequences of scFv anti-PSMA VL-VH. FIG. 20A presents one embodiment of an amino acid sequence of an scFv anti-PSMA having the N-terminal to C-terminal order: VL-VH, and components as follows: anti-PSMA VL, linker, and anti-PSMA VH (SEQ ID NO: 168). FIG. 20B presents one embodiment of an optimized nucleic acid sequence encoding an scFv anti-PSMA having the N-terminal to C-terminal order: VL-VH (SEQ ID NO: 169).

FIGS. 21A-21B present embodiments of amino acid and nucleic acid sequences of scFv anti-PSMA VH-VL. FIG. 21A presents one embodiment of an amino acid sequence of an scFv anti-PSMA having the N-terminal to C-terminal order: VH-VL, and components as follows: anti-PSMA VH, linker, and anti-PSMA VL (SEQ ID NO: 170). FIG. 21B presents one embodiment of an optimized nucleic acid sequence encoding an scFv anti-PSMA having the N-terminal to C-terminal order: VH-VL (SEQ ID NO: 171).

FIGS. 22A-22B present embodiments of amino acid and nucleic acid sequences of scFv anti-5T4 VL-VH. FIG. 22A presents one embodiment of an amino acid sequence of an scFv anti-5T4 having the N-terminal to C-terminal order: VL-VH, and components as follows: anti-5T4 VL, linker, and anti-5T4 VH (SEQ ID NO: 172). FIG. 22B presents one embodiment of an optimized nucleic acid sequence encoding an scFv anti-5T4 having the N-terminal to C-terminal order: VL-VH (SEQ ID NO: 173).

FIGS. 23A-23B present embodiments of amino acid and nucleic acid sequences of scFv anti-5T4 VH-VL. FIG. 23A presents one embodiment of an amino acid sequence of an scFv anti-5T4 having the N-terminal to C-terminal order: VH-VL, and components as follows: anti-5T4 VH, linker, and anti-5T4 VL (SEQ ID NO: 174). FIG. 23B presents one embodiment of an optimized nucleic acid sequence encoding an scFv anti-5T4 having the N-terminal to C-terminal order: VH-VL (SEQ ID NO: 175).

FIGS. 24A-24B present embodiments of an amino acid sequence of a Heavy Chain (HC) polypeptide of precursor tri-specific (tri-body) antibody construct. Amino acid sequences are presented N-terminal to C-terminal. FIGS. 24A and 24B presents embodiments of an amino acid sequence of a Heavy Chain (HC) polypeptide of a precursor construct, having the N-terminal to C-terminal order and components as follows: CAP (Bold), linker (italicized), human serum albumin (underlined); protease cleavage sequence (bold and italicized); VH1/CH1 of anti-CD3e Fab (Bold and underlined), VL (italicized and underlined) VH (italicized, bold, and underlined) (EGFR scFv). The two marked cysteine residues (double underline), may participate in disulfide double bonds. The protease cleavage sequence in FIG. 24A is a multiple, protease cleavage sequence. The protease cleavage sequence in FIG. 24B is a MMP2/9 protease cleavage sequence. The amino acid sequence of FIG. 24A is set forth in SEQ ID NO: 28. The amino acid sequence of FIG. 24B is set forth in SEQ ID NO: 31. Embodiments of sequences of the component parts are described throughout this application.

FIG. 25 presents an embodiment of an amino acid sequence of a Light Chain (LC) polypeptide of a precursor tri-specific (tri-body) antibody construct. The amino acid sequence is presented N-terminal to C-terminal. FIG. 25 presents one embodiment of an amino acid sequence of a Light Chain (LC) polypeptide of a precursor construct, having the N-terminal to C-terminal order and components as follows: Linker (italicized), VL1-CL of anti-CD3e Fab (double underline and bold), VL (italicized and underlined), and VH (italicized bold and underlined) wherein the VH is an EGFR scFv). The two marked cysteine residues (double underline), may participate in disulfide double bonds. The amine acid sequence of the LC shown in FIG. 25 is set forth in SEQ ID NO: 32. Embodiments of amino acid sequences of the component parts of the LC polypeptide are described throughout the application.

FIGS. 26A-26B present embodiments of an amino acid sequence of a Heavy Chain (HC) polypeptide of precursor tri-specific (tri-body) antibody construct described herein. Amino acid sequences are presented N-terminal to C-terminal. FIGS. 26A and 26B presents embodiments of an amino acid sequence of a Heavy Chain (HC) polypeptide of a precursor construct, having the N-terminal to C-terminal order and components as follows: CAP (Bold), linker (italicized), human serum albumin (underlined); protease cleavage sequence (bold and italicized); VH1/CH1 of anti-CD3e Fab (Bold and underlined), VL (italicized and underlined) VH (italicized, bold, and underlined) (5T4 scFv). The two marked cysteine residues (double underline), may participate in disulfide double bonds. The protease cleavage sequence in FIG. 26A is a multiple, protease cleavage sequence. The protease cleavage sequence in FIG. 26B is a MMP2/9 protease cleavage sequence. The amino acid sequence of FIG. 26A is set forth in SEQ ID NO: 118. The amino acid sequence of FIG. 26B is set forth in SEQ ID NO: 176. Embodiments of sequences of the component parts are described throughout this application.

FIG. 27 presents an embodiment of an amino acid sequence of a Light Chain (LC) polypeptide of a precursor tri-specific (tri-body) antibody construct as described herein. The amino acid sequence is presented N-terminal to C-terminal. FIG. 27 presents one embodiment of an amino acid sequence of a Light Chain (LC) polypeptide of a precursor construct, having the N-terminal to C-terminal order and components as follows: Linker (italicized), VL1-CL of anti-CD3e Fab (double underline and bold), VL (italicized and underlined), and VH (italicized bold and underlined) wherein the VH is an 5T4 scFv) The two marked cysteine residues (double underline), may participate in disulfide double bonds. The amine acid sequence of the LC shown in FIG. 27 is set forth in SEQ ID NO: 177. Embodiments of amino acid sequences of the component parts of the LC polypeptide are described throughout the application.

FIG. 28 presents one embodiment of a precursor tri-specific antibody (tri-body) construct having antibody binding domains and regulatory domains as described herein, the Fab portion recognizes a CD3 surface antigen, one scFv recognizes a tumor associated antigen (TAA), and one scFv recognizes a NK cell surface antigen (e.g., NKG2A or NKG2D). The regulatory domain N-terminal to the Fab comprises a CAP domain, a HSA sequence, and a protease cleavable linker on the same polypeptide.

FIG. 29 presents another embodiment of the tri-body construct of FIG. 28, wherein the regulatory domain does not contain a protease cleavable linker.

FIG. 30 presents one embodiment of a precursor tri-specific antibody (tri-body) construct having antibody binding domains and regulatory domains as described herein, the Fab portion recognizes a CD3 surface antigen, one scFv recognizes a tumor associated antigen (TAA), and one scFv recognizes a NK cell surface antigen (e.g. NKG2A or NKG2D). The anti-NK scFv further comprises a regulatory domain comprising a CAP domain and a protease cleavable linker.

FIG. 31 presents one embodiment of a precursor tri-specific antibody (tri-body) construct having antibody binding domains and regulatory domains as described herein, the Fab portion recognizes a CD3 surface antigen, one scFv recognizes a tumor associated antigen (TAA), and one scFv recognizes a NK cell surface antigen (e.g. NKG2A or NKG2D). The anti-NK scFv and anti-CD3 Fab each further comprise a regulatory domain comprising a CAP domain without a protease cleavable linker.

FIG. 32 presents one embodiment of a precursor tri-specific antibody (tri-body) construct having antibody binding domains and regulatory domains as described herein, the Fab portion recognizes a CD3 surface antigen, one scFv recognizes a tumor associated antigen (TAA), and one scFv region comprising two scFv that recognize one or two NK cell surface antigen (e.g. NKG2A and/or NKG2D). The two scFv can bind the same or different NK cell surface antigens.

FIGS. 33A-33B present some other embodiments of the tri-body construct of FIG. 32, further having a regulatory domain N-terminal to the Fab comprising a CAP domain, a HSA sequence, and a protease cleavable linker on the same polypeptide. FIG. 33A shows the anti-TAA scFv is located C-terminal to the CL of the anti-CD3 Fab, whereas the 2 anti-NK scFvs are located C-terminal to the CH of the anti-CD3 Fab. FIG. 33B shows the anti-TAA scFv is located C-terminal to the CH of the anti-CD3 Fab, whereas the 2 anti-NK scFvs are located C-terminal to the CL of the anti-CD3 Fab. In either embodiment, the two scFv can bind the same or different NK cell surface antigens.

FIG. 34A presents another embodiment of the tri-body construct of FIG. 33A, wherein the regulatory domain does not contain a protease cleavable linker. FIG. 34B presents another embodiment of the tri-body construct of FIG. 33B, wherein the regulatory domain does not contain a protease cleavable linker.

FIG. 35 presents one embodiment of a precursor tri-specific antibody (tri-body) construct having antibody binding domains and regulatory domains as described herein, the Fab portion recognizes a CD3 surface antigen, one scFv recognizes a tumor associated antigen (TAA), and one binding domain comprising a cytokine receptor engager (e.g. IL-15).

FIG. 36 presents another embodiment of the tri-body construct of FIG. 35, further having a regulatory domain N-terminal to the Fab comprising a CAP domain, a HSA sequence, and a protease cleavable linker on the same polypeptide.

FIG. 37 presents another embodiment of the tri-body construct of FIG. 36, wherein the regulatory domain does not contain a protease cleavable linker.

FIG. 38A shows IM-1062 Tribody protein characterization by SDS-PAGE. FIG. 38B shows IM-1062 Tribody protein characterization by SEC-HPLC.

FIG. 39A shows IM-1184 ProTribody protein characterization by SDS-PAGE. FIG. 39B shows IM-1184 ProTribody protein characterization by SEC-HPLC.

FIGS. 40A-40C show MS analysis of IM-1062 Tribody. FIG. 40A shows MS analysis of light chain. FIG. 40B shows MS analysis of heavy chain. FIG. 40C shows MS analysis of intact Tribody.

FIGS. 41A-41B show MS analysis of IM-1184 ProTribody. FIG. 41A shows MS analysis of light chain. FIG. 41B shows MS analysis of heavy chain.

FIGS. 42A-42B show results of protease cleavage assay of various ProTribody constructs under non-reducing (FIG. 42A) or reducing condition (FIG. 42B). Lane M: Maker; Lane 1: CD⅗T4/NKG2A(VL-VH) (IM1062); Lane 2: Cap137_HSA_MC3_CD⅗T4/NKG2A (IM1188); Lane 3: IM1188+ Matriptase/ST14; Lane 4: IM1188+ MMMP-9; Lane 5: IM1188+UPA; Lane 6: Cap137_HSA_NC_CD⅗T4/NKG2A (IM1193); Lane 7: IM1193 + Matriptase/ST14; Lane 8: IM1193 + MMMP-9; Lane 9: IM1193+UPA; Lane 10: Cap137_HSA_NC_CD⅗T4/NKG2A (IM1193); Lane 11: IM1184 + MMMP-9; Lane 12: IM1184+UPA; Lane 13: Cap137_HSA_MC2_CD⅗T4/NKG2A (IM1184); Lane 14: CD⅗T4/NKG2A(VL-VH) (IM1062).

FIGS. 43A-43D show ELISA binding of various Tribody and Tribody mutants on recombinant protein CD3 (FIG. 43A), 5T4 (FIG. 43B), or NKG2A (FIGS. 43C-43D). FIG. 43D shows binding to human NKG2A by bi-NKG2A scFv Tribodies variants that harbors two anti NKG2A scFv in tandem (IM-1272, IM-1273).

FIGS. 44A-44C show ELISA binding of various cleaved and non-cleaved ProTribody on recombinant CD3 protein. FIG. 44A: IM-1184 ProTribody; FIG. 44B: IM-1188 ProTribody; FIG. 44C: IM-1193 ProTribody.

FIGS. 45A-45F show FACS binding of Tribody and Tribody mutants on different cell lines. FIG. 45A: Jurkat cells; FIG. 45B: NCI-H226 cells; FIG. 45C: NK92 cells; FIG. 45D: CHO cells over-expressing 5T4; FIG. 45E: CHO cells over-expressing NKG2A/CD94. FIG. 45F: CHO cells over expressing human NKG2A tested with the bi-NKG2A scFv Tribody variants that harbor two anti NKG2A scFv in tandem (IM-1272, IM-1273).

FIGS. 46A-46C show FACS binding of various cleaved and non-cleaved ProTribody on Jurkat cell line. FIG. 46A: IM-1184 ProTribody; FIG. 46B: IM-1188 ProTribody; FIG. 46C: IM-1193 ProTribody.

FIGS. 47A-47F show in vitro killing function of various Tribody and Tribody mutants. FIG. 47A: T cell-mediated cytotoxicity on NCI-H226 cells; FIG. 47B: NK92-mediated cytotoxicity against LCL721.221 cells; FIG. 47C: PBMC-mediated cytotoxicity on A549 cells; FIGS. 47D-F: PBMC-mediated cytotoxicity on MDA-MB231 cells with Tribody concentrations at 1pM or 10pM (FIG. 47D), 100 nM (FIG. 47E) or 100pM (FIG. 47F).

FIG. 48 shows IM-1062 Tribody induces IFNy secretion in various cell lines.

FIG. 49 shows the ability of various Tribody and Tribody mutants to block NKG2A-HLA interaction.

FIGS. 50A-50B show the in vivo efficacy of IM-1062 Tribody in MDA-MB-231 xenograft mouse model. FIG. 50A shows average tumor volume over time. FIG. 50B shows tumor volume of individual mice treated with vehicle control (left panel) or IM-1062 Tribody (right panel).

FIGS. 51A-51B show the in vivo efficacy of a IM-1062 Tribody and a Tribody mutant in the A549 tumor model. FIG. 51A shows average tumor volume over time. FIG. 51B shows tumor volume of individual mice treated with vehicle control (left panel), IM-1062 Tribody (middle panel), or IM-1093 Tribody (right panel).

FIGS. 52A-52B show the percentage of immune cells in a tumor microenvironment. FIG. 52A shows %CD45+ cells. FIG. 52B shows % activated CD3 cells.

FIGS. 53A-53C show the in vivo efficacy of IM-1062 Tribody on HCT116 cells. FIG. 53A shows average tumor volume over time. FIG. 53B shows tumor volume of individual mice treated with control (left panel) or IM-1062 Tribody (right panel). FIG. 53C shows the in vivo efficacy of IM-1062 Tribody, IM-1184 and IM-1193 ProTribody on MDA-MB-231 cells.

FIG. 54 lists the SEQ ID NOs for the various components of the Tribody or ProTribody construct, such as VH, VL, CDRs, regulatory domains, protease cleavage sequences, and linkers.

FIG. 55 lists the names and SEQ ID NOs of various embodiments of the Tribody or ProTribody constructs disclosed herein, each construct comprising polypeptide A (HC) and polypeptide B (LC). AA: amino acid sequence; NA: nucleotide sequence.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following detailed description, numerous specific details are set forth in order to provide a thorough understanding of precursor tri-specific antibody constructs. However, it will be understood by those skilled in the art that the precursor constructs presented herein, the production of, and the use thereof may be practiced without these specific details. In other instances, well-known methods, procedures, and components have not been described in detail so as not to obscure the disclosure.

Described herein are precursor tri-specific antibody constructs comprising separate cleavable masking and half-life prolonging domains, wherein these cleavable regulatory domains provide reduced binding to T-cells or NK cells by the precursor tri-specific constructs when outside the tumor micro-environment (TME) and provide extended half-life. Half-life extension may be limited to the time a precursor tri-specific construct is outside the cancer microenvironment or it may extend to the time a precursor tri-specific construct resides within a cancer microenvironment. An advantage of the precursor tri-specific antibody constructs described herein, have a protease cleavable masking domain and a protease cleavable half-life prolonging (HLP) domain may be the improved protease-activated controlled release of the masking CAP and the HLP domain.

Reduction in T-cell or NK cell binding may lead to a reduction in T-cell or NK cell activation. In some embodiments, the precursor tri-specific antibody constructs described herein are regulatable precursor constructs. The regulatable precursor tri-specific antibody constructs describe herein may have an extended half-life, or reduced T-cell/NK cell binding, or reduced T-cell/NK cell activation, or any combination thereof.

As used herein, the terms “half-life prolonging”, “HLP”, “serum half-life prolonging”, “extended half-life”, “extended serum half-life”, “increased half-life”, “increased serum half-life”, and other similar terms may be used interchangeably having all the same qualities and meanings. In some embodiments, a half-life prolonging domain comprise a human serum albumin (HSA). As used herein, the term “human serum albumin” may in some embodiments be used interchangeably with “HSA” or “ALB” having all the same meanings and qualities.

In some embodiments, the precursor tri-specific antibody constructs described herein provide for a regulatable T-cell/NK cell activation, wherein the precursor construct provides that T-cell/NK cell activation is restricted to a tumor microenvironment. In some embodiments, the precursor tri-specific antibody construct described herein have an increased half-life and provide that T-cell/NK cell activation is restricted to a tumor microenvironment, compared with non-precursor always active multi-valent antibodies. In some embodiments, the precursor tri-specific antibody constructs described herein have reduced T-cell/NK cell activation in non-tumor microenvironments, compared with non-precursor always active multi-valent antibodies.

In some embodiments, the precursor tri-specific antibody constructs described herein have an extended half-life in non-tumor microenvironments, compared with non-precursor tri-specific antibodies. In some embodiments, the precursor tri-specific antibody constructs described herein have reduced T-cell/NK cell binding and/or activation in non-tumor microenvironments and an extended half-life in a non-tumor microenvironment, compared with non-precursor always active multi-valent antibodies.

In some embodiments, the precursor tri-specific antibody constructs described herein may bind to T-cells and to Natural Killer (NK) cells. In some embodiments, the precursor tri-specific antibody constructs described herein may bind to T-cells and to Natural Killer (NK) cells while also binding to a tumor associated antigen (TAA). In some embodiments, the precursor tri-specific antibody constructs described herein may bind to one NK cell surface antigen with one binding domain and bind to another NK cell surface antigen with another binding domain while also binding to a tumor associated antigen (TAA) with yet another binding domain. In some embodiments, the precursor tri-specific antibody constructs described herein may bind to one NK cell surface antigen with one binding domain and bind to a TAA with another binding domain while also carrying a cytokine receptor engager comprising an agent that binds to a cytokine receptor (e.g. the agent can be a cytokine such as IL-15). Examples of TAA and other antigens that can be recognized by the tri-specific antibody constructs are disclosed herein.

In some embodiments, described herein are pharmaceutical compositions comprising a precursor tri-specific antibody construct that provides a regulatable T-cell and/or NK cell activation in non-tumor microenvironments. In some embodiments, described herein are pharmaceutical compositions comprising a precursor tri-specific antibody construct having an increased half-life and providing that T-cell/NK cell activation is restricted to a tumor microenvironment. In some embodiments, described herein are pharmaceutical compositions comprising a precursor tri-specific antibody construct comprising an extended half-life in non-tumor microenvironments. In some embodiments, described herein are pharmaceutical compositions comprising a precursor tri-specific antibody construct comprising an extended half-life in non-tumor microenvironments, wherein the half-life is reduced in a tumor microenvironment compared with the half-life in a non-tumor microenvironment. In some embodiments, the pharmaceutical compositions comprising a precursor antibody construct that recognizes a T-cell, an NK cell, and a TAA. In some embodiments, the pharmaceutical compositions comprising a precursor antibody construct that recognizes two NK cell surface antigens and a TAA. In some embodiments, the pharmaceutical compositions comprising a precursor antibody construct having binding domains for a NK cell surface antigen, a TAA, and carrying a cytokine receptor engager comprising an agent that binds to a cytokine receptor (e.g. the engager can be a cytokine such as IL-15).

In some embodiment, described herein are methods of use of a precursor tri-specific antibody construct, as disclosed herein, for use treating, preventing, inhibiting the growth of, delaying disease progression, reducing the tumor load, or reducing the incidence of a cancer or tumor in a subject, or any combination thereof. In some embodiments, the method of treating disclosed herein reduces the minimal residual disease, increases remission, increases remission duration, reduces tumor relapse rate, prevents metastasis of the tumor or the cancer, or reduces the rate of metastasis of the tumor or the cancer, or any combination thereof, in the treated subject compared with a subject not administered with the pharmaceutical composition.

### Precursor Tri-specific Antibody Constructs

In some embodiments, a precursor tri-specific antibody construct comprises 1) a first binding domain binding to a TAA; 2) a second binding domain binding to an extracellular epitope of a Natural Killer (NK) cell antigen; 3) a third binding domain binding to a T cell surface antigen, e.g. an extracellular epitope of human CD3ε; and 4) a regulatory domain. The regulatory domain may comprise: (1) a first and a second sub-regulatory domain, the first sub-regulatory domain comprising a first protease cleavage domain and a half-life prolonging (HLP) domain, and the second sub-regulatory domain comprising a second protease cleavage domain and a CAP component that reduces the ability of the third binding domain to bind to its target antigen; or (2) a single regulatory domain comprising a protease cleavage domain, a half-life prolonging (HLP) domain, and a CAP component that reduces the ability of the third binding domain to bind to its target antigen. In one embodiment, when the second binding domain binds to a NK cell surface antigen, the second binding domain further comprises a third regulatory domain comprising a third protease cleavage domain and a CAP component that reduces the ability of the second binding domain to bind to the NK cell surface antigen.

In some embodiments, a precursor tri-specific antibody construct comprises 1) a first binding domain binding to a tumor associated antigen (TAA); 2) a second binding domain binding to an extracellular epitope of a first Natural Killer (NK) cell antigen; 3) a third binding domain binding to an extracellular epitope of a second NK cell surface antigen; and 4) a regulatory domain. The regulatory domain may comprise: (1) a first and a second sub-regulatory domain, the first sub-regulatory domain comprising a first protease cleavage domain and a half-life prolonging (HLP) domain, and the second sub-regulatory domain comprising a second protease cleavage domain and a CAP component that reduces the ability of the third binding domain to bind to its target antigen; or (2) a single regulatory domain comprising a protease cleavage domain, a half-life prolonging (HLP) domain, and a CAP component that reduces the ability of the third binding domain to bind to its target antigen. In one embodiment, when the second binding domain binds to a NK cell surface antigen, the second binding domain further comprises a third regulatory domain comprising a third protease cleavage domain and a CAP component that reduces the ability of the second binding domain to bind to the NK cell surface antigen. The first and second NK cell surface antigens can be the same or different antigens..

In some embodiments, a precursor tri-specific antibody construct comprises 1) a first binding domain binding to a tumor associated antigen (TAA); 2) a second binding domain having a cytokine receptor engager comprising an agent that binds to a cytokine receptor (e.g. the agent can be a cytokine such as IL-15); 3) a third binding domain binding to an extracellular epitope of a NK cell surface antigen; and 4) a regulatory domain. The regulatory domain may comprise: (1) a first and a second sub-regulatory domain, the first sub-regulatory domain comprising a first protease cleavage domain and a half-life prolonging (HLP) domain, and the second sub-regulatory domain comprising a second protease cleavage domain and a CAP component that reduces the ability of the third binding domain to bind to its target antigen; or (2) a single regulatory domain comprising a protease cleavage domain, a half-life prolonging (HLP) domain, and a CAP component that reduces the ability of the third binding domain to bind to its target antigen.

### Cytokine Receptor Engager

In some embodiments, a precursor tri-specific antibody construct comprises a binding domain having a cytokine receptor engager comprising an agent that binds to a cytokine receptor. In one embodiment, the cytokine receptor engager is a cytokine that binds to a cytokine receptor. As it is generally known in the art, cytokines is a general name; other names include lymphokine (cytokines made by lymphocytes), monokine (cytokines made by monocytes), chemokine (cytokines with chemotactic activities), and interleukin (cytokines made by one leukocyte and acting on other leukocytes). Cytokines may act on the cells that secrete them (autocrine action), on nearby cells (paracrine action), or in some instances on distant cells (endocrine action). Numerous cytokines are known in the art. There are pro-inflammatory cytokines and anti-inflammatory cytokines. Examples of pro-inflammatory cytokines include, but are not limited to, IL-1β, IL-6, IL-12, and TNF-α. Examples of anti-inflammatory cytokines include, but are not limited to, IL-1 receptor antagonist, IL-4, IL-10, IL-11, and IL-13. Depending on the circumstances, certain cytokines can be categorized as either anti-inflammatory or pro-inflammatory cytokines, for example, Leukemia inhibitory factor, interferon-alpha, IL-6, and transforming growth factor (TGF)-β. In one embodiment, the cytokine receptor engager is a pro-inflammatory cytokine. In another embodiment, the cytokine receptor engager is an anti-inflammatory cytokine.

In some embodiments, the cytokine receptor engager is a lymphokine. In some embodiments, the cytokine receptor engager is a monokine. In some embodiments, the cytokine receptor engager is a chemokine. In some embodiments, the cytokine receptor engager is a interleukin. In some embodiments, the cytokine receptor engager is a pro-inflammatory cytokine. In some embodiments, the cytokine receptor engager is an anti-inflammatory cytokine.

In some embodiments, a precursor tri-specific antibody construct comprises a binding domain having a cytokine receptor engager comprising an IL-15 molecule. In one embodiment, the cytokine receptor engager is a cytokine that binds to its cytokine receptor. Examples of cytokines have been disclosed herein. For example, the engager can be IL-15. In some embodiments, the IL-15 engager binds to IL-15 receptor. The cytokine can be from human or other non-human animals. The cytokine can be purified from native source, or constructed by recombinant techniques. In some embodiments, binding of the IL-15 engager domain to a target induces the proliferation of natural killer cells. It is known in the art that IL-15 plays important role on NK cell development and homeostasis without stimulating regulatory T cells. Results from clinical trials show that IL-15 administration was associated with a 38-fold increase in the number and activation status of circulating natural killer (NK) cells and activation of macrophages which together are ADCC effectors. Furthermore, IL-15 greatly enhanced the therapeutic efficacy of both rituximab and alemtuzumab in tumor models. In some embodiments, binding of the IL-15 engager domain to a target induces increase of the cytotoxic function of natural killer cells. In some embodiments, binding of the IL-15 engager domain to a target induces both the proliferation of natural killer cells and increases their cytotoxic function. In some embodiments, binding of the IL-15 engager domain to a target enhances the anti-tumor response by an activated precursor tri-specific antibody construct comprising an IL-15 engager. In some embodiments, binding of the IL-15 engager domain to a target enhances the efficacy in inducing tumor regression by an activated precursor tri-specific antibody construct comprising an IL-15 engager.

Regarding cytokine requirements for natural killer (NK) cell development and function, it is known that NK cell development from hematological stem cells (HSCs) is regulated by multiple cytokines in fetal liver, bone marrow, and thymus. The sequential expression of receptors for different cytokines implies functional maturation of NK cells. The proliferation and differentiation of HSCs requires FL (fms-like tyrosine kinase 3 ligand), KL (kit ligand), IL-3, and IL-7, which interact with their respective receptors. The acquisition of CD122 expression is indicative of the commitment of NK cells. IL-15 is indispensable for NK cell differentiation from common lymphoid progenitors to mature NK cells. Mature NK cells are shaped by cytokine signals from the diverse tissue environments in which they reside. In the peripheral blood or spleen, the abundance of stimulatory cytokines, such as IL-2, IL-12, IL-15, IL-18, and IL-21, may maintain NK cells in a cytotoxic state to combat infections. Tolerant NK cells that reside in the liver, and regulatory NK cells that reside in the uterus, are primarily regulated by TGF-β and IL-10 or by TGF-β and IL-15, respectively.

### Embodiments of Precursor Tri-specific Antibody Constructs

For the precursor constructs described throughout, the skilled artisan will appreciate that the modular structure of said constructs allows for different binding partners based on the amino acid sequences comprised in the first, second and third binding domains. In some embodiments, the first binding domain and the second binding domain each comprises a single chain variable fragment (scFv). In another embodiment, the third binding domain comprises a Fab antigen binding fragment.

In some embodiments, a binding domain comprises two scFv in tandem. For example, in some embodiments, a second binding domain comprises two scFv targeting NK cells. In some embodiments, the two scFv target the same antigen on an NK cell. In some embodiments, the two scFv target different antigens on an NK cell.

A skilled artisan would recognize that a precursor antibody construct is a precursor form or “Pro” form of the active antibody protein. In some embodiments, the term “Pro” is used interchangeably with the term “Precursor” having all the same meanings and qualities.

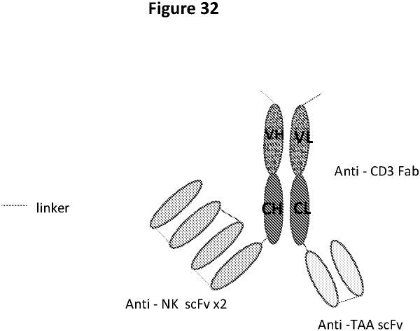

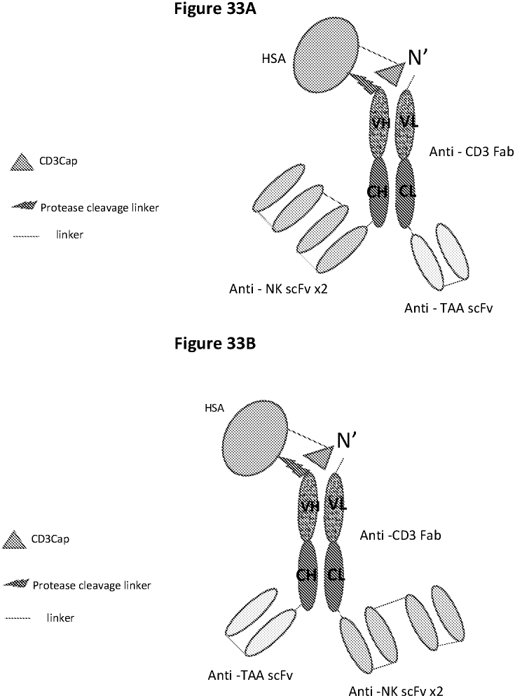

As used herein, the term “precursor tri-specific antibody” or “ProTribody antibody” refers to a tri-specific antibody comprising one or more regulatory domains that regulate antibody binding to one or more target antigens. (See for examples, FIGS. 32 and 33A-33B)

A skilled artisan would appreciate that as used throughout, in some embodiments the terms “precursor tri-specific antibody construct”, “precursor antibody”, “precursor construct”, “precursor antibody construct”, “precursor tri-specific antibody”, “tri-specific antibody”, “antibody”, “Tribody”, “tri-specific antibody construct”, and “tri-specific construct” may be used interchangeably having all the same qualities and meanings. Additionally, in some embodiments the term “tri-specific” may be replaced with the term “tri-body” or “Tribody” with the recognition that the antibody constructs disclosed herein have three binding regions, wherein each region may bind a different antigen (tri-body or tri-specific) or two of the three binding regions may bind the same antigen (tri-body or tri-specific wherein two of the specific binding antigens are the same). Thus, terms as listed above, for example “precursor tri-specific antibody construct” in some embodiments may be used interchangeably with the term “precursor tri-body construct”, having all the same meanings and qualities.

A skilled artisan would appreciate that in some embodiments, the term “tumor associated antigen” (TAA) may encompass a molecule or a portion thereof, which is displayed on the surface of a cell or a molecule which is present within the milieu of a tumor, that is within the tumor micro-environment. In some embodiments, a TAA encompasses a cell surface tumor associated antigen (TAA). In some embodiments, the cell is a tumor cell. In some embodiments, the cell is a non-tumor cell present in the milieu of a tumor, for example but not limited to a cell present within vasculature tissue associated with a tumor or cancer. In some embodiments, a TAA is an angiogenic antigen in a tumor micro-environment. In some embodiments, a TAA is an antigen on a blood vessel in a tumor micro-environment. In some embodiments, the cell is a stromal cells present in the milieu of a tumor. In some embodiments, a TAA is a stromal cell antigen within a tumor micro-environment. In some embodiments, a TAA encompasses an extracellular epitope of a tumor-cell-surface antigen. In some embodiments, a TAA encompasses an extracellular matrix antigen.

In some embodiments, an angiogenic antigen comprises a bFGF. In some embodiments, an angiogenic antigen comprises a INF. In some embodiments, an angiogenic antigen comprises a VEGF. In some embodiments, an angiogenic antigen comprises a bFGF, a INF, or a VEGF.

In some embodiments, a TAA comprises an antigen present in a TME. In some embodiments, a TAA comprises a cytokine antigen in a TME. In some embodiments, a TAA comprises a molecule secreted by a tumor cell into the TME. In some embodiments, a TAA comprises an effector molecule secreted by a tumor cell into the TME. In some embodiments, a TAA comprises an effector molecule secreted by a tumor cell into the TME in order to downregulate or inhibit the activity of cytotoxic natural killed (NK) cells. In some embodiments, a TAA comprises an effector molecule secreted by a tumor cell into the TME in order to downregulate or inhibit the activity of NK cells. In some embodiments, a TAA comprises soluble activating receptor ligand secreted by a tumor cell into the TME in order to block the recognition of the tumor cell by an NK cell. In some embodiments, a TAA comprises a suppressive immune cell in the TME that would otherwise inhibit NK cell activation. In some embodiments, a TAA comprises a suppressive molecule in the TME that would otherwise inhibit NK cell activation. In some embodiments, the effector molecule comprises a cytokine antigen. In some embodiments, the effector molecule comprises a cytokine antigen in the TME.

In some embodiments, a cytokine antigen in the TME comprises a TNF-alpha, an IL-6, a TGF-beta, an IL-10, an IL-8, an IL-17, an IL-21, an INF, or a VEGF. In some embodiments, a TAA is selected from a TNF-alpha, an IL-6, a TGF-beta, an IL-10, an IL-8, an IL-17, an IL-21, an INF, or an VEGF. In some embodiments, a cytokine antigen for use as a TAA comprises a cytokine antigen known in the art.

A skilled artisan would appreciate that the terms “tumor micro-environment” (TME), “cancer microenvironment” and “tumor milieu” may be used interchangeably having the same qualities and meanings and encompassing the microenvironment to tumor development. While the normal cellular microenvironment can inhibit malignant cell growth, the modifications that occur in the tumor microenvironment may synergistically support cell proliferation.

In some embodiments, the second binding domain and the third binding domain of a precursor construct disclosed herein bind to the same NK cell surface antigen. In some embodiments, the second binding domain and the third binding domain of a precursor construct disclosed herein bind to different NK cell surface antigens. In some embodiments, the second binding domain and the third binding domain of a precursor construct disclosed herein bind to the same or different NK cell surface antigens on the same cell. In some embodiments, the second binding domain and the third binding domain of a precursor construct disclosed herein bind to the same or different NK cell surface antigens on different NK cells.

In some embodiments, the second binding domain binds to an NK cell and the first binding domain binds to a TAA. The TAA may be for example, but not limited to an extracellular epitope of a tumor-cell surface antigen, a TME antigen, a stomal antigen in a TME, an angiogenic antigen in a TME, an antigen on a blood vessel in a TME, or a cytokine in a TME.

In some embodiments, binding to an NK cell comprises binding to an extracellular epitope of the NK cell antigen.

In some embodiments, the second binding domain binds to a negative effector molecule of an NK cell and the first binding domain binds to a TAA. The TAA may be for example, but not limited to an extracellular epitope of a tumor-cell surface antigen, a TME antigen, a stomal antigen in a TME, an angiogenic antigen in a TME, an antigen on a blood vessel in a TME, or a cytokine in a TME.

A skilled artisan would appreciate that the terms “antigen” or “immunogen” encompass a peptide, protein, or a polypeptide, or any fragment thereof, which is immunogenic. In some embodiments, an antigen is capable of eliciting an immune response in a mammal, and therefore contains at least one and may contain multiple epitopes. An “antigen” molecule or a portion of a molecule is capable of being bound by a selective binding agent, such as an antigen-binding portion of a Fab fragment or an antigen-binding portion of a single chain variable fragment (scFv). Additionally, an “antigen” is capable of being used in an animal to produce antibodies capable of binding to an epitope of that antigen. In some embodiments, a CAP component comprises the portion of an antigen to which a binding domain binds.

The term “epitope” includes any determinant, in certain embodiments, a polypeptide determinant, capable of specific binding to an anti-TAA binding domain or an anti-NK cell binding domain or an anti-T-cell receptor binding domain. An epitope is a region of an antigen that is bound by an antibody or an antigen-binding fragment thereof. In some embodiments, a CAP component comprises the epitope to which a binding domain binds.

In certain embodiments, epitope determinants include chemically active surface groupings of molecules such as amino acids, sugar side chains, phosphoryl or sulfonyl, and may in certain embodiments have specific three-dimensional structural characteristics, and/or specific charge characteristics. In certain embodiments, a precursor tri-specific antibody construct is said to specifically bind an antigen when it preferentially recognizes its target antigen in a complex mixture of proteins and/or macromolecules. A precursor tri-specific antibody construct is said to specifically bind an antigen when the equilibrium dissociation constant is ≤ 10⁻⁵, 10⁻⁶ or 10⁻⁷ M. In some embodiments, the equilibrium dissociation constant may be ≤ 10⁻⁸ M or 10⁻⁹ M. In some further embodiments, the equilibrium dissociation constant may be ≤ 10⁻¹⁰ M or 10⁻¹¹ M. Antigens disclosed herein included but are not limited to TAA, CAP components, NK cells, and immuno-effector molecules such as a human CD3 epsilon polypeptide.

In some embodiments, the tumor associated antigen (TAA) is a tumor antigen. In some embodiments, tumor antigens comprise those antigens are presented on tumor cells. In some embodiments, the tumor antigen is present on a cell of solid tumor. In some embodiments, the tumor antigen is a cancer antigen, present on a cell of a non-solid tumor.

In some embodiments, when the TAA is a tumor cell antigen, the tumor cell comprises a cell from a solid tumor. Solid tumors may be benign (not cancer), or malignant (cancer). Different types of solid tumors are named for the type of cells that form them. Examples of solid tumors are sarcomas, carcinomas, and lymphomas. In some embodiments, solid tumors are neoplasms (new growth of cells) or lesions (damage of anatomic structures or disturbance of physiological functions) formed by an abnormal growth of body tissue cells other than blood, bone marrow or lymphatic cells. In some embodiments, a solid tumor consists of an abnormal mass of cells which may stem from different tissue types such as liver, colon, breast, or lung, and which initially grows in the organ of its cellular origin. However, such cancers may spread to other organs through metastatic tumor growth in advanced stages of the disease.

In some embodiments, the solid tumor comprises a sarcoma or a carcinoma, a fibrosarcoma, a myxosarcoma, a liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, an Ewing’s tumor, a leiomyosarcoma, a rhabdomyosarcoma, a colon carcinoma, a pancreatic cancer or tumor, a breast cancer or tumor, an ovarian cancer or tumor, a prostate cancer or tumor, a squamous cell carcinoma, a basal cell carcinoma, an adenocarcinoma, a sweat gland carcinoma, a sebaceous gland carcinoma, a papillary carcinoma, a papillary adenocarcinomas, a cystadenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilm’s tumor, a cervical cancer or tumor, a uterine cancer or tumor, a testicular cancer or tumor, a lung carcinoma, a small cell lung carcinoma, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodenroglioma, a schwannoma, a meningioma, a melanoma, a neuroblastoma, or a retinoblastoma. In some embodiments, the solid tumor comprises an Adrenocortical Tumor (Adenoma and Carcinoma), a Carcinoma, a Colorectal Carcinoma, a Desmoid Tumor, a Desmoplastic Small Round Cell Tumor, an Endocrine Tumor, an Ewing Sarcoma, a Germ Cell Tumor, a Hepatoblastoma a Hepatocellular Carcinoma, a Melanoma, a Neuroblastoma, an Osteosarcoma, a Retinoblastoma, a Rhabdomyosarcoma, a Soft Tissue Sarcoma Other Than Rhabdomyosarcoma, and a Wilms Tumor. In some embodiments, the solid tumor is a breast tumor. In another embodiment, the solid tumor is a prostate cancer. In another embodiment, the solid tumor is a colon cancer. In some embodiments, the tumor is a brain tumor. In another embodiment, the tumor is a pancreatic tumor. In another embodiment, the tumor is a colorectal tumor.

In some embodiments, the tumor cell comprises a cell from a non-solid tumor, that is a non-solid cancer. In some embodiments, a cancer may be a diffuse cancer, wherein the cancer is widely spread; not localized or confined. In some embodiments, a diffuse cancer may comprise a non-solid tumor. Examples of diffuse cancers include leukemias. Leukemias comprise a cancer that starts in blood-forming tissue, such as the bone marrow, and causes large numbers of abnormal blood cells to be produced and enter the bloodstream.

In some embodiments, a diffuse cancer comprises a B-cell malignancy. In some embodiments, the diffuse cancer comprises leukemia. In some embodiments, the cancer is lymphoma. In some embodiments, the lymphoma is large B-cell lymphoma.

In some embodiments, the diffuse cancer or tumor comprises a hematological tumor. In some embodiments, hematological tumors are cancer types affecting blood, bone marrow, and lymph nodes. Hematological tumors may derive from either of the two major blood cell lineages: myeloid and lymphoid cell lines. The myeloid cell line normally produces granulocytes, erythrocytes, thrombocytes, macrophages, and mast cells, whereas the lymphoid cell line produces B, T, NK and plasma cells. Lymphomas (e.g. Hodgkin’s Lymphoma), lymphocytic leukemias, and myeloma are derived from the lymphoid line, while acute and chronic myelogenous leukemia (AML, CML), myelodysplastic syndromes and myeloproliferative diseases are myeloid in origin.

In some embodiments, a non-solid (diffuse) cancer or tumor comprises a hematopoietic malignancy, a blood cell cancer, a leukemia, a myelodysplastic syndrome, a lymphoma, a multiple myeloma (a plasma cell myeloma), an acute lymphoblastic leukemia, an acute myelogenous leukemia, a chronic myelogenous leukemia, a Hodgkin lymphoma, a non-Hodgkin lymphoma, or plasma cell leukemia.

In some embodiments, the tumor or cancer comprises a metastasis of a tumor or cancer.

In some embodiments a cell surface TAA is located in or on the plasma membrane of the cell, such that at least part of this molecule remains accessible from outside the cell in tertiary form. In some embodiments, a cell surface TAA that is located in the plasma membrane is a transmembrane protein comprising, in its tertiary conformation, regions of hydrophilicity and hydrophobicity.

These antigens can be presented on the cell surface with an extracellular part which is often combined with a transmembrane and cytoplasmic part of the molecule. These antigens can sometimes be presented only by tumor cells and never by the normal ones. Tumor antigens can be exclusively expressed on tumor cells or might represent a tumor specific mutation compared to normal cells. In this case, they are called tumor-specific antigens. More common are antigens that are presented by tumor cells and normal cells. In some embodiments, TAA include antigens exclusively expressed on a tumor cell. In some embodiments, TAA include antigens expressed on both tumor and normal cells.

In some embodiments, TAA can be overexpressed on tumor cells compared to normal cells or are accessible for antibody binding in tumor cells due to the less compact structure of the tumor tissue compared to normal tissue.

In some embodiments, a precursor tri-specific antibody constructs described herein comprises (a) an scFv fragment comprising a first binding domain, binding to a TAA (TAA binding domain); (b) an scFv fragment comprising a second binding domain, binding to a NK cell antigen (NK cell binding domain); (c) an Fab fragment comprising a third binding domain, binding to an extracellular epitope of a T cell surface antigen such as human CD3ε (CD3 binding domain); and (d) a regulatory domain. The regulatory domain may comprise (1) a first and a second sub-regulatory domain, the first sub-regulatory domain comprising a first protease cleavage domain and a half-life prolonging (HLP) domain, and the second sub-regulatory domain comprising a second protease cleavage domain and a CAP component that reduces the ability of the third binding domain to bind to its target antigen; or (2) a single regulatory domain comprising a protease cleavage domain, a half-life prolonging (HLP) domain, and a CAP component that reduces the ability of the third binding domain to bind to its target antigen.

In some embodiments, a precursor tri-specific antibody constructs described herein comprises (a) an scFv fragment comprising a first binding domain, binding to a TAA (TAA binding domain); (b) an scFv fragment comprising a second binding domain, binding to a NK cell surface antigen, or the second binding domain comprises a cytokine receptor engager having an agent that binds to a cytokine receptor (e.g. the agent can be a cytokine such as IL-15); (c) an Fab fragment comprising a third binding domain, binding to an extracellular epitope of another NK cell antigen; and (d) a regulatory domain. In one embodiment, a The regulatory domain may comprise (1) a first and a second sub-regulatory domain, the first sub-regulatory domain comprising a first protease cleavage domain and a half-life prolonging (HLP) domain, and the second sub-regulatory domain comprising a second protease cleavage domain and a CAP component that reduces the ability of the third binding domain to bind to its target antigen; or (2) a single regulatory domain comprising a protease cleavage domain, a half-life prolonging (HLP) domain, and a CAP component that reduces the ability of the third binding domain to bind to its target antigen.

In some embodiments, when the second binding domain bind to an NK antigen, the precursor constructs further comprise additional regulatory domains linked to the second binding domain. For example, when the binding site binds to an NK antigen, in some embodiments, a third regulatory domain comprising a protease cleaving domain and a CAP component that reduces the ability of the second binding domain to bind the extracellular epitope of the NK is linked to the precursor construct C-terminal to the second binding domain and having the order N-terminal to C-terminal: Linker (L)-protease cleavage peptide-L-CAP. In some embodiments, the amino acid sequences of the CAP components that reduce the ability of the second and third binding domains to bind to their target NK cell antigens are the same. In some embodiments, the amino acid sequences of the CAP components that reduce the ability of the second and third binding domains to bind to their target NK cell antigens are different.

A skilled artisan would appreciate that in some embodiments, a precursor antibody construct encompasses a precursor or derivative form of a pharmaceutically active antibody. In some embodiments, a medicinal preparation comprises a precursor antibody construct. In some embodiments, a formulation comprises a precursor antibody construct. In some embodiments, a precursor antibody construct has reduced adverse effects compared to the activated antibody. In some embodiments, a precursor antibody construct has reduced adverse effects compared to the activated antibody, wherein the precursor antibody may be enzymatically activated or converted into the active form of the antibody.

In certain embodiments, a precursor antibody construct has a prolonged half-life compared to the activated antibody. In certain embodiments, a precursor antibody construct has a prolonged half-life compared to the activated antibody, wherein the precursor antibody may be enzymatically activated or converted into the active form of the antibody and the active form has a decreased half-life compared with the precursor antibody construct.

In some embodiments, a precursor antibody construct has reduced ability to bind a T-cell. In certain embodiments, a precursor antibody construct has a reduced ability to activate T-cells compared to the activated antibody. In some embodiments, a precursor antibody construct has reduced ability to bind a NK cell. In certain embodiments, a precursor antibody construct has a reduced ability to activate NK cells compared to the activated antibody, wherein the precursor antibody may be enzymatically activated or converted into the active form of the antibody.

In certain embodiments, a precursor antibody construct has both a prolonged half-life and a reduced ability to activate T-cells compared to the activated antibody. In certain embodiments, a precursor antibody construct has both a prolonged half-life and a reduced ability to bind NK cells compared to the activated antibody. In certain embodiments, a precursor antibody construct has both a prolonged half-life and a reduced ability to activate T-cells compared to the activated antibody, wherein the precursor antibody may be enzymatically activated or converted into the active form of the antibody. In certain embodiments, a precursor antibody construct has both a prolonged half-life and a reduced ability to bind NK cells compared to the activated antibody, wherein the precursor antibody may be enzymatically activated or converted into the active form of the antibody.

In some embodiments, a precursor antibody has reduced ability to bind a T-cell, wherein the regulatory domain comprising the CAP component is cleaved but the regulatory domain comprising the HLP has not been cleaved, wherein the “partially” activated antibody may bind to a T-cell and retain an extended half-life. In some embodiments, binding of a partially activated precursor antibody to a T-cell is reduced compared to a fully activated antibody that has both regulatory arms proteolytically cleaved. In some embodiments, a precursor antibody has reduced ability to activate a NK cell, wherein the regulatory domain comprising the CAP component is cleaved but the regulatory domain comprising the HLP has not been cleaved, wherein the “partially” activated antibody may activate a NK cell and retain an extended half-life. In some embodiments, activation of a NK cell is reduced following binding of a partially activated precursor construct, compared to a fully activated antibody that has both regulatory arms proteolytically cleaved.

In some embodiments, a precursor antibody construct is synthesized in vitro. In some embodiments, a precursor antibody construct is not converted to an active form of the antibody, when the precursor is present in vivo (e.g., in circulation) in a non-tumor microenvironment.

In some embodiments, a precursor antibody construct comprises multiple regulatory domains, in addition to antigen binding domains. In some embodiments, a precursor antibody construct comprises two regulatory domains, in addition to antigen binding domains. In some embodiments, a precursor antibody construct comprises enzymatically cleavable regulatory domains, in addition to antigen binding domains. In some embodiments, a precursor antibody construct comprises multiple regulatory domains in addition to antigen binding domains, wherein a portion of said regulatory domains is enzymatically cleavable. In some embodiments, a precursor antibody construct comprises two regulatory domains in addition to antigen binding domains, wherein a portion of said regulatory domains is enzymatically cleavable. In some embodiments, a precursor antibody construct comprises two regulatory domains in addition to three antigen binding domains, wherein said regulatory domains are enzymatically cleavable.

In some embodiments, a precursor tri-specific antibody described herein comprises enhanced selectivity at targeting tumor cells over normal cells prior to cytotoxic activation of T-cells or NK cells.

Immunological binding generally refers to the non-covalent interactions of the type which occur between an immunoglobulin molecule and an antigen for which the immunoglobulin is specific, for example by way of illustration and not limitation, as a result of electrostatic, ionic, hydrophilic and/or hydrophobic attractions or repulsion, steric forces, hydrogen bonding, van der Waals forces, and other interactions. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (kₒₙ) can be determined by calculation of the concentrations and the actual rates of association and the “off rate constant” (k_{off}) and can be determined by the actual rates of dissociation. The ratio of k_{off}/kₒₙ is thus equal to the dissociation constant K_{D}. See, generally, Davies et al. (1990) Annual Rev. Biochem. 59:439-473.

A skilled artisan would appreciate that a “binding domain” or related expressions such as a domain that “binds” or has “reactivity with/to” a specific target encompasses the ability of the domain to discriminate between the respective antigens and to specifically associate with a target antigen. A “binding domain” or “binding region” according to the present disclosure may be, for example, any protein, polypeptide, oligopeptide, or peptide that possesses the ability to specifically recognize and bind to a biological molecule (e.g., a cell surface receptor or tumor protein, or a component thereof, e.g., an extracellular component thereof). A binding domain includes any naturally occurring, synthetic, semi-synthetic, or recombinantly produced binding partner for a biological molecule of interest. For example, and as further described herein, a binding domain may be antibody light chain and heavy chain variable regions, or the light and heavy chain variable regions can be joined together in a single chain and in either orientation (e.g., VL-VH or VH-VL). A variety of assays are known for identifying binding domains of the present disclosure that specifically bind with a particular target, including Western blot, ELISA, flow cytometry, or surface plasmon resonance analysis (e.g., using BIACORE.TM. analysis).

In some embodiments, binding domain or a portion thereof “specifically binds” to a target molecule if it binds to or associates with a target molecule with an affinity or Ka (i.e., an equilibrium association constant of a particular binding interaction with units of ⅟M) of, for example, greater than or equal to about 10⁵M⁻¹. In certain embodiments, a binding domain or a portion thereof binds to a target with a Kₐ greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹, “High affinity” binding domains may encompass those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater. Alternatively, affinity may be defined as an equilibrium dissociation constant (Kd) of a particular binding interaction with units of M (e.g., 10⁻⁵ M to 10⁻¹³ M, or less). Affinities of binding domain polypeptides and portions thereof, as described herein can be readily determined using conventional techniques (see, e.g., Scatchard et al. (1949) Ann. N.Y. Acad. Sci. 51:660; and U.S. Pat. Nos. 5,283,173; 5,468,614, or the equivalent, which are incorporated herein in full).

Illustrative binding domains are described herein. In certain embodiments, the target molecule may be a cell surface expressed protein, such as a receptor or a tumor antigen. In some embodiments, the target molecule is a tumor associated antigen (TAA). In some embodiments, the target molecule is a surface antigen of a Natural Killer (NK) cell. Illustrative binding domains include immunoglobulin antigen-binding domains such as scFv, scTCR, extracellular domains of receptors, ligands for cell surface molecules/receptors, or receptor binding domains thereof, and tumor binding proteins. In certain embodiments, the antigen binding domains can be an scFv, a VH, a VL, a domain antibody variant (dAb), a camelid antibody (VHH), a fibronectin 3 domain variant, an ankyrin repeat variant and other antigen-specific binding domain derived from other protein scaffolds (Owen, B. (2017) Nat Biotechnol Jul 12:35(7):602-603).

Thus, in certain embodiments, a binding domain comprises an antibody-derived binding domain but can be a non-antibody derived binding domain. An antibody-derived binding domain can be a fragment of an antibody or a genetically engineered product of one or more fragments of the antibody, which fragment is involved in binding with the antigen. Examples include, without limitation, a complementarity determining region (CDR), a variable region (Fv), a heavy chain variable region (VH), a light chain variable region (VL), a heavy chain, a light chain, a single chain variable region (scFv), a Fab, a single domain camel antibody (camelid VHH), and single domain antibodies (dAb).

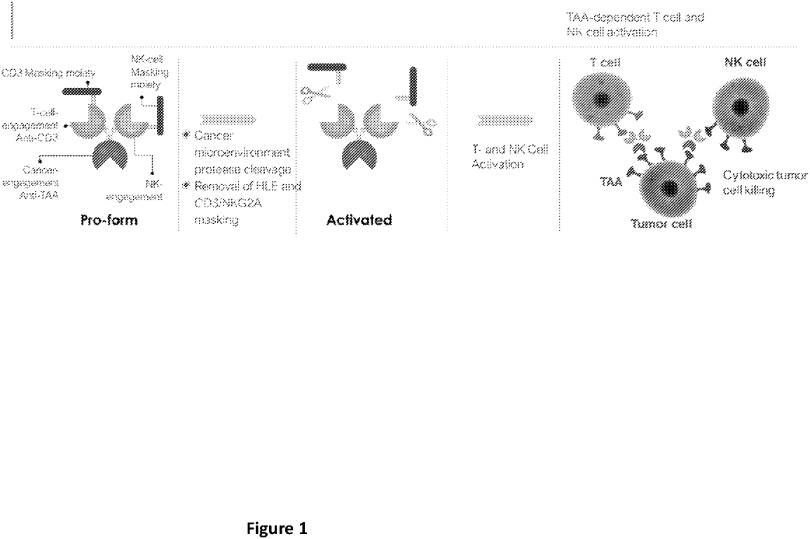

FIG. 1 shows flow diagrams of protease specific activation within tumor tissue or within a tumor environment of precursor tri-specific antibody constructs, wherein T cell and/or NK cell engagement and activation is limited to tumor sites. In one embodiment, the precursor tri-specific antibody construct comprises binding sites to T cells and NK cells. These binding sites are blocked or masked in the precursor antibody (the Pro-form). Entry into a cancer microenvironment, which is known to be rich in cancer-cell secreted proteases, results in protease cleavage and removable of the masking moieties. In some embodiments, the protease cleavable masking moieties may be cleavable by the same or difference proteases. The resultant activated antibody (activated tri-specific antibody construct) may now bind and activate T cell and NK cell. Were the precursor construct to bind to a tumor associated antigen (TAA) not in a tumor microenvironment, protease cleavage would not occur, and neither would activation of the tri-specific antibody. The activated tri-specific antibody construct antibody has a reduced half-life of hours compared with days to weeks for the precursor tri-specific antibody construct (data not shown). Furthermore, in some embodiments, a cleaved and activated tri-specific antibody comprises a smaller size than the precursor construct, which may improve tumor penetration of the activated tri-specific antibody.

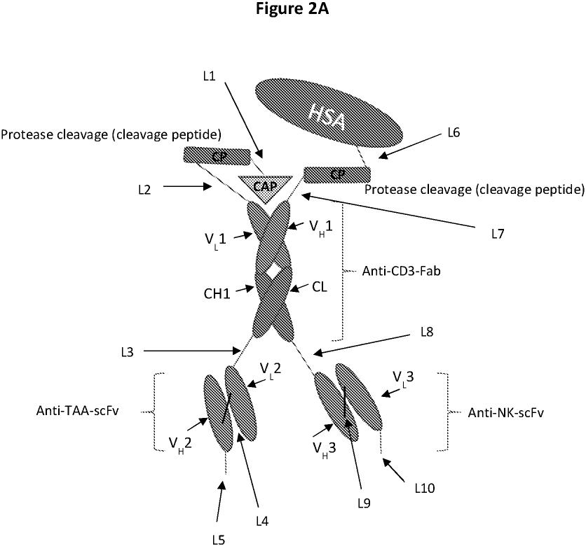

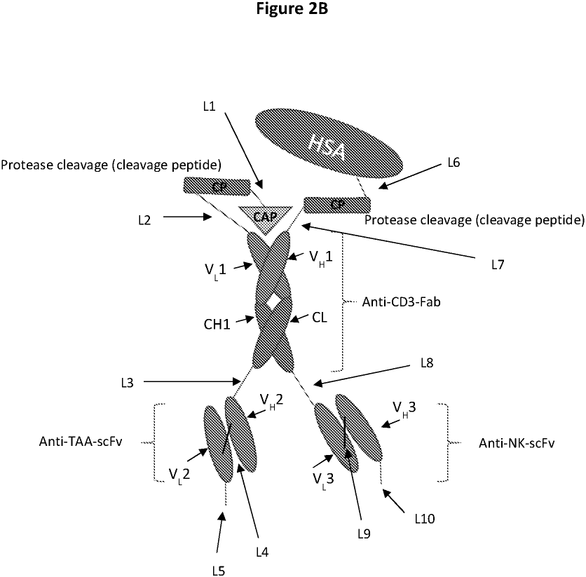

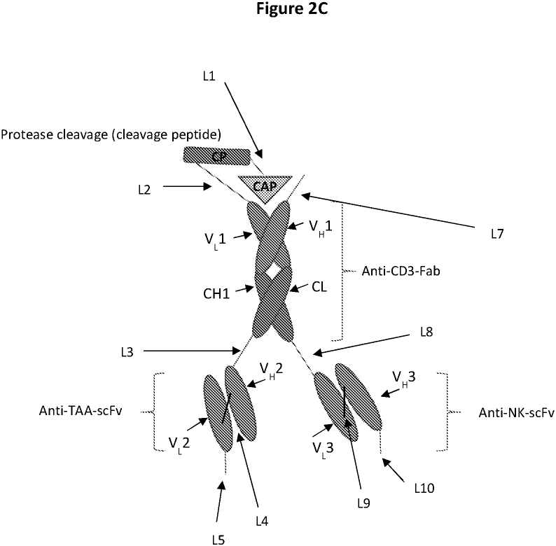

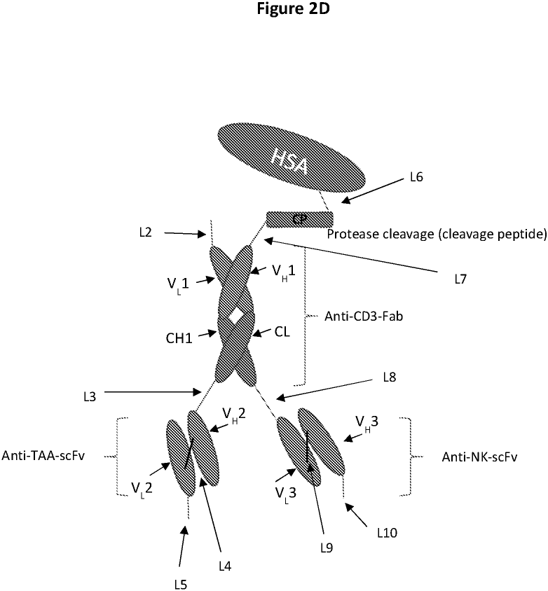

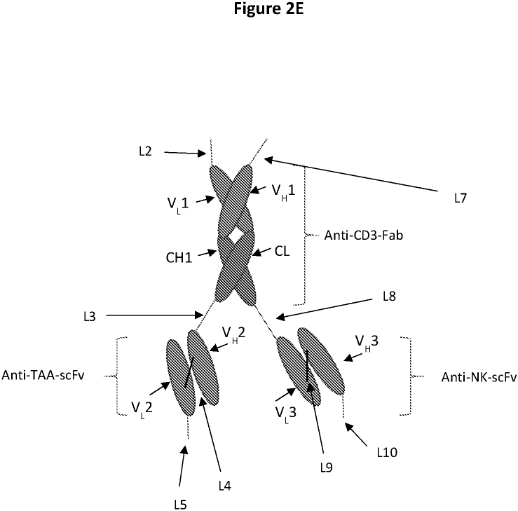

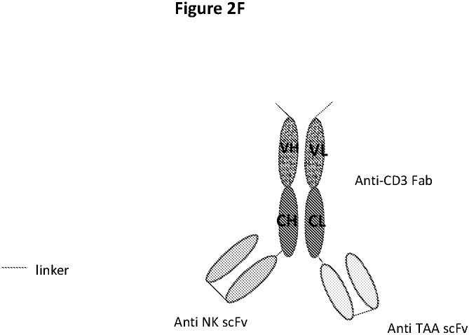

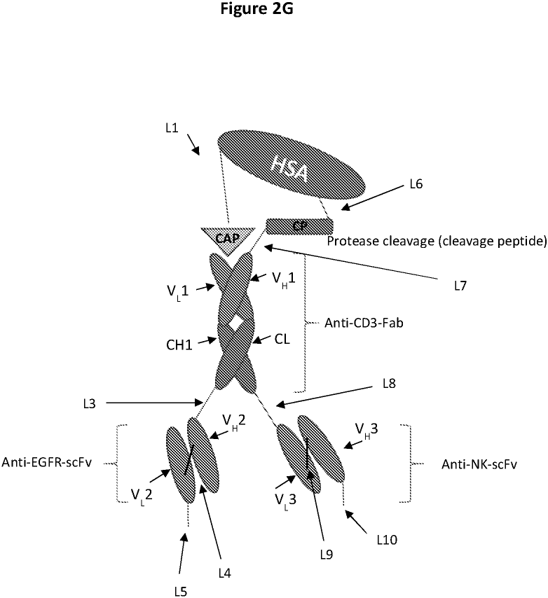

FIGS. 2-5 and 28-37 present schematic embodiments of precursor tri-specific (tri-body) antibody constructs having modular components, for example but not limited to: (1) modular regulatory domains including in certain embodiments, modular functional components and modular protease cleavage peptides, and (2) modular binding domains including in certain embodiments modular anti-tumor associated antigen binding domains, modular anti-T cell extracellular antigen binding domains, and modular anti-Natural Killer (NK) cell extracellular antigen binding domains.

FIG. 2A presents one embodiment of a precursor tri-specific antibody (tri-body) construct having three antibody binding domains, the Fab portion recognizes a CD3 surface antigen, one scFv recognizes a TAA, and the other scFv recognizes a NK cell surface antigen. In the embodiment shown here, the precursor tri-body construct is formed by two polypeptides. The anti-TAA and anti-NK binding domains are C-terminal to the anti-CD3 Fab binding domain. Furthermore, each polypeptide includes a regulatory domain N-terminal to the anti-CD3 Fab binding domain, wherein a first sub-regulatory domain comprises a protease cleavage domain and a serum half-life prolonging domain (in this embodiment, a human serum albumin HSA), and a second sub-regulatory domain comprising a protease cleavage domain and a CAP component that reduces the ability of the Fab binding domain to bind the surface antigen of CD3. In embodiments not shown, the two sub-regulatory domains N-terminal to the Fab region could be linked to a polypeptide chain different from the one shown here, for example the HSA regulatory domain could be linked N-terminal to the Light chain variable region (VL1) of the Fab fragment and the CAP regulatory domain could be linked N-terminal to the Heavy chain variable region (VH1) of the Fab fragment. As shown here, the anti-TAA and anti-NK cell binding domains are single chain variable fragments (scFv). In one embodiment, the N-terminal to C-terminal order of one of the scFv is a Light chain variable region (VL2) followed by a linker (L4) followed by a Heavy chain variable region (VH2) followed by a linker (L5), while the N-terminal to C-terminal order of the other scFv is a Heavy chain variable region (VH3) followed by a linker (L9) followed by a Light chain variable region (VL3) followed by a linker (L10). The order of the light chain variable region (VL) and heavy chain variable region (VH) regions for the two scFv domains, from N-terminal to C-terminal, may be different from one another (as shown here), or be the same as one another (not shown). The linkers between components and between domains are identified by an “L” followed by a numeral, e.g., L1, L2, L3, L4, L5, L6, L7, L8, L9, L10. Linkers may or may not be present. VL1 is a variable light-chain region of the Fab binding site, VH1 is a variable heavy-chain region of the Fab binding site. The oval shape indicated as HSA is the human serum albumin component. The shape indicated as CP is the protease cleavable peptide. The triangle shape is the CAP component.

FIG. 2B presents another embodiment of the precursor tri-specific antibody (tri-body) construct of FIG. 2A, showing a different order of VH, VL in the scFv domains. As shown here, the anti-TAA and anti-NK cell binding domains are single chain variable fragments (scFv). In one embodiment, the N-terminal to C-terminal order of one of the scFv is a Variable Heavy chain region (VH2) followed by a linker (L4) followed by a Variable Light chain region (VL2) followed by a linker (L5), while the N-terminal to C-terminal order of the other scFv is a Variable Light chain region (VL3) followed by a linker (L9) followed by a Variable Heavy chain region (VH3) followed by a linker (L10). Other components in the figure are the same as in FIG. 2A.

FIG. 2C presents one embodiment of a precursor tri-specific antibody construct as described above, having the regulatory domain comprising a CAP component that reduces the ability of the Fab binding domain to bind its target, but lacking the regulatory domain comprising the serum half-life extending component (HSA).

FIG. 2D presents one embodiment of a precursor tri-specific antibody construct as described above, having the regulatory domain comprising the serum half-life extending component (HSA), but lacking the regulatory domain comprising the CAP domain that reduces the ability of the Fab binding domain to bind its target.

FIG. 2E presents one embodiment of an active tri-specific (tribody) antibody construct derived from the precursor tri-specific antibody construct as described above, wherein the active construct lacks the two regulatory domains having the serum half-life extending component and the CAP domain.

FIG. 2F presents one embodiment of a precursor tri-specific antibody construct as described above, wherein the regulatory domain N-terminal to the Fab comprises a single regulatory domain comprising a CAP domain, a HSA sequence, and a protease cleavable linker on the same polypeptide.

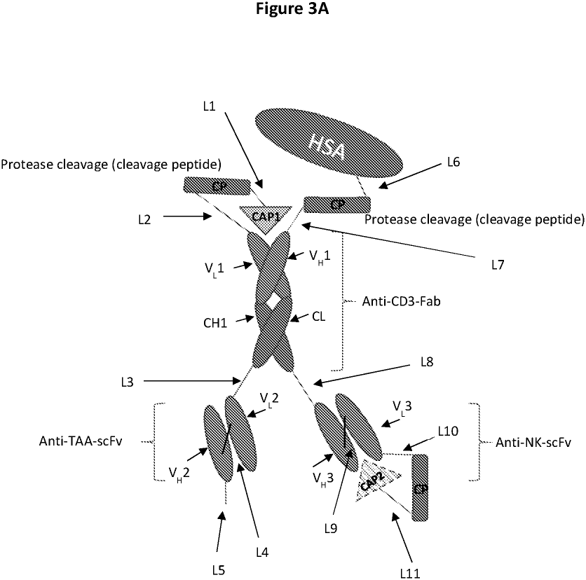

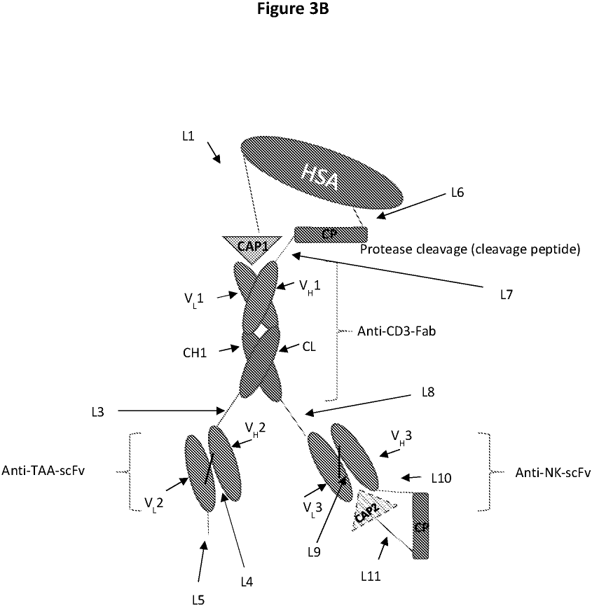

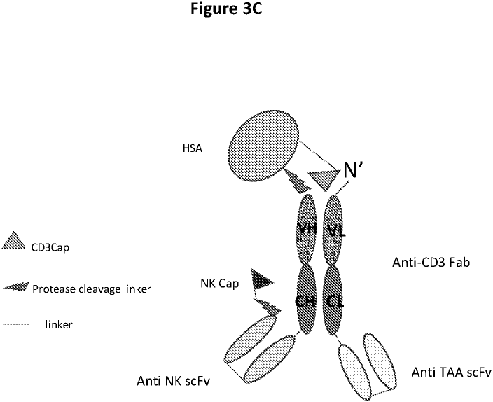

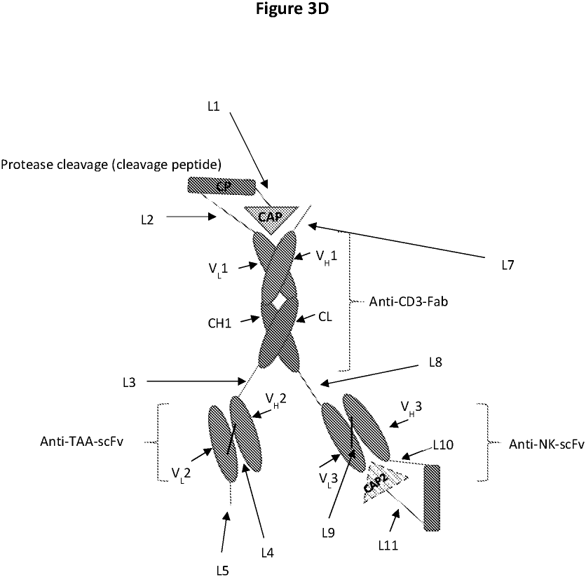

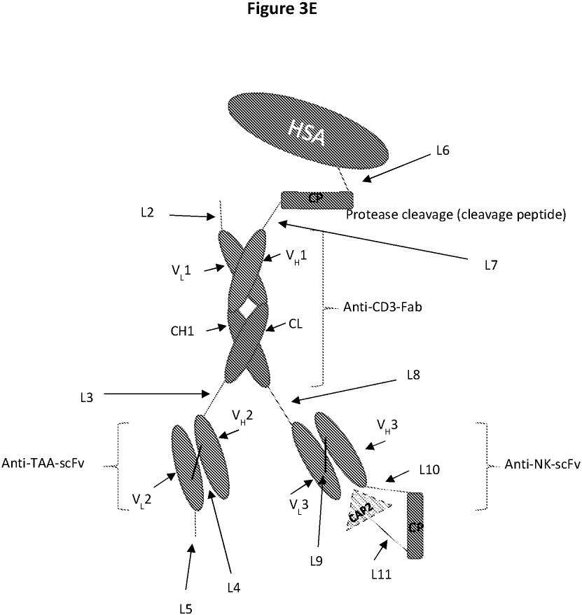

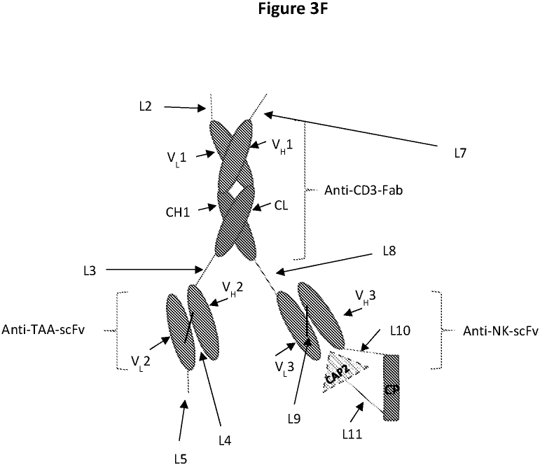

FIG. 3A presents one embodiment of a precursor tri-specific antibody (tri-body) having regulated anti-T cell and anti-NK cell binding. The precursor construct comprises three antibody binding domains, the Fab portion recognizes a CD3 surface antigen, one scFv recognizes a TAA, and the other scFv recognizes a NK cell surface antigen. In the embodiment shown here, the precursor tri-body construct is formed by two polypeptides. The anti-TAA and anti-NK binding domains are C-terminal to the anti-CD3 Fab binding domain as described above. In one embodiment, the anti-NK binding domain may further include a regulatory module (third regulatory domain) comprising linkers (L10, L11), a protease cleavable peptide (CP) domain, and a CAP component that reduces the ability of the anti-NK binding domain to bind the surface antigen of an NK cell. There are regulatory domains N-terminal to the anti-CD3 Fab binding domain as described above. In the embodiment shown here, the anti-TAA binding domain and the anti-NK antigen binding domain are each single chain variable fragments (scFv). In the embodiment shown here for the anti-TAA-scFv, the N-terminal to C-terminal order is a Variable Light chain region (VL2) followed by a linker (L4) followed by a Variable Heavy chain region (VH2) followed by a linker (L5) while the N-terminal to C-terminal order of the anti-NK-scFv is a Variable Heavy chain region (VH3) followed by a linker (L9) followed by a Variable Light chain region (VL3) followed by a linker (L10) followed by protease cleavable domain, linker (L11) which is followed by a CAP component that reduces the ability of the anti-NK binding domain to bind a surface antigen on an NK cell. The order of the light chain variable (VL) and heavy chain variable (VH) regions for the two scFv domains, from N-terminal to C-terminal, may be different from one another (as shown here), or be the same as one another (not shown). In embodiments not shown, the two sub-regulatory domains N-terminal to the Fab region could be linked to a polypeptide chain different from the one shown here, for example the HSA regulatory domain could be linked N-terminal to the Light chain variable region (VL1) of the Fab fragment and the CAP regulatory domain could be linked N-terminal to the Heavy chain variable region (VH1) of the Fab fragment. Other components are the same as described above.

FIG. 3B presents another embodiment of a precursor tri-specific antibody construct having regulated anti-T cell and anti-NK cell binding, wherein the regulatory domain N-terminal to the Fab comprises a single regulatory domain comprising a CAP domain, a HSA sequence, and a protease cleavable linker on the same polypeptide. The precursor tri-specific antibody (tri-body) construct has three antibody binding domains, the Fab portion recognizes a CD3 surface antigen, one scFv recognizes a TAA, and the other scFv recognizes a NK cell surface antigen as described above. The regulatory domain N-terminal to the anti-CD3 Fab binding domain comprises a single regulatory domain comprising a protease cleavage domain, a serum half-life prolonging (HLP) domain, and a CAP component that reduces the ability of the Fab to bind to its target antigen. In the embodiment shown here for the anti-TAA-scFv, the N-terminal to C-terminal order is a Variable Heavy chain region (VH2) followed by a linker (L4) followed by a Variable Light chain region (VL2) followed by a linker (L5) while the N-terminal to C-terminal order of the anti-NK-scFv is a Variable Light chain region (VL3) followed by a linker (L9) followed by a Variable Heavy chain region (VH3) followed by a linker (L10) followed by protease cleavable domain, linker (L11) which is followed by a CAP component that reduces the ability of the anti-NK binding domain to bind a surface antigen on an NK cell. In embodiments not shown, the regulatory domains N-terminal to the Fab region could be linked to a polypeptide chain different from the one shown here, for example the HSA/CAP regulatory domain could be linked N-terminal to the Variable Light chain (VL1) of the Fab fragment.

FIG. 3C presents one embodiment of a precursor tri-specific antibody construct having the regulatory domain comprising a CAP component that reduces the ability of the Fab binding domain to bind its target, but lacking the regulatory domain comprising the serum half-life extending component (HSA). The Fab portion, the anti-TAA scFv and the anti-NK scFv are the same as shown in FIG. 3B.

FIG. 3D presents one embodiment of a precursor tri-specific antibody construct having the serum half-life extending component (HSA), but lacking the regulatory domain comprising a CAP component that reduces the ability of the Fab binding domain to bind its target. The Fab portion, the anti-TAA scFv and the anti-NK scFv are the same as shown in FIG. 3B.

FIG. 3E presents one embodiment of an active tri-specific (tribody) antibody construct, lacking the two regulatory domains that include the serum half-life extending component and the CAP domain. The Fab portion, the anti-TAA scFv and the anti-NK scFv are the same as shown in FIG. 3B.

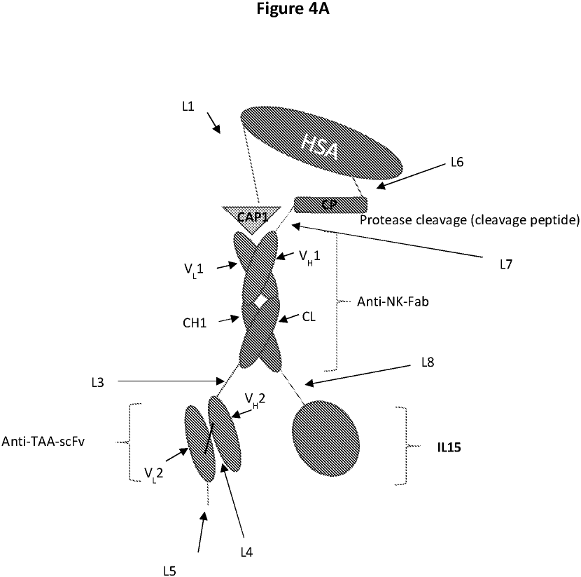

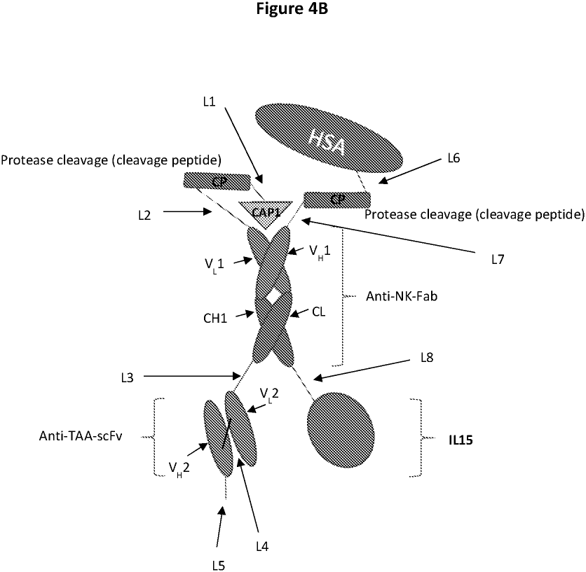

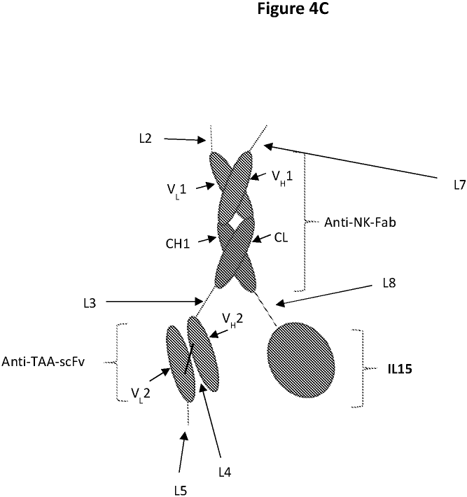

FIG. 4A presents one embodiment of a precursor tri-specific antibody (tri-body) construct having a cytokine receptor engager. The precursor construct also comprises regulatory domains all contained in one polypeptide chain. The precursor construct comprises a Fab portion recognizing a NK cell surface antigen (e.g. NKG2A or NKG2D etc), an anti-TAA scFv, and a cytokine receptor engager (e.g. IL-15). In the embodiment shown here, the precursor tri-body construct is formed by two polypeptides. The anti-TAA scFv and the cytokine receptor engager are located C-terminal to the Fab region. The regulatory domain N-terminal to the Fab comprises a single regulatory domain comprising a CAP domain, a HSA sequence, and a protease cleavable linker on the same polypeptide. In the embodiment shown here for the anti-TAA-scFv, the N-terminal to C-terminal order is a Variable Heavy chain region (VH2) followed by a linker (L4) followed by a Variable Light chain region (VL2) followed by a linker (L5). In embodiments not shown, the N-terminal to C-terminal order of the anti-TAA scFv may be reversed, i.e. VL2-L4-VH2-L5. In embodiments not shown, the regulatory domains N-terminal to the Fab region could be linked to a polypeptide chain different from the one shown here, for example the HSA/CAP regulatory domain could be linked N-terminal to the Variable Light chain (VL1) of the Fab fragment. Other components in the figure are the same as those shown above.

FIG. 4B presents another embodiment of a precursor tri-specific antibody construct having a cytokine receptor engager. The precursor construct comprises a Fab portion recognizing a NK cell surface antigen (e.g. NKG2A or NKG2D etc), an anti-TAA scFv, and a cytokine receptor engager (e.g. IL-15). In the embodiment shown here, the precursor tri-body construct is formed by two polypeptides. The anti-TAA scFv and the cytokine receptor engager are located C-terminal to the Fab region. Each polypeptide includes a regulatory domain N-terminal to the Fab binding domain, wherein a first sub-regulatory domain comprises a protease cleavage domain and a half-life prolonging domain (in this embodiment, a human serum albumin HSA), and a second sub-regulatory domain comprising a protease cleavage domain and a CAP component that reduces the ability of the Fab binding domain to bind its target. In embodiments not shown, the two sub-regulatory domains N-terminal to the Fab region could be linked to a polypeptide chain different from the one shown here, for example the HSA regulatory domain could be linked N-terminal to the Variable Light chain (VL1) of the Fab fragment and the CAP regulatory domain could be linked N-terminal to the Heavy chain variable region (VH1) of the Fab fragment. In the embodiment shown here for the anti-TAA-scFv, the N-terminal to C-terminal order is a Light chain variable region (VL2) followed by a linker (L4) followed by a Heavy chain variable region (VH2) followed by a linker (L5). In embodiments not shown, the N-terminal to C-terminal order of the anti-TAA scFv may be reversed, i.e. VH2-L4-VL2-L5. Other components in the figure are the same as those shown above.

FIG. 4C presents one embodiment of an active tri-specific (tribody) antibody construct derived from the precursor construct of FIG. 4A.

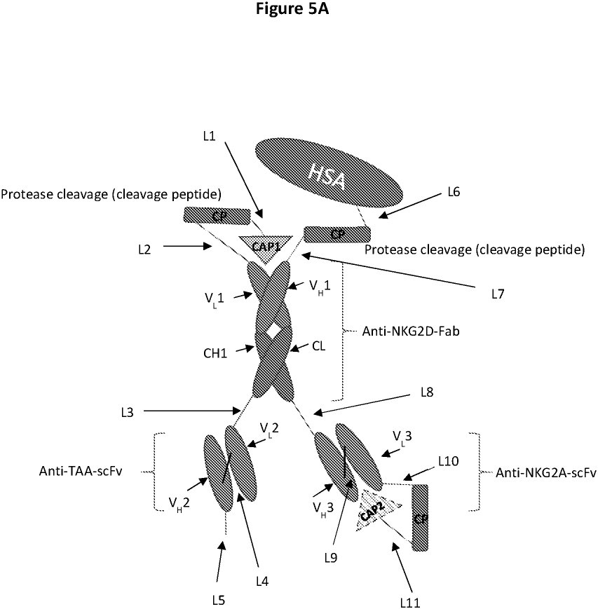

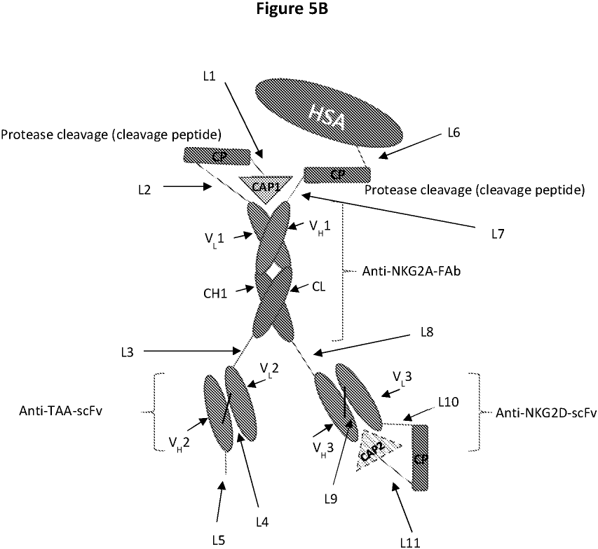

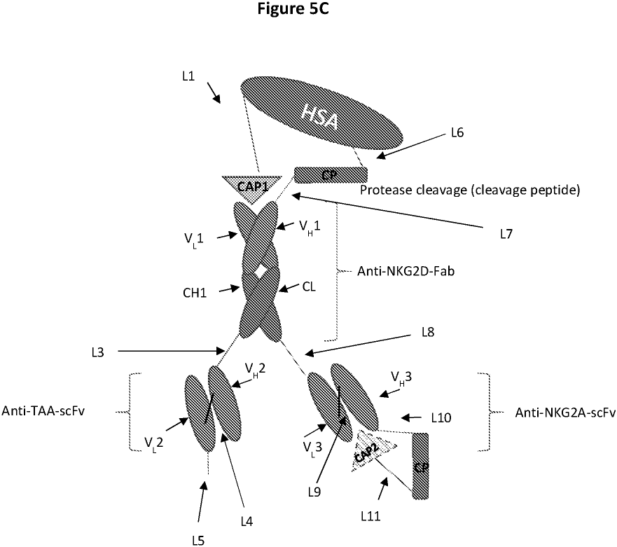

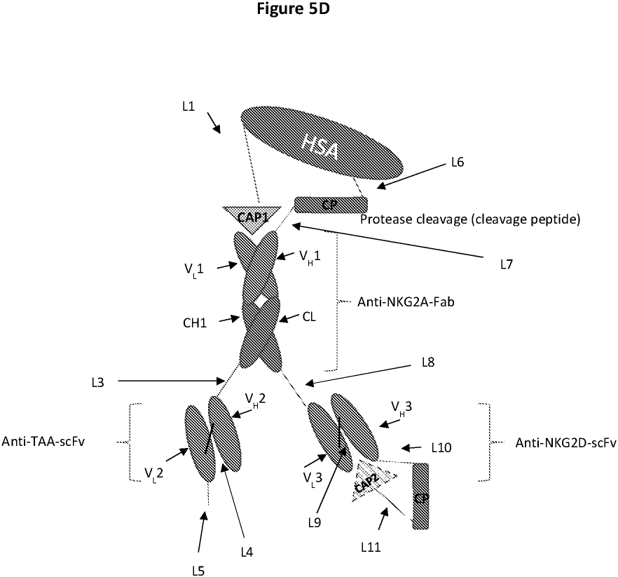

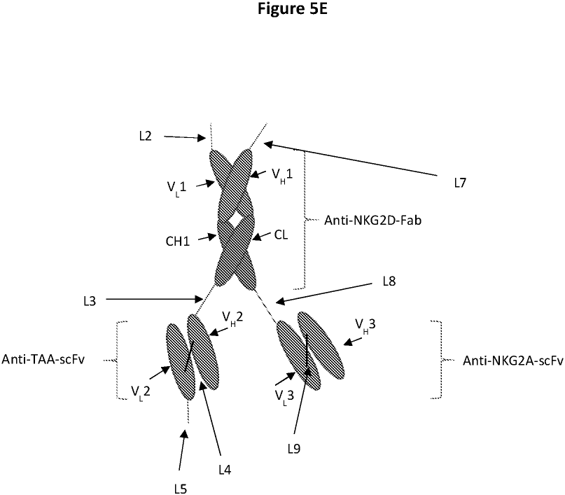

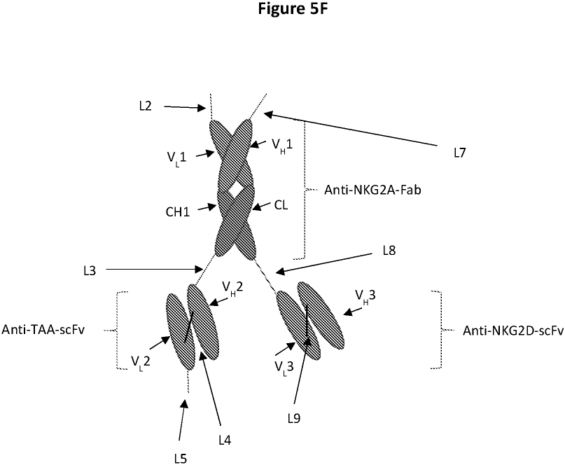

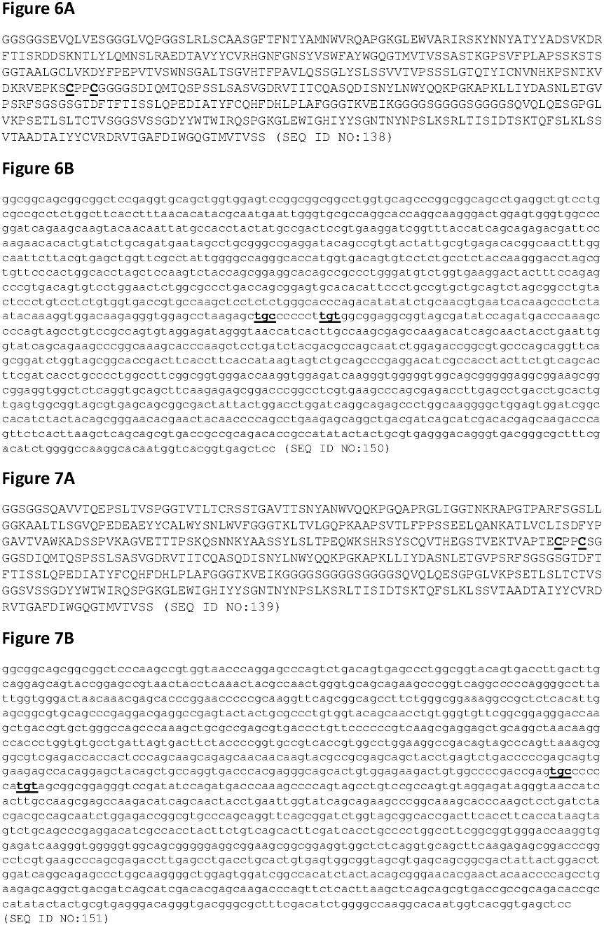

FIG. 5A presents one embodiment of a precursor tri-specific antibody (tri-body) construct having dual specificities to NK cells, both with regulated anti-NK cell binding. The precursor construct comprises three antibody binding domains, the Fab portion recognizes a Natural Killer (NK) cell surface antigen (e.g. NKG2D), a scFv recognizes a TAA, and another scFv recognizes another NK cell surface antigen (e.g. NKG2A). In the embodiment shown here, the precursor tri-body construct is formed by two polypeptides, the two scFv binding domains are C-terminal to the anti-NK Fab binding domain. In one embodiment, the anti-NK scFv binding domain may further include a regulatory module (third regulatory domain) comprising linkers (L10, L11), a protease cleavable peptide (CP) domain, and a CAP component that reduces the ability of the anti-NK binding domain to bind the surface antigen of a NK cell. There are regulatory domains N-terminal to the anti-NK Fab binding domain, wherein a first sub-regulatory domain comprises a protease cleavage domain and a half-life prolonging domain (in this embodiment, a human serum albumin HSA), and a second sub-regulatory domain comprising a protease cleavage domain and a CAP component that reduces the ability of the Fab binding domain to bind its target. In embodiments not shown, the two sub-regulatory domains N-terminal to the Fab region could be linked to a polypeptide chain different from the one shown here, for example the HSA regulatory domain could be linked N-terminal to the Light chain variable region (VL1) of the Fab fragment and the CAP regulatory domain could be linked N-terminal to the Heavy chain variable region (VH1) of the Fab fragment. In the embodiment shown here for the anti-TAA-scFv, the N-terminal to C-terminal order is a Light chain variable region (VL2) followed by a linker (L4) followed by a Heavy chain variable region (VH2) followed by a linker (L5), whereas the N-terminal to C-terminal order of the anti-NK-scFv is a Heavy chain variable region (VH3) followed by a linker (L9) followed by a Light chain variable region (VL3) followed by a linker (L10) followed by protease cleavable domain, linker (L11) which is followed by a CAP component that reduces the ability of the anti-NK binding domain to bind a surface antigen on an NK cell. The order of the light chain variable (VL) and heavy chain variable (VH) regions for the two scFv domains, from N-terminal to C-terminal, may be different from one another (as shown here), or be the same as one another (not shown).

FIG. 5B presents another embodiment of a precursor tri-specific antibody construct of FIG. 5A, wherein the Fab portion recognizes a Natural Killer (NK) cell surface antigen (e.g. NKG2A), a scFv recognizes a TAA, and another scFv recognizes another NK cell surface antigen (e.g. NKG2D). Other components in the figure are the same as those in FIG. 5A.

FIG. 5C presents one embodiment of an active tri-specific (tribody) antibody construct having dual specificities to NK cells, both with regulated anti-NK cell binding, wherein the regulatory domain N-terminal to the Fab comprises a single regulatory domain comprising a CAP domain, a HSA sequence, and a protease cleavable linker on the same polypeptide. The precursor construct comprises three antibody binding domains, the Fab portion recognizes a Natural Killer (NK) cell surface antigen (e.g. NKG2D), a scFv recognizes a TAA, and another scFv recognizes another NK cell surface antigen (e.g. NKG2A). In the embodiment shown here, the precursor tri-body construct is formed by two polypeptides, The two scFv binding domains are C-terminal to the anti-NK Fab binding domain. In one embodiment, the anti-NK scFv binding domain may further include a regulatory module (third regulatory domain) comprising linkers (L10, L11), a protease cleavable peptide (CP) domain, and a CAP component that reduces the ability of the anti-NK binding domain to bind the surface antigen of a NK cell. The regulatory domain N-terminal to the Fab comprises a single regulatory domain comprising a CAP domain, a HSA sequence, and a protease cleavable linker on the same polypeptide. In embodiments not shown, the regulatory domains N-terminal to the Fab region could be linked to a polypeptide chain different from the one shown here, for example the HSA/CAP regulatory domain could be linked N-terminal to the Variable Light chain (VL1) of the Fab fragment. In the embodiment shown here for the anti-TAA-scFv, the N-terminal to C-terminal order is a Variable Heavy chain region (VH2) followed by a linker (L4) followed by a Variable Light chain region (VL2) followed by a linker (L5), whereas the N-terminal to C-terminal order of the anti-NK-scFv is a Variable Light chain region (VL3) followed by a linker (L9) followed by a Variable Heavy chain region (VH3) followed by a linker (L10) followed by protease cleavable domain, linker (L11) which is followed by a CAP component that reduces the ability of the anti-NK binding domain to bind a surface antigen on an NK cell. The order of the light chain variable (VL) and heavy chain variable (VH) regions for the two scFv domains, from N-terminal to C-terminal, may be different from one another (as shown here), or be the same as one another (not shown).

FIG. 5D presents another embodiment of a precursor tri-specific antibody construct of FIG. 5C, wherein the Fab portion recognizes a Natural Killer (NK) cell surface antigen (e.g. NKG2A), a scFv recognizes a TAA, and another scFv recognizes another NK cell surface antigen (e.g. NKG2D). Other components in the figure are the same as those in FIG. 5C.

FIG. 5E presents one embodiment of an active tri-specific (tribody) antibody construct derived from the precursor of FIG. 5C, wherein the regulatory domains N-terminal to the Fab domain and the regulatory domain linked to the anti-NK scFv have all been removed.

FIG. 5F presents one embodiment of an active tri-specific (tribody) antibody construct derived from the precursor of FIG. 5D, wherein the regulatory domains N-terminal to the Fab domain and the regulatory domain linked to the anti-NK scFv have all been removed.

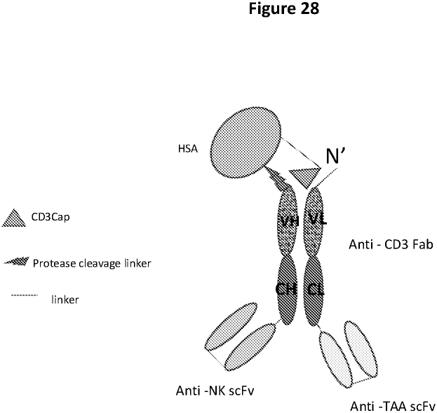

FIG. 28 presents one embodiment of a precursor tri-specific antibody (Tribody) construct having antibody binding domains and regulatory domains as described herein, the Fab portion recognizes a CD3 surface antigen, one scFv recognizes a tumor associated antigen (TAA), and one scFv recognizes a NK cell surface antigen (e.g. NKG2A or NKG2D). The regulatory domain N-terminal to the Fab comprises a CAP domain, a HSA sequence, and a protease cleavable linker on the same polypeptide. The principles and description discussed in the embodiments above also apply to the embodiments disclosed herein and below. For example, as discussed above, the regulatory domain in this embodiment can be N-terminal to the VH or VL region of the Fab; and the VH and VL of the scFv can be in the order of, from N-terminal to C-terminal, VH-VL or VL-VH as discussed above.

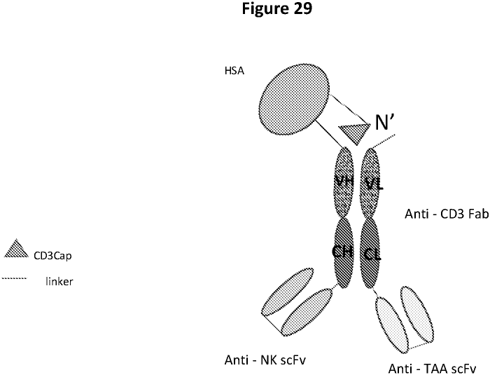

FIG. 29 presents another embodiment of the Tribody construct of FIG. 28, in that the regulatory domain comprises a CAP domain that inhibits binding to CD3 and a HSA sequence without a protease cleavable linker. As discussed above, there are a number of permutations on arranging the various components of the Tribody construct; for example, the regulatory domain in this embodiment can be N-terminal to the VH or VL region of the Fab; and/or the VH and VL of the scFv can be in the order of, from N-terminal to C-terminal, VH-VL or VL-VH as discussed above.

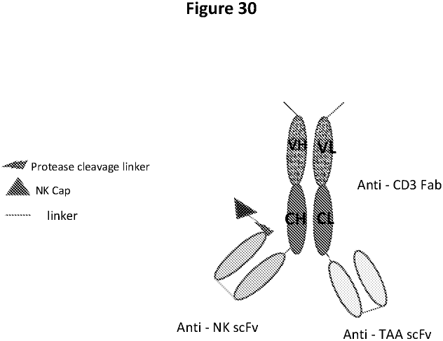

FIG. 30 presents one embodiment of a precursor Tribody construct having antibody binding domains and regulatory domains as described herein, the Fab portion recognizes a CD3 surface antigen, one scFv recognizes a tumor associated antigen (TAA), and one scFv recognizes a NK cell surface antigen (e.g. NKG2A or NKG2D). The anti-NK scFv further comprises a regulatory domain comprising a protease cleavable linker and a CAP domain that inhibits binding to the NK cell surface antigen. As discussed above, the VH and VL in each of the scFv can be in the order of, from N-terminal to C-terminal, VH-VL or VL-VH.

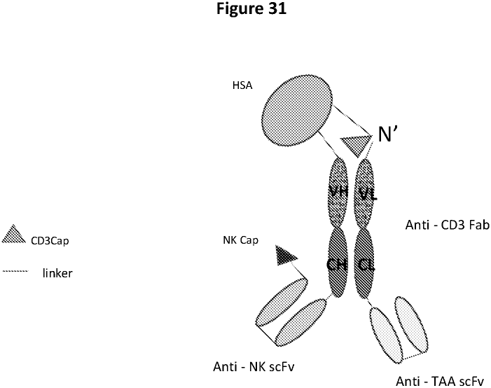

FIG. 31 presents one embodiment of a precursor Tribody construct having antibody binding domains and regulatory domains as described herein, the Fab portion recognizes a CD3 surface antigen, one scFv recognizes a tumor associated antigen (TAA), and one scFv recognizes a NK cell surface antigen (e.g. NKG2A or NKG2D). The Fab portion further comprises a regulatory domain comprising a CAP domain that inhibits binding to CD3, and the anti-NK scFv further comprises a CAP domain that inhibits binding to the NK cell surface antigen. In one embodiment, one or both of the regulatory domains do not comprise a protease cleavable linker. In another embodiment, one or both of the regulatory domains may further comprise a protease cleavable linker. Moreover, other permutations discussed above can also be applied here; for example, the regulatory domain N-terminal to the Fab can be linked to the VH or VL of the Fab; and/or the VH and VL in each of the scFv can be in the order of, from N-terminal to C-terminal, VH-VL or VL-VH.

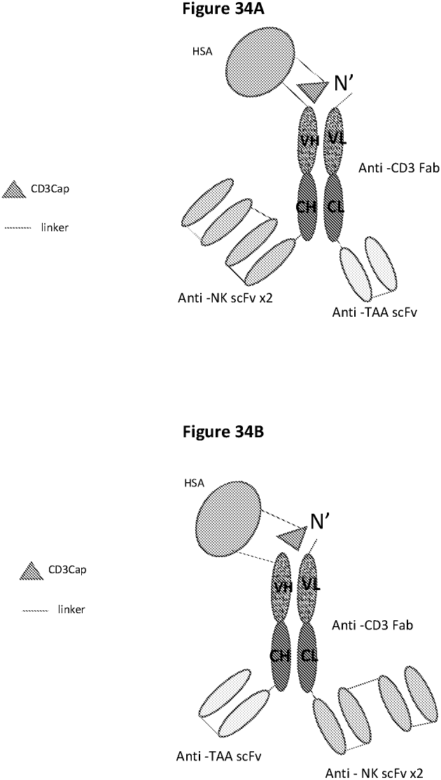

FIG. 32 presents one embodiment of a precursor Tribody construct having antibody binding domains and regulatory domains as described herein, the Fab portion recognizes a CD3 surface antigen, one scFv recognizes a tumor associated antigen (TAA), and one scFv region comprising two scFvs that recognize one or two NK cell surface antigen (e.g. NKG2A and/or NKG2D). As discussed above, the VH and VL of the scFv can be in the order of, from N-terminal to C-terminal, VH-VL or VL-VH. Thus, the VH and VL of the 2 anti-NK scFvs can be in the order of, from N-terminal to C-terminal, VH-VL-VH-VL, VH-VL-VL-VH, VL-VH-VH-VL, or VL-VH-VL-VH. These VH/VL arrangements for the 2 scFv can also be applied to the embodiments shown in FIGS. 33-34.

FIGS. 33A-33B present some other embodiments of the Tribody construct of FIG. 32, further having a regulatory domain N-terminal to the Fab comprising a CAP domain, a HSA sequence, and a protease cleavable linker on the same polypeptide. FIG. 33A shows the anti-TAA scFv is located C-terminal to the CL of the anti-CD3 Fab, whereas the 2 anti-NK scFvs are located C-terminal to the CH of the anti-CD3 Fab. FIG. 33B shows the anti-TAA scFv is located C-terminal to the CH of the anti-CD3 Fab, whereas the 2 anti-NK scFvs are located C-terminal to the CL of the anti-CD3 Fab. The regulatory domain N-terminal to the Fab can be linked to the VH or VL of the Fab.

FIG. 34A presents another embodiment of the Tribody construct of FIG. 33A, wherein the regulatory domain does not contain a protease cleavable linker. FIG. 34B presents another embodiment of the Tribody construct of FIG. 33B, wherein the regulatory domain does not contain a protease cleavable linker. The regulatory domain N-terminal to the Fab can be linked to the VH or VL of the Fab.

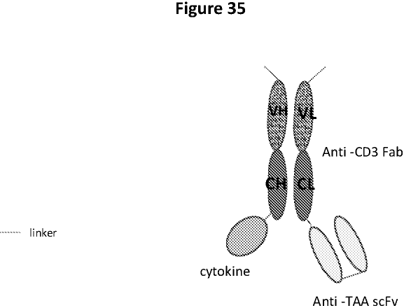

FIG. 35 presents one embodiment of a precursor Tribody construct having antibody binding domains and regulatory domains as described herein, the Fab portion recognizes a CD3 surface antigen, one scFv recognizes a tumor associated antigen (TAA), and one binding domain comprising a cytokine receptor engager (e.g. IL-15). As discussed above, the VH and VL of the scFv can be arranged in the order of VH-VL or VL-VH.

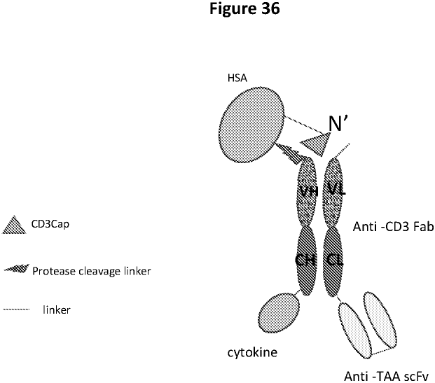

FIG. 36 presents another embodiment of the Tribody construct of FIG. 35, further having a regulatory domain N-terminal to the Fab comprising a CAP domain, a HSA sequence, and a protease cleavable linker on the same polypeptide. As discussed above, the regulatory domain in this embodiment can be N-terminal to the VH or VL region of the Fab.

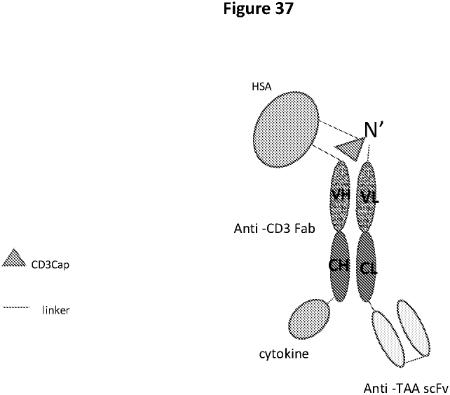

FIG. 37 presents another embodiment of the Tribody construct of FIG. 36. The components shown in the figure are the same as those in FIG. 36, except the regulatory domain N-terminal to the Fab does not comprise a protease cleavable linker.

### Examples of Full Length Sequences for Precursor Tri-specific Antibody Constructs

As discussed herein, the present disclosure provides precursor Tribody construct polypeptides comprising various components. In one embodiment, the precursor Tribody construct comprises two polypeptides: polypeptide A and polypeptide B, each comprising various binding domains and regulatory components disclosed herein. These binding domains and regulatory components would have various alternative placement, order and arrangement as disclosed herein. For example, the regulatory domain can be linked to polypeptide A or polypeptide B, and the VH and VL of each binding domain can be arranged in the order of VH-VL or VL-VH. Thus, there would be various permutations of arranging the binding domains and regulatory components on polypeptide A and polypeptide B. In view of the disclosure provided herein, one of ordinary skill in the art would readily combine these various components into the precursor tri-specific antibody construct polypeptides described herein. The present disclosure encompasses all the possible permutations of these binding domains and regulatory components on polypeptide A and polypeptide B.

In some other embodiments, the present disclosure encompasses tri-specific antibodies (Tribodies) derived from the ProTribody constructs disclosed herein, each Tribody comprises two polypeptides: polypeptide A and polypeptide B, each comprising various binding domains but without regulatory components disclosed herein. The binding domains would have various alternative placement, order and arrangement as disclosed herein.

In one embodiment of the present Tribody construct, the Fab portion recognizes the CD3 surface antigen, one scFv recognizes the tumor associated antigen EGFR, and one scFv recognizes the NK cell surface antigen NKG2A (see FIG. 2F). Examples of amino acid sequences for polypeptide A and polypeptide B of this Tribody construct include, but are not limited to, SEQ ID NOs:180 and 32, or SEQ ID NOs:180 and 141 (IM1064 or IM1065).

In one embodiment of the present Tribody construct, the Fab portion recognizes the CD3 surface antigen, one scFv recognizes the tumor associated antigen EGFR, and one scFv recognizes the NK cell surface antigen NKG2D (see FIG. 2F). Examples of amino acid sequences for polypeptide A and polypeptide B of this Tribody construct include, but are not limited to, SEQ ID NOs:178 and 32, or SEQ ID NOs:178 and 141 (IM1066 or IM1067).

In one embodiment of the present Tribody construct, the Fab portion recognizes the CD3 surface antigen, one scFv recognizes the tumor associated antigen 5T4, and one scFv recognizes the NK cell surface antigen NKG2A (see FIG. 2F). Examples of amino acid sequences for polypeptide A and polypeptide B of this Tribody construct include, but are not limited to, SEQ ID NOs:180 and 177, SEQ ID NOs:181 and 177, SEQ ID NOs:184 and 177, SEQ ID NOs:185 and 177, SEQ ID NOs:180 and 354, SEQ ID NOs:180 and 355, SEQ ID NOs:180 and 356, SEQ ID NOs:180 and 357, SEQ ID NOs:186 and 358, SEQ ID NOs:188 and 359, SEQ ID NOs:180 and 363, SEQ ID NOs:180 and 368, SEQ ID NOs:180 and 369, SEQ ID NOs:180 and 370, SEQ ID NOs:180 and 371, SEQ ID NOs:180 and 372, SEQ ID NOs:180 and 373, SEQ ID NOs:180 and 374, SEQ ID NOs:180 and 375, SEQ ID NOs:180 and 376, SEQ ID NOs:180 and 377, SEQ ID NOs:180 and 378, SEQ ID NOs:180 and 379, SEQ ID NOs:180 and 380, SEQ ID NOs:180 and 381, SEQ ID NOs:180 and 382, SEQ ID NOs:180 and 383, SEQ ID NOs:180 and 384, SEQ ID NOs:180 and 385, SEQ ID NOs:180 and 386, SEQ ID NOs:180 and 387, SEQ ID NOs:180 and 388, SEQ ID NOs:180 and 389, SEQ ID NOs:180 and 390, SEQ ID NOs:180 and 391, SEQ ID NOs:180 and 392, SEQ ID NOs:180 and 393, SEQ ID NOs:180 and 394, SEQ ID NOs:180 and 395, SEQ ID NOs:180 and 396, SEQ ID NOs:180 and 397, or SEQ ID NOs:219 and 358 (any one of IM1062, IM1063, IM1093, IM1094, IM1127. IM1128, IM1129, IM1130, IM1153, IM1155, IM1198, IM1199, IM1200, IM1201, IM1202, IM1203, IM1204, IM1205, IM1206, IM1207, IM1208, IM1209, IM1210, IM1211, IM1212, IM1213, IM1214, IM1215, IM1216, IM1217, IM1218, IM1219, IM1220, IM1221, IM1222, IM1223, IM1224, IM1225, IM1226, IM1227, IM1230, IM1236).

In one embodiment of the present Tribody construct, the Fab portion recognizes the CD3 surface antigen, one scFv recognizes the tumor associated antigen 5T4, and one scFv recognizes the NK cell surface antigen NKG2D (see FIG. 2F). Examples of amino acid sequences for polypeptide A and polypeptide B of this Tribody construct include, but are not limited to, SEQ ID NOs:178 and 177, SEQ ID NOs: 179 and 177, SEQ ID NOs:182 and 177, SEQ ID NOs:183 and 177, SEQ ID NOs:178 and 344, SEQ ID NOs:178 and 345, SEQ ID NOs:178 and 346, SEQ ID NOs:178 and 347, SEQ ID NOs:178 and 348, SEQ ID NOs:178 and 349, SEQ ID NOs:178 and 350, SEQ ID NOs:178 and 351, SEQ ID NOs:178 and 352, SEQ ID NOs:178 and 353, SEQ ID NOs:178 and 354, SEQ ID NOs:178 and 355, SEQ ID NOs:178 and 356, SEQ ID NOs:178 and 357, SEQ ID NOs:187 and 358, SEQ ID NOs:189 and 359, SEQ ID NOs:178 and 360, SEQ ID NOs:178 and 361, SEQ ID NOs:178 and 362, SEQ ID NOs:178 and 363, SEQ ID NOs:178 and 364, SEQ ID NOs:178 and 365, SEQ ID NOs:178 and 366, SEQ ID NOs:178 and 367, SEQ ID NOs:218 and 358, SEQ ID NOs:223 and 392, SEQ ID NOs:224 and 392, SEQ ID NOs:225 and 392, SEQ ID NOs:226 and 392, SEQ ID NOs:227 and 392, SEQ ID NOs:228 and 392, SEQ ID NOs:229 and 392, or SEQ ID NOs:230 and 392 (any one of IM1060, IM1061, IM1091, IM1092, IM1100, IM1101, IM1102, IM1103, IM1104, IM1105, IM1106, IM1107, IM1108, IM1109, IM1132, IM1133, IM1134, IM1135, IM1154, IM1156, IM1175, IM1176, IM1177, IM1178, IM1179, IM1180, IM1181, IM1182, IM1229, IM1244, IM1245, IM1246, IM1247, IM1248, IM1249, IM1250, IM1251).

In one embodiment of the present Tribody construct, the Fab portion recognizes the CD3 surface antigen, one scFv recognizes the tumor associated antigen 5T4, and one scFv recognizes the NK cell surface antigen CD16 (see FIG. 2F). Examples of amino acid sequences for polypeptide A and polypeptide B of this Tribody construct include, but are not limited to, SEQ ID NOs:252 and 177, or SEQ ID NOs:254 and 358 (IM1280 or IM1282).

In one embodiment of the present precursor Tribody construct, the Fab portion recognizes the CD3 surface antigen, one scFv recognizes the tumor associated antigen 5T4, and one scFv recognizes the NK cell surface antigen NKG2A. The anti-CD3 Fab further comprise a regulatory domain comprising a CAP domain, a HSA sequence, and a protease cleavable linker (see FIG. 28). Examples of amino acid sequences for polypeptide A and polypeptide B of this Tribody construct include, but are not limited to, SEQ ID NOs:206 and 177, SEQ ID NOs:207 and 177, SEQ ID NOs:208 and 177, SEQ ID NOs:209 and 177, SEQ ID NOs:210 and 177, SEQ ID NOs:211 and 177, SEQ ID NOs:212 and 177, SEQ ID NOs:213 and 177, SEQ ID NOs:206 and 363, SEQ ID NOs:210 and 363, SEQ ID NOs:206 and 398, SEQ ID NOs:210 and 398, SEQ ID NOs:256 and 392, SEQ ID NOs:257 and 392, SEQ ID NOs:258 and 392, SEQ ID NOs:259 and 392, or SEQ ID NOs:260 and 392 (any one of IM1184, IM1185, IM1186, IM1187, IM1188, IM1189, IM1190, IM1191, IM1232, IM1233, IM1237, IM1238, IM1240, IM1241, IM1284, IM1285, IM1286, IM1287, IM1288).

In some embodiments, a precursor tri-specific antibody construct comprises polypeptide A and polypeptide B, wherein polypeptide A and polypeptide B comprise amino acid sequences have the sequences of SEQ ID NOs: 206 and 398, respectively.

In one embodiment of the present precursor Tribody construct, the Fab portion recognizes the CD3 surface antigen, one scFv recognizes the tumor associated antigen 5T4, and one scFv recognizes the NK cell surface antigen CD16. The anti-CD3 Fab further comprises a regulatory domain comprising a CAP domain, a HSA sequence, and a protease cleavable linker (see FIG. 28). Examples of amino acid sequences for polypeptide A and polypeptide B of this Tribody construct include, but are not limited to, SEQ ID NOs:253 and 177 (IM1281).

In one embodiment of the present precursor Tribody construct, the Fab portion recognizes the CD3 surface antigen, one scFv recognizes the tumor associated antigen 5T4, and one scFv recognizes the NK cell surface antigen NKG2A. The anti-CD3 Fab further comprise a regulatory domain comprising a CAP domain, a HSA sequence, but without a protease cleavable linker (see FIG. 29). Examples of amino acid sequences for polypeptide A and polypeptide B of this Tribody construct include, but are not limited to, SEQ ID NOs:214 and 177, SEQ ID NOs:215 and 177, SEQ ID NOs:216 and 177, SEQ ID NOs:217 and 177, SEQ ID NOs:222 and 177, SEQ ID NOs:214 and 363, or SEQ ID NOs:214 and 398 (any one of IM1193, IM1194, IM1195, IM1196, IM1234, IM1239, IM1242).

In one embodiment of the present precursor Tribody construct, the Fab portion recognizes the CD3 surface antigen, one scFv recognizes the tumor associated antigen 5T4, and one scFv recognizes the NK cell surface antigen CD16. The anti-CD3 Fab further comprises a regulatory domain comprising a CAP domain, a HSA sequence, but without a protease cleavable linker (see FIG. 29). Examples of amino acid sequences for polypeptide A and polypeptide B of this Tribody construct include, but are not limited to, SEQ ID NOs:255 and 177 (IM1283).

In one embodiment of the present precursor Tribody construct, the Fab portion recognizes the CD3 surface antigen, one scFv recognizes the tumor associated antigen 5T4, and one scFv recognizes the NK cell surface antigen NKG2A. The anti-NK scFv further comprises a regulatory domain comprising a CAP domain and a protease cleavable linker (see FIG. 30). Examples of amino acid sequences for polypeptide A and polypeptide B of this Tribody construct include, but are not limited to, SEQ ID NOs: 198 and 177, SEQ ID NOs: 199 and 177, SEQ ID NOs:200 and 177, SEQ ID NOs:201 and 177, SEQ ID NOs:202 and 177, SEQ ID NOs:203 and 177, SEQ ID NOs:204 and 177, SEQ ID NOs:205 and 177, SEQ ID NOs: 231 and 392, SEQ ID NOs: 232 and 392, SEQ ID NOs: 233 and 392, SEQ ID NOs: 234 and 392, or SEQ ID NOs: 235 and 392 (any one of IM1166, IM1167, IM1168, IM1169, IM1170, IM1171, IM1172, IM1173, IM1253, IM1254, IM1255, IM1256, IM1257).

In one embodiment of the present precursor Tribody construct, the Fab portion recognizes the CD3 surface antigen, one scFv recognizes the tumor associated antigen 5T4, and one scFv recognizes the NK cell surface antigen NKG2D. The anti-NK scFv further comprises a regulatory domain comprising a CAP domain and a protease cleavable linker (see FIG. 30). Examples of amino acid sequences for polypeptide A and polypeptide B of this Tribody construct include, but are not limited to, SEQ ID NOs: 190 and 177, SEQ ID NOs: 191 and 177, SEQ ID NOs:192 and 177, SEQ ID NOs:193 and 177, SEQ ID NOs:194 and 177, SEQ ID NOs:195 and 177, SEQ ID NOs:196 and 177, SEQ ID NOs:197 and 177 (any one of IM1158, IM1159, IM1160, IM1161, IM1162, IM1163, IM1164, IM1165).

In one embodiment of the present precursor Tribody construct, the Fab portion recognizes the CD3 surface antigen, one scFv recognizes the tumor associated antigen 5T4, and one scFv recognizes the NK cell surface antigen NKG2A. The anti-NK scFv further comprises a regulatory domain comprising a CAP domain and a protease cleavable linker, and the anti-CD3 Fab further comprises a regulatory domain comprising a CAP domain, a HSA sequence, and a protease cleavable linker (see FIG. 3C). As discussed herein, the VH and VL domain of the scFv can be arranged, from the N-terminal to the C-terminal, in the order of VH-VL or VL-VH. Examples of amino acid sequences for polypeptide A and polypeptide B of this Tribody construct include, but are not limited to, SEQ ID NOs:236 and 392, SEQ ID NOs:237 and 392, SEQ ID NOs:238 and 392, SEQ ID NOs:239 and 392, or SEQ ID NOs:240 and 392 (any one of IM1258, IM1259, IM1260, IM1261, IM1262).

In one embodiment of the present precursor Tribody construct, the Fab portion recognizes the CD3 surface antigen, one scFv recognizes the tumor associated antigen 5T4, and one scFv recognizes the NK cell surface antigen NKG2A. The anti-NK scFv further comprises a regulatory domain comprising a CAP domain without a protease cleavable linker, and the anti-CD3 Fab further comprises a regulatory domain comprising a CAP domain, a HSA sequence, without a protease cleavable linker (see FIG. 31). Examples of amino acid sequences for polypeptide A and polypeptide B of this Tribody construct include, but are not limited to, SEQ ID NOs:241 and 392, SEQ ID NOs:242 and 392, SEQ ID NOs:243 and 392, SEQ ID NOs:244 and 392, or SEQ ID NOs:245 and 392 (any one of IM1263, IM1264, IM1265, IM1266, IM1267).

In one embodiment of the present Tribody construct, the Fab portion recognizes the CD3 surface antigen, one scFv recognizes the tumor associated antigen 5T4, and one binding region comprising two scFv that recognize the NK cell surface antigen NKG2A (see FIG. 32). Examples of amino acid sequences for polypeptide A and polypeptide B of this Tribody construct include, but are not limited to, SEQ ID NOs:246 and 177, or SEQ ID NOs:247 and 358 (IM1272 or IM1273). In some embodiments, a precursor tri-specific antibody construct upon cleavage of cleavable peptides yields a tri-specific antibody derived from the precursor tri-specific antibody construct comprising a polypeptide A and a polypeptide B, wherein said polypeptide A and polypeptide B comprise amino acid sequences having the sequences of SEQ ID NOs: 246 and 177 (IM1272). A tri-specific antibody derived from a precursor tri-specific antibody construct disclosed herein comprises polypeptide A and polypeptide B, said polypeptide A and polypeptide B comprising amino acid sequences having the sequences of SEQ ID NOs: 246 and 177, respectively (IM1272).

In one embodiment of the present precursor Tribody construct, the Fab portion recognizes the CD3 surface antigen, one scFv recognizes the tumor associated antigen 5T4, and one binding region comprising two scFv that recognize the NK cell surface antigen NKG2A, and further having a regulatory domain N-terminal to the Fab comprising a CAP domain, a HSA sequence, and a protease cleavable linker on the same polypeptide. In one embodiment, the anti-5T4 scFv is located C-terminal to the CL of the anti-CD3 Fab, whereas the 2 anti-NKG2A scFvs are located C-terminal to the CH of the anti-CD3 Fab (see FIG. 33A). Examples of amino acid sequences for polypeptide A and polypeptide B of this Tribody construct include, but are not limited to, SEQ ID NOs:248 and 177, or SEQ ID NOs:248 and 392 (IM1274 or IM1276).

In one embodiment of the present precursor Tribody construct, the Fab portion recognizes the CD3 surface antigen, one scFv recognizes the tumor associated antigen 5T4, and one binding region comprising two scFv that recognize the NK cell surface antigen NKG2A, and further having a regulatory domain N-terminal to the Fab comprising a CAP domain, a HSA sequence, and a protease cleavable linker on the same polypeptide. In one embodiment, the anti-5T4 scFv is located C-terminal to the CH of the anti-CD3 Fab, whereas the 2 anti-NKG2A scFvs are located C-terminal to the CL of the anti-CD3 Fab (see FIG. 33B). Examples of amino acid sequences for polypeptide A and polypeptide B of this Tribody construct include, but are not limited to, SEQ ID NOs:250 and 399 (IM1278).

In one embodiment of the present precursor Tribody construct, the Fab portion recognizes the CD3 surface antigen, one scFv recognizes the tumor associated antigen 5T4, and one binding region comprising two scFv that recognize the NK cell surface antigen NKG2A, and further having a regulatory domain N-terminal to the Fab comprising a CAP domain, a HSA sequence, without a protease cleavable linker. In one embodiment, the anti-5T4 scFv is located C-terminal to the CL of the anti-CD3 Fab, whereas the 2 anti-NKG2A scFvs are located C-terminal to the CH of the anti-CD3 Fab (see FIG. 34A). Examples of amino acid sequences for polypeptide A and polypeptide B of this Tribody construct include, but are not limited to, SEQ ID NOs:249 and 177, or SEQ ID NOs:249 and 392 (IM1275 or IM1277).

In one embodiment of the present precursor Tribody construct, the Fab portion recognizes the CD3 surface antigen, one scFv recognizes the tumor associated antigen 5T4, and one binding region comprising two scFv that recognize the NK cell surface antigen NKG2A, and further having a regulatory domain N-terminal to the Fab comprising a CAP domain, a HSA sequence, without a protease cleavable linker. In one embodiment, the anti-5T4 scFv is located C-terminal to the CH of the anti-CD3 Fab, whereas the 2 anti-NKG2A scFvs are located C-terminal to the CL of the anti-CD3 Fab (see FIG. 34B). Examples of amino acid sequences for polypeptide A and polypeptide B of this Tribody construct include, but are not limited to, SEQ ID NOs:251 and 399 (IM1279).

In some embodiments, the first and second binding domains each comprise a single chain variable fragment (scFv). A skilled artisan would appreciate that a scFv is not actually a fragment of an antibody, but instead is a fusion polypeptide comprising the heavy chain variable (VH) and light chain variable (VL) regions of an immunoglobulin, connected by a short linker peptide of ten to about 25 amino acids.

In some embodiments, the third binding domain comprises a Fab fragment, wherein the first binding domain is attached to the C-terminal end of the light chain constant region (CL) of the Fab and the second domain is attached at the C-terminal end of the heavy chain constant region (CH) of the Fab. Alternatively, the third binding domain comprises a Fab fragment, wherein the first binding domain is attached to the C-terminal end of the heavy chain constant region (CH) of the Fab and the second domain is attached at the C-terminal end of the light chain constant region (CL) of the Fab.

In some embodiments, the third binding domain comprises a Fab fragment, wherein a first regulatory domain, for example a CAP masking domain, is attached to the N-terminal end of the VL chain of the Fab and a second regulatory domain, for example an HSA HLP domain, is attached at the N-terminal end of the VH chain of the Fab. In some embodiments, the third binding domain comprises a Fab fragment, wherein a first regulatory domain, for example a CAP masking domain, is attached to the N-terminal end of the VH chain and a second regulatory domain, for example an HSA HLP domain, is attached at the N-terminal end of the VL chain.

In some embodiments, between the scFv of the first binding domain and the CL of the third binding domain there may be a linker sequence. In some embodiments, between the scFv of the first binding domain and the CH of the third binding domain there may be a linker sequence. In some embodiments, between the scFv of the second binding domain and the CL of the third binding domain there may be a linker sequence. In some embodiments, between the scFv of the second binding domain and the CH of the third binding domain there may be a linker sequence. In some embodiments, between the scFv of the first and second binding domains, and the CL and CH of the third binding domains, respectively, there may be linker sequences.

In some embodiments, between the first sub-regulatory domain and the VH chain of the third binding domain, there may be a linker sequence which is cleavable. In some embodiments, between the first sub-regulatory domain and the VL chain of the third binding domain, there may be a linker sequence which is cleavable. In some embodiments, between the second sub-regulatory domain and the VH chain of the third binding domain, there may be a linker sequence which is cleavable. In some embodiments, between the second sub-regulatory domain and the VL chain of the third binding domain, there may be a linker sequence which is cleavable. In some embodiments, between the first and second sub-regulatory domains, and the VH and VL chains of the third binding domains, respectively, there may be linker sequences which are cleavable.

These general formats are the basic structure that can be built upon to construct the precursor tri-specific (tribody) antibody constructs described herein (FIGS. 2-5).

In some embodiments, the regulatory domain comprises two sub-domains: a first sub-regulatory domain comprising a protease cleavable linker component and a serum half-life prolonging (HLP) domain (e.g. human serum albumin polypeptide sequence component), and the second sub-regulatory domain comprising protease cleavable linker and a CAP amino acid (masking) component (see e.g. FIG. 2A). In some embodiments, in addition to the first and second sub-regulatory domains, there is a third regulatory domain comprising a third protease cleavage domain and a CAP component that reduces the ability of the second binding domain to bind to its target antigen (see e.g. FIG. 3A). In some embodiments, there is only a single regulatory domain comprising a protease cleavage domain, a half-life prolonging (HLP) domain, and a CAP component that reduces the ability of a binding domain to bind to its target antigen (see FIG. 2E). The skilled artisan would appreciate that the presence of linkers, for example anyone of the linkers displayed in the constructs shown in FIGS. 2-5 provide flexibility to a polypeptide while not necessarily providing essential regulatory feature to the regulatory domain, such as is provided by a CAP (a masking activity) or by an HSA component (increased half-life).

In some embodiments, the third binding domain having a binding specificity to an immune effector molecule, for example but not limited to a CD3 epsilon chain (CD3ε) extracellular epitope, binds specifically to the CAP amino acid component. In some embodiments, the CAP component effectively blocks binding of the precursor tri-specific antibody construct to the immune effector target molecule, for example an antigen on T-cell or NK cell. In some embodiments, activation of cytotoxicity to a target is specifically masked by the CAP component. In some embodiments, wherein the regulatory domain comprises a cleavable CAP component, activation of cytotoxicity is limited to a tumor milieu (see FIG. 1).

In some embodiments, the CAP component comprises an amino acid sequence present within the human CD3 epsilon polypeptide chain. In some embodiments, the CAP component comprises an amino acid sequence present as part of the extracellular portion of the human CD3 epsilon chain. In some embodiments, the CAP component comprises an amino acid sequence selected from the amino acid sequence of the N-terminal end of human CD3 epsilon precursor polypeptide. In some embodiments, the CAP component comprises an amino acid sequence selected from the amino acid sequence of the N-terminal end of human CD3 epsilon mature polypeptide. In some embodiments, the CAP component comprises an amino acid sequence present as part of the extracellular portion of a NK cell surface antigen.

In one embodiment, the amino acid sequence of the precursor human CD3 epsilon is set for in SEQ ID NO: 1. Human CD3 epsilon is expressed in a precursor form, wherein amino acids 1-21 form the signal peptide. The amino acid sequence of the mature human CD3 epsilon is set forth in amino acids 22-207 of SEQ ID NO: 1, as set forth herein in SEQ ID NO: 2. In some embodiments, the extracellular epitope of human CD3 epsilon is located within the precursor sequence, as set forth in SEQ ID NO: 3. In some embodiments, the extracellular epitope of a mature human CD3 epsilon is located within amino acids 1-27 of the precursor sequence, and is set forth in SEQ ID NO: 4. In some embodiments, the extracellular epitope of human CD3 epsilon is located within amino acids QDGNEEMGGITQTPYKVSISGTTVILT (SEQ ID NO: 5; AA1-27).

In some embodiments, the amino acid sequence of a CAP component is set forth in SEQ ID NO: 5, or a homolog thereof. In some embodiments, the amino acid sequence of a CAP component is a selected contiguous sequence within SEQ ID NO: 4, or a homolog thereof.

In some embodiments, homologues of SEQ ID NO: 5 or of a CAP sequence selected from SEQ ID NO: 4, comprise polypeptides which are at least 50%, at least 55%, at least 60%, at least 65%, at least 70%, at least 75%, at least 80%, at least 85%, at least 87%, at least 89%, at least 91%, at least 93%, at least 95%, at least 96%, at least 97%, at least 98%, or at least 99% homologous to the amino acid sequence.

In some embodiments, homologues comprise polypeptides which are at least 50%, at least 55%, at least 60%, at least 65%, at least 70%, at least 75%, at least 80%, at least 85%, at least 87%, at least 89%, at least 91%, at least 93%, at least 95%, at least 96%, at least 97%, at least 98%, or at least 99% homologous to a human CD3 epsilon polypeptide or a portion thereof, as determined using BlastP software of the National Center of Biotechnology Information (NCBI) using default parameters.

A skilled artisan would appreciate that the term “homology”, and grammatical forms thereof, encompasses the degree of similarity between two or more structures. The term “homologous sequences” refers to regions in macromolecules that have a similar order of monomers. When used in relation to nucleic acid sequences, the term “homology” refers to the degree of similarity between two or more nucleic acid sequences (e.g., genes) or fragments thereof. Typically, the degree of similarity between two or more nucleic acid sequences refers to the degree of similarity of the composition, order, or arrangement of two or more nucleotide bases (or other genotypic feature) of the two or more nucleic acid sequences. The term “homologous nucleic acids” generally refers to nucleic acids comprising nucleotide sequences having a degree of similarity in nucleotide base composition, arrangement, or order. The two or more nucleic acids may be of the same or different species or group. The term “percent homology” when used in relation to nucleic acid sequences, refers generally to a percent degree of similarity between the nucleotide sequences of two or more nucleic acids.

When used in relation to polypeptide (or protein) sequences, the term “homology” refers to the degree of similarity between two or more polypeptide (or protein) sequences (e.g., genes) or fragments thereof. Typically, the degree of similarity between two or more polypeptide (or protein) sequences refers to the degree of similarity of the composition, order, or arrangement of two or more amino acid of the two or more polypeptides (or proteins). The two or more polypeptides (or proteins) may be of the same or different species or group. The term “percent homology” when used in relation to polypeptide (or protein) sequences, refers generally to a percent degree of similarity between the amino acid sequences of two or more polypeptide (or protein) sequences. The term “homologous polypeptides” or “homologous proteins” generally refers to polypeptides or proteins, respectively, that have amino acid sequences and functions that are similar. Such homologous polypeptides or proteins may be related by having amino acid sequences and functions that are similar but are derived or evolved from different or the same species using the techniques described herein.

In some embodiments, homologues comprise polypeptides which are at least 50%, at least 55%, at least 60%, at least 65%, at least 70%, at least 75%, at least 80%, at least 85%, at least 87%, at least 89%, at least 91%, at least 93%, at least 95%, at least 96%, at least 97%, at least 98%, or at least 99% homologous to a polypeptide or a portion thereof disclosed herein, as determined using BlastP software of the National Center of Biotechnology Information (NCBI) using default parameters. In some embodiments, homologues comprise a nucleotide sequences which is at least 50%, at least 55%, at least 60%, at least 65%, at least 70%, at least 75%, at least 80%, at least 85%, at least 87%, at least 89%, at least 91%, at least 93%, at least 95%, at least 96%, at least 97%, at least 98%, or at least 99% homologous to a nucleotide sequence or a portion thereof disclosed herein, as determined using BlastN software of the National Center of Biotechnology Information (NCBI) using default parameters.

In some embodiments, homology also encompasses deletion, insertion, or substitution variants, including an amino acid substitution, thereof and biologically active polypeptide fragments thereof. In one embodiment, the variant comprises conservative substitutions, or deletions, insertions, or substitutions that do not significantly alter the three-dimensional structure of the polypeptide component of interest described herein. In some embodiments, the deletion, insertion, or substitution does not alter the function of interest of the polypeptide component of interest disclosed herein.

In some embodiments, homology also encompasses deletion, insertion, or substitution variants, including an amino acid substitution thereof and biologically active polypeptide fragments thereof. In one embodiment, the variant comprises conservative substitutions, or deletions, insertions, or substitutions that do not significantly alter the three-dimensional structure of the CAP component, e.g., the portion of a human CD3 epsilon polypeptide present in the CAP component, particularly in the areas of the epitope recognized and bound by the third binding domain. In some embodiments, the deletion, insertion, or substitution does not alter the function of interest of the CAP component, which in some embodiment, is binding to the third binding domain, or reducing T-cell binding, or reducing T-cell activation, or any combination thereof.

In some embodiments, the CAP component comprises an amino acid sequence comprising a portion of a polypeptide binding domain binding to an NK antigen, particularly in the areas of the epitope recognized and bound by the NK binding domain. Thus, in some embodiments, a CAP component reduces the ability of the second and/or third binding domain to bind the extracellular epitope of an NK antigen.

In some embodiments, a CAP component is 6-110 amino acids long. In some embodiments, a CAP component is between about 6-10 amino acids long. In some embodiments, a CAP component is between about 10-20 amino acids long. In some embodiments, a CAP component is between about 20-30 amino acids long. In some embodiments, a CAP component is between about 20-40 amino acids long. In some embodiments, a CAP component is between about 30-40 amino acids long. In some embodiments, a CAP component is between about 40-60 amino acids long. In some embodiments, a CAP component is between about 60-80 amino acids long. In some embodiments, a CAP component is between about 80-100 amino acids long. In some embodiments, a CAP component is between about 80-110 amino acids long.

In some embodiments, a CAP component is 6 amino acids long. In some embodiments, a CAP component is 7 amino acids long. In some embodiments, a CAP component is 8 amino acids long. In some embodiments, a CAP component is 9 amino acids long. In some embodiments, a CAP component is 10 amino acids long. In some embodiments, a CAP component is 11 amino acids long. In some embodiments, a CAP component is 12 amino acids long. In some embodiments, a CAP component is 13 amino acids long. In some embodiments, a CAP component is 14 amino acids long.

In some embodiments, a CAP component is 15 amino acids long. In some embodiments, a CAP component is 16 amino acids long. In some embodiments, a CAP component is 17 amino acids long. In some embodiments, a CAP component is 18 amino acids long. In some embodiments, a CAP component is 19 amino acids long. In some embodiments, a CAP component is 20 amino acids long. In some embodiments, a CAP component is 21 amino acids long. In some embodiments, a CAP component is 22 amino acids long. In some embodiments, a CAP component is 23 amino acids long. In some embodiments, a CAP component is 24 amino acids long. In some embodiments, a CAP component is 25 amino acids long. In some embodiments, a CAP component is 26 amino acids long. In some embodiments, a CAP component is 27 amino acids long. In some embodiments, a CAP component is 28 amino acids long. In some embodiments, a CAP component is 29 amino acids long. In some embodiments, a CAP component is 30 amino acids long. In some embodiments, a CAP component is 31, 32, 33, 34, 35, 36, 37, 38, 39, or 40 amino acids long.

In some embodiments, the CAP component specifically binds to the third binding region, thereby reducing T-cell binding of the precursor construct. In some embodiments, the CAP component specifically binds to the third binding region, thereby inhibiting NK cell binding of the precursor construct. In some embodiments, the CAP component specifically binds to the third binding region, thereby reducing T-cell activation by the precursor construct. In some embodiments, the CAP component specifically binds to the third binding region, thereby inhibiting NK cell activation by the precursor construct.

In some embodiments, the CAP component specifically binds to the second binding region, thereby reducing NK cell binding of the precursor construct. In some embodiments, the CAP component specifically binds to the third binding region, thereby inhibiting NK cell binding of the precursor construct. In some embodiments, the CAP component specifically binds to the second binding region, thereby reducing NK cell activation of the precursor construct. In some embodiments, the CAP component specifically binds to the third binding region, thereby inhibiting NK cell activation by the precursor construct.

Examples of CAP components that inhibit binding to CD3 include, but are not limited to, polypeptides having the sequences of SEQ ID NO: 695, 696, or 697, or homologous sequences thereof as explained and determined above. Examples of CAP components that inhibit binding to NKG2A include, but are not limited to, polypeptides having the sequences of SEQ ID NO: 698, 699, 700, 701, 702, 703, or 704, or homologous sequences thereof as explained and determined above. Examples of CAP components that inhibit binding to NKG2D include, but are not limited to, polypeptides having the sequences of SEQ ID NO:705, 706, 707, or 708, or homologous sequences thereof as explained and determined above.

In some embodiments, a regulatory domain comprises a cleavable half-life prolonging domain. In some embodiments, a cleavable half-life prolonging domain comprises an HSA polypeptide.

In some embodiments, there is a linker between the components of the regulatory domains. In some embodiments, there is a linker between a regulatory domain and the N-terminus of the VH chain of the Fab fragment. In some embodiments, there is a linker between a regulatory domain and the N-terminus of the VL chain of the Fab fragment. In some embodiments, there is a linker between a regulatory domain and the N-terminus of the VH chain of the Fab fragment and a linker between a regulatory domain and the N-terminus of the VL chain of the Fab fragment. In some embodiments, a linker between components of the regulatory domain and the N-terminus of an Fab fragment polypeptide is a cleavable linker. In some embodiments, any of the linkers between components of the regulatory domain and the Fab polypeptide is a cleavable linker. In some embodiments, a linker between components of the regulatory domain and the Fab polypeptide is not cleavable.

In some embodiments, a regulatory domain comprises a cleavable serum half-life prolonging domain comprising a protease cleavable domain and a human serum albumin polypeptide (HSA). In some embodiments, the order of components in the regulatory domain is (N-terminal to C-terminal) HSA-L-protease cleavable domain, wherein L is a possible linker amino acid sequence (see e.g. FIG. 2A). In some embodiments, wherein the protease cleavable domain is C-terminal to an HSA polypeptide sequence, the precursor construct has a regulatable enhanced half-life wherein the precursor construct has an enhanced half-life in circulation in vivo and in the absence of a tumor microenvironment.

In some embodiments, a regulatory domain comprises a cleavable half-life prolonging domain comprising a protease cleavable domain and a CAP masking domain. In some embodiments, the order of components in the regulatory domain is (N-terminal to C-terminal) CAP-L-protease cleavable domain, wherein L is a possible linker amino acid sequence (see FIG. 2E). Prior to entry of the precursor antibody into a tumor microenvironment, the tri-specific precursor construct is effectively blocked from binding with an immune effector target molecule

In some embodiments, there are two sub-regulatory domains: one comprising a cleavable serum half-life prolonging domain and one comprising a cleavable CAP masking domain. In some embodiments, there are three regulatory domains: one comprising a cleavable half-life prolonging domain and two comprising a cleavable CAP masking domain. A precursor tri-specific construct with HSA regulatory domain and at least one CAP regulatory domain, has a regulatable enhances half-life wherein the precursor tri-specific antibody construct has an enhanced half-life and is effectively blocked from binding with at least one immune effector target molecule. Half-life may be enhanced in circulation in vivo and in the absence of a tumor milieu. In some embodiments, activation of cytotoxicity by a precursor tri-specific antibody construct is limited to the tumor milieu. In some embodiments, the precursor construct maintains an enhanced half-life in circulation in vivo and is effectively blocked from binding with an immune effector target molecule in circulation in vivo within a non-tumor milieu (FIG. 1). In some embodiments, activation of cytotoxicity to target is specifically masked by a CAP component of the precursor construct in circulation and in the absence of a tumor milieu, and the precursor construct comprises an enhance half-life in circulation in vivo and in the absence or presence of a tumor milieu. In some embodiments, activation of cytotoxicity is limited to the tumor milieu.

In some embodiments, activation of cytotoxicity to target is specifically masked by the CAP component in circulation and in a non-tumor milieu. In some embodiments, activation of cytotoxicity is limited to the tumor milieu. In some embodiments, activation of a T-cell is specifically masked by a CAP component. In some embodiments, activation of an NK cell is specifically masked by a CAP component. In some embodiments, activation of both a T-cell and an NK cell is specifically masked by a CAP component.

In some embodiments, the amino acid sequence of the HSA component is set forth in SEQ ID NO: 6. In some embodiments, the amino acid sequence of the HSA component is set forth in SEQ ID NO: 7.

In some embodiments, the amino acid sequence of the HSA components is any HSA polypeptide sequence known in the art or a portion thereof, or a homolog thereof. In some embodiments, the HSA component of a precursor tri-specific antibody construct comprises, for example but not limited to, any human albumin protein sequence disclosed in a known database such as the protein data base of National Center of Biotechnology Information (NCBI) or Swiss-Prot, wherein the sequence might be identified specifically as human or may be identified as a synthetic construct.

In some embodiments, the HSA component is encoded by the nucleotide sequence set forth in SEQ ID NO: 8.

In some embodiments, the nucleic acid sequence of the HSA components is any HSA nucleotide sequence known in the art or a portion thereof, or a homolog thereof. In some embodiments, the HSA component of a precursor tri-specific antibody construct comprises a nucleic acid sequence that encodes, for example but not limited to, any human albumin protein sequence disclosed in a known database such as the protein data base the is part of National Center of Biotechnology Information (NCBI) or Swiss-Prot, wherein the sequence might be identified specifically as human or may be identified as a synthetic construct.

In some embodiments, homologues of an HSA component comprise polypeptides which are at least 50%, at least 55%, at least 60%, at least 65%, at least 70%, at least 75%, at least 80%, at least 85%, at least 87%, at least 89%, at least 91%, at least 93%, at least 95%, at least 96%, at least 97%, at least 98%, or at least 99% homologous to the amino acid sequence. In some embodiments, homologues comprise polypeptides which are at least 50%, at least 55%, at least 60%, at least 65%, at least 70%, at least 75%, at least 80%, at least 85%, at least 87%, at least 89%, at least 91%, at least 93%, at least 95%, at least 96%, at least 97%, at least 98%, or at least 99% homologous to an HSA polypeptide or a portion thereof, as determined using BlastP software of the National Center of Biotechnology Information (NCBI) using default parameters. In some embodiments, homologues encoding an HSA component comprise nucleotides which are at least 50%, at least 55%, at least 60%, at least 65%, at least 70%, at least 75%, at least 80%, at least 85%, at least 87%, at least 89%, at least 91%, at least 93%, at least 95%, at least 96%, at least 97%, at least 98%, or at least 99% homologous to the nucleic acid sequence. In some embodiments, homologues encode polypeptides which are at least 50%, at least 55%, at least 60%, at least 65%, at least 70%, at least 75%, at least 80%, at least 85%, at least 87%, at least 89%, at least 91%, at least 93%, at least 95%, at least 96%, at least 97%, at least 98%, or at least 99% homologous to an HSA polypeptide or a portion thereof, as determined using BlastP software of the National Center of Biotechnology Information (NCBI) using default parameters.

In some embodiments, homology also encompasses deletion, insertion, or substitution variants, including an amino acid substitution, thereof and biologically active polypeptide fragments thereof. In one embodiment, the variant comprises conservative substitutions, or deletions, insertions, or substitutions that do not significantly alter the three-dimensional structure of the HSA component. In some embodiments, the deletion, insertion, or substitution does not alter the function of interest of the HSA component, which in some embodiment, is providing half-life prolonging domain.

Linear representation of embodiments of regulatory domains of a precursor tri-specific antibody construct disclosed herein include but are not limited to (N-terminal to C-terminal)
(1) CAP-L-protease cleavable domain-L, wherein the L may or may not be present;
(2) HSA-L-protease cleavable domain-L, wherein the L may or may not be present;
(3) CAP-L-non-cleavable domain-L, wherein the L may or may not be present;
(4) HSA-L-non-cleavable domain-L, wherein the L may or may not be present;
(5) Protease cleavable domain-L-CAP;
(6) Protease cleavable domain-L-HSA;
(7) Non-cleavable domain-L-CAP; and
(8) Non-cleavable domain-L-HSA.

In some embodiments, a precursor tri-specific antibody construct disclosed herein comprises a precursor construct having an increased therapeutic window, wherein its restricted presence provides the ability to target a wide array of new targets or provide improved activities or a combination thereof, for example but not limited to, the ability to activate T-cells and/or NK cells in the cancer microenvironment and targeting cancer-specific TAAs depending on a cancer type and the specific TAAs that are uniquely expressed by this cancer type in conjunction with the proteases produced by this cancer type. In some embodiments, the precursor construct has the ability to activate T-cells only in the TME and targets a cancer specific TAA and a NK cell. In some embodiments, the precursor construct has the ability to activate T-cells only in the TME and targets a TAA present in the TME and a NK cell.

As used herein, the “C-terminal” of a polypeptide and the like, e.g., carboxyl-terminus, carboxy-terminus, C-terminal tail, C-terminal end, or COOH-terminus) is the end of an amino acid chain (protein or polypeptide), terminated by a free carboxyl group (-COOH). When the protein is translated from messenger RNA, it is created from N-terminus to C-terminus. The convention for writing peptide sequences is to put the C-terminal end on the right and write the sequence from N- to C-terminus. In some embodiments, the C-terminal end of a polypeptide encompasses to the last amino acid residue of the polypeptide which donates its amine group to form a peptide bond with the carboxyl group of its adjacent amino acid residue.

As used herein, the “N-terminal” of a polypeptide and the like, e.g., amino-terminus, NH₂-terminus, N-terminal end or amine-terminus) is the start of a protein or polypeptide referring to the free amine group (-NH₂) located at the end of a polypeptide. Normally the amine group is bonded to another carboxylic group in a protein to make it a chain, but since the end of a protein has only 1 out of 2 areas chained, the free amine group is referred to the N-terminus. As stated above, by convention, peptide sequences are written N-terminus to C-terminus, left to right in LTR languages. This correlates the translation direction to the text direction (because when a protein is translated from messenger RNA, it is created from N-terminus to C-terminus - amino acids are added to the carbonyl end). In some embodiments, the N-terminal end of a polypeptide encompasses the first amino acid of the polypeptide which donates its carboxyl group to form a peptide bond with the amine group of its adjacent amino acid residue.

A skilled artisan would appreciate that a linker component may encompass an amino acid peptide linking through one or more chemical bonds or indirect linking through one or more linkers. Any suitable chemical bonds can be used to make a direct link, including without limitation, covalent bonds such as peptide bond and disulfide bond, non-covalent bonds such as hydrogen bond, hydrophobic bond, ionic bond, and Van der Waals bond.

A “covalent bond” refers herein to a stable association between two atoms which share one or more electrons. Examples of the covalent bonds include, without limitation, a peptide bond and a disulfide bond. “Peptide bond” as used herein refers to the covalent bond formed between the carboxyl group of an amino acid and the amine group of the adjacent amino acid. “Disulfide bond” as used herein refers to a covalent bond formed between two sulfur atoms. A disulfide bond can be formed from oxidation of two thiol groups. In certain embodiments, the covalently link is direct link through a covalent bond. In certain embodiments, the covalently link is direct link through a peptide bond or a disulfide bond.

A “non-covalent bond” refers herein to an attractive interaction between two molecules or two chemical groups that does not involve sharing of electrons. Examples of non-covalent bonds include, without limitation, a hydrogen bond, a hydrophobic bond, an ionic bond, and a Van der Waals bond. A “hydrogen bond” refers herein to attractive force between a hydrogen atom of a first molecule/group and an electronegative atom of a second molecule/group. A “hydrophobic bond” refers herein to a force that causes hydrophobic or nonpolar molecules/groups to aggregate or associate together in an aqueous environment. An “ionic bond” refers herein to an attraction between a positive ion and a negative ion. A “Van der Waals bond” refers herein to a non-specific attraction force between two adjacent molecules/groups which have momentary random fluctuations in the distribution of electrons. In certain embodiments, the covalently link is direct link through a non-covalent bond. In certain embodiments, the covalently link is direct link through a hydrogen bond, a hydrophobic bond, an ionic bond, or a Van der Waals bond.

A skilled artisan would appreciate that a protease cleavable domain described herein encompasses linker comprising a protease cleavage site. Thus, the terms “protease cleavable domain” and protease cleavable linker” may be used interchangeably herein having all the same meanings and qualities.

A skilled artisan would appreciate that the terms “tumor microenvironment”, “cancer microenvironment”, “TME”, and “tumor milieu” may be used interchangeably having the same qualities and meanings and encompassing the microenvironment to tumor development. While the normal cellular microenvironment can inhibit malignant cell growth, the modifications that occur in the tumor microenvironment may synergistically support cell proliferation.

Tumors shape their microenvironment and support the development of both tumor cells and non-malignant cells. The tumor microenvironment affects angiogenesis by interfering with the signaling pathways required for cell recruitment and vascular construction. Endothelial progenitor cells (EPCs) that are recruited under hypoxic conditions for angiogenesis have been associated as well with metastasis. In some embodiments, TAA comprise cell surface antigens associated with angiogenesis. In some embodiments, a TAA is overexpressed by a cancer cell. In some embodiments, a TAA is expressed on an embryonic cell. In some embodiments, a TAA is expressed on an embryonic cell and on a cancer cell but has no or only minimal expression on normal adult cells. In some embodiments, a TAA is expressed on a solid tumor cell. In some embodiments, a TAA is expression on a non-solid cancerous cell. In some embodiments, a TAA is expressed on an angiogenic tissue cell.

In addition. proteins secreted by the tumor modify the microenvironment by contributing growth factors and proteases that degrade the extracellular matrix and affect cell motility and adhesion. Stromal cells secrete ECM proteins, cytokines, growth factors, proteases, protease inhibitors, and endoglycosidases such as heparanase. Matrix metalloproteinases (MMP) are important secreted proteins closely associated with cancer development. MMP are expressed at higher levels by tumor-associated epithelial cells than by normal epithelial cells. In some embodiments, the microenvironment of a tumor comprises increased protease activity compared with a non-tumor environment.

In some embodiments, a protease cleavable domain comprises a protease cleavable amino acid sequence (cleavable peptide/cleavable linker; CP) comprises a peptide cleavable by a serine protease, a cysteine protease, an aspartate protease, or a matrix metalloprotease (MMP) cleavable sequence. In some embodiments, a protease cleavable domain comprises a protease cleavable amino acid sequence (cleavable peptide/cleavable linker; CP) comprises a peptide, which is a substrate for cleavage by multiple difference proteases. In some embodiments, a protease cleavable domain comprises a protease cleavable amino acid sequence (cleavable peptide/cleavable linker; CP) comprises a peptide, which is a substrate for cleavage by a MMP2/MMP9 protease, or a urokinase-type plasminogen activator (uPA) protease, or a matriptase, or a legumain protease. In some embodiments, the serine protease, cysteine protease, aspartate protease, uPA protease, matriptase, legumain protease, or matrix metalloprotease (MMP) is expressed at higher levels in a tumor microenvironment. In some embodiments, the matrix metalloprotease is expressed at higher levels in a tumor microenvironment.

In some embodiments, the protease cleavable sequence is an MMP cleavable sequence. In some embodiments, the matrix metalloprotease cleavable sequence may be a matrix metalloprotease 1 (MMP-1), a matrix metalloprotease 2 (MMP-2), a matrix metalloprotease 9 (MMP-9), or a matrix metalloprotease 14 (MMP-14) cleavable sequence.

In some embodiments, the protease cleavable sequence is a uPA (urokinase-type plasminogen activator) cleavable sequence. In some embodiments, the protease cleavable sequence is a MT-SP1 (matripase) cleavable sequence.

In some embodiments, the protease cleavable sequence is an MMP, uPA, matriptase, and legumain cleavable sequence.

In some embodiments, the protease cleavable domain comprises an amino acid sequence 1 to 10 amino acids long. In some embodiments, the protease cleavable domain is 1 to 20 amino acids long.

In some embodiments, a protease cleavable domain comprises a protease substrate cleavage sequence, for example but not limited to, an MMP substrate cleavage sequence. A well-known peptide sequence of PLGLAG (SEQ ID NO: 9) in a substrate can be cleaved by most MMPs. Substrate sequences that can be cleaved by MMPs have been extensively studied. A protease substrate cleavage sequence refers to a peptide sequence that can be cleaved by protease treatment. An MMP substrate sequence refers to a peptide sequence that can be cleaved by incubation with an MMP. SEQ ID NO: 9 is a commonly used MMP substrate cleavage sequence (see e.g., Jiang, PNAS (2004) 101:17867-72; Olson, PNAS (2010) 107:4311-6). In another embodiment, the protease cleavage site is recognized by MMP-2, MMP-9, or a combination thereof. In yet another embodiment, the protease site comprises the sequence set forth as GPLGMLSQ (SEQ ID NO: 10), GPLGLWAQ (SEQ ID NO: 11), GPLGLAG (SEQ ID NO: 12), KKNPAELIGPVD (SEQ ID NO: 13), KKQPAANLVAPED (SEQ ID NO: 14), GPLGIAGQ (SEQ ID NO: 15), or PVGLIG (SEQ ID NO: 16). In some embodiments, the protease cleavage site comprises any protease cleavage site (protease cleavable peptide; CP) known in the art to be susceptible to proteases present in a tumor environment, for example by not limited to the protease cleavage sites disclosed in Eckhard, U, et al., (2016) Matrix Biol. Jan;49:37-60.

In some embodiments, a protease cleavable sequence comprising a uPA cleavable sequence comprises the sequence set forth as NSGRAV (SEQ ID NO: 17), SGRSA (SEQ ID NO: 18), LGGSGRSANAILE (SEQ ID NO: 19), SGRS (SEQ ID NO: 20), GGSGRSANK (SEQ ID NO: 21), LGGSGRSANAILEC (SEQ ID NO: 22), GGGRR (SEQ ID NO: 23), TGRGPS (SEQ ID NO: 24), LSGRSDNH (SEQ ID NO: 25), or PLTGRSGG (SEQ ID NO: 26).

In some embodiments, a protease cleavable sequence comprising a matripase cleavable sequence comprises the sequence set forth as QRRVVGG (SEQ ID NO: 27), QAR, AANL (SEQ ID NO: 29), PTNL (SEQ ID NO: 30), PTN, or SAN. In some embodiments, a protease cleavable sequence comprises the sequences of SEQ ID NO:709, 710, 711, 712, 713, 714, or 715. As discussed herein, any combination of protease cleavable sequences can be found in a Tribody or ProTribody construct; for example, the protease cleavable sequences could be cleaved by the same or different proteases, or the protease cleavable sequences could all have the same or different sequences.

In some embodiments, a cleavable peptide is encoded by the nucleic acid sequence set forth in SEQ ID NO: 33: CCACTGGGCCTGGCCGGC.

In some embodiments, the amino acid sequence of a protease cleavable sequence that serves as a substrate for MMP2/9, uPA, matriptase, and legumain cleavable sequence is set forth as PLGLAGSGRSDNH (SEQ ID NO: 35). In some embodiments, all of the protease cleavable sequences comprised in a precursor construct comprise SEQ ID NO: 35. In some embodiments, at least one of the protease cleavable sequences comprised in a precursor construct comprise SEQ ID NO: 35. In some embodiments, at least 2 of the protease cleavable sequences comprised in a precursor construct comprise SEQ ID NO: 35. In some embodiments, at least 3 of the protease cleavable sequences comprised in a precursor construct comprise SEQ ID NO: 35.

In some embodiments, the sequence of the protease cleavable peptide component of regulatory domain one is the same as the protease cleavable peptide component of regulatory domain two. In some embodiments, the sequence of the protease cleavable peptide component of regulatory domain one is not the same as the protease cleavable peptide component of regulatory domain two. In some embodiments, the protease cleaving the cleavable peptide component of regulatory domain one is the same protease as is cleaving the protease cleavable peptide component of regulatory domain two. In some embodiments, the protease cleaving the cleavable peptide component of regulatory domain one is not the same protease as is cleaving the protease cleavable peptide component of regulatory domain two.

In some embodiments, the protease cleaving the first and second regulatory domains is an MMP protease. In some embodiments, the protease cleaving the first and second regulatory domains is a uPA protease. In some embodiments, the protease cleaving the first and second regulatory domains is a matripase protease. In some embodiments, one of the first or second regulatory domains is cleaved by an MMP protease, while the other regulatory domain is cleaved by a non-MMP protease. In some embodiments, one of the first or second regulatory domains is cleaved by an MMP protease, while the other regulatory domain is cleaved by a uPA protease. In some embodiments, one of the first or second regulatory domains is cleaved by an MMP protease, while the other regulatory domain is cleaved by a matripase protease. In some embodiments, one of the first or second regulatory domains is cleaved by one MMP protease, while the other regulatory domain is cleaved by another MMP protease.

A stable linker or a protease non-cleavable linker refers to a linker peptide sequence that does not belong to the known protease substrate sequences and thus does not lead to significant cleavage product formation upon incubation with a protease.

In some embodiments, the cleavage substrate (or cleavage sequence) of the linker may include an amino acid sequence that can serve as a substrate for a protease, usually an extracellular protease. In other embodiments, the cleavage sequence comprises a cysteine-cysteine pair capable of forming a disulfide bond, which can be cleaved by action of a reducing agent. In other embodiments the cleavage sequence comprises a substrate capable of being cleaved upon photolysis.

The cleavage substrate is positioned within the protease cleavable domain such that when the cleavage substrate is cleaved by a cleaving agent (e.g., a cleavage substrate of a linker is cleaved by the protease and/or the cysteine-cysteine disulfide bond is disrupted via reduction by exposure to a reducing agent) or by light-induced photolysis, in the presence of a target, resulting in cleavage products having various functional properties as described herein. In some embodiments, cleavage products have decreased half-life. In some embodiments, cleavage product has the ability to activate T-cell (FIG. 1).

The cleavage substrate of a cleavage domain may be selected based on a protease that is co-localized in the diseased tissue, or on the surface of the cell that expresses the target antigen of interest of a binding domain of a fusion moiety. A variety of different conditions are known in which a target of interest is co-localized with a protease, where the substrate of the protease is known in the art. In the example of cancer, the target tissue can be a cancerous tissue, particularly cancerous tissue of a solid tumor. There are reports in the literature of increased levels of proteases having known substrates in a number of cancers, e.g., solid tumors. See, e.g., [La Rocca et al, (2004) British J. of Cancer 90(7): 1414-1421. Radisky ES, Front Biosci (Landmark Ed). 2015 Jun 1;20:1144-63; Miao C, et al., Oncotarget. 2017 May 9;8(19):32309-32321]. Non-limiting examples of disease include: all types of cancers (breast, lung, colorectal, prostate, head and neck, pancreatic, etc), rheumatoid arthritis, Crohn’s disease, melanomas, SLE, cardiovascular damage, ischemia, etc. Furthermore, anti-angiogenic targets, such as VEGF, are known.

In some embodiments, where the TAA of the first or second binding domain is selected such that it is capable of binding a tumor antigen, a suitable cleavage substrate sequence for the linker will be one which comprises a peptide substrate that is cleavable by a protease that is present at the cancerous treatment site, that is the tumor microenvironment that is particularly present at elevated levels at the cancer treatment site as compared to non-cancerous tissues.

In some embodiments, the first or second, or both the first and second binding domain of a precursor construct disclosed herein can bind a TAA, e.g., EGFR and the cleavage substrate sequence can be a matrix metalloprotease (MMP) substrate, and thus is cleavable by an MMP. In other embodiments, a TAA comprises ROR1 and the cleavage substrate sequence can be a matripase (MT-SP1, TADG-15, epithin, ST14) substrate, and thus is cleavable by a matriptase. In other embodiments, the first or second, or both the first and second binding domain of a precursor construct can bind a target of interest and the cleavage substrate present in the cleavable domain can be, for example, legumain, plasmin, TMPRSS-¾, MMP-9, MT1-MMP, cathepsin, caspase, human neutrophil elastase, beta-secretase, uPA, or PSA. In other embodiments, the cleavage domain is cleaved by other disease-specific proteases, in diseases other than cancer such as multiple sclerosis or rheumatoid arthritis.

In some embodiments, a precursor tri-specific antibody construct may bind to a TAA by way of the first binding domain, wherein the cleavable domain of the regulatory arm remains uncleaved and therefore the third binding domain of a precursor construct or a partially cleaved precursor construct may be specifically unavailable to a T cell or NK cell target antigen due to the presence of the CAP component. In some embodiments, a precursor tri-specific antibody construct may bind to a TAA by way of the first binding domain, wherein the cleavable domain of the regulatory arm remains uncleaved, wherein the precursor construct or partially cleaved precursor construct has enhanced half-life due to a half-life prolonging domain (e.g., an HSA polypeptide sequence) and the third binding domain is available or partially available to a T cell or NK cell target antigen. In some embodiments, a precursor tri-specific antibody construct may bind to a TAA by way of the first binding domain, wherein the cleavable domain of both regulatory arms remain uncleaved, wherein the precursor construct has enhanced half-life due to a half-life prolonging domain (e.g., an HSA polypeptide sequence) and the third binding domain remains specifically unavailable to a T cell or NK cell target antigen due to the presence of the CAP component.

In some embodiments, there are linkers (L) between any of the component parts of the precursor tri-specific antibody construct. In some embodiments, the linkers of the precursor construct (e.g., the linkers between the VL and VH of the Fab and the regulatory domains) comprise cleavable domain linkers. In some embodiments, the ability to be cleaved is independently selected for each linker. In some embodiments, the ability to be cleaved by a protease is independently selected for each linker. In some embodiments, a linker is cleavable by a protease. In some embodiments, a linker is not cleavable by a protease. In some embodiments, the linker between the CH or CL of the Fab and the scFv of the first binding domain comprises a non-cleavable linker. In some embodiments, the linker between the CH or CL of the Fab and the scFv of the second binding domain comprises a non-cleavable linker.

A skilled artisan would appreciate that in some embodiments, a linker comprises a spacer between two active components or between two regions of an active component.

A skilled artisan would appreciate that the cleavable domain comprises a linear amino acid sequence comprising an enzyme cleavage site and may, in certain embodiments, be termed a “cleavable linker” or a “linker” or a “cleavable peptide” or a “CP”, wherein linkers disclosed herein may be cleavable or non-cleavable.

In some embodiments, a linker is present C-terminal to the Heavy chain constant region (CH) of the Fab fragment. In some embodiments, a linker is present C-terminal to the Light chain constant region (CL) of the Fab fragment. In some embodiments, the linker C-terminal to the CH is cleavable. In some embodiments, the linker C-terminal to the CH is non-cleavable. In some embodiments, the linker C-terminal to the CL is cleavable. In some embodiments, the linker C-terminal to the CL is non-cleavable.

In some embodiments, a linker is a single amino acid. In some embodiments, a cleavable linker comprises the amino acid sequence set forth in any of SEQ ID NOs: 9-17, or 29-30 or amino acid sequences QAR, PTN, or SAN. In some embodiments, a cleavable linker is encoded by the nucleic acid sequence set forth in SEQ ID NO: 33. In some embodiments, a cleavable linker is encoded by the nucleic acid sequence set forth in SEQ ID NO: 35.

In some embodiments, a non-cleavable linker comprises the amino acid sequence set forth in SEQ ID NOs: 162. In some embodiments, a non-cleavable linker is encoded by the nucleic acid sequence set forth in SEQ ID NO: 163.

For specific cleavage by an enzyme protease, contact between the enzyme and the cleavage substrate is made. When the precursor construct comprising a first and a second binding domain binding to a TAA or an extracellular NK antigen, or any combination thereof as described herein in detail, a third binding domain binding to an extracellular epitope of a T cell or NK cell surface antigen, and two regulatory domains comprising cleavable linkers is in the presence of sufficient enzyme activity, the cleavable domains can be cleaved. Sufficient enzyme activity can refer to the ability of the enzyme to make contact with a protease cleavable domain having the cleavage site and effect cleavage. In some embodiments, an enzyme may be in the vicinity of the precursor construct but unable to cleave because of other cellular factors or protein modification of the enzyme. In some embodiments, an anti-TAA comprises an anti-NK antigen.

In some embodiments, cleavable domain substrates can include but are not limited to substrates cleavable by one or more of the following enzymes or proteases: ADAM10; Caspase 8, Cathepsin S, MMP 8, ADAM12, Caspase 9, FAP, MMP 9, ADAM17, Caspase 10, Granzyme B, MMP 13, ADAMTS, Caspase 11, Guanidinobenzotase (GB), MMP 14, ADAMTS5. Caspase 12, Hepsin, MT-SP1, BACE, Caspase 13, Human Neutrophil Elastase Neprilysin (HNE), Caspases, Caspase 14, Legumain, NS¾A, Caspase 1, Cathepsins, Matriptase 2, Plasmin, Caspase 2, Cathepsin A, Meprin, PSA, Caspase 3, Cathepsin B, MMP 1, PSMA, Caspase 4, Cathepsin D, MMP 2, TACE, Caspase 5, Cathepsin E, MMP 3, TMPRSS ¾, Caspase 6, Cathepsin K, MMP 7, uPA, Caspase 7, Matripase (MT-SP1, TADG-15, epithin, ST14) and MT1-MMP.

In another embodiment, the cleavage substrate can involve a disulfide bond of a cysteine pair, which is thus cleavable by a reducing agent such as, for example, but not limited to a cellular reducing agent such as glutathione (GSH), thioredoxins, NADPH, flavins, ascorbate, and the like, which can be present in large amounts in tissue of or surrounding a solid tumor.

Other appropriate protease cleavage sites for use in the cleavable linkers herein are known in the art or may be identified using methods such as those described by Turk et al., 2001 Nature Biotechnology 19, 661-667.

In some embodiments, both the first binding domain, the second binding domain, and the third binding domain of the precursor tri-specific antibody constructs can bind to their respective human and non-chimpanzee primate target molecules. The first binding domain, thus, binds to a human cell surface tumor associated antigen (TAA) and to the corresponding homolog of the cell surface TAA in a non-chimpanzee primate. The identification and determination of homologs of human cell surface TAA in non-chimpanzee primates is well known to the person skilled in the art and can be carried out e.g. by sequence alignments. The third binding domain can bind to a T cell or NK cell surface antigen, e.g. an human CD3ε extracellular epitope, and can bind to the corresponding homolog of the CD3ε in a non-chimpanzee primate. In some embodiments, the first or second or third binding domains, or any combination thereof, also bind to their respective chimpanzee target molecules.

A skilled artisan would appreciate that in some embodiments, a cell surface tumor associated antigen (TAA) encompasses a molecule which is displayed on the surface of a cell. In some embodiments, the cell is a tumor cell. In some embodiments, the cell is a non-tumor cell present in the milieu of a tumor, for example but not limited to a cell present within vasculature tissue associated with a tumor or cancer. In some embodiments, the cell is a non-tumor cell present in the milieu of a tumor, for example but not limited to an NK cell.

A skilled artisan would appreciate that the terms “antigen” or “immunogen” encompass a peptide, protein, polypeptide which is immunogenic. In some embodiments, an antigen is capable of eliciting an immune response in a mammal, and therefore contains at least one and may contain multiple epitopes. An “antigen” molecule or a portion of a molecule is capable of being bound by a selective binding agent, such as an antigen-binding portion of a Fab fragment or an antigen-binding portion of an scFv fragment. Additionally, an “antigen” is capable of being used in an animal to produce antibodies capable of binding to an epitope of that antigen. In some embodiments, a CAP component comprises the portion of an antigen to which the second binding domain binds.

The term “epitope” includes any determinant, in certain embodiments, a polypeptide determinant, capable of specific binding to a TAA or an immunoglobulin or T-cell receptor, or an NK surface antigen. An epitope is a region of an antigen that is bound by an antibody or an antigen-binding fragment thereof. In some embodiments, a CAP component comprises the epitope to which the third binding domain binds.

In certain embodiments, epitope determinants include chemically active surface groupings of molecules such as amino acids, sugar side chains, phosphoryl or sulfonyl, and may in certain embodiments have specific three-dimensional structural characteristics, and/or specific charge characteristics. In certain embodiments, a precursor tri-specific antibody construct is said to specifically bind an antigen when it preferentially recognizes its target antigen in a complex mixture of proteins and/or macromolecules. A precursor tri-specific antibody construct is said to specifically bind an antigen when the equilibrium dissociation constant is ≤ 10-5, 10-6 or 10-7 M. In some embodiments, the equilibrium dissociation constant may be ≤ 10-8 M or 10-9 M. In some further embodiments, the equilibrium dissociation constant may be ≤ 10-10 M or 10-11 M. Antigens disclosed herein included but are not limited to TAA, CAP components, NK antigens, and immuno-effector molecules such as a human CD3 epsilon polypeptide.

In some embodiments, the tumor associated antigen (TAA) is a tumor antigen. In some embodiments, tumor antigens comprise those antigens are presented on tumor cells. In some embodiments, the tumor antigen is present on a cell of solid tumor. In some embodiments, the tumor antigen is a cancer antigen, present on a cell of a non-solid tumor.

Precursor Tri-specific antibodies can be designed to bind at least one tumor associated antigen (TAA), which in some embodiments comprises a tumor cell surface antigen, a T-cell antigen, and either an NK cell antigen or a second antigen (e.g. a cytokine), with the goal of the antibody being to bind and kill tumor cells more selectively over normal cells, and ultimately to increase the efficacy and safety over a monospecific reagent, wherein a tri-specific antibody comprises a binding domain binding at least one cell surface tumor associated antigen (TAA), a binding domain binding a cell surface NK cell antigen or second TAA, and a binding domain binding an extracellular epitope of a T-cell. However, such tri-specific antibodies fail to regulate the order of binding and therefore, may bind a T-cell prior to or in the absence of binding a TAA, wherein cytotoxicity provided by the activated T-cell may actually cause harmful side effects by non-specifically causing non-tumor cell death. In some embodiments, a TAA comprises a human antigen.

In some embodiments, a second or a third binding site binds to an antigen comprising an NK extracellular surface antigen. In some embodiments, an NK surface antigen comprises an extracellular portion of CD56 or an extracellular portion of CD16. In some embodiments, an NK surface antigen comprises an extracellular portion of a CD16 (FcγRIII), a CD16A (FcγRIIIa), a CD56, a sMICA/B, an ILT, a SLAMF7, a NKp44, a NKp30, a DNAM-1, a NKG2D, a NKG2C/CD94, or a NKp46 antigen. In some embodiments, the NK cell antigen comprises a NK cell activating receptor, a NK cell inhibitory receptor, or a NK cell co-stimulatory receptor. Examples of NK cell activating receptors, NK cell inhibitory receptors, and NK cell co-stimulatory receptors have been discussed above.

In some embodiments, a first binding domain binds to a polypeptide target which in some embodiments is associated with a particular cancer or cancers or disease condition, for example but not limited to a TAA comprising FcγRI, FcγRIIa FcγRIIb FcγRIIIa FcγRIIIb, CD28, CD137, CTLA-4, FAS, fibroblast growth factor receptor 1 (FGFR1), FGFR2, FGFR3, FGFR4, glucocorticoid-induced TNFR-related (GITR) protein, lymphotoxin-beta receptor (LTβR), toll-like receptors (TLR), tumor necrosis factor-related apoptosis-inducing ligand-receptor 1 (TRAIL receptor 1; multiple malignancies including ovarian and colorectal carcinomas) and TRAIL receptor 2, prostate-specific membrane antigen (PSMA; prostate carcinoma) protein, prostate stem cell antigen (PSCA) protein (prostate adenocarcinoma), CA125 (multiple cancers including ovarian carcinoma), tumor-associated protein carbonic anhydrase IX (CAIX; multiple cancers including renal cell carcinoma), epidermal growth factor receptor 1 (EGFR1; epithelial malignancies), EGFR (non-small cell lung cancer, epithelial ovarian cancer, colorectal cancer, head & neck cancer, breast cancer, lung cancer, esophageal cancer), EGFRvIII, human epidermal growth factor receptor 2 (Her2/neu; Erb2; epithelial malignancies), ErbB3 also known as HER3 (epithelial malignancies), Folate receptor, ephrin receptors, PDGFRa (epithelial malignancies), ErbB-2, CD20 (B cells, autoimmune, allergic or malignant), CD22 (B cells, autoimmune or malignant), CD30 (B cell malignancies), CD33 (myeloid malignancies), CD40, CD37, CD38, CD70 (B cells, autoimmune, allergic or malignant), CD74 (B cells, autoimmune, allergic or malignant), CD56 (T cell or NK cell lymphomas), CD40 (B cells, autoimmune, allergic or malignant); CD80 (B cells, autoimmune, allergic or malignant), CD86 (B cells, autoimmune, allergic or malignant), CD2 (T cell or NK cell lymphomas), p53, cMet also known as tyrosine-protein kinase Met or hepatocyte growth factor receptor (HGFR; Gastrointestinal tract and hepatic malignancies), MAGE-A1, MAGE-A2, MAGE-A3, MAGE-A4, MAGE-A6, MAGE-A10, MAGE-A12, BAGE, DAM-6, -10, GAGE-1, -2, -8, GAGE-3, -4, -5, -6, -7B, NA88-A, NY-ESO-1, BRCA1, BRCA2, MART-1, MC1R, Gp100, PSA, PSM, Tyrosinase, Wilms’ tumor antigen (WT1), TRP-1, TRP-2, ART-4, CAMEL, Cyp-B, hTERT, hTRT, iCE, MUC1 (epithelial malignancies), MUC2, P-cadherin (Epithelial malignancies, including breast adenocarcinoma), Myostatin (GDF8) (many tumors including sarcoma and ovarian and pancreatic adenocarcinoma), Cripto (TDGF1) (Epithelial malignancies including colon, breast, lung, ovarian, and pancreatic cancers), ACVRL1/ALK1 (multiple malignancies including leukemias and lymphomas), MUC5AC (Epithelial malignancies, including breast adenocarcinoma), PRAME, P15, RU1, RU2, SART-1, SART-3, WT1, AFP, β-catenin/m, Caspase-8/m, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, ETV6/AML, LDLR/FUT, Pml/RARα, TEL/AML1, CD28, CD137 (B cells or T cells, autoimmune, allergic or malignant), CanAg (tumors such as carcinomas of the colon and pancreas), Mesothelin (many tumors including mesothelioma and ovarian and pancreatic adenocarcinoma), DR5 (multiple malignancies including ovarian and colorectal carcinoma), PD-1 (B cells, autoimmune, allergic or malignant), PD1L (Multiple malignancies including epithelial adenocarcinoma), IGF-1R (Most malignancies including epithelial adenocarcinoma), CXCR4 (B cells or T cells, autoimmune, allergic or malignant), Neuropilin 1 (Epithelial malignancies, including lung cancer), Glypicans (multiple cancers including liver, brain and breast cancers), EphA2 (multiple cancers including neuroblastoma, melanoma, breast cancer, and small cell lung carcinoma), CD138 (Myeloma), B7-H3 (CSC, stroma, NSCLC, Bladder tumors, mesothelioma, melanoma), gpA33 (colorectal cancers), GPC3 (liver, lung, esophageal, gastric, head and neck cancers), SSTR2 (Neuroendocrine tumors, GIST), ROR1 (Hematological, pancreatic, ovarian, renal cell carcinoma, NSCLC, and triple negative breast cancer), 5T4 (mesothelioma, gastic, ovarin, renal cancer, cancer stem cells in NSCLC, head and neck cancer), or a VEGF-R2 (vasculature associated with the majority of malignancies including epithelial adenocarcinomas). Examples of the unwanted target cells or cancer cells associated with the TAA presented are included in italics in parenthesis.

In some embodiments, a TAA is selected from the group consisting of EGFR, ROR1, PSMA, and 5T4. In some embodiments, a first binding domain comprises an scFv that binds to a human EGFR (anti-hEGFR), or a human ROR1 (anti-ROR1), or a human PSMA (anti-PSMA), or a human 5T4 (anti-5T4).

In some embodiments, the TAA is EGFR. In some embodiments, a first binding domain comprises a scFv that binds to human EGFR (anti-hEGFR). In some embodiments, the amino acid sequence of an anti-hEGFR-scFv light chain variable region (VL) is set forth in SEQ ID NO: 34. In some embodiments, an anti-hEGFR scFv VL sequence comprises a homolog of SEQ ID NO: 34.

In some embodiments, the anti-hEGFR-scFv light chain variable region (VL) is encoded by the nucleic acid sequence set forth in SEQ ID NO: 36. In some embodiments, the anti-hEGFR-scFv light chain variable region (VL) is encoded by a homolog of the nucleic acid sequence set forth in SEQ ID NO: 36.

In some embodiments, the amino acid sequence of an anti-hEGFR-scFv heavy chain variable region (VH) set forth in SEQ ID NO: 37. In some embodiments, an anti-hEGFR scFv VH sequence comprises a homolog of SEQ ID NO: 37.

In some embodiments, the anti-hEGFR-scFv heavy chain variable region (VH) is encoded by the nucleic acid sequence set forth in SEQ ID NO: 38. In some embodiments, the anti-hEGFR-scFv heavy chain variable region (VH1) is encoded by a homolog of the nucleic acid sequence set forth in SEQ ID NO: 38.

In some embodiments, an anti-EGFR scFv comprises a linker between a VL and a VH region. In some embodiments, the linker between a VL and a VH region comprises any linker disclosed herein. In some embodiments, the amino acid sequence of a linker between a VL and VH region of an anti-EGFR scFv is set forth by SEQ ID NO: 39. In some embodiments, the linker between a VL and a VH region of an anti-EGFR scFv comprises a homolog of SEQ ID NO: 39. In some embodiments, a linker between a VL and VH region of an anti-EGFR scFv is encoded by the nucleic acid sequence set forth in SEQ ID NO:40. In some embodiments, a linker between a VL and VH region of an anti-EGFR scFv is encoded by a homolog by the nucleic acid sequence set forth in SEQ ID NO: 40.

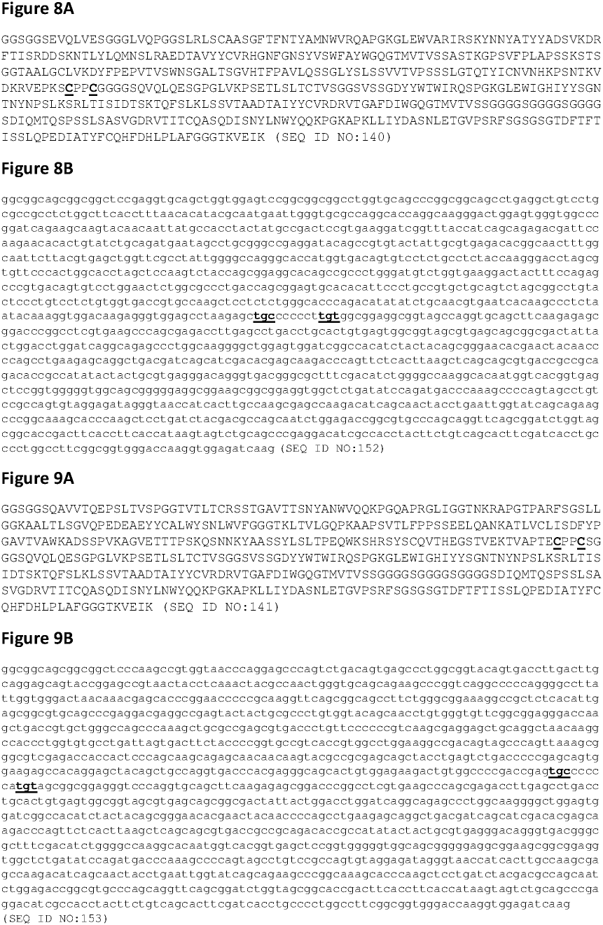

In some embodiments, components of an anti-EGFR scFv comprises a VL-linker-VH order (N-terminal to C-terminal) (FIGS. 8A and 9A). In some embodiments, components of an anti-EGFR scFv comprises a VH-linker-VL order (N-terminal to C-terminal) (FIGS. 8B and 9B).

In some embodiments, an anti-EGFR scFv sequence including a linker sequence comprises the sequence SEQ ID NO: 41. In some embodiments, an anti-EGFR scFv including a linker sequence comprises a homolog of SEQ ID NO: 41.

In some embodiments, an anti-EGFR scFv sequence including a linker sequence comprises the sequence SEQ ID NO: 42. In some embodiments, an anti-EGFR scFv including a linker sequence comprises a homolog of SEQ ID NO: 42.

In some embodiments, the amino acid sequence of an anti-hROR1-scFv, or an anti-PSMA-scFv, or an anti-5T4-scFv light chain variable region are set forth in in Table 1 below:

TABLE 1

Amino acid Sequences of and Optimized Nucleotide Acid sequences encoding anti-hROR1-scFv, or an anti-PSMA-scFv, or an anti-5T4-scFv

| Antigen Binding (anti-antigen) | SEQ ID NO: |
|---|---|
| ROR1 (VL-VH) | 156 |
| ROR1 (VL-VH) | 157 |
| ROR1 (VH-VL) | 166 |
| ROR1 (VH-VL) | 167 |
| PSMA (VL-VH) | 168 |
| PSMA (VL-VH) | 169 |
| PSMA (VH-VL) | 170 |
| PSMA (VH-VL) | 171 |
| 5T4 (VL-VH) | 172 |
| 5T4 (VL-VH) | 173 |
| 5T4 (VH-VL) | 174 |
| 5T4 (VH-VL) | 175 |

In some embodiments, homologues comprise polypeptides which are at least 50%, at least 55%, at least 60%, at least 65%, at least 70%, at least 75%, at least 80%, at least 85%, at least 87%, at least 89%, at least 91%, at least 93%, at least 95%, at least 96%, at least 97%, at least 98%, or at least 99% homologous to the amino acid sequence of an anti-EGFR scFv. In some embodiments, homologues comprise polypeptides which are at least 50%, at least 55%, at least 60%, at least 65%, at least 70%, at least 75%, at least 80%, at least 85%, at least 87%, at least 89%, at least 91%, at least 93%, at least 95%, at least 96%, at least 97%, at least 98%, or at least 99% identical to an amino acid sequence of an anti-EGFR scFv, or any anti-ROR1 scFv, or an anti-PSMA scFv, or an anti-5T4 scFv.

In some embodiments, a nucleotide sequence encoding an anti-EGFR scFv including a linker sequence comprises the sequence SEQ ID NO: 43. In some embodiments, an anti-EGFR scFv including a linker sequence comprises a homolog of SEQ ID NO: 43.

In some embodiments, a nucleotide sequence encoding an anti-EGFR scFv including a linker sequence comprises the sequence SEQ ID NO: 44. In some embodiments, an anti-EGFR scFv including a linker sequence comprises a homolog of SEQ ID NO: 44.

In some embodiments, homologues comprise nucleotides which are at least 50%, at least 55%, at least 60%, at least 65%, at least 70%, at least 75%, at least 80%, at least 85%, at least 87%, at least 89%, at least 91%, at least 93%, at least 95%, at least 96%, at least 97%, at least 98%, or at least 99% homologous to the nucleic acid sequence of an anti-EGFR scFv, . In some embodiments, homologues comprise nucleotides which are at least 50%, at least 55%, at least 60%, at least 65%, at least 70%, at least 75%, at least 80%, at least 85%, at least 87%, at least 89%, at least 91%, at least 93%, at least 95%, at least 96%, at least 97%, at least 98%, or at least 99% identical to the nucleic acid sequence of an anti-EGFR scFv or any anti-ROR1 scFv, or an anti-PSMA scFv, or an anti-5T4 scFv.

In some embodiments, disclosed herein are homologues of an anti-hEGFR scFv VL (SEQ ID NO: 34 or SEQ ID NO: 35) or anti-hEGFR scFv VH (SEQ ID NO: 37) or an anti-hEGFR scFv (SEQ ID NO: 41) or an anti-hEGFR scFv (SEQ ID NO: 42), respectively, as determined using BlastP software of the National Center of Biotechnology Information (NCBI) using default parameters. In some embodiments, disclosed herein are homologues of nucleotide sequences encoding an anti-hEGFR scFv VL (SEQ ID NO: 36) or anti-hEGFR scFv VH (SEQ ID NO: 38) or an anti-hEGFR scFv (SEQ ID NO: 43) or an anti-hEGFR scFv (SEQ ID NO: 44), respectively, as determined using BlastP software of the National Center of Biotechnology Information (NCBI) using default parameters.

In some embodiments, disclosed herein are homologues of an anti-hROR1 scFv VL-VH (SEQ ID NO: 156) or an anti-hROR1 scFv VH-VL (SEQ ID NO: 169), as determined using BlastP software of the National Center of Biotechnology Information (NCBI) using default parameters. In some embodiments, disclosed herein are homologues of nucleotide sequences encoding an anti-hROR1 scFv VL-VH (SEQ ID NO: 157) or an anti-hROR1 scFv VH-VL (SEQ ID NO:167), as determined using BlastP software of the National Center of Biotechnology Information (NCBI) using default parameters.

In some embodiments, disclosed herein are homologues of an anti-hPSMA scFv VL-VH (SEQ ID NO: 168) or an anti-hPSMA scFv VH-VL (SEQ ID NO: 170), as determined using BlastP software of the National Center of Biotechnology Information (NCBI) using default parameters. In some embodiments, disclosed herein are homologues of nucleotide sequences encoding an anti-hPSMA scFv VL-VH (SEQ ID NO: 169) or an anti-hPSMA scFv VH-VL (SEQ ID NO: 171), as determined using BlastP software of the National Center of Biotechnology Information (NCBI) using default parameters.

In some embodiments, disclosed herein are homologues of an anti-h5T4 scFv VL-VH (SEQ ID NO: 172) or an anti-h5T4 scFv VH-VL (SEQ ID NO: 174), as determined using BlastP software of the National Center of Biotechnology Information (NCBI) using default parameters. In some embodiments, disclosed herein are homologues of nucleotide sequences encoding an anti-h5T4 scFv VL-VH (SEQ ID NO: 173) or an anti-h5T4 scFv VH-VL (SEQ ID NO: 174), as determined using BlastP software of the National Center of Biotechnology Information (NCBI) using default parameters.

In some embodiments, homology also encompasses deletion, insertion, or substitution variants, including an amino acid substitution, thereof and biologically active polypeptide fragments thereof. In one embodiment, the variant comprises conservative substitutions, or deletions, insertions, or substitutions that do not significantly alter the three-dimensional structure of the polypeptide of interest, e.g., VL or VH region of the first binding domain, particularly in the areas of the CDR epitope binding regions. In some embodiments, the deletion, insertion, or substitution does not alter the function of interest of the anti-hEGFR VL or anti-hEGFR VH or an anti-EGFR scFv, present in the first, or the second binding domain, or in both the first and the second binding domains of a precursor construct, which in some embodiment, is binding to an EGFR on a target tumor cell. In some embodiments, the deletion, insertion, or substitution does not alter the function of interest of an anti-ROR1 scFv, or an anti-PSMA, or an anti-5T4 scFv present in the first, or the second binding domain, or in both the first and the second binding domains of a precursor construct, which in some embodiment, is binding to an ROR1, PSMA, or 5T4, respectively on a target tumor cell.

In some embodiments, a first or second binding domain, or both a first and second binding domain binding to a cell surface tumor associated antigen includes the sequence set forth in SEQ ID NO: 34, or a homolog thereof. In some embodiments, a first or second binding domain, or both a first and second binding domain binding to a cell surface tumor associated antigen includes the sequence set forth in SEQ ID NO: 37, or a homolog thereof.

In some embodiments, a first or second binding domain, or a first and second binding domain binding to a cell surface tumor associated antigen includes the sequence set forth in any of SEQ ID NO: 34, 37, 156, 166, 168, 170, 172, or 174 or a homolog thereof.

In some embodiments, a first or second binding domain, or a first and second binding domain binding to a cell surface tumor associated antigen includes the sequence set forth in SEQ ID NO: 41 or a homolog thereof. In some embodiments, a first or second binding domain, or a first and second binding domain binding to a cell surface tumor associated antigen includes the sequence set forth in SEQ ID NO: 42 or a homolog thereof.

In some embodiments, a first or second binding domain, or both a first and second binding domain binding to a cell surface tumor associated antigen and encoded by a nucleotide sequence including the sequences set forth in any of SEQ ID NO: 36, 38, 157, 167, 169, 171, 173, or 175, or a homolog thereof.

In some embodiments, a first or second binding domain, or a first and second binding domain binding to a cell surface tumor associated antigen is encoded by a nucleotide sequence that includes the sequence set forth in SEQ ID NO: 36 or a homolog thereof, and the sequence set forth in SEQ ID NO: 38 or a homolog thereof.

In some embodiments, a first or second binding domain, or a first and second binding domain binding to a cell surface tumor associated antigen in encoded by a nucleotide sequence that includes the sequence set forth in SEQ ID NO: 43 or a homolog thereof. In some embodiments, a first or second binding domain, or a first and second binding domain binding to a cell surface tumor associated antigen includes the sequence set forth in SEQ ID NO: 44 or a homolog thereof.

In some embodiments, the nucleotide sequences encoding a precursor tri-specific antibody construct polypeptide is optimized for mammalian transcription and translation. In some embodiments, the nucleotide sequences encoding a first binding domain or a second binding domain or both a first and a second binding domain of a precursor tri-specific antibody construct polypeptides is optimized for mammalian transcription and translation. In some embodiments, the nucleotide sequence of a VL or VH, or of both a VL and VH regions of a first binding domain or a second binding domain, or a first and a second binding domain are optimized for mammalian transcription and translation.

In another embodiment, the TAA provided herein is an angiogenic antigen which is expressed on both activated pericytes and pericytes in tumor angiogenic vasculature, which is associated with neovascularization in vivo. Angiogenic antigens are known in the art see for example WO2010/102140, which is incorporated by reference herein. For example, an angiogenic antigen may be selected from; Angiopoietin-1 (Ang1), Angiopoietin 3, Angiopoietin 4, Angiopoietin 6; Del-1; Fibroblast growth factors: acidic (aFGF) and basic (bFGF); Follistatin; Granulocyte colony-stimulating factor (G-CSF); Hepatocyte growth factor (HGF) /scatter factor (SF); Interleukin-8 (IL-8); Leptin; Midkine; Placental growth factor; Platelet-derived endothelial cell growth factor (PD-ECGF); Platelet-derived growth factor-BB (PDGF-BB); Pleiotrophin (PTN); Progranulin; Proliferin; survivin; Transforming growth factor-alpha (TGF-alpha); Transforming growth factor-beta (TGF-beta); Tumor necrosis factor-alpha (TNF-alpha); Vascular endothelial growth factor (VEGF)/vascular permeability factor (VPF).

As described above and throughout, in some embodiments the first binding domain or the second binding domain, or the first and a second binding domain (TAA binding domain) comprises a single chain variable fragment (scFv). In some embodiments the first binding domain or the second binding domain, but not the first and the second binding domain (TAA binding domain) comprises a single chain variable fragment (scFv) that binds to an extracellular epitope of a Natural Killer (NK) cell antigen.

In some embodiments, the third binding domain comprises a Fab fragment. The specific structural order of components of a precursor tri-specific antibody construct, for example comprised in a polypeptide A and a polypeptide B, is described throughout in more detail.

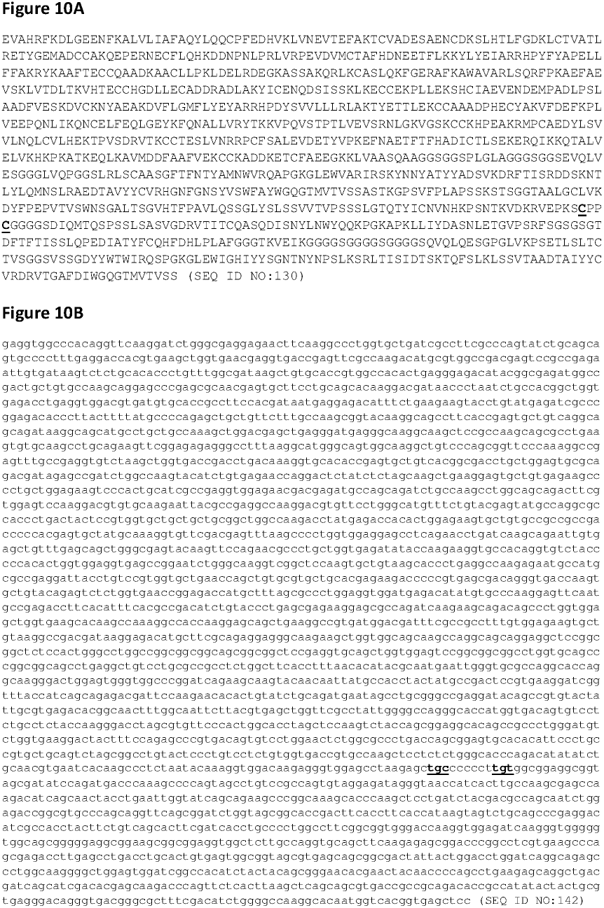

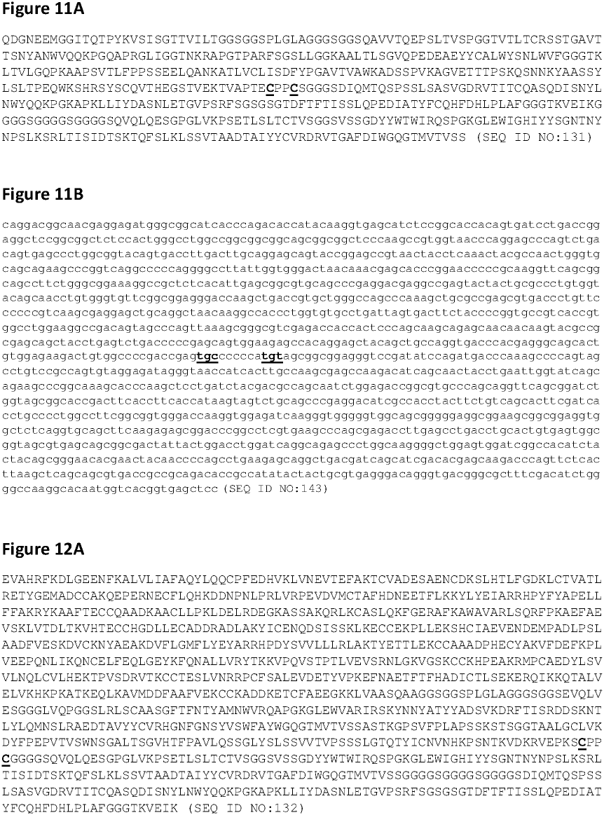

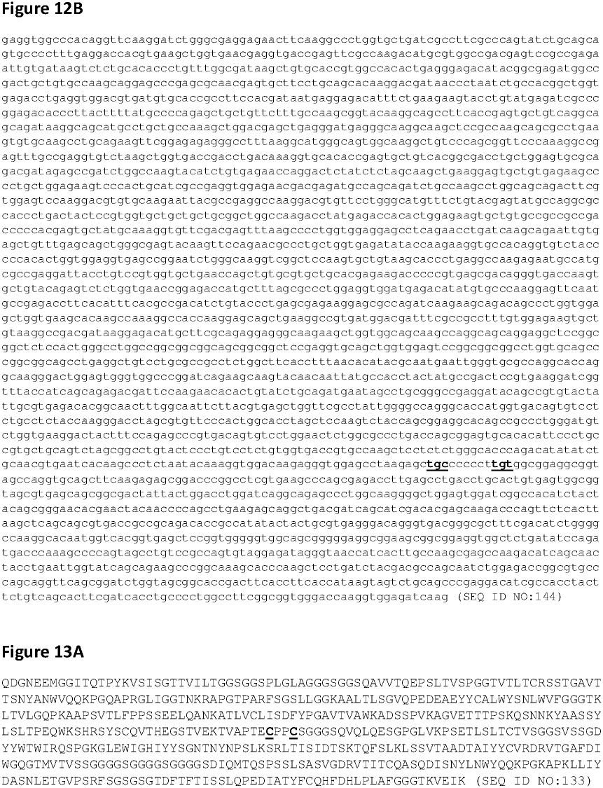

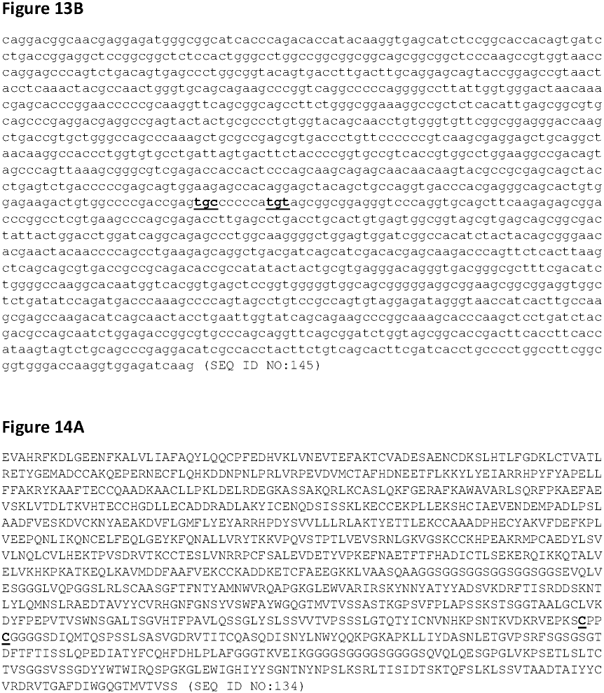

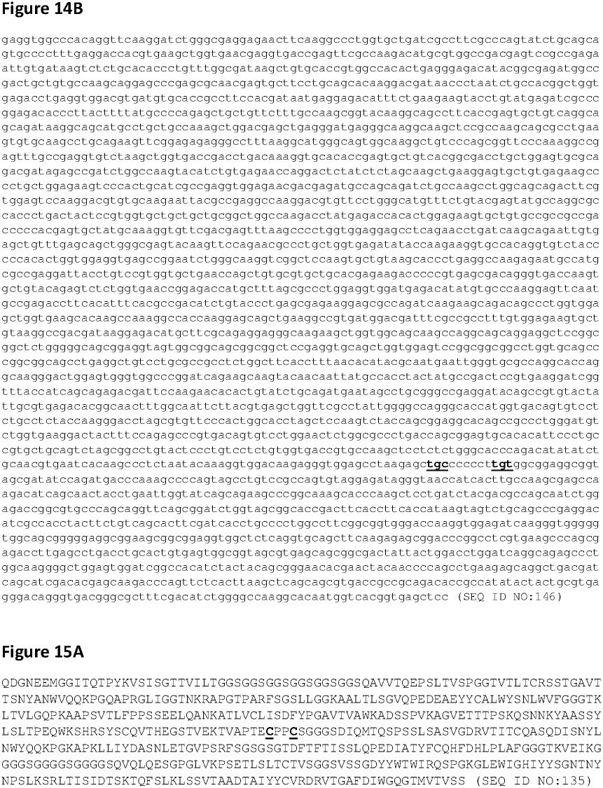

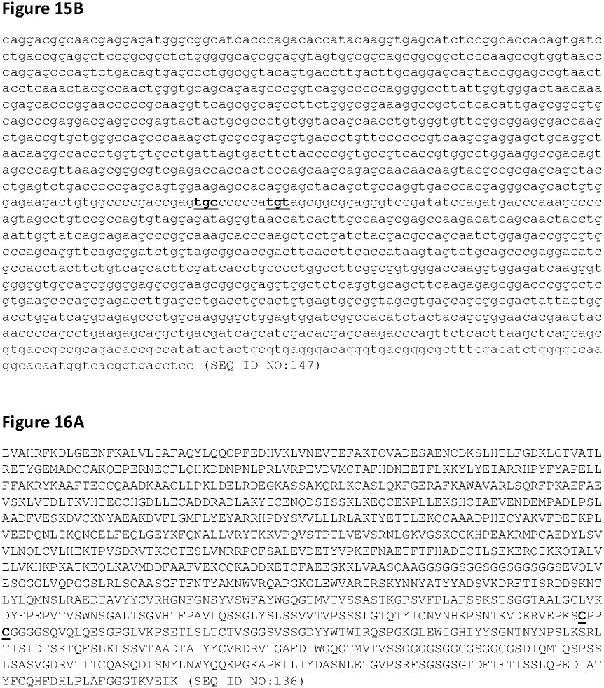

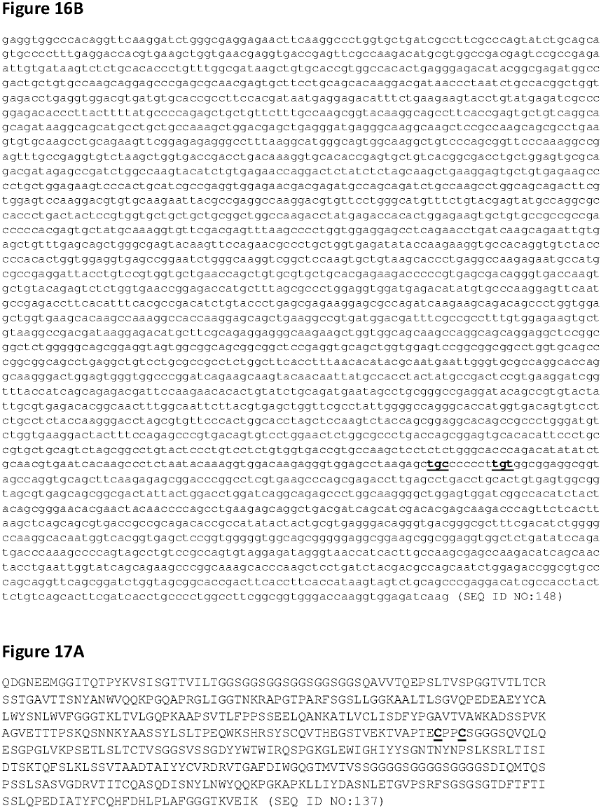

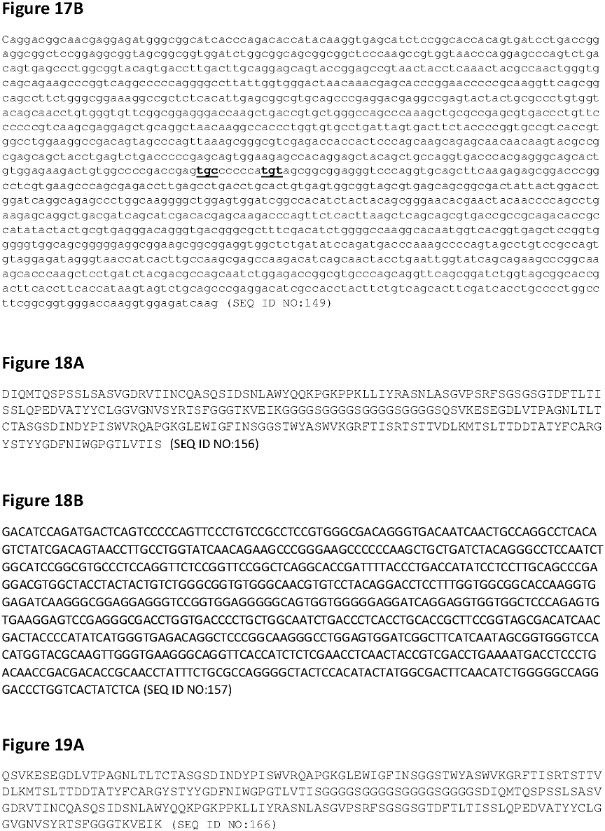

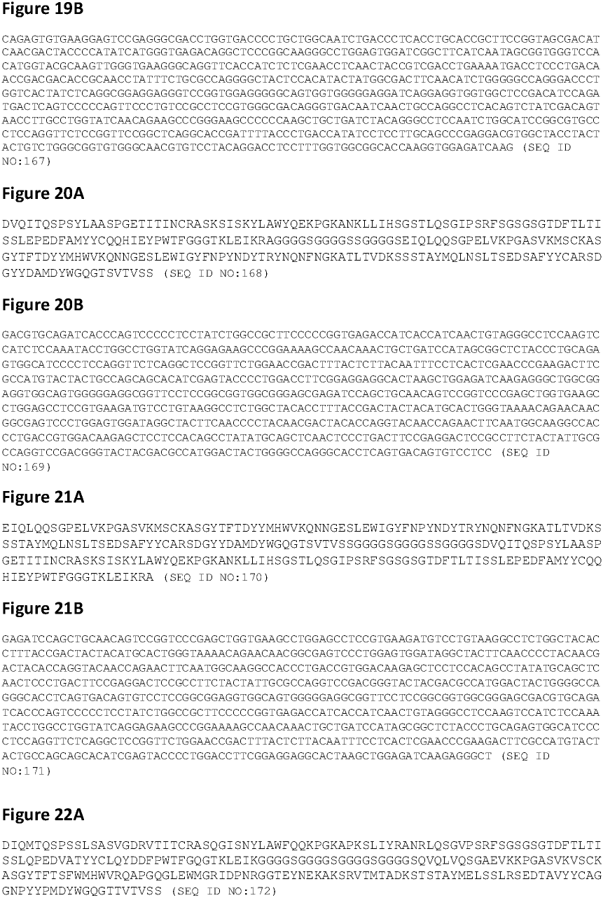

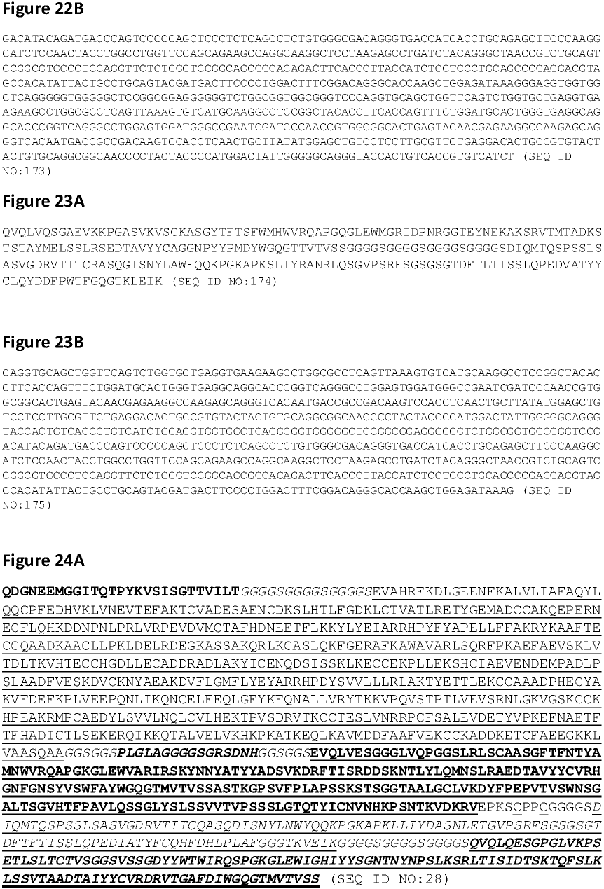

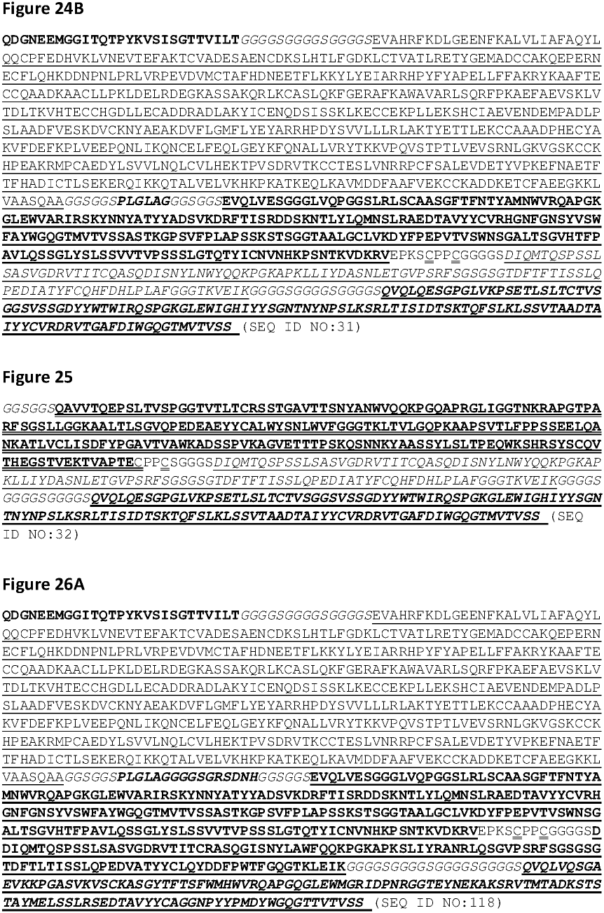

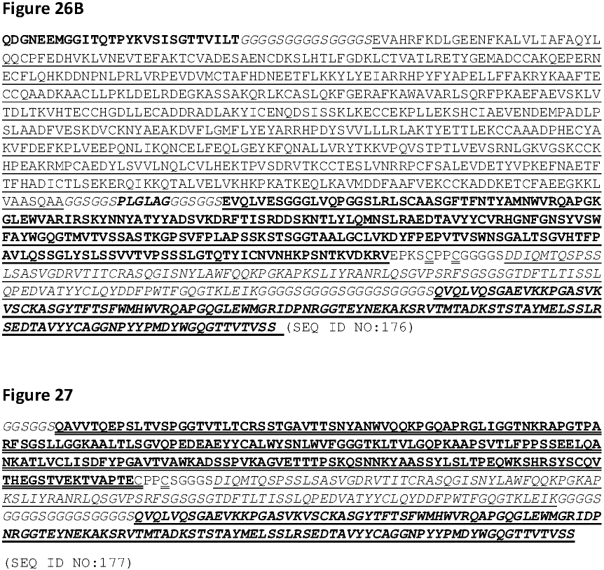

In some embodiments, a precursor tri-specific antibody construct comprises at its core, an Fab fragment, which in some embodiments, comprises the third binding domain. As would be understood by the skilled person, a Fab fragment is the antigen-binding fragment of an antibody. The Fab is composed of one constant and one variable region of an immunoglobulin heavy and an immunoglobulin light chain. The heavy chain constant (CH) and variable (VH) regions heterodimerize with the light chain variable (VL) and constant (CL) regions and are usually covalently linked by a disulfide bond between the heavy and light chain constant regions (see e.g., the amino acid sequences presented in FIGS. 8A, 9A, 10A, 11A, 24A, 24B, 25, 26A, 26B, and 27 wherein the cysteine residues that may form a disulfide bond (Cys-S-S-Cys bond) between polypeptides A and B of a precursor construct are indicated). The codons encoding these Cys residues are indicated in the nucleic acid sequences presented in FIGS. 8B, 9B, 10B, and 11B. Thus, a skilled artisan would appreciate that the term “Fab” with regard to an antibody generally encompasses that portion of the antibody consisting of a single light chain (both variable and constant regions) bound to the variable region and first constant region of a single heavy chain by a disulfide bond.

As would be recognized by the skilled person, a disulfide bond between the heavy and light chain is preferable, but not essential for function (Orcutt, et al. (2010), PEDS, 23:221-228). Thus, in certain embodiments the Fab fragment disclosed herein may not comprise a disulfide bond. In this regard, the heavy and light chains may be engineered in such a way so as to stably interact without the need for disulfide bond. For example, in certain embodiments, the heavy or light chain can be engineered to remove a cysteine residue and wherein the heavy and light chains still stably interact and function as a Fab. In some embodiments, mutations are made to facilitate stable interaction between the heavy and light chains. For example, a “knobs into holes” engineering strategy can be used to facilitate dimerization between the heavy and light chains of a Fab (see e.g., 1996 Protein Engineering, 9:617-621). Using this strategy, “knobs” are created by replacing small amino acid side chains at the interface between interacting domains with larger ones. Corresponding “holes” are made at the interface between interacting molecules by replacing large side chains with smaller ones. Thus, also contemplated for use herein are variant Fab fragments designed for a particular purpose, for example, amino acid changes in the constant domains of CH1 and or CL, and removal of a disulfide bond or addition of tags for purification.

In some embodiments, the configuration of the variable and constant regions within the Fab fragment may be different from what is found in a native Fab. In other words, in one embodiment, the orientation of the variable and constant regions may be VH-CL in one chain and in another VL-CH (Shaefer et al. (2011), PNAS, 108:111870-92). Such modified Fab fragments still function to bind their particular target antigen and are contemplated for use in the precursor construct disclosed herein. Thus, in this regard the variable regions and constant regions that make up the Fab are considered modular.

In certain embodiments, the Fab fragments of this disclosure are derived from monoclonal antibodies and may be derived from antibodies of any type, including IgA, IgM, IgD, IgG, IgE and subtypes thereof, such as IgG1, IgG2, IgG3, and IgG4. The light chain domains may be derived from the kappa or lambda chain. The Fab fragments for use herein may be made recombinantly.

As is well known in the art, an antibody is an immunoglobulin molecule capable of specific binding to a target, such as a carbohydrate, polynucleotide, lipid, polypeptide, etc., through at least one epitope recognition site, located in the variable region of the immunoglobulin molecule. A skilled artisan would appreciate that the term “antibody” encompasses not only intact polyclonal or monoclonal antibodies, but also humanized antibodies, chimeric antibodies, antibody fragments including antibody fragments lacking an Fc region, and any other modified configuration of the immunoglobulin molecule that comprises an antigen-binding site or fragment (epitope recognition site) of the required specificity, including scFv fragments and Fab fragments. In some embodiments, the precursor antibody constructs described herein lack an Fc region.

The Fab fragment as disclosed herein, comprises an antigen-binding portion (third binding domain) comprised of an immunoglobulin heavy chain variable region and an immunoglobulin light chain variable region (VH and VL, respectively). Similarly, the scFv fragment described above (first or second binding domain), comprises an antigen-binding portion comprised of an immunoglobulin heavy chain variable region and an immunoglobulin light chain variable region (VH and VL, respectively). More specifically, the term “antigen-binding portion” as used herein refers to a polypeptide fragment that contains at least one CDR of an immunoglobulin heavy and/or light chain that binds to the target antigen of interest, such as the TAA of the first or second binding region, or a CD3 molecule of the third binding region. In this regard, an antigen-binding portion of the herein described precursor constructs may comprise 1, 2, 3, 4, 5, or all 6 CDRs of a VH and VL sequence of a parent antibody that binds to a target antigen of interest. In certain embodiments, the antigen-binding portion of the scFv fragment (first or second binding domain, or both first and second binding domains) of a precursor tri-specific antibody construct binds to a TAA, for example but not limited to a human EGFR. In certain embodiments, the antigen-binding portion of the Fab fragment of a precursor tri-specific antibody construct binds to CD3.

In certain embodiments, a specific VH and/or VL of the precursor tri-specific antibody construct described herein may be used to screen a library of the complementary variable region to identify VH/VL with desirable properties, such as increased affinity for a target antigen of interest. Such methods are described, for example, in Portolano et al., J. Immunol. (1993) 150:880-887; Clarkson et al., Nature (1991) 352:624-628.

Other methods may also be used to mix and match CDRs to identify Fab having desired binding activity (such as binding to CD3, or other target antigen of interest as described herein for other binding domains present in the precursor tri-specific antibody construct). For example: Klimka et al., British Journal of Cancer (2000) 83: 252-260, describe a screening process using a mouse VL and a human VH library with CDR3 and FR4 retained from the mouse VH. After obtaining antibodies, the VH was screened against a human VL library to obtain antibodies that bound antigen. Beiboer et al., J. Mol. Biol. (2000) 296:833-849 describe a screening process using an entire mouse heavy chain and a human light chain library. After obtaining antibodies, one VL was combined with a human VH library with the CDR3 of the mouse retained. Antibodies capable of binding antigen were obtained. Rader et al., PNAS (1998) 95:8910-8915 describe a process similar to Beiboer et al above.

These just-described techniques are, in and of themselves, known as such in the art. The skilled person will, however, be able to use such techniques to obtain antigen-binding fragments of antibodies according to several embodiments of the disclosure described herein, using routine methodology in the art.

Also disclosed herein is a method for obtaining an antibody antigen binding domain specific for a target antigen (e.g., CD3 or any target antigen described elsewhere herein for targets of binding domains described herein), the method comprising providing by way of addition, deletion, substitution or insertion of one or more amino acids in the amino acid sequence of a VH domain set out herein a VH domain which is an amino acid sequence variant of the VH domain, optionally combining the VH domain thus provided with one or more VL domains, and testing the VH domain or VH/VL combination or combinations to identify a specific binding member or an antibody antigen binding domain specific for a target antigen of interest (e.g., CD3) and optionally with one or more desired properties. The VL domains may have an amino acid sequence which is substantially as set out herein. An analogous method may be employed in which one or more sequence variants of a VL domain disclosed herein are combined with one or more VH domains.

A skilled artisan would appreciate that an epitope that “specifically binds” or “preferentially binds” (used interchangeably herein) to an antibody or a polypeptide is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody, or Fab or scFv thereof, “specifically binds” or “preferentially binds” to a target if it binds with greater affinity, avidity, more readily, and/or with greater duration than it binds to other substances. For example, an antibody that specifically or preferentially binds to a CD3 epitope is an antibody that binds one CD3 epitope with greater affinity, avidity, more readily, and/or with greater duration than it binds to other CD3 epitopes or non-CD3 epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

In certain embodiments, antigen-binding portions of the Fab fragment (third binding domain) as described herein include a heavy chain and a light chain CDR set, respectively interposed between a heavy chain and a light chain framework region (FR) set which provide support to the CDRs and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures-regardless of the precise CDR amino acid sequence. Further, certain FR residues are known to participate in non-covalent interdomain contacts which stabilize the interaction of the antibody heavy and light chains.

The structures and locations of immunoglobulin variable regions may be determined by reference to Kabat, E. A. et al., Sequences of Proteins of Immunological Interest. 4^{th} Edition. US Department of Health and Human Services. 1987, and updates thereof, now available on the Internet (94mmune.bme.nwu.edu).

A skilled artisan would recognize that the term “monoclonal antibody” encompasses a homogeneous antibody population wherein the monoclonal antibody is comprised of amino acids (naturally occurring and non-naturally occurring) that are involved in the selective binding of an epitope. Monoclonal antibodies are highly specific, being directed against a single epitope. The term “monoclonal antibody” encompasses not only intact monoclonal antibodies and full-length monoclonal antibodies, but also fragments thereof (such as Fab, Fab′, F(ab′)₂, Fv), single chain (scFv), variants thereof, fusion proteins comprising an antigen-binding portion, humanized monoclonal antibodies, chimeric monoclonal antibodies, and any other modified configuration of the immunoglobulin molecule that comprises an antigen-binding fragment (epitope recognition site) of the required specificity and the ability to bind to an epitope. It is not intended to be limited as regards the source of the antibody or the manner in which it is made (e.g., by hybridoma, phage selection, recombinant expression, transgenic animals, etc.). The term includes whole immunoglobulins as well as the fragments etc. as described herein.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)2 fragment which comprises both antigen-binding sites. An Fv fragment for use according to certain embodiments as disclosed herein, can be produced by preferential proteolytic cleavage of an IgM, and on rare occasions of an IgG or IgA immunoglobulin molecule. Fv fragments are, however, more commonly derived using recombinant techniques known in the art. The Fv fragment includes a non-covalent VH::VL heterodimer including an antigen-binding site which retains much of the antigen recognition and binding capabilities of the native antibody molecule. Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

In some embodiments of the present disclosure, the Fab fragment comprising a third binding domain binds to CD3. In some embodiments of the present disclosure, the Fab fragment comprising a third binding domain binds to CD3epsilon.

“T-cell receptor” (TCR) is a molecule found on the surface of T-cells that, along with CD3, is generally responsible for recognizing antigens bound to major histocompatibility complex (MHC) molecules. It consists of a disulfide-linked heterodimer of the highly variable (alpha) and (beta) chains in most T-cells. In other T-cells, an alternative receptor made up of variable Y and (delta) chains is expressed. Each chain of the TCR is a member of the immunoglobulin superfamily and possesses one N-terminal immunoglobulin variable region, one immunoglobulin constant region, a transmembrane region, and a short cytoplasmic tail at the C-terminal end (see, Abbas and Lichtman, Cellular and Molecular Immunology (5^{th} Ed.), Editor: Saunders, Philadelphia, 2003; Janeway et ai, Immunobiology: The Immune System in Health and Disease, 4^{th} Ed., Current Biology Publications, p 148, 149, and 172, 1999). TCR as used in the present disclosure may be from various animal species, including human, mouse, rat, or other mammals.

“Anti-TCR Fab” or “Anti-TCR precursor bispecific antibody construct”, refers to a Fab or a precursor tri-specific antibody construct comprising an Fab that specifically binds to a TCR molecule or one of its individual chains (e.g., TCR (alpha), TCR (beta), TCRY or TCR (delta) chain). In certain embodiments, an anti-TCR Fab binds to a TCR (alpha), a TCR (beta), or both. A skilled person would appreciate that the term “Anti-TCR Fab”, may in some embodiments encompass the third binding domain of a precursor tri-specific antibody construct described herein. In some embodiments, the term “Anti-TCR Fab” may encompass the precursor construct, wherein reference is being made to the binding attributes of the third binding domain.

“CD3” is known in the art as a multi-protein complex of six chains (see, Smith-Garvin et al., Annu Rev Immunol. 2009;27:591-619 )). In mammals, the complex comprises a CD3(gamma) chain, a CD3(delta) chain, two CD3(epsilon; ε) chains, and a homodimer of CD3(zeta) chains. The CD3(gamma), CD3(delta), and CD3(epsilon) chains are highly related cell surface proteins of the immunoglobulin superfamily containing a single immunoglobulin domain. The transmembrane regions of the CD3(gamma), CD3(delta), and CD3(epsilon) chains are negatively charged, which is a characteristic that allows these chains to associate with the positively charged T-cell receptor chains. The intracellular tails of the CD3(gamma), CD3(delta), and CD3(epsilon) chains each contain a single conserved motif known as an immunoreceptor tyrosine-based activation motif or ITAM, whereas each CD3(zeta) chain has three. Without wishing to be bound by theory, it is believed the ITAMs are important for the signaling capacity of a TCR complex. CD3 as used in the present disclosure may be from various animal species, including human, mouse, rat, or other mammals.

“Anti-CD3 Fab” as used herein, refers to a Fab comprising a third binding domain that specifically binds to individual CD3 chains (e.g., CD3(gamma) chain, CD3(delta) chain, or CD3(epsilon; ε) chain) or a complex formed from two or more individual CD3 chains (e.g., a complex of more than one CD3(epsilon) chains, a complex of a CD3(gamma) and CD3(epsilon) chain, a complex of a CD3(delta) and CD3(epsilon) chain). In certain embodiments, an anti-CD3 Fab specifically binds to a CD3(gamma), a CD3(delta), or a CD3(epsilon), or any combination thereof, and in certain embodiments, a CD3(epsilon). In some embodiments, an anti-CD3 Fab binds to the N-terminus of CD3 epsilon. In some embodiments, an anti-CD3 Fab binds to an extracellular epitope of CD3 epsilon.

In some embodiments, the anti-CD3 Fab binds to an epitope comprised within amino acids 1-27 of CD3 epsilon. In some embodiments, the anti-CD3 Fab binds to amino acids 1-27 of CD3 epsilon. In some embodiments, the anti-CD3 Fab binds to amino acids 1-27 of a human CD3 epsilon. Amino acids 1-27 of CD3 epsilon are set forth in SEQ ID NO: 5.

A skilled person would appreciate that the term “Anti-CD3 Fab”, may in some embodiments encompass the third binding domain of a precursor tri-specific antibody construct described herein. In some embodiments, the term “Anti-CD3 Fab” may encompass the precursor construct, wherein reference is being made to the binding attributes of the third binding domain.

In some embodiments, a third binding domain of a precursor construct comprises a Fab. In some embodiments, when referring to a third binding domain of a precursor construct the term “Fab” will be used, wherein the term encompasses a third bind domain of a precursor construct. In some embodiments, the term “Fab” may be used interchangeably with the phrase “third binding domain” having all the same qualities and meanings.

In some embodiments, a precursor tri-specific antibody construct comprises a third binding domain that binds to an extracellular epitope of CD3 epsilon. In some embodiments, a precursor tri-specific antibody construct comprises a third binding domain that binds to the N-terminus of CD3 epsilon. In some embodiments, a precursor tri-specific antibody construct comprises a third binding domain that binds to an epitope with amino acids 1-27 of CD3 epsilon. In some embodiments, the anti-CD3 Fab binds to amino acids 1-27 of CD3 epsilon. In some embodiments, the anti-CD3 Fab binds to amino acids 1-27 of a human CD3 epsilon. Amino acids 1-27 of CD3 epsilon are set forth in SEQ ID NO: 5.

“TCR complex,” as used herein, refers to a complex formed by the association of CD3 with TCR. For example, a TCR complex can be composed of a CD3(gamma) chain, a CD3(delta) chain, two CD3(epsilon) chains, a homodimer of CD3(zeta) chains, a TCR(alpha) chain, and a TCR(beta) chain. Alternatively, a TCR complex can be composed of a CD3(gamma) chain, a CD3(delta) chain, two CD3(epsilon) chains, a homodimer of CD3(zeta) chains, a TCRY chain, and a TCR(delta) chain.

“A component of a TCR complex,” as used herein, refers to a TCR chain (i.e., TCR(alpha), TCR(beta), TCRY or TCR(delta)), a CD3 chain (i.e., CD3(gamma), CD3(delta), CD3(epsilon) or CD3(zeta)), or a complex formed by two or more TCR chains or CD3 chains (e.g., a complex of TCR(alpha) and TCR(beta), a complex of TCRY and TCR(delta), a complex of CD3(epsilon) and CD3(delta), a complex of CD3(gamma) and CD3(epsilon), or a sub-TCR complex of TCR(alpha), TCR(beta), CD3(gamma), CD3(delta), and two CD3(epsilon) chains).

By way of background, the TCR complex is generally responsible for initiating a T-cell response to antigen bound to MHC molecules. It is believed that binding of a peptide:MHC ligand to the TCR and a co-receptor (i.e., CD4 or CD8) brings together the TCR complex, the co-receptor, and CD45 tyrosine phosphatase. This allows CD45 to remove inhibitory phosphate groups and thereby activate Lck and Fyn protein kinases. Activation of these protein kinases leads to phosphorylation of the ITAM on the CD3(zeta) chains, which in turn renders these chains capable of binding the cytosolic tyrosine kinase ZAP-70. The subsequent activation of bound ZAP-70 by phosphorylation triggers three signaling pathways, two of which are initiated by the phosphorylation and activation of PLC-(gamma), which then cleaves phosphatidylinositol phosphates (PIPs) into diacylglycerol (DAG) and inositol trisphosphate (IP3). Activation of protein kinase C by DAG leads to activation of the transcription factor NFKB. The sudden increase in intracellular free Ca2+ as a result of IP3 action activates a cytoplasmic phosphatase, calcineurin, which enables the transcription factor NFAT (nuclear factor of activated T-cells) to translocate form the cytoplasm to the nucleus. Full transcriptional activity of NFAT also requires a member of the AP-1 family of transcription factors; dimers of members of the Fos and Jun families of transcription regulators.

A third signaling pathway initiated by activated ZAP-70 is the activation of Ras and subsequent activation of a MAP kinase cascade. This culminates in the activation of Fos and hence of the AP-1 transcription factors. Together, NFKB, NFAT, and AP-1 act on the T-cell chromosomes, initiating new gene transcription that results in the differentiation, proliferation and effector actions of T-cells. See, Pitcher et al., 2003., TRENDS in Immunol. 24, 554-560; Smith-Garvin et al., Annu Rev Immunol. 2009;27:591-619 .

In certain embodiments, the Fab specifically binds to an individual human CD3 chain (e.g., human CD3(gamma) chain, human CD3(delta) chain, or human CD3(epsilon) chain) or a combination of two or more of the individual human CD3 chains (e.g., a complex of human CD3(gamma) and human CD3(epsilon) or a complex of human CD3(delta) and human CD3(epsilon)). In certain embodiments, the Fab specifically binds to a human CD3(epsilon) chain. In certain embodiments, the Fab specifically binds to an extracellular epitope of a human CD3(epsilon) chain. In certain embodiments, the Fab specifically binds to an epitope within SEQ ID NO: 3.

In certain embodiments, the third binding domain specifically binds to an individual human CD3 chain (e.g., human CD3(gamma) chain, human CD3(delta) chain, or human CD3(epsilon) chain) or a combination of two or more of the individual human CD3 chains (e.g., a complex of human CD3(gamma) and human CD3(epsilon) or a complex of human CD3(delta) and human CD3(epsilon)). In certain embodiments, the third binding domain specifically binds to a human CD3(epsilon) chain. In certain embodiments, the third binding domain specifically binds to an extracellular epitope of a human CD3(epsilon) chain. In certain embodiments, the third binding domain specifically binds to an epitope within SEQ ID NO: 3.

In certain other embodiments, a Fab of the present disclosure comprising a third binding domain specifically binds to TCR(alpha), TCR(beta), or a heterodimer formed from TCR(alpha) and TCR(beta). In certain embodiments, a Fab specifically binds to one or more of human TCR(alpha), human TCR(beta), or a heterodimer formed from human TCR(alpha) and human TCR(beta).

In certain embodiments, a Fab of the present disclosure comprising a third binding domain binds to a complex formed from one or more CD3 chains with one or more TCR chains, such as a complex formed from a CD3(gamma) chain, a CD3(delta) chain, a CD3(epsilon) chain, a TCR(alpha) chain, or a TCR(beta) chain, or any combination thereof. In other embodiments, a Fab of the present disclosure binds to a complex formed from one CD3(gamma) chain, one CD3(delta) chain, two CD3(epsilon) chains, one TCR(alpha) chain, and one TCR(beta) chain. In further embodiments, a Fab of the present disclosure binds to a complex formed from one or more human CD3 chains with one or more human TCR chains, such as a complex formed from a human CD3(gamma) chain, a human CD3(delta) chain, a human CD3(epsilon), a human TCR(alpha) chain, or a human TCR(beta) chain, or any combination thereof. In certain embodiments, a Fab of the present disclosure binds to a complex formed from one human CD3(gamma) chain, one human CD3(delta) chain, two human CD3(epsilon) chains, one human TCR(alpha) chain, and one human TCR(beta) chain.

Fabs of this disclosure can be generated as described herein or by a variety of methods known in the art (see, e.g., U.S. Pat. Nos. 6,291,161; 6,291,158). Sources of Fabs include monoclonal antibody nucleic acid sequences from various species (which can be formatted as antibodies, Fvs, scFvs or Fabs, such as in a phage library), including human, camelid (from camels, dromedaries, or llamas; Hamers-Casterman et al. (1993) Nature, 363:446 and Nguyen et al. (1998) J. Mol. Biol., 275:413), shark (Roux et al. (1998) Proc. Nat′l. Acad. Sci. (USA) 95:11804), fish (Nguyen et al. (2002) Immunogenetics, 54:39), rodent, avian, or ovine.

An anti-human CD3 antibody with cross reactivity to monkey CD3 is particularly desirable, such as the SP34 mouse monoclonal antibody, which binds specifically to human CD3 in denatured form (Western blot or dot blot) and in native form (on T-cells) (Pressano, S. The EMBO J. 4:337-344, 1985; Alarcon, B. EMBO J. 10:903-912, 1991). SP34 mouse monoclonal antibody also binds to CD3c singly transfected COS cells as well as CD3ε/γ or CD3.ε/δ double transfectants (Salmeron A. et al., J. Immunol. 147:3047-52, 1991). SP34 antibody also cross reacts non-human primates (Yoshino N. et al., Exp. Anim 49:97-110, 2000; Conrad M L. et al., Cytometry 71A:925-33, 2007). In addition, SP34 activates T-cell when cross-linked (Yang et al., J. Immunol. 137:1097-1100, 1986). Cross-reactivity to monkey CD3 is important as this allows toxicity studies to be carried out in non-human primates using the clinical candidate directly, rather than in chimpanzee or using a surrogate molecule. Thus, toxicity studies using such cross-reactive anti-CD3 Fab in a precursor bispecific antibody construct of the present disclosure provide more relevant safety assessments.

Other illustrative anti-CD3 antibodies include the Cris-7 monoclonal antibody (Reinherz, E. L. et al. (eds.), Leukocyte typing II., Springer Verlag, New York, (1986)), BC3 monoclonal antibody (Anasetti et al. (1990) J. Exp. Med. 172:1691), OKT3 (Ortho multicenter Transplant Study Group (1985) N. Engl. J. Med. 313:337) and derivatives thereof such as OKT3 ala-ala (Herold et al. (2003) J. Clin. Invest. 11:409), visilizumab (Carpenter et al. (2002) Blood 99:2712), and 145-2C11 monoclonal antibody (Hirsch et al. (1988) J. Immunol. 140: 3766). Further CD3 binding molecules contemplated for use herein include UCHT-1 (Beverley, P C and Callard, R. E. (1981) Eur. J. Immunol. 11: 329-334) and CD3 binding molecules described in WO2004/106380; WO2010/037838; WO2008/119567; WO2007/042261; WO2010/0150918, which are incorporated herein in their entirety.

In some embodiments, the amino acid sequence of a third binding region comprising an anti-CD3 epsilon binding activity comprises any anti-CD3epsilon sequence known in the art. In some embodiments, the amino acid sequence of a third binding region comprising binding activity to an anti-CD3 epsilon or a derivative thereof or an antibody fragment thereof, comprises any anti-CD3epsilon sequence known in the art. Examples of known anti-CD3 epsilon amino acid sequences may be found for example but no limit to U.S. Pats. Nos.: 9,822,180; 9,493,563; 9,587,021; 9,562,073; U.S. Published Application Nos.: 2013/0129729; 2017/0247476; 2016/0194399; 2010/0150918; 2018/0112011; and WO2017/162587, which all included herein in their entirety.

An exemplary anti-TCR antibody is H57 monoclonal antibody (Lavasani et al. (2007) Scandinavian Journal of Immunology 65:39-47).

Antigen binding fragment sequences (e.g., heavy and light chain variable region sequences) for Fab fragments may be available in public databases or using traditional strategies for hybridoma development using a CD3 chain, TCR component, or other Fab binding target as an immunogen in convenient systems (e.g., mice, HuMAb Mouse.RTM., TC Mouse.TM., KM-Mouse.RTM., llamas, chicken, rats, hamsters, rabbits, etc.) can be used to develop Fabs for use herein. As would be understood by the skilled person, Fab fragments may be generated using various technologies known in the art, including antibody display technologies such as phage, yeast, ribosome and mRNA display technologies; B cell culture technology such as SLAM technology; or using high throughput gene sequencing technologies on B cells or plasma B cells isolated from an immunized animal subject or immunized human subject.

In some embodiments, a third binding domain (an Fab) disclosed herein, comprises humanized FR amino acid sequence and native sequence of a mouse monoclonal antibody for the CDR amino acid sequences. Examples of anti-CD3 epsilon amino acid sequences wherein the FR sequences have been humanized while the CDR amino acid sequences remain those of the SP34 mouse monoclonal antibody, are disclosed in International Application Publication No. WO 2007/042261, which is incorporated here in its entirety.

Illustrative third binding domains (for example but not limited to anti-CD3 epsilon Fabs) sequences comprised within a precursor bispecific antibody construct of the present disclosure include the VH, CH1, VL, and CL amino acid sequences, and the polynucleotides encoding them, as set forth in Tables 1 and 2, respectively. Amino acid sequences comprising a third binding domain include those set forth as: SEQ ID NOs: 46-72 and 114 (VH) and 75-103 and 116 (VL) including CDRs thereof, such as those set forth in SEQ ID NOs: 104-112. In some embodiments, third binding domains (e.g., Fabs) sequences comprised within a precursor tri-specific antibody construct of the present disclosure include the VH, CH1, VL, and CL amino acid sequences, as set forth in Table 2, or a homolog thereof. In some embodiments, homologs of SEQ ID NOs: 46-72 and 114, and 75-103 and 116, maintain their CDR regions, for example as set for the in SEQ ID NOs: 104-112.

TABLE 2

Amino Acid Sequences of Anti-CD3 VH, VL, HC, LC, and CDR, and Combinations Thereof.

| SEQ ID NOs | Description |
|---|---|
| 45 | Mouse monoclonal SP34(mu) variable heavy chain |
| 46-72 | Variable Heavy chains |
| 73 | Variable Light chain from mouse monoclonal antibody SP34VL(mu) |
| 74, 461, 466, 468 | Variable Light chain and Constant Light Chain |
| 457, 465, 467 | Variable Heavy chain and Constant Heavy Chain |
| 75-103 | Variable Light chains |
| 104, 458 | CDR1 of Heavy Chain (CDR-H1) |
| 105, 459 | CDR2 of Heavy Chain (CDR-H2) |
| 106, 460 | CDR3 of Heavy Chain (CDR-H3) |
| 107, 108, 109, 462 | CDR1 of Light Chain (CDR-L1) |
| 110, 463 | CDR2 of Light Chain (CDR-L2) |
| 111, 112, 464 | CDR3 of Light Chain (CDR-L3) |
| 113 | anti-CD3 Variable Heavy chain and heavy constant region 1 |
| 114 | anti-CD3 epsilon VH |
| 115 | anti-CD3 epsilon Constant Heavy Chain. |
| 116 | anti-CD3 epsilon VL |
| 117 | anti-CD3 epsilon Constant Light Chain. |

In some embodiments, a third binding domain binds a CD3 epsilon polypeptide. In some embodiments, a third binding domain binds an extracellular domain of a human CD3 epsilon polypeptide. In some embodiments, a third binding domain comprises an Fab fragment comprising a variable heavy chain region (VH) comprising a CDR-H1, a CDR-H2, and a CDR-H3, and a variable light chain region (VL) comprising a CDR-L1, a CDR-L2, and a CDR-L3, wherein the third binding domain binds an extracellular domain of a human CD3 epsilon polypeptide. In some embodiments, a third binding domain binds to an epitope within SEQ ID NO: 3. In some embodiments, a third binding domain binds SEQ ID NO: 5.

In some embodiments, the amino acid sequence of an anti-human CD3 epsilon CDR-H1 is set forth in SEQ ID NO: 104. In some embodiments, the amino acid sequence of an anti-human CD3 epsilon CDR-H2 is set forth in SEQ ID NO: 105. In some embodiments, the amino acid sequence of an anti-human CD3 epsilon CDR-H3 is set forth in SEQ ID NO: 106. In some embodiments, the amino acid sequence of an anti-human CD3 epsilon CDR-L1 is set forth in any one of SEQ ID NOs: 107-109. In some embodiments, the amino acid sequences of an anti-human CD3 epsilon CDR-L2 is set forth in SEQ ID NO: 110. In some embodiments, the amino acid sequences of an anti-human CD3 epsilon CDR-L3 is set forth in SEQ ID NO: 111-112.

In some embodiments, a third binding domain comprises an Fab fragment comprising a variable heavy chain region (VH) and a variable light chain region (VL) that binds an extracellular domain of a human CD3 epsilon polypeptide. In some embodiments, the amino acid sequence of VH and VL for an anti-human CD3 epsilon are selected from the amino acid sequences set forth in any of SEQ ID NO: 46-72 and 114 (VH), and 75-103 and 116 (VL). In some embodiments, the amino acid sequence of VH and VL for an anti-human CD3 epsilon comprises a homolog of sequences selected from the amino acid sequences set forth in any of SEQ ID NO: 46-72 and 114 (VH), and 75-103 and 116 (VL).

In some embodiments, the amino acid sequence of a VH for a human CD3 epsilon third binding domain (VH1) are selected from the amino acid sequences set forth in any of SEQ ID NOs: 46-72 and 114, or a homolog thereof. In some embodiments, the amino acid sequence of a VL for a human CD3 epsilon third binding domain (VL1) are selected from the amino acid sequences set forth in any of SEQ ID NOs: 75-103 and 116, or a homolog thereof.

In some embodiments, homologues comprise polypeptides which are at least 50%, at least 55%, at least 60%, at least 65%, at least 70%, at least 75%, at least 80%, at least 85%, at least 87%, at least 89%, at least 91%, at least 93%, at least 95%, at least 96%, at least 97%, at least 98%, or at least 99% homologous to the amino acid sequence of variable light or variable heavy chains of anti-CD3epsilon. In some embodiments, homologues comprise polypeptides which are at least 50%, at least 55%, at least 60%, at least 65%, at least 70%, at least 75%, at least 80%, at least 85%, at least 87%, at least 89%, at least 91%, at least 93%, at least 95%, at least 96%, at least 97%, at least 98%, or at least 99% identical to the amino acid sequence of variable light or variable heavy chains of anti-CD3epsilon. In some embodiments, homologues comprise polypeptides which are at least 50%, at least 55%, at least 60%, at least 65%, at least 70%, at least 75%, at least 80%, at least 85%, at least 87%, at least 89%, at least 91%, at least 93%, at least 95%, at least 96%, at least 97%, at least 98%, or at least 99% homologous to an anti-human CD3 epsilon VH or an anti-human CD3 epsilon VL, respectively, as determined using BlastP software of the National Center of Biotechnology Information (NCBI) using default parameters.

In some embodiments, homology also encompasses deletion, insertion, or substitution variants, including an amino acid substitution, thereof and biologically active polypeptide fragments thereof. In one embodiment, the variant comprises conservative substitutions, or deletions, insertions, or substitutions that do not significantly alter the three-dimensional structure of the polypeptide of interest, e.g., VL or VH region, particularly in the areas of the CDR epitope binding regions. In some embodiments, the deletion, insertion, or substitution does not alter the function of interest of the anti-human CD3 epsilon Fab, which in some embodiments, is binding to a CD3 epsilon sequence on a target T-cell.

In some embodiments, a third binding domain binding to a CD3 epsilon extracellular epitope VH1 includes the sequences set forth in SEQ ID NOs: 46-72 and 114, or a homolog thereof. In some embodiments, a second binding domain binding to a CD3 epsilon extracellular epitope VL1 includes the sequences set forth in SEQ ID NO: 75-103 and 116, or a homolog thereof. In some embodiments, a third binding domain binding to a CD3 epsilon extracellular epitope comprises a sequence selected from the sequences set forth in SEQ ID NOs: 46-72, 74, and 114 or a homolog thereof, and a sequence selected from the sequences set forth in SEQ ID NOs: 75-103, 113, 115, and 116, or a homolog thereof. In some embodiments, a third binding domain binding to a CD3 epsilon extracellular epitope comprises the sequence set forth in SEQ ID NO: 113 or a homolog thereof, and the sequence set forth in SEQ ID NO: 74 or a homolog thereof.

In some embodiments, the third binding domain VL region comprises amino acid sequences as set forth for CDR-L1 (selected from SEQ ID NOs: 107-109, 462), CDR-L2 (SEQ ID NOs: 110, 463), and CDR-L3 (selected from SEQ ID NOs:111, 112, or 464), and the third binding domain VH region comprises CDR-H1 (SEQ ID NOs: 104, 458), CDR-H2 (SEQ ID NOs: 105, 459), and CDR-H3 (SEQ ID NOs: 106, 460).

In some embodiments of a precursor tri-specific antibody construct, the VL region of the third binding domain comprises the amino acid sequence set forth in any of SEQ ID NO: 75-103 and 116, or an amino acid sequence having at least 80% homology thereto. In some embodiments, a VL region of the third binding comprising an amino acid sequence having at least 80% homology thereto, comprises framework sequences having at least 80% homology, wherein the CDR regions are “as is” in the selected amino acid sequence (e.g. SEQ ID NOs: 107-112).

In some embodiments of a precursor tri-specific antibody construct, the VH region of the third binding comprises the amino acid sequence set forth in any of SEQ ID NO: 46-72 and 114, or an amino acid sequence having at least 80% homology thereto. In some embodiments, a VH region of the third binding comprising an amino acid sequence having at least 80% homology thereto, comprises framework sequences having at least 80% homology, wherein the CDR regions are “as is” in the selected amino acid sequence (e.g. SEQ ID NOs: 104-106).

In some embodiments, a first binding domain comprises a humanized binding domain. In some embodiments, a second binding domain comprises a humanized binding domain. In some embodiments, a third binding domain comprises a humanized binding domain. In some embodiments, a first, or a second or a third binding domain, or any combination thereof, comprises a humanized binding domain.

As would be understood by the skilled person and as described herein, in some embodiments, a complete antibody comprises two heavy chains and two light chains Each heavy chain consists of a variable region and a first, second, and third constant region, while each light chain consists of a variable region and a constant region. Mammalian heavy chains are classified as α, δ. E, γ, and µ, and mammalian light chains are classified as λ or κ. Immunoglobins comprising the α, δ. E, γ, and µ, heavy chains are classified as immunoglobin (Ig)A, IgD, IgE, IgG, and IgM. The complete antibody forms a “Y” shape. The stem of the Y consists of the second and third constant regions (and for IgE and IgM, the fourth constant region) of two heavy chains bound together and disulfide bonds (inter-chain) are formed in the hinge. Heavy chains γ, α, and δ have a constant region composed of three tandem (in a line) Ig domains, and a hinge region for added flexibility; heavy chains µ and ε have a constant region composed of four immunoglobulin domains. The second and third constant regions are referred to as “CH2 domain” and “CH3 domain”, respectively. Each arm of the Y includes the variable region and first constant region of a single heavy chain bound to the variable and constant regions of a single light chain. The variable regions of the light and heavy chains are responsible for antigen binding.

“Complementarity determining region” or “CDR” with regard to an antibody refers to a highly variable loop in the variable region of the heavy chain or the light chain of an antibody. CDRs can interact with the antigen conformation and largely determine binding to the antigen (although some framework regions are known to be involved in binding). The heavy chain variable region and the light chain variable region each contain 3 CDRs. The CDRs can be defined or identified by conventional methods, such as by sequence according to Kabat et al (Wu, T T and Kabat, E. A., J Exp Med. 132(2):211-50, (1970); Borden, P. and Kabat E. A., PNAS, 84: 2440-2443 (1987); Kabat, E. A. et al, Sequences of proteins of immunological interest, Published by DIANE Publishing, 1992), or by structure according to Chothia et al (Choithia, C. and Lesk, A. M., J. Mol. Biol., 196(4): 901-917 (1987), Choithia, C. et al, Nature, 342: 877-883 (1989)).

“Heavy chain variable region” or “VH” with regard to an antibody refers to the fragment of the heavy chain that contains three CDRs interposed between flanking stretches known as framework regions, which are more highly conserved than the CDRs and form a scaffold to support the CDRs.

“Light chain variable region” or “VL” with regard to an antibody refers to the fragment of the light chain that contains three CDRs interposed between framework regions.

“Fv” with regard to an antibody refers to the smallest fragment of the antibody to bear the complete antigen binding site. An Fv fragment consists of the variable region of a single light chain bound to the variable region of a single heavy chain.

“Single-chain Fv antibody” or “scFv” with regard to an antibody refers to an engineered antibody consisting of a light chain variable region and a heavy chain variable region connected to one another directly or via a peptide linker sequence.

“Single domain camel antibody” or “camelid VHH” as used herein refers to the smallest known antigen-binding unit of a heavy chain antibody (Koch-Nolte, et al, FASEB J., 21: 3490-3498 (2007)). A “heavy chain antibody” or a “camelid antibody” refers to an antibody that contains two VH domains and no light chains (Riechmann L. et al, J. Immunol. Methods 231:25-38 (1999); WO94/04678; WO94/25591; U.S. Pat. No. 6,005,079).

“Single domain antibody” or “dAb” refers to an antibody fragment that consists of the variable region of an antibody heavy chain (VH domain) or the variable region of an antibody light chain (VL domain) (Holt, L., et al, Trends in Biotechnology, 21(11): 484-490).

The term “disulfide bond” as used herein refers to the binding of a heavy chain fragment and a light chain fragment through one or more disulfide bonds. The one or more disulfide bonds can be formed between the two fragments by linking the thiol groups in the two fragments. In certain embodiments, the one or more disulfide bonds can be formed between one or more cysteine residues in the heavy chain fragment and the light chain fragment, respectively.

A “variable region linking sequence” is an amino acid sequence that connects a heavy chain variable region to a light chain variable region and provides a linker function compatible with interaction of the two sub-binding domains so that the resulting polypeptide retains a specific binding affinity to the same target molecule as an antibody that comprises the same light and heavy chain variable regions. In certain embodiments, a hinge useful for linking a binding domain to an immunoglobulin CH2 or CH3 region polypeptide may be used as a variable region linking sequence.

In some embodiments, a third binding domain comprises a heavy chain variable (VH) region and a light chain variable (VL) region, wherein said first or second regulatory domain is located N-terminally to said VL region or VH region of said third binding domain. In some embodiments, a third binding domain comprises a heavy chain variable (VH) region and a light chain variable (VL) region, wherein said first regulatory domain is located N-terminally to said VL region of said third binding domain, and a second regulatory domain is located N-terminally to said VH region of said third binding domain. In some embodiments, a third binding domain comprises a heavy chain variable (VH) region and a light chain variable (VL) region, wherein said first regulatory domain is located N-terminally to said VH region of the third binding domain, and the second regulatory domain is located N-terminally to said VL region of the third binding domain.

In some embodiments, a third binding domain comprises a heavy chain constant (CH1) region and a light chain constant (CL) region, wherein said first or second binding domain is located C-terminally to said CH1 region or CL region of said third binding domain. In some embodiments, a third binding domain comprises a heavy chain constant (CH1) region and a light chain constant (CL) region, wherein said first binding domain is located C-terminally to said CL region of said third binding domain, and a second binding domain is located C-terminally to said CH1 region of said third binding domain. In some embodiments, a third binding domain comprises a heavy chain constant (CH1) region and a light chain constant (CL) region, wherein said first binding domain is located C-terminally to said CH1 region of the third binding domain, and the second binding domain is located C-terminally to said CL region of the third binding domain. A skilled artisan would appreciate that the first and second regulatory domains are located N-terminally to the VH and VL, wherein when a first regulatory domain is located N-terminally to VH than the second regulatory domain is located N-terminally to the VL, and vice-versa when the second regulatory domain is located N-terminal to the VH of the third binding domain, the first regulatory domain is located N-terminally to the VL of the third binding domain. Similarly, the skilled artisan would appreciate that the first and second binding domains are located C-terminally to the CH1 and CL domains of the third binding domain, wherein when the first binding domain is located C-terminally to the CH1 the second binding domain is location C-terminally to the CL, and vice-versa, when the second binding domain is located C-terminally to the CH1, the first binding domain is located C-terminally to the CL of the third binding domain.

An alternative source of binding domains may include sequences that encode random peptide libraries or sequences that encode an engineered diversity of amino acids in loop regions of alternative non-antibody scaffolds, such as fibrinogen domains (see, e.g., Weisel et al. (1985) Science 230:1388), Kunitz domains (see, e.g., U.S. Pat. No. 6,423,498), lipocalin domains (see, e.g., WO 2006/095164), V-like domains (see, e.g., U.S. Pat. Application Publication No. 2007/0065431), C-type lectin domains (Zelensky and Gready (2005) FEBS J. 272:6179), or Fcab.TM. (see, e.g., PCT Patent Application Publication Nos. WO 2007/098934; WO 2006/072620), or the like.

As depicted in FIGS. 4-5, Fab third binding domains may comprise anti-NK cell surface antigen binding domains. For example, but not limited to anti-NKG2A or anti-NKG2D Fab third binding domains.

As depicted in the FIGS. 2-5, scFv are particularly illustrative binding domains. A first or second binding domain, or both a first and second binding domain, which in some embodiments comprises an scFv fragment, may bind to any of a variety of target molecules, including but not limited to a FcγRI, FcγRIIa FcγRIIb FcγRIIIa FcγRIIIb, CD28, CD137, CTLA-4, FAS, fibroblast growth factor receptor 1 (FGFR1), FGFR2, FGFR3, FGFR4, glucocorticoid-induced TNFR-related (GITR) protein, lymphotoxin-beta receptor (LTβR), toll-like receptors (TLR), tumor necrosis factor-related apoptosis-inducing ligand-receptor 1 (TRAIL receptor 1) and TRAIL receptor 2, prostate-specific membrane antigen (PSMA) protein, prostate stem cell antigen (PSCA) protein, tumor-associated protein carbonic anhydrase IX (CAIX), epidermal growth factor receptor 1 (EGFR1), EGFRvIII, human epidermal growth factor receptor 2 (Her2/neu; Erb2), ErbB3 also known as HER3, Folate receptor, ephrin receptors, PDGFRa, ErbB-2, CD20, CD22, CD30, CD33, CD40, CD37, CD38, CD70, CD74, CD56, CD40), CD80, CD86, CD2, p53, cMet also known as tyrosine-protein kinase Met or hepatocyte growth factor receptor (HGFR), MAGE-A1, MAGE-A2, MAGE-A3, MAGE-A4, MAGE-A6, MAGE-A10, MAGE-A12, BAGE, DAM-6, -10, GAGE-1, -2, -8, GAGE-3, -4, -5, -6, -7B, NA88-A, NY-ESO-1, BRCA1, BRCA2, MART-1, MC1R, Gp100, PSA, PSM, Tyrosinase, Wilms’ tumor antigen (WT1), TRP-1, TRP-2, ART-4, CAMEL, Cyp-B, hTERT, hTRT, iCE, MUC1, MUC2, P-cadherin, Myostatin (GDF8), Cripto (TDGF1), MUC5AC, PRAME, P15, RU1, RU2, SART-1, SART-3, WT1, AFP, β-catenin/m, Caspase-8/m, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, ETV6/AML, LDLR/FUT, Pml/RARa, TEL/AML1, CD28, CD137, CanAg, Mesothelin, DR5, PD-1, PD1L, IGF-1R, CXCR4, Neuropilin 1, Glypicans, EphA2, CD138, B7-H3, B7-H4, gpA33, GPC3, SSTR2, ROR1, 5T4, an NK surface antigen, or a VEGF-R2. In some embodiments, a TAA comprises a PSMA, CD30, B7-H3, B7-H4, gpA33, HER2, P-cadherin, gp100, DR5, GPC3, SSTR2, Mesothelin, ROR1, 5T4, Folate receptor, an NK surface antigen, or an EGFR. In some embodiments, a TAA is selected from the group consisting of a PSMA, an ROR1, a 5T4, and an EGFR. These and other tumor proteins or tumor associated proteins are known to the skilled artisan.

In certain embodiments, the first or second binding domain, or both the first and second binding domain specifically binds to an antigen target that is associated with a disease condition. The disease condition may include a physiological condition, a pathological condition and a cosmetic condition. Examples of illustrative conditions include, without limitation, cancer, inflammatory disorders, allograft transplantation, type I diabetes, type II diabetes, and multiple sclerosis.

In some embodiments, the specific structural components of a precursor tri-specific antibody construct comprise a first and second binding domain, for example but not limited to an scFv fragment, a third binding domain, for example but not limited to an Fab fragment, linker regions, and a first and second regulatory domain, where said regulatory domains may each comprise a protease cleavable domain and an HSA polypeptide sequence or a protease cleavable domain and a CAP component, and linkers, or any combination thereof, as have been described herein detail.

In some embodiments, a precursor tri-specific antibody construct comprises two polypeptides. In some embodiments, these polypeptides may be identified based on the Heavy chain (HC) or Light chain (LC) components based of the third binding domain. In some embodiments, these polypeptides may be identified as polypeptide A and polypeptide B. In some embodiments, polypeptide A comprises a HC polypeptide and polypeptide B comprises a LC polypeptide. In other embodiments, polypeptide A comprises a LC polypeptide and polypeptide B comprises a HC polypeptide.

In some embodiments, a precursor tri-specific antibody construct described herein, comprises a third binding domain comprising a heavy chain variable (VH) region and a light chain variable (VL) region; wherein a first binding domain is located C-terminally to said CL or said CH1 region of the third binding domain; wherein when said first binding domain is located C-terminally to said CL region, said second binding domain is located C-terminally to said CH1 region, and when said first binding domain is located C-terminally to said CH1 region, said second binding domain is located C-terminally to said CL region. In some embodiments, a precursor tri-specific antibody construct described herein, comprises a third binding domain comprising a heavy chain variable (VH) region and a light chain variable (VL) region;
wherein said first and second binding domains are located as described above, and
wherein said first regulatory domain, comprising said HLP domain located N-terminally to said protease cleavage domain, is located N-terminally to said VH region or to said VL region of said third binding domain;
wherein (a) when said first regulatory domain is located N-terminally to said VL region, said second regulatory domain, comprising said CAP component located N-terminally to said protease cleavage domain, is located N-terminally to said VH region, and (b) when said first regulatory domain is located N-terminally to said VH region, said second regulatory domain comprising said CAP component located N-terminally to said protease cleavage domain, is located N-terminally to said VL region.

A skilled artisan would appreciate that the designations “Polypeptide A” and “Polypeptide B” are merely names indicating two heterologous polypeptide chains, and as such the names themselves may be interchanged or change, e.g., Polypeptide 1 and Polypeptide 2. Further, encompassed by this terminology are two structurally different polypeptide chains that together form a precursor tri-specific antibody construct, as described herein. Further, the skilled artisan would appreciate the modular nature of the precursor constructs described herein, wherein modules may be substituted one for another, wherein they provide similar or different activities. For example, but not limited to, an scFv may have to order N-terminus to C-terminus VL-VH or VH-VL; or a regulatory domain may comprise a CAP component or a HLP domain.

One skilled in the art would appreciate that a masking regulatory domain linked may be linked to the C-terminal region of Polypeptide A if the binding region were to an NK antigen, and could in another embodiment, be linked instead to polypeptide B if said binding region were to an NK antigen, or in yet another embodiment, a regulatory domain with a CAP could be linked at both the C-terminal of a polypeptide A and a polypeptide B if the first and second binding regions were both to NK antigens.

In some embodiments, the first or second regulatory domains are components of either the HC or LC and may be positioned N-terminal to the third binding domain, or C-terminal to a first or second binding domain, and the first or second binding domains comprises scFv, wherein the components of the scFv (VL, L, and VH) are independently ordered VL-L-VH or VH-L-VL, are positioned C-terminal to the third binding domain, and are components of either the HC or LC, respectively.

A skilled artisan would appreciate that different regulatory domains may be included with a precursor construct depending on the desired functionality of the precursor construct. For example, a tri-specific precursor construct may comprise just a single regulatory domain, either an HLP domain or a CAP domain (See FIGS. 2-5, respectively).

In some embodiments, the precursor tri-specific antibody construct comprises two polypeptides, polypeptide A and polypeptide B, each of which comprising one or more heavy chain variable region (VH) and one or more light chain variable region (VL), with linkers where appropriate, and each polypeptide is linked to a regulatory domain such as a HLP domain or a CAP component. Examples of these polypeptide A and polypeptide B include:
(a) polypeptide A comprises components having an order N-terminal to C-terminal: HLP domain, protease cleavage domain, VH of the third binding domain, first binding domain comprising first VH-first VL; and polypeptide B comprises components having an order N-terminal to C-terminal: CAP component, protease cleavage domain, VL of the third binding domain, second binding domain comprising second VH-second VL; or
(b) polypeptide A comprises components having an order N-terminal to C-terminal: HLP domain, protease cleavage domain, VH of the third binding domain, first binding domain comprising first VL-first VH; and polypeptide B comprises components having an order N-terminal to C-terminal: CAP component, protease cleavage domain, VL of the third binding domain, second binding domain comprising second VH-second VL; or
(c) polypeptide A comprises components having an order N-terminal to C-terminal: HLP domain, protease cleavage domain, VH of the third binding domain, first binding domain comprising first VH-first VL; and polypeptide B comprises components having an order N-terminal to C-terminal: CAP component, protease cleavage domain, VL of the third binding domain, second binding domain comprising second VL-second VH; or
(d) polypeptide A comprises components having an order N-terminal to C-terminal: HLP domain, protease cleavage domain, VH of the third binding domain, first binding domain comprising first VL-first VH; and polypeptide B comprises components having an order N-terminal to C-terminal: CAP component, protease cleavage domain, VL of the third binding domain, second binding domain comprising second VL-second VH; or
(e) polypeptide A comprises components having an order N-terminal to C-terminal: CAP component, protease cleavage domain, VH of the third binding domain, first binding domain comprising first VH-first VL; and polypeptide B comprises components having an order N-terminal to C-terminal: HLP domain, protease cleavage domain, VL of the third binding domain, second binding domain comprising second VH-second VL; or
(f) polypeptide A comprises components having an order N-terminal to C-terminal: CAP component, protease cleavage domain, VH of the third binding domain, first binding domain comprising first VL-first VH; and polypeptide B comprises components having an order N-terminal to C-terminal: HLP domain, protease cleavage domain, VL of the third binding domain, second binding domain comprising second VH-second VL; or
(g) polypeptide A comprises components having an order N-terminal to C-terminal: CAP component, protease cleavage domain, VH of the third binding domain, first binding domain comprising first VH-first VL; and polypeptide B comprises components having an order N-terminal to C-terminal: HLP domain, protease cleavage domain, VL of the third binding domain, second binding domain comprising second VL-second VH; or
(h) polypeptide A comprises components having an order N-terminal to C-terminal: CAP component, protease cleavage domain, VH of the third binding domain, first binding domain comprising first VL-first VH; and polypeptide B comprises components having an order N-terminal to C-terminal: HLP domain, protease cleavage domain, VL of the third binding domain, second binding domain comprising second VL-second VH.

In one embodiment, the third binding domain is a Fab, and the first and second binding domains are scFv. In one embodiment, the Fab binds to a T cell surface antigen, one scFv binds to a TAA and the other scFv binds to a NK cell surface antigen. In another embodiment, the Fab binds to a NK cell surface antigen, one scFv binds to a TAA and the other scFv binds to another NK cell surface antigen.

In other embodiments, the precursor tri-specific antibody construct comprises two polypeptides, polypeptide A and polypeptide B, and instead of linking a regulatory domain to each polypeptide, only one polypeptide is linked to a regulatory domain, for example,
(a) polypeptide A comprises components having an order N-terminal to C-terminal: VH of the third binding domain, first binding domain comprising first VH-first VL; and polypeptide B comprises components having an order N-terminal to C-terminal: VL of the third binding domain, second binding domain comprising second VH-second VL, wherein a regulatory domain is located N-terminal to either polypeptide A or B, the regulatory domain comprises components having an order N-terminal to C-terminal: a CAP component, a half-life prolonging (HLP) domain, and a protease cleavage domain; or
(b) polypeptide A comprises components having an order N-terminal to C-terminal: VH of the third binding domain, first binding domain comprising first VL-first VH; and polypeptide B comprises components having an order N-terminal to C-terminal: VL of the third binding domain, second binding domain comprising second VH-second VL, wherein a regulatory domain is located N-terminal to either polypeptide A or B, the regulatory domain comprises components having an order N-terminal to C-terminal: a CAP component, a half-life prolonging (HLP) domain, and a protease cleavage domain; or
(c) polypeptide A comprises components having an order N-terminal to C-terminal: VH of the third binding domain, first binding domain comprising first VH-first VL; and polypeptide B comprises components having an order N-terminal to C-terminal: VL of the third binding domain, second binding domain comprising second VL-second VH, wherein a regulatory domain is located N-terminal to either polypeptide A or B, the regulatory domain comprises components having an order N-terminal to C-terminal: a CAP component, a half-life prolonging (HLP) domain, and a protease cleavage domain; or
(d) polypeptide A comprises components having an order N-terminal to C-terminal: VH of the third binding domain, first binding domain comprising first VL-first VH; and polypeptide B comprises components having an order N-terminal to C-terminal: VL of the third binding domain, second binding domain comprising second VL-second VH, wherein a regulatory domain is located N-terminal to either polypeptide A or B, the regulatory domain comprises components having an order N-terminal to C-terminal: a CAP component, a half-life prolonging (HLP) domain, and a protease cleavage domain.

In some embodiments as described above, the third binding domain may bind to a T cell surface antigen, the first binding domain may bind to a TAA and the second binding domain may bind to a NK cell surface antigen. In another embodiment, the third binding domain may bind to a NK cell surface antigen, the first binding domain may bind to a TAA and the second binding domain may bind to another NK cell surface antigen. In some embodiments, when the second binding domain bind to a NK cell surface antigen, the second binding domain may further comprise a regulatory CAP component, for example:
(a) polypeptide A comprises components having an order N-terminal to C-terminal: HLP domain, protease cleavage domain, VH of the third binding domain, first binding domain comprising first VH-first VL; and polypeptide B comprises components having an order N-terminal to C-terminal: CAP component, protease cleavage domain, VL of the third binding domain, second binding domain comprising second VH-second VL, protease cleavage domain, and a second CAP component; or
(b) polypeptide A comprises components having an order N-terminal to C-terminal: HLP domain, protease cleavage domain, VH of the third binding domain, first binding domain comprising first VL-first VH; and polypeptide B comprises components having an order N-terminal to C-terminal: CAP component, protease cleavage domain, VL of the third binding domain, second binding domain comprising second VH-second VL, protease cleavage domain, and a second CAP component; or
(c) polypeptide A comprises components having an order N-terminal to C-terminal: HLP domain, protease cleavage domain, VH of the third binding domain, first binding domain comprising first VH-first VL; and polypeptide B comprises components having an order N-terminal to C-terminal: CAP component, protease cleavage domain, VL of the third binding domain, second binding domain comprising second VL-second VH, protease cleavage domain, and a second CAP component; or
(d) polypeptide A comprises components having an order N-terminal to C-terminal: HLP domain, protease cleavage domain, VH of the third binding domain, first binding domain comprising first VL-first VH; and polypeptide B comprises components having an order N-terminal to C-terminal: CAP component, protease cleavage domain, VL of the third binding domain, second binding domain comprising second VL-second VH, protease cleavage domain, and a second CAP component; or
(e) polypeptide A comprises components having an order N-terminal to C-terminal: CAP component, protease cleavage domain, VH of the third binding domain, first binding domain comprising first VH-first VL; and polypeptide B comprises components having an order N-terminal to C-terminal: HLP domain, protease cleavage domain, VL of the third binding domain, second binding domain comprising second VH-second VL, protease cleavage domain, and a second CAP component; or
(f) polypeptide A comprises components having an order N-terminal to C-terminal: CAP component, protease cleavage domain, VH of the third binding domain, first binding domain comprising first VL-first VH; and polypeptide B comprises components having an order N-terminal to C-terminal: HLP domain, protease cleavage domain, VL of the third binding domain, second binding domain comprising second VH-second VL, protease cleavage domain, and a second CAP component; or
(g) polypeptide A comprises components having an order N-terminal to C-terminal: CAP component, protease cleavage domain, VH of the third binding domain, first binding domain comprising first VH-first VL; and polypeptide B comprises components having an order N-terminal to C-terminal: HLP domain, protease cleavage domain, VL of the third binding domain, second binding domain comprising second VL-second VH, protease cleavage domain, and a second CAP component; or
(h) polypeptide A comprises components having an order N-terminal to C-terminal: CAP component, protease cleavage domain, VH of the third binding domain, first binding domain comprising first VL-first VH; and polypeptide B comprises components having an order N-terminal to C-terminal: HLP domain, protease cleavage domain, VL of the third binding domain, second binding domain comprising second VL-second VH, protease cleavage domain, and a second CAP component.

In another embodiment, the second binding domain may comprise a regulatory CAP component as described above, wherein only one polypeptide (A or B) is linked to a regulatory domain comprising another CAP component, for example:
(a) polypeptide A comprises components having an order N-terminal to C-terminal: VH of the third binding domain, first binding domain comprising first VH-first VL; and polypeptide B comprises components having an order N-terminal to C-terminal: VL of the third binding domain, second binding domain comprising second VH-second VL, protease cleavage domain, and a CAP component, wherein a regulatory domain is located N-terminal to either polypeptide A or B, the regulatory domain comprises components having an order N-terminal to C-terminal: a second CAP component, a half-life prolonging (HLP) domain, and a protease cleavage domain; or
(b) polypeptide A comprises components having an order N-terminal to C-terminal: VH of the third binding domain, first binding domain comprising first VL-first VH; and polypeptide B comprises components having an order N-terminal to C-terminal: VL of the third binding domain, second binding domain comprising second VH-second VL, protease cleavage domain, and a CAP component, wherein a regulatory domain is located N-terminal to either polypeptide A or B, the regulatory domain comprises components having an order N-terminal to C-terminal: a second CAP component, a half-life prolonging (HLP) domain, and a protease cleavage domain; or
(c) polypeptide A comprises components having an order N-terminal to C-terminal: VH of the third binding domain, first binding domain comprising first VH-first VL; and polypeptide B comprises components having an order N-terminal to C-terminal: VL of the third binding domain, second binding domain comprising second VL-second VH, protease cleavage domain, and a CAP component, wherein a regulatory domain is located N-terminal to either polypeptide A or B, the regulatory domain comprises components having an order N-terminal to C-terminal: a second CAP component, a half-life prolonging (HLP) domain, and a protease cleavage domain; or
(d) polypeptide A comprises components having an order N-terminal to C-terminal: VH of the third binding domain, first binding domain comprising first VL-first VH; and polypeptide B comprises components having an order N-terminal to C-terminal: VL of the third binding domain, second binding domain comprising second VL-second VH, protease cleavage domain, and a CAP component, wherein a regulatory domain is located N-terminal to either polypeptide A or B, the regulatory domain comprises components having an order N-terminal to C-terminal: a second CAP component, a half-life prolonging (HLP) domain, and a protease cleavage domain.

In certain embodiments, the first and third binding domains are those as described above, whereas the second binding domain comprises a cytokine receptor engager, for example:
(a) polypeptide A comprises components having an order N-terminal to C-terminal: HLP domain, protease cleavage domain, VH of the third binding domain, first binding domain comprising first VH-first VL; and polypeptide B comprises components having an order N-terminal to C-terminal: CAP component, protease cleavage domain, VL of the third binding domain, and a second binding domain comprising a cytokine receptor engager; or
(b) polypeptide A comprises components having an order N-terminal to C-terminal: HLP domain, protease cleavage domain, VH of the third binding domain, first binding domain comprising first VL-first VH; and polypeptide B comprises components having an order N-terminal to C-terminal: CAP component, protease cleavage domain, VL of the third binding domain, and a second binding domain comprising a cytokine receptor engager; or
(c) polypeptide A comprises components having an order N-terminal to C-terminal: CAP component, protease cleavage domain, VH of the third binding domain, first binding domain comprising first VH-first VL; and polypeptide B comprises components having an order N-terminal to C-terminal: HLP domain, protease cleavage domain, VL of the third binding domain, and a second binding domain comprising a cytokine receptor engager; or
(d) polypeptide A comprises components having an order N-terminal to C-terminal: CAP component, protease cleavage domain, VH of the third binding domain, first binding domain comprising first VL-first VH; and polypeptide B comprises components having an order N-terminal to C-terminal: HLP domain, protease cleavage domain, VL of the third binding domain, and a second binding domain comprising a cytokine receptor engager.

In some embodiments, the second binding domain comprises a cytokine receptor engager, wherein only one polypeptide (A or B) is linked to a regulatory domain, for example:
(a) polypeptide A comprises components having an order N-terminal to C-terminal: VH of the third binding domain, first binding domain comprising first VH-first VL; and polypeptide B comprises components having an order N-terminal to C-terminal: VL of the third binding domain, and a second binding domain comprising a cytokine receptor engager, wherein a regulatory domain is located N-terminal to either polypeptide A or B, the regulatory domain comprises components having an order N-terminal to C-terminal: a CAP component, a half-life prolonging (HLP) domain, and a protease cleavage domain; or
(b) polypeptide A comprises components having an order N-terminal to C-terminal: VH of the third binding domain, first binding domain comprising first VL-first VH; and polypeptide B comprises components having an order N-terminal to C-terminal: VL of the third binding domain, and a second binding domain comprising a cytokine receptor engager, wherein a regulatory domain is located N-terminal to either polypeptide A or B, the regulatory domain comprises components having an order N-terminal to C-terminal: a CAP component, a half-life prolonging (HLP) domain, and a protease cleavage domain; or
(c) polypeptide A comprises components having an order N-terminal to C-terminal: VH of the third binding domain, first binding domain comprising first VH-first VL; and polypeptide B comprises components having an order N-terminal to C-terminal: VL of the third binding domain, and a second binding domain comprising a cytokine receptor engager, wherein a regulatory domain is located N-terminal to either polypeptide A or B, the regulatory domain comprises components having an order N-terminal to C-terminal: a CAP component, a half-life prolonging (HLP) domain, and a protease cleavage domain; or
(d) polypeptide A comprises components having an order N-terminal to C-terminal: VH of the third binding domain, first binding domain comprising first VL-first VH; and polypeptide B comprises components having an order N-terminal to C-terminal: VL of the third binding domain, and a second binding domain comprising a cytokine receptor engager, wherein a regulatory domain is located N-terminal to either polypeptide A or B, the regulatory domain comprises components having an order N-terminal to C-terminal: a CAP component, a half-life prolonging (HLP) domain, and a protease cleavage domain.

In some embodiments, the second binding domain comprises an IL-15 polypeptide, wherein only one polypeptide (A or B) is linked to a regulatory domain, for example:
(a) polypeptide A comprises components having an order N-terminal to C-terminal: VH of the third binding domain, first binding domain comprising first VH-first VL; and polypeptide B comprises components having an order N-terminal to C-terminal: VL of the third binding domain, and a second binding domain comprising a IL-15 polypeptide, wherein a regulatory domain is located N-terminal to either polypeptide A or B, the regulatory domain comprises components having an order N-terminal to C-terminal: a CAP component, a half-life prolonging (HLP) domain, and a protease cleavage domain; or
(b) polypeptide A comprises components having an order N-terminal to C-terminal: VH of the third binding domain, first binding domain comprising first VL-first VH; and polypeptide B comprises components having an order N-terminal to C-terminal: VL of the third binding domain, and a second binding domain comprising an IL-15 polypeptide, wherein a regulatory domain is located N-terminal to either polypeptide A or B, the regulatory domain comprises components having an order N-terminal to C-terminal: a CAP component, a half-life prolonging (HLP) domain, and a protease cleavage domain; or
(c) polypeptide A comprises components having an order N-terminal to C-terminal: VH of the third binding domain, first binding domain comprising first VH-first VL; and polypeptide B comprises components having an order N-terminal to C-terminal: VL of the third binding domain, and a second binding domain comprising an IL-15 polypeptide, wherein a regulatory domain is located N-terminal to either polypeptide A or B, the regulatory domain comprises components having an order N-terminal to C-terminal: a CAP component, a half-life prolonging (HLP) domain, and a protease cleavage domain; or
(d) polypeptide A comprises components having an order N-terminal to C-terminal: VH of the third binding domain, first binding domain comprising first VL-first VH; and polypeptide B comprises components having an order N-terminal to C-terminal: VL of the third binding domain, and a second binding domain comprising an IL-15 polypeptide, wherein a regulatory domain is located N-terminal to either polypeptide A or B, the regulatory domain comprises components having an order N-terminal to C-terminal: a CAP component, a half-life prolonging (HLP) domain, and a protease cleavage domain.

In certain embodiments, a first or second binding domain, or both a first and second binding domain, or a first or second regulatory domain, or both a first and second regulatory domain, or a combination thereof are linked directly to the respective termini of the VH-CH1 or VL-CL of the third binding domain, e.g., an Fab (i.e., with no additional amino acids added between). In other embodiments, the linking with the third binding domain, e.g., an Fab, comprises use of a linker as described above (with additional amino acids as described below). In some embodiments, it may be necessary to delete several amino acids (e.g., from 1-3 amino acids or from 1-10 amino acids; e.g., 1, 2, 3, 4, 5, 6, 7, 8, 9, or 10 amino acids) from the C-terminus of a given first or second binding domain or both, and/or a first or second regulatory domain or both, depending on the third binding domain target and the surrounding space of the first and second binding domain targets on the cell surface (i.e., for example, accessibility of the CD3 epsilon target on the cell surface of a T-cell).

In other embodiments, it may be necessary to delete several amino acids (e.g., from 1-3 amino acids or from 1-10 amino acids; e.g., 1, 2, 3, 4, 5, 6, 7, 8, 9, or 10 amino acids) from the N-terminus of the heavy and/or light chain of the third binding domain. In yet further embodiments, it may be necessary to delete several amino acids (e.g., from 1-3 amino acids or from 1-10 amino acids; e.g., 1, 2, 3, 4, 5, 6, 7, 8, 9, or 10 amino acids) from the N-terminus of the first or second binding domains or both, and/or the C-terminus of the first or second regulatory domains or both, and at the same time, to delete several amino acids (e.g., from 1-3 amino acids or from 1-10 amino acids; e.g., 1, 2, 3, 4, 5, 6, 7, 8, 9, or 10 amino acids) from the N-terminus and/or C-terminus of a third binding domain chain (VH-CH1 or VL-CL). The length and the sequence of the junction between a first or second binding domain, and/or a first or second regulatory domain, and the third binding domain VH-CH1 and VL-CL chains can be the same or different.

The junction between the first or second binding domain or both, and /or the first or second regulatory domain or both, and the third binding domain VH-CH1 and VL-CL chains may make use of a combination of deletions and linkers as needed. As would be understood by the skilled artisan, the junction between the third binding domain VH-CH1 and VL-CL chains and the first or second binding domain or both and/or the first or second regulatory domain or both, can be adjusted accordingly and tested for desired functionality (e.g., binding affinity, T-cell activity) using methods known in the art and described herein.

As described herein, junctions between domain or between components within domains comprises linkers. In some embodiments, a linker is present between domains. In some embodiments, there is not a linker between domains. In some embodiments, a linker is present between components that comprise a domain. In some embodiments, there is not a linker between components that comprise a domain.

FIGS. 2-5 show graphically where linkers may exist in an embodiment of precursor tri-specific antibody constructs disclosed herein.

In some embodiments, the linker between a first or second binding domain or both and a third binding domain VH-CH1 or VL-CL is 1-10 amino acids long. In other embodiments, the linker between a first or second binding domain or both, and a third binding domain VH-CH1 or VL-CL is 1-20 or 20 amino acids long. In this regard, the linker may be 1, 2, 3, 4, 5, 6, 7, 8, 9, 10, 11, 12, 13, 14, 15, 16, 17, 18, 19 or 20 amino acids long. In further embodiments, the linker may be 21, 22, 23, 24, 25, 26, 27, 28, 29 or 30 amino acids long.

In some embodiments, the linker between a first or second regulatory domain or both and a third binding domain VH-CH1 or VL-CL is 1-10 amino acids long. In other embodiments, the linker between a first or second regulatory domain or both, and a third binding domain VH-CH1 or VL-CL is 1-20 or 20 amino acids long. In this regard, the linker may be 1, 2, 3, 4, 5, 6, 7, 8, 9, 10, 11, 12, 13, 14, 15, 16, 17, 18, 19 or 20 amino acids long. In further embodiments, the linker may be 21, 22, 23, 24, 25, 26, 27, 28, 29 or 30 amino acids long.

In some embodiments, the linker between components within a first or second binding domain or both is 1-10 amino acids long. In other embodiments, the linker between components within a first or second binding domain or both is 1-20 or 20 amino acids long. In this regard, the linker between components may be 1, 2, 3, 4, 5, 6, 7, 8, 9, 10, 11, 12, 13, 14, 15, 16, 17, 18, 19 or 20 amino acids long. In further embodiments, the linker between components may be 21, 22, 23, 24, 25, 26, 27, 28, 29 or 30 amino acids long.

In some embodiments, the linker between components within a first or second regulatory domain or both is 1-10 amino acids long, wherein it should be understood that a linker between different components need not be the same length. In other embodiments, the linker between components within a first or second regulatory domain or both is 1-20 or 20 amino acids long, wherein it should be understood that a linker between different components need not be the same length. In this regard, the linker between each set of components may be 1, 2, 3, 4, 5, 6, 7, 8, 9, 10, 11, 12, 13, 14, 15, 16, 17, 18, 19 or 20 amino acids long. In further embodiments, the linker between each set of components may be 21, 22, 23, 24, 25, 26, 27, 28, 29 or 30 amino acids long.

In some embodiments, there are linkers between components within an HC or LC Fab polypeptide components VH and CH1, and polypeptide components VL and CL, respectively. In some embodiments, a linker is 1-10 amino acids long, wherein it should be understood that a linker between different components need not be the same length. In other embodiments, the linker between components within a third binding domain is 1-20 or 20 amino acids long, wherein it should be understood that a linker between different components need not be the same length. In this regard, the linker between each set of components may be 1, 2, 3, 4, 5, 6, 7, 8, 9, 10, 11, 12, 13, 14, 15, 16, 17, 18, 19 or 20 amino acids long. In further embodiments, the linker between each set of components may be 21, 22, 23, 24, 25, 26, 27, 28, 29 or 30 amino acids long.

In certain embodiments, linkers suitable for use in the precursor constructs described herein are flexible linkers. Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

In some embodiments, flexible linkers include glycine polymers (G)n, glycine-serine polymers (including, for example, (GS)n, (GSGGS)n (SEQ ID NO: 119) and (GGGS)n (SEQ ID NO: 120), where n is an integer of at least one), glycine-alanine polymers, alanine-serine polymers, and other flexible linkers known in the art. Glycine and glycine-serine polymers are relatively unstructured, and therefore may be able to serve as a neutral tether between components. Glycine accesses significantly more phi-psi space than even alanine and is much less restricted than residues with longer side chains (see Scheraga, Rev. Computational Chem. 11173-142 (1992)). In some embodiments, flexible linkers include, but are not limited to Gly-Gly-Ser-Gly (GGSG; SEQ ID NO: 121), Gly-Gly-Ser-Gly-Gly (GGSGG; SEQ ID NO: 122), Gly-Ser-Gly-Ser-Gly (GSGSG; SEQ ID NO: 123), Gly-Ser-Gly-Gly-Gly (GSGGG; SEQ ID NO: 124), Gly-Gly-Gly-Ser-Gly (GGGSG; SEQ ID NO: 125), Gly-Ser-Ser-Ser-Gly (GSSSG; SEQ ID NO: 126), Gly-Gly-Ser-Gly-Gly-Ser (GGSGGS; SEQ ID NO: 165) and the like. The ordinarily skilled artisan will recognize that design of a precursor tri-specific antibody construct can include linkers that are all or partially flexible, such that the linker can include a flexible linker as well as one or more portions that confer less flexible structure to provide for a desired precursor tri-specific antibody construct structure.

In some embodiments, a flexible linker used in a precursor construct comprises any flexible linker known in the art. In some embodiments, a flexible linker comprises a flexible unstructured linker. Linkers known in the art have been describe at least in Chengcheng Liu, Ju Xin Chin, Dong-Yup Lee; SynLinker: an integrated system for designing linkers and synthetic fusion proteins, Bioinformatics, Volume 31, Issue 22, 15 Nov. 2015, Pages 3700-3702; Fusion protein linkers: property, design and functionality Chen X et al, Adv Drug Deliv Rev. 2013 Oct;65(10):1357-69; The Linker Data base provided by The Centre for Integrative Bioinformatics vrije Universiteit Amsterdam (http://www.ibi.vu.nl/programs/linkerdbwww); and the CSD Linker Database provided by The Cambridge Crystallographic Data Centre (https://www.ccdc.cam.ac.uk/solutions/partnersoftware/csdlinkerdatabase/).

In certain embodiments, the linker between the third binding domain and the first or second binding domain or both, or the first or second regulatory domain or, or both binding and regulatory domains is a stable linker (not cleavable by protease, especially MMPs). In certain embodiments, the linker is a peptide linker.

In some embodiments, the linker between the third binding domain VH-CH1 or VL-CL chains and a first or second regulatory domain or both, comprises a protease substrate cleavage sequence, for example, an MMP substrate cleavage sequence. In some embodiments, the linker between the third binding domain VH-CH1 or VL-CL chains and a first or second regulatory domain or both, comprises a protease substrate cleavage sequence, for example, an MMP2/9, uPA, matriptase, and legumain substrate cleavage sequence. A peptide sequence of SEQ ID NO: 9 in a substrate can be cleaved by most MMPs. A peptide sequence of SEQ ID NO: 35 in a substrate can be cleaved by MMP2/9, uPA, matriptase, and legumain.

A protease substrate cleavage sequence refers to a peptide sequence that can be cleaved by protease treatment. An MMP substrate sequence refers to a peptide sequence that can be cleaved by incubation with a MMP. SEQ ID NO: 9 is a commonly used MMP substrate cleavage sequence (see e.g., Jiang, PNAS (2004) 101:17867-72; Olson, PNAS (2010) 107:4311-6). In another embodiment, the protease cleavage site is recognized by MMP-2, MMP-9 or a combination thereof. In yet another embodiment, the protease site comprises the sequence selected from the group consisting of (SEQ ID NO: 9, SEQ ID NO: 10, SEQ ID NO: 11, and SEQ ID NO: 35). In a further embodiment, the protease site comprises the sequence set forth in SEQ ID NO: 35.

In some embodiments, all of the protease sites comprise the same proteolytic sequence. In other embodiments, the protease sites of a precursor construct differ. Differences of the proteolytic sequences within a precursor construct may provide for additional regulation of function of the precursor or partially activated construct.

A stable linker or a protease non-cleavable linker refers to a linker peptide sequence that does not belong to the known protease substrate sequences and thus does not lead to significant cleavage product formation upon incubation with a protease.

In some embodiments, the cleavage substrate (or cleavage sequence or protease cleavage domain) of the linker may include an amino acid sequence that can serve as a substrate for a protease, usually an extracellular protease. In other embodiments, the cleavage sequence comprises a cysteine-cysteine pair capable of forming a disulfide bond, which can be cleaved by action of a reducing agent. In other embodiments the cleavage sequence comprises a substrate capable of being cleaved upon photolysis.

The cleavage substrate is positioned in the linker such that when the cleavage substrate is cleaved by a cleaving agent (e.g., a cleavage substrate of a linker is cleaved by the protease and/or the cysteine-cysteine disulfide bond is disrupted via reduction by exposure to a reducing agent) or by light-induced photolysis, in the presence of a target, resulting in cleavage products having various functional properties as described herein.

The cleavage substrate of a linker may be selected based on a protease that is co-localized in the diseased tissue, or on the surface of the cell that expresses the target antigen of interest of a binding domain of a fusion moiety. A variety of different conditions are known in which a target of interest is co-localized with a protease, where the substrate of the protease is known in the art. In the example of cancer, the target tissue can be a cancerous tissue, particularly cancerous tissue of a solid tumor. There are reports in the literature of increased levels of proteases having known substrates in a number of cancers, e.g., solid tumors. (See, e.g., La Rocca et al, (2004) British J. of Cancer 90(7): 1414-1421). Non-limiting examples of disease include: all types of cancers (breast, lung, colorectal, prostate, head and neck, pancreatic, etc), rheumatoid arthritis, Crohn’s disease, melanomas, SLE, cardiovascular damage, ischemia, etc. Furthermore, anti-angiogenic targets, such as VEGF, are known. As such, where the binding domains of a fusion moiety of the precursor tri-specific antibody construct of the present disclosure is selected such that it is capable of binding a TAA/NK, a suitable cleavage substrate sequence for a protease cleavable linker will be one which comprises a peptide substrate that is cleavable by a protease that is present at the cancerous treatment site, particularly that is present at elevated levels at the cancer treatment site as compared to non-cancerous tissues.

In some embodiments, the first or second binding domain or both of a precursor tri-specific antibody construct can bind, e.g., Her2, and the cleavage substrate sequence can be a matrix metalloprotease (MMP) substrate, and thus is cleavable by an MMP. In other embodiments, the first or second binding domain or both of a fusion moiety in the precursor tri-specific antibody construct can bind a target of interest or two targets of interest, and the cleavage substrate present in the linker can be, for example, legumain, plasmin, TMPRSS-¾, MMP-9, MT1-MMP, cathepsin, caspase, human neutrophil elastase, beta-secretase, uPA, or PSA. In other embodiments, the first or second binding domain or both of a fusion moiety in the precursor tri-specific antibody construct can bind a target of interest or two targets of interest, and the cleavage substrate present in the linker can be, for example, a combination of MMP2/9, legumain, uPA, and matriptase. In some embodiments, the first or second binding domain or both of a fusion moiety in the precursor tri-specific antibody construct can bind a target of interest or two targets of interest, and the cleavage substrate present in the linker comprises a combination of MMP2/9, legumain, uPA, and matriptase as set forth in SEQ ID NO: 35. In other embodiments, the cleave substrate is cleaved by other disease-specific proteases, in diseases other than cancer such as multiple sclerosis or rheumatoid arthritis.

The unmodified or uncleaved linker can allow for tethering the binding domains (first, second, or third, or a combination thereof) and regulatory domains (first or second, or both).

The linkers of the precursor tri-specific antibody construct (e.g., the linker between the CH 1 or CL of the third binding domain and a first or second binding domain or both, and the linker between the VH or VL of the third binding domain and a first or second regulatory domain or both, can comprise the same cleavage substrate or may comprise different cleavage substrates, e.g., the first linker may comprise a first cleavage substrate and the second linker may comprise a second cleavage substrate, etc. The cleavage substrates can be different substrates for the same enzyme (for example exhibiting different binding affinities to the enzyme), or different substrates for different enzymes, or one of the cleavage substrates can be an enzyme substrate and another of the cleavage substrate can be a photolysis substrate, or another cleavage substrate can be a substrate for reduction, or any combination thereof.

In some embodiments, some of the linkers may be non-cleavable while the others of the linkers are cleavable linker. For example, but not limited to the linkers between the Fab CH1 and CL and the first and second binding domain are non-cleavable, while the linkers between the Fab VH and VL and the first and second regulatory domains are cleavable. A skilled artisan would appreciate that there are a limited number of combinations of the linkers, and in some embodiments, each linker may be cleavable or non-cleavable. Thus, in some embodiments, a linker between the third binding domain and a first or second regulatory domain or both is cleavable while the linker between the third binding domain and the first or second binding domain or both is not cleavable.

For specific cleavage by an enzyme, contact between the enzyme and the cleavage substrate is made. When the precursor tri-specific antibody construct is present within a microenvironment comprising sufficient enzyme activity, the cleavage substrate can be cleaved. Sufficient enzyme activity can refer to the ability of the enzyme to make contact with the linker having the cleavage substrate and effect cleavage. It can readily be envisioned that an enzyme may be in the vicinity of the precursor tri-specific antibody construct but unable to cleave because of other cellular factors or protein modification of the enzyme.

In some embodiments, substrates can include but are not limited to substrates cleavable by one or more of the following enzymes or proteases: ADAM10; Caspase 8, Cathepsin S, MMP 8, ADAM12, Caspase 9, FAP, MMP 9, ADAM17, Caspase 10, Granzyme B, MMP-13, ADAMTS, Caspase 11, Guanidinobenzotase (GB), MMP 14, ADAMTS5. Caspase 12, Hepsin, MT-SP1, BACE, Caspase 13, Human Neutrophil Elastase Neprilysin (HNE), Caspases, Caspase 14, Legumain, NS¾A, Caspase 1, Cathepsins, Matriptase 2, Plasmin, Caspase 2, Cathepsin A, Meprin, PSA, Caspase 3, Cathepsin B, MMP 1, PSMA, Caspase 4, Cathepsin D, MMP 2, TACE, Caspase 5, Cathepsin E, MMP 3, TMPRSS ¾, Caspase 6, Cathepsin K, MMP 7, uPA, Caspase 7, Matripase (MT-SP1, TADG-15, epithin, ST14), and MT1-MMP.

In other embodiments, the cleavage substrate can involve a disulfide bond of a cysteine pair, which is thus cleavable by a reducing agent such as, for example, but not limited to a cellular reducing agent such as glutathione (GSH), thioredoxins, NADPH, flavins, ascorbate, and the like, which can be present in large amounts in tissue of or surrounding a solid tumor.

Other appropriate protease cleavage sites for use in the cleavable linkers herein are known in the art or may be identified using methods such as those described by Turk et al., 2001 Nature Biotechnology 19, 661-667.

In certain embodiments, the linker can be a peptide linker, a thiol residue-containing peptide linker, such as a cysteine residue, a polymer linker or a chemical linker. In certain embodiments, the precursor tri-specific antibody construct comprises a linker where one end of the linker is covalently linked to the N-terminal of a first or second binding domain or both fusion moiety, and the other end of the linker is covalently linked to the C-terminal of the CH1 or CL of the third binding domain.

In some embodiment, there is just one or a few amino acids between domains or components within domains. In certain embodiments, there may be one or a few amino acid residues between two domains of a precursor tri-specific antibody construct, such as between a binding domain and a linker polypeptide, such as amino acid residues resulting from construct design of the precursor construct (e.g., amino acid residues resulting from the use of a restriction enzyme site during the construction of a nucleic acid molecule encoding the polypeptide chains (polypeptide A and polypeptide B). As described herein, such amino acid residues may be referred to “junction amino acids” or “junction amino acid residues”, or “peptide linkers”.

In certain illustrative embodiments, a peptide linker is between 1 to 5 amino acids, between 5 to 10 amino acids, between 5 to 25 amino acids, between 5 to 50 amino acids, between 10 to 25 amino acids, between 10 to 50 amino acids, between 10 to 100 amino acids, or any intervening range of amino acids. In other illustrative embodiments, a peptide linker comprises about 1, 5, 10, 15, 20, 25, 30, 35, 40, 45, 50 or more amino acids in length.

Such junctional amino acids link any of the domains or components within domain of the precursor tri-specific antibody construct. In certain embodiments, the junctional amino acid(s) is a hinge, or a part of a hinge as defined herein. In certain embodiments, a variable region linking sequence useful for connecting a heavy chain variable region to a light chain variable region may be used as a peptide linker.

In one illustrative embodiment, peptide linker sequences contain, for example, Gly, Asn and Ser residues. Other near neutral amino acids, such as Thr and Ala, may also be included in the linker sequence.

Other amino acid sequences which may be usefully employed as linkers include those disclosed in Maratea et al., Gene 40:39 46 (1985); Murphy et al., Proc. Natl. Acad. Sci. USA 83:8258 8262 (1986); U.S. Pat. No. 4,935,233 and U.S. Pat. No. 4,751,180, incorporated herein in their entirety.

Other illustrative linkers may include, for example, Glu-Gly-Lys-Ser-Ser-Gly-Ser-Gly-Ser-Glu-Ser-Lys-Val-Asp (EGKSSGSGSESKVD; SEQ ID NO: 127) (Chaudhary et al., 1990, Proc. Natl. Acad. Sci. U.S.A. 87:1066-1070) and Lys-Glu-Ser-Gly-Ser-Val-Ser-Ser-Glu-Gln-Leu-Ala-Gln-Phe-Arg-Ser-Leu-Asp (KESGSVSSEQLAQFRSLD; SEQ ID NO: 128) (Bird et al., 1988, Science 242:423-426).

In some embodiments, linker sequences are not required when the HC and LC polypeptides (polypeptides A and B) have non-essential N-terminal amino acid regions that can be used to separate the functional domains and prevent steric interference. Coding sequences or domains of the precursor tri-specific antibody construct of the present disclosure can be fused directly without any junctional amino acids or by using a flexible polylinker composed, for example, of the pentamer Gly-Gly-Gly-Gly-Ser (GGGGS; SEQ ID NO: 129) repeated 1 to 3 times. Such a linker has been used in constructing single chain antibodies (scFv) by being inserted between VH and VL (Bird et al., 1988, Science 242:423-426; Huston et al., 1988, Proc. Natl. Acad. Sci. U.S.A. 85:5979-5883).

Examples of linkers include, but are not limited to, those having the sequences of (GS)n, SEQ ID NO:716; (GS)n(GGS)n, SEQ ID NO:119; (GS)n(GGGS)n, SEQ ID NO:717; (GS)n(GGGGS)n, SEQ ID NO:718; (GGS)n, SEQ ID NO:719; (GGS)n(GGGS)n, SEQ ID NO:720; (GGS)n(GGGGS)n, SEQ ID NO:721; (GGGS)n, SEQ ID NO:120; (GGGS)n(GGGGS)n, SEQ ID NO:722; (GGGGS)n, SEQ ID NO: 129; or (SGGGS)n, SEQ ID NO:723, wherein the “n” in these sequences equal to 1, 2, 3, 4, 5, 6,7, 8, 9 or 10.

A peptide linker, in certain embodiments, is designed to enable the correct interaction between two beta-sheets forming the variable region of the single chain antibody. Any suitable linkers can be used to make an indirect link, such as without limitation, peptide linker, polymer linker, and chemical linker. In certain embodiments, the covalent link is an indirect link through a peptide linker.

A distinguishing characteristic of the precursor tri-specific antibody construct, as described herein, is that the precursor construct does not depend on steric hindrance to reduce or inhibit the binding affinity of the third binding domain. In the case of the precursor constructs described herein, reduction or inhibition of binding affinity is due to specific binding between a CAP component and a third binding domain of the precursor construct. On the contrary, the proteins described in US 2012/0321626 depend solely on three-dimensional structure for reduced specificity, wherein the polypeptides may or may not form such a three-dimensional structure, and thus they lack any specificity for the reduction or inhibition of binding to the antibody target of the second binding region. Further distinguishing characteristics in comparison with other multi- or tri-specific antibodies that include a mask, is that the reduction or inhibition of binding to a target of the third binding domain by precursor tri-specific antibody construct, may also be temporally controlled, wherein reduction or inhibition of binding to a target of the third binding domain may be maintained when the precursor construct is in circulation in vivo or within a non-tumor microenvironment. This may reduce negative side effects caused by the use of multi- or bi-specific antibodies lacking this temporal regulation.

In some embodiments, precursor tri-specific antibody constructs of the present disclosure comprise a Polypeptide A (HC polypeptide) comprising the amino acid sequences set forth in SEQ ID NO: 130, or a homologue thereof; and a Polypeptide B (LC polypeptide) comprising the amino acid sequence set forth in SEQ ID NO: 131. In some embodiments, precursor tri-specific antibody constructs of the present disclosure comprise a Polypeptide A (HC polypeptide) comprising the amino acid sequences set forth in SEQ ID NO:132, or a homologue thereof; and a Polypeptide B (LC polypeptide) comprising the amino acid sequence set forth in SEQ ID NO: 133.

In some embodiments, precursor tri-specific antibody constructs of the present disclosure comprise a Polypeptide A (HC polypeptide) encoded by the nucleotide sequence set forth in SEQ ID NO: 142, or a homologue thereof; and comprise a Polypeptide B (LC polypeptide) encoded by the nucleotide sequence set forth in SEQ ID NO: 143. In some embodiments, precursor tri-specific antibody constructs of the present disclosure comprise a Polypeptide A (HC polypeptide) encoded by the nucleotide sequence set forth in SEQ ID NO: 144, or a homologue thereof; and comprise a Polypeptide B (LC polypeptide) encoded by the nucleotide sequence set forth in SEQ ID NO: 145.

### Functionality of Precursor Tri-specific Antibody Constructs

In some embodiments, a precursor tri-specific antibody construct disclosed herein possesses many unique features and these features can be utilized to develop human therapeutics with desirable attributes in drug safety, efficacy and manufacturability. In some embodiments, the precursor tri-specific antibody constructs of this disclosure comprising a first and a second binding domain binding to cell surface tumor associated antigens (TAA) and or to an extracellular epitope of a Natural Killer (NK) cell, a third binding domain binding to an extracellular epitope of human CD3 epsilon, and two regulatory domains, possesses many unique features and these features can be utilized to develop human therapeutics with desirable attributes in drug safety, efficacy and manufacturability. These features have been described in detail above and will not necessarily be repeated herein. A skilled artisan would appreciate that the uses as described herein below, include use of the many embodiments of precursor tri-specific antibody constructs as described above.

As described herein, the property of a precursor construct comprising a regulatable extended half-life, a regulatory reduction of T-cell binding (reduction of T-cell activation), or a combination thereof, may be used advantageously in the precursor tri-specific antibody construct of the present disclosure to mask T-cell binding until the precursor tri-specific antibody construct are in an appropriate microenvironment (e.g., in the vicinity of a tumor). In some embodiments, a pharmaceutical composition comprises a precursor tri-specific antibody construct, as described herein, and a pharmaceutically acceptable carrier.

A skilled artisan would recognize that in some embodiments, the term “precursor tri-specific antibody construct” may be used interchangeably with the term “drug” having all the same meanings and qualities. In some embodiments, a drug comprising a precursor tri-specific antibody construct comprises a pharmaceutical composition.

In some embodiments, a precursor tri-specific antibody disclosed herein comprises a first binding domain binding to a TAA, a second binding domain binding to a NK cell surface antigen, and a third binding domain binding to a T cell surface antigen or a NK cell surface antigen. A precursor tri-specific antibody comprising these three binding domains and regulatory domains comprising for example, a cleavable half-life prolonging domain comprising a protease cleavable domain and a human serum albumin (HSA) polypeptide (a first sub-regulatory domain), and a cleavable masking domain comprising a protease cleavable domain and a CAP region (a second sub-regulatory domain), provides unique properties as described throughout.

The precursor tri-specific antibody construct of the present disclosure functions to enhance drug stability, specificity, selectivity, potency, and safety and the convenience of drug administration. In certain embodiments, the third binding domain, when expressed without the regulatory domain comprising said CAP region linked to its N-terminus (VH or VL chain), is able to bind to its target antigen in soluble recombinant form (usually the extracellular domain of a receptor protein, e.g., a T-cell receptor component such as CD3) as well as on the cell surface. In certain embodiments, the third binding domain, when expressed with a regulatory domain fused to its N-terminus (VL or VH chain) and comprising a CAP region, and a first and a second binding domain fused to the C-terminus (CL or CH1 chains), has no binding or has reduced binding to its specific antigen presented on a T or NK cell surface at pharmacological concentrations of the drug (concentration of the polypeptide in treated patients) compared with a third binding domain present in a construct lacking a regulatory domain comprising a CAP region. Lack of binding or greatly reduced binding to a surface antigen in the absence of the target antigen binding by a third binding domain may be explained by the dramatically reduced affinity resulting from the specific blocking of the antigen binding site by a CAP component.

Lack of binding or greatly reduced binding to cell surface antigen, for example an antigen on a T-cell or NK cell, in the absence of the TAA target antigen binding by a first binding domain, may be viewed as a desirable property for use of a precursor tri-specific antibody construct as a human therapeutic. It is important to note that lack of binding or significantly reduced binding of the precursor tri-specific antibody construct alone (in the absence of tumor target cells) to, e.g., T or NK cells can, 1) dramatically improve the undesirable systematic T or NK cell activation, therefore to dramatically improve the drug safety profile; 2) dramatically improve the feasibility of subcutaneous route of drug administration; and 3) dramatically increase the drug tolerability of high drug concentration in blood circulation. Further, the regulatable temporal regulation provided by a second regulatory domain comprising a half-life prolonging component (e.g., an HSA polypeptide) of a precursor construct may ensure the extended presence of the precursor construct in circulation until such time as the drug is present in the environment of TAA target cells (e.g., tumor target cell microenvironment).

It is important to note that T-cell binding by antibodies such as OKT3 or UCHT-1 via conformational epitopes may transduce partial signaling, leading either to unwanted T-cell activation (causing cytokine storm) or T-cell anergy (resulting in T-cells unable to kill tumor cells). Mu-lF3, hu-1F3 and its variants binding to a linear epitope of CD3 is conceivably less likely to induce T-cell signaling in the absence of cross linking of the CD3. This property may be advantageous for reducing systemic side effects that occur when using OKT3 and UCHT-1 like antibodies.

It is also important to note that once a regulatory domain comprising a CAP region or a portion thereof comprising the CAP region in a precursor tri-specific antibody construct is cleaved by protease, its function such that the specific binding inhibition at the third antigen-binding site (e.g., CD3 epsilon binding site) is removed so that it can then bind to its target with high affinity, particularly target antigens expressed on the cell surface. Therefore, following cleavage at the cleavage substrate sequence in a protease cleavable linker (thereby releasing a regulatory domain comprising a CAP region and releasing a regulatory domain comprising a HLP) the precursor tri-specific antibody construct is converted into a more potent cross linker between tumor and T-cells (FIG. 1). In some embodiments, the regulatory domain comprising the HLP is not cleaved, yet the precursor construct is converted into a more potent cross linker between tumor and T-cells.

Similarly, once a regulatory domain comprising a CAP region able to associated with a binding region in a precursor tri-specific antibody construct is cleaved by protease, its function such that the specific binding inhibition at the antigen-binding site (e.g., NK antigen binding site) is removed so that it can then bind to its target with high affinity. In some embodiments, the precursor tri-specific antibody construct is converted into a more potent cross linker between NK cells, tumor cells and T-cells (FIG. 1). In some embodiments, when the precursor tri-specific antibody construct comprises an NK engager only (no other TAA binding site) the construct is converted into a more potent cross linker between NK cells and T-cells. In some embodiments, the regulatory domain comprising the HLP is not cleaved, yet the precursor construct is converted into a more potent cross linker between tumor, NK cells, and T-cells, or NK cells and T-cells.

Furthermore, it is important to note that once a TAA first binding domain binds to its target antigen, the precursor tri-specific antibody construct molecules become highly concentrated on a tumor cell surface to create high avidity based binding toward the third binding domain target on T cells or NK cells. Therefore, only in the presence of the TAA first binding domain is the third antigen-binding domain able to bind its target, for precursor tri-specific antibody construct to function as a cross-linker between tumor, NK cells, and T-cells.

The properties of the precursor tri-specific antibody construct of the present disclosure allow for relatively high dose of the precursor tri-specific antibody construct in circulation for an enhanced period of time, without unwanted side-effects, e.g., the precursor tri-specific antibody construct does not bind to the third binding domain target antigen (e.g., CD3) when in circulation. This also allows for reduced dosing frequency and promotes tissue penetration by diffusion driven by concentration gradient.

The properties of the precursor tri-specific antibody construct of the present disclosure also allow the potential for the subcutaneous administration, which can enhance access to the target. Further, although in certain embodiments the precursor tri-specific antibody construct are permissive for cross-linking without protease treatment, in certain embodiments, the binding activity and the tumor killing potency increase dramatically after protease treatment.

In one embodiment, the third binding domain antigen binding domain formed by VH and VL is stabilized by the CH1 and CL heterodimerizing domain, and is further stabilized by the disulfide bond, or other stabilizing interaction (e.g., knobs/hole interaction), between CH1 and CL.

In some embodiments, the third binding domain in the precursor tri-specific antibody construct is specifically blocked by the CAP regulatory domain at its N-terminus, such that binding to the third binding domain target antigen (especially when cell surface target antigens are concerned) is specifically reduced or inhibited in a statistically significant manner (i.e., relative to an appropriate control as will be known to those skilled in the art; e.g., as compared to the same third binding domain in a format without a regulatory domain comprising a CAP component, at its N-termini (either VH and VL)). In a further embodiment, the third binding domain in the precursor tri-specific antibody construct is specifically blocked so that binding to the desirable antigen (especially when cell surface target antigens are concerned) is reduced by at least 2 fold, 3 fold, 4 fold, 5 fold, 6 fold, 7 fold, 8 fold, 9 fold, 10 fold, 11 fold, 12 fold, 13 fold, 14 fold, 15 fold, 20 fold, 30 fold, or 100 fold, or 1000 fold, or 10,000 fold as compared to the same third binding domain in a format without a regulatory domain comprising a CAP component at its N-terminus (either VH and VL).

In certain embodiments, the affinity of the third binding domain to T cells or NK cells in the precursor construct is below 500 nM. In further embodiments, the affinity of the third binding domain of the precursor construct demonstrates no significant detectible binding as measured using FACS or other binding measurement method (e.g., cell binding ELISA) at concentration ranges of the therapeutics used in humans. In one embodiment, less than 1% of a population of target T or NK cells will be bound by the third binding domain of a precursor construct at a therapeutic concentration (this is in the absence of a tumor cell microenvironment). In one embodiment, less than 5% population of the target cells will be bound by the precursor tri-specific antibody construct at a therapeutic concentration. In yet another embodiment, less than 10% population of the target cells will be bound by the precursor tri-specific antibody construct at a therapeutic concentration.

The elevated level of proteases, especially MMPs, present in tumor tissues (tumor microenvironment) will generate cleavage products at the MMP substrate cleavage site of a protease cleavable linker. Because the cleavage of the protease substrate sequence of a linker results in the release of a CAP component that may be bound at the third binding domain antigen-binding region, the binding to the third binding domain cell surface target will be fully restored or at least partially restored. The restored binding can be demonstrated using techniques of FACS, cell-based ELISA) or other cell binding techniques known to the skilled person.

A skilled artisan would appreciate that the term “dramatically reduced affinity” may encompass at least 30% reduction in the binding of the third binding domain antigen-binding domain, as compared to the binding in the absence of a CAP component of a regulatory domain present at the N-terminus of the third binding domain. The percentage of reduction can be, for example without limitation, 30%, about 40%, about 50%, about 60%, about 70%, about 80%, about 90%, or about 99% or greater. Methods for detecting binding are known to the skilled person and can be performed using FACS, cell binding ELISA or cell binding using radio-isotype labeled antibodies.

An embodiment of a third binding domain for use in the precursor tri-specific antibody construct of the present disclosure as described herein binds to a T cell or NK cell surface antigen. In this regard, the precursor tri-specific antibody construct functions such that, when the anti-TAA first binding domain binds to a tumor associated antigen, the precursor construct is present within a tumor microenvironment comprising proteases, wherein the cleavable regulatory domains are cleaved releasing a CAP component such that the third binding domain is now able to bind to T or NK cells, thereby redirecting the T or NK cells and activating them to kill the tumor cell or tumor associate cell. (FIG. 1) In another embodiment, an activated tri-specific antibody construct (also referred to herein as activated tri-specific antibody construct where the third binding domain fragment binds to T or NK cells) can exhibit an avidity effect when clustered on tumor cell surface via TAA (e.g., tumor antigen) binding by the first binding domain. As such, the apparent binding to immune cells by the third binding domain can increase due to avidity. As such, an activated tri-specific antibody construct becomes capable of bridging immune and tumor cells thereby mediating anti-tumor activity. Additionally, an activated Tri-specific antibody construct may have improved tumor penetration compared with the precursor construct, due to decreased size.

In certain embodiments, a precursor tri-specific antibody construct is separately bound to a TAA, and not bound to a T cell or NK cell, and thus the T-cells or NK cells will not be activated. In some embodiments, this lack of binding to immune cells may be a result of the TAA(s) present on a non-tumor cell. In some embodiments, this lack of binding to immune cells may be the results of the TAA(s) present on a cell in a non-tumor microenvironment. As such, the precursor construct bound to a TAA(s) in a non-tumor environment and in certain embodiments, can therefore not be activated (i.e., there will be no cleavage of the regulatable domain comprising the CAP or a portion thereof). This avoids significant side effects and tissue damage that may occur were the precursor construct to activate T or NK cells in a non-tumor cell environment. However, within a tumor microenvironment, when the T or NK cells and tumor surface antigen are simultaneously bound to the activated tri-specific antibody construct and when multiple copies of the bound complexes are anchored and clustered on tumor cell surface, the T or NK cells are activated in the vicinity of cancer cells bearing the tumor surface antigen, and therefore significantly enhance the tumor killing efficiency of T or NK cells locally and avoid the side effects due to cytokine storm.

In certain embodiments, the combination of a third binding domain targeting T or NK cells and a first binding domain targeting a cell surface tumor associated antigen comprised within a precursor construct, which is temporally regulated by a half-life enhancing regulatory domain and activity regulated by a CAP regulatory domain, the combination of which provide for enhanced tumor killing effects by T or NK cells once the precursor construct has been located to a tumor microenvironment. In certain embodiments, the combination of the third binding domain antigen target and the first binding domain antigen target can be FcyR and TAA, respectively, which combination can induce FcγR-expressing immune cells to kill tumor cells once the precursor construct has been located to a tumor microenvironment. In certain embodiments, the combination of the third binding domain antigen target and the first and second binding domain antigen targets can be CD3ε, a TAA and NKG2D, respectively, which combination can activate T cells and induce natural killer (NK) cell to kill tumor cells, once the precursor construct has been located to a tumor microenvironment. In another embodiment, the third binding domain may bind to a NK cell, the first binding domain may bind to a TAA, and the second binding domain binds to another NK cell surface antigen. In another embodiment, the third binding domain may bind to a NK cell, the first binding domain may bind to a TAA, and the second binding domain comprises a cytokine receptor engager as described herein.

Thus, in some embodiments, the precursor tri-specific antibody construct of the present disclosure comprises a third binding domain that binds to T cells or NK cells, e.g. the TCR or a component thereof, such as a CD3 polypeptide. As noted above, the precursor tri-specific antibody construct of the present disclosure does not bind to the third binding domain target antigen except following a linker cleavage event, wherein a CAP component is release or in the absence of a CAP component comprised within the precursor construct.

Thus, in certain embodiments, a precursor tri-specific antibody construct of the present disclosure does not activate T-cells or NK cells in the absence of target antigen engagement at the third binding domain. A precursor tri-specific antibody construct “does not or minimally or nominally activates T or NK cells” if the precursor tri-specific antibody construct does not cause a statistically significant increase in the percentage of activated T or NK cells as compared to activation of such immune cells in the presence of cells expressing TAA recognized by the first binding domain (e.g., an appropriate tumor cell/cell line; tumor micro-environment), as measured in at least one in vitro or in vivo assay. Such assays are known in the art and include, without limitation, proliferation assays, CTL chromium release assays (see e.g., Lavie et al., (2000) International Immunology 12(4):479-486), ELISPOT assays, intracellular cytokine staining assays, and others as described, for example, in Current Protocols in Immunology, John Wiley & Sons, New York, N.Y. (2009). In certain embodiments, T-cell activation is measured using an in vitro primed T-cell activation assay.

In a related aspect, therefore, the present disclosure provides a method for detecting T or NK cell activation induced by an activated precursor tri-specific antibody construct that comprises a first binding domain that specifically binds to a TAA, a third binding domain that specifically binds to T or NK cells, and two sub-regulatory domains, wherein the precursor construct is activated in the presence of a tumor microenvironment. In some embodiments, activation of a precursor construct comprises cleavage of a both regulatory domains. In some embodiments, activation of a precursor construct comprises cleavage of one sub-regulatory domain. In some embodiments, activation of a precursor construct comprises cleavage of a complete regulatory domain comprising a CAP component. In some embodiments, activation of a precursor construct comprises cleavage of a portion of a regulatory domain, wherein the portion of the regulatory domain comprises a CAP component. In some embodiments, activation of a precursor construct comprises cleavage of a complete regulatory domain comprising a HLP component. In some embodiments, activation of a precursor construct comprises cleavage of a portion of a regulatory domain, wherein the portion of the regulatory domain comprises a HLP component. In some embodiments, activation of a precursor construct comprises cleavage of both sub-regulatory domains, one comprising a CAP component and the other comprising an HSA component.

In some embodiments, a method for detecting T cell activation comprises (a) providing antigen or mitogen-primed T cells, (b) treating the primed T cells of step (a) with the precursor tri-specific antibody construct that comprises a third binding domain that specifically binds to a TCR complex or a component thereof (following exposure to a tumor microenvironment and cleavage of a regulatory CAP domain or a portion thereof), and (c) detecting activation of the primed T cells that have been treated in step (b).

The term “mitogen” as used herein refers to a chemical substance that induces mitosis in lymphocytes of different specificities or clonal origins. Exemplary mitogens that may be used to prime T-cells include phytohaemagglutinin (PHA), concanavalin A (ConA), lipopolysaccharide (LPS), pokeweed mitogen (PWM), and phorbol myristate acetate (PMA). Antigen-loaded beads or PBMC can also be used to prime T-cells.

In certain embodiments of methods for detecting T-cell activation provided herein, the precursor tri-specific antibody construct comprising a third binding domain that specifically binds to a TCR complex or a component thereof comprises a first and a second binding domain that bind to tumor associated antigens and a NK surface antigen, and two sub-regulatory domains, wherein one provides enhanced half-life prolonging properties, and the second provide reduction in T-cell binding properties, reduction in T-cell activation properties, or any combination thereof. In certain embodiments, methods for detecting T-cell activation provided herein, are performed in tumor and non-tumor microenvironments.

T-cell activation may be detected by measuring the expression of activation markers known in the art, such as CD25, CD40 ligand, and CD69. Activated T-cells may also be detected by cell proliferation assays, such as CFSE labeling and thymidine uptake assays (Adams (1969) Exp. Cell Res. 56:55). T-cell effector function (e.g., cell killing) can be measured, for example, by chromium release assays or FACS based assays using fluorescent dyes (e.g. TP3). In a related aspect, T-cell activation and cytolytic activity can be measured by lytic synapse formation between T-cell and tumor cell. Effector molecules such as Granzymes and porforin can be detected in the cytolytic synapse.

In another related aspect, T-cell activation may be measured by cytokine release. A method for detecting cytokine release induced by a precursor tri-specific antibody construct that comprises a third binding domain that specifically binds to a TCR complex or a component thereof, may comprise: (a) providing primed T-cells, (b) treating the primed T-cells of step (a) with the precursor tri-specific antibody construct that comprises a third binding domain that specifically binds to a TCR complex or a component thereof, (c) incubating the precursor construct in a tumor microenvironment e.g., with tumor cells associated with the antigen target of the anti-TAA first binding domain, and (d) detecting release of a cytokine from the primed T-cells that have been treated in step (b). In some embodiments, experiments are carried out in the presence or absence of appropriate cancer cells or cell lines expressing target tumor antigens bound by binding domains present in the first binding domain of the precursor tri-specific antibody construct (step c).

In certain embodiments of methods for detecting cytokine release provided herein, the precursor tri-specific antibody construct that comprises a third binding domain that specifically binds to a TCR complex or a component thereof is performed in the presence or absence of appropriate cancer cells or cell lines expressing target tumor antigens bound by binding domains present in the first binding domain of the precursor tri-specific antibody construct

In some embodiment, the precursor tri-specific antibody construct disclosed herein that contains at least one binding domain to NK cells can be used to activate NK cells, and NK cell activation can be measured by experiments similar to the ones described above. In some embodiments, the precursor tri-specific antibody construct disclosed herein that contains an IL-15 polypeptide can be used to activate NK cells, and NK cell activation and proliferation can be measured by experiments similar to the ones described above. In general, NK cell activation can be measured by cytolytic activity or degranulation assay. Such assays are known in the art and include, without limitation, chromium release assays, ELISPOT assays, intracellular cytokine staining assays, and others as described, for example, in Current Protocols in Immunology, John Wiley & Sons, New York, N.Y. (2009).

In some embodiments, the precursor tri-specific antibody construct disclosed herein that contains an IL-15 polypeptide can be used to increase proliferation of NK cells, and NK cell proliferation can be measured by experimental methods known in the art, for example but not limited to methods as provided in J. Immunol. 2015, 195:4810-4821. In some embodiments, the precursor tri-specific antibody construct disclosed herein that contains an IL-15 polypeptide can be used to increase cytotoxicity of NK cells, and NK cell cytotoxicity can be measured by experimental methods known in the art, for example but not limited to methods as provided in J. Immunol. 2015, 195:4810-4821.

In certain embodiments, the precursor tri-specific antibody construct of the present disclosure does not induce a cytokine storm or does not induce a cytokine release sufficient to induce toxic side-effects. A precursor tri-specific antibody construct “does not induce a cytokine storm” (also referred to as “inducing an undetectable, nominal, or minimal cytokine release” or “does not induce or induces a minimally detectable cytokine release”) if, in the absence of TAA target cells or appropriate linker cleavage agents (such as proteases), it does not cause a statistically significant increase in the amount of at least one cytokine including IFNγ.; In certain embodiments at least two cytokines including IFNγ and TNFα or IL-6 and TNFα.; in one embodiment three cytokines including IL-6, IFNγ and TNFα; in another embodiment four cytokines including IL-2, IL-6, IFNγ, and TNFα; and in yet a further embodiment at least five cytokines including IL-2, IL-6, IL-10,, IFNγ, and TNFα; released from treated cells in the absence of TAA target cells (e.g., an appropriate cancer cell line) or appropriate linker cleavage agents, as compared to from treated cells in the presence of appropriate TAA target cells or linker cleavage agents, in at least one in vitro or in vivo assay known in the art or provided herein. Clinically, cytokine-release syndrome is characterized by fever, chills, rash, nausea, and sometimes dyspnea and tachycardia, which is in parallel with maximal release of certain cytokines, such as IFNγ, as well as IL-2, IL-6, and TNFα. Cytokines that may be tested for release in an in vitro assay or in vivo include G-CSF, GM-CSF, IL-2, IL-4, IL-5, IL-6, IL-10, IL-13, IL-17, IP-10, KC, MCP1, IFNγ, and TNFα; and in another embodiment include IL-2, IL-6, IL-10, IFNγ, and TNFα.

In further embodiments, a precursor tri-specific antibody construct of the present disclosure causes an increase in calcium flux in cells, such as immune cells. A precursor tri-specific antibody construct causes an “increase in calcium” if, when used to activate immune cells in the presence of an appropriate TAA target cell (e.g., cancer cell) or linker cleavage agents, it causes a statistically significant, rapid increase in calcium flux of the treated cells (within 300 seconds, or within 200 seconds, or within 100 seconds of treatment) as compared to cells treated in the absence of an appropriate TAA target cell or linker cleavage agents, as measured in an in vitro assay known in the art or provided herein.

In further embodiments, a precursor tri-specific antibody construct of the present disclosure induces phosphorylation of a molecule in the TCR signal transduction pathway. The “TCR signal transduction pathway” refers to the signal transduction pathway initiated via the binding of a peptide:MHC ligand to the TCR and its co-receptor (CD4 or CD8). A “molecule in the TCR signal transduction pathway” refers to a molecule that is directly involved in the TCR signal transduction pathway, such as a molecule whose phosphorylation state (e.g., whether the molecule is phosphorylated or not), whose binding affinity to another molecule, or whose enzymatic activity, has been changed in response to the signal from the binding of a peptide:MHC ligand to the TCR and its co-receptor. Exemplary molecules in the TCR signal transduction pathway include the TCR complex or its components (e.g., CD3 chains), ZAP-70, Fyn, Lck, phospholipase c-γ, protein kinase C, transcription factor NFκB, phosphatase calcineurin, transcription factor NFAT, guanine nucleotide exchange factor (GEF), Ras, MAP kinase kinase kinase (MAPKKK), MAP kinase kinase (MAPKK), MAP kinase (ERK½), and Fos.

A precursor tri-specific antibody construct of this disclosure “induces phosphorylation of a molecule in the TCR signal transduction pathway” if it causes a statistically significant increase in phosphorylation of a molecule in the TCR signal transduction pathway (e.g., CD3 chains, ZAP-70, and ERK½) only in the presence of cells expressing TAA antigen (e.g., cancer cells expressing tumor antigens bound by a first binding domains, or when the TAA is present on a non-tumor cell, tumor cells expressing proteases able to cleave the protease cleavable domain of a regulatory domain are present) or linker cleavage agents, in an in vitro or in vivo assay or receptor signaling assays known in the art. Results from most receptor signaling assays known in the art are determined using immunohistochemical methods, such as western blots or fluorescence microscopy.

Similarly, an activated precursor tri-specific antibody construct of the present disclosure may induce killing of TAA target cell, such as tumor cells or vascular cells which support the growth and maintenance of tumor cells, by T-cells and/or NK cells following exposure to a tumor cell microenvironment or exposure to a protease or proteases able to cleave the protease cleavable component of a regulatory domain(s). Such cell killing can be measured using a variety of assays known in the art, including chromium release assays.

The specificity and function of a precursor tri-specific antibody construct of the present disclosure may be tested by contacting the precursor tri-specific antibody construct with appropriate test sample and, in certain embodiments, treating the precursor tri-specific antibody construct with an appropriate protease which is thought to be specific for the cleavage recognition site in the linker and assaying for cleavage products. Proteases may be isolated, for example from cancer cells or they may be prepared recombinantly, for example following the procedures in Darket et al. (J. Biol. Chem. 254:2307-2312 (1988)). The cleavage products may be identified for example based on size, antigenicity or activity. The toxicity of the precursor tri-specific antibody construct may be investigated by subjecting the precursor tri-specific antibody construct and cleavage products thereof to in vitro cytotoxicity, proliferation, binding, or other appropriate assays known to the skilled person. Toxicity of the cleavage products may be determined using a ribosomal inactivation assay (Westby et al., Bioconjugate Chem. 3:377-382 (1992)). The effect of the cleavage products on protein synthesis may be measured in standardized assays of in vitro translation utilizing partially defined cell free systems composed for example of a reticulocyte lysate preparation as a source of ribosomes and various essential cofactors, such as mRNA template and amino acids. Use of radiolabeled amino acids in the mixture allows quantitation of incorporation of free amino acid precursors into trichloroacetic acid precipitable proteins. Rabbit reticulocyte lysates may be conveniently used (O′Hare, FEBS Lett. 273:200-204 (1990)).

The ability of an activated precursor tri-specific antibody construct as disclosed herein, to destroy cancer cells and/or activate T-cells and/or NK cells may be readily tested in vitro using cancer cell lines, T or NK cells or isolated PBMC. The effects of the precursor tri-specific antibody construct of the present disclosure may be determined, for example, by demonstrating by selective lysis of cancer cells. In addition, the protease specificity can be tested by comparing the inhibition of cellular proliferation using a precursor bispecific antibody construct of the present disclosure alone or in the presence of protease-specific inhibitors. Such protease inhibitors may include MMP-2/MMP-9 inhibitors GM1489, GM6001 and GI-I to GI-IV.

Toxicity may also be measured based on cell viability, for example the viability of normal and cancerous cell cultures exposed to the precursor tri-specific antibody construct may be compared. Cell viability may be assessed by known techniques, such as trypan blue exclusion assays. Toxicity may also be measured based on cell lysis, for example the lysis of normal and cancerous cell cultures exposed to the precursor bispecific antibody construct may be compared. Cell lysis may be assessed by known techniques, such as Chromium (Cr) release assays or dead cell indicator dyes (propidium Iodide, TO-PRO-3 Iodide).

### Precursor Bispecific Antibody Construct Components

The present disclosure provides precursor tri-specific antibody construct polypeptides comprising various components as described herein. As described in detail above, in some embodiments a precursor tri-specific antibody construct comprises two polypeptides: polypeptide A and polypeptide B, each comprising various binding domains and regulatory components disclosed herein. The present disclosure has disclosed various examples of VH and VL for anti-T cell, anti-NK cell, or anti-TAA Fab or scFv; sequences for linkers; and sequences for various regulatory domains. In view of the disclosure provided herein, one of ordinary skill in the art would readily combine these various components into the precursor tri-specific antibody construct polypeptides described herein.

A skilled artisan would appreciate that terms “polypeptide” “protein” and “peptide” and “glycoprotein” are used interchangeably and encompass a polymer of amino acids not limited to any particular length. The term does not exclude modifications such as myristylation, sulfation, glycosylation, phosphorylation and addition or deletion of signal sequences. The terms “polypeptide” or “protein” may encompass one or more chains of amino acids, wherein each chain comprises amino acids covalently linked by peptide bonds, and wherein said polypeptide or protein can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. The terms “polypeptide” and “protein” may encompass a polypeptide A or a polypeptide B of a precursor tri-specific antibody construct and heterodimers thereof of the present disclosure, or sequences that have deletions from, additions to, and/or substitutions of one or more amino acid of a precursor bispecific antibody construct as disclosed herein. Thus, a “polypeptide” or a “protein” can comprise one (termed “a monomer”) or a plurality (termed “a multimer”) of amino acid chains.

The term “isolated protein” referred to herein encompasses a subject protein (1) is free of at least some other proteins with which it would typically be found in nature, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is not associated (by covalent or noncovalent interaction) with portions of a protein with which the “isolated protein” is associated in nature, (6) is operably associated (by covalent or noncovalent interaction) with a polypeptide with which it is not associated in nature, or (7) does not occur in nature. Such an isolated protein can be encoded by genomic DNA, cDNA, mRNA or other RNA, of may be of synthetic origin, or any combination thereof. In certain embodiments, the isolated protein is substantially free from proteins or polypeptides or other contaminants that are found in its natural environment that would interfere with its use (therapeutic, diagnostic, prophylactic, research or otherwise).

The term “polypeptide fragment” encompasses a polypeptide, which can be monomeric or multimeric, that has an amino-terminal deletion, a carboxyl-terminal deletion, and/or an internal deletion or substitution of a naturally-occurring or recombinantly-produced polypeptide. In certain embodiments, a polypeptide fragment can comprise an amino acid chain at least 5 to about 500 amino acids long. It will be appreciated that in certain embodiments, fragments are at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, or 450 amino acids long. Particularly useful polypeptide fragments include functional domains, including antigen-binding domains or fragments of antibodies. In the case of an anti-CD3, or other antibody, useful fragments include, but are not limited to: a CDR region, especially a CDR3 region of the heavy or light chain; a variable region of a heavy or light chain; a portion of an antibody chain or just its variable region including two CDRs; and the like.

Polypeptides may comprise a signal (or leader) sequence at the N-terminal end of the protein, which co-translationally or post-translationally directs transfer of the protein. The polypeptide may also be fused in-frame or conjugated to a linker or other sequence for ease of synthesis, purification or identification of the polypeptide (e.g., poly-His), or to enhance binding of the polypeptide to a solid support.

Amino acid sequence modification(s) of the precursor tri-specific antibody constructs described herein are contemplated. For example, it may be desirable to improve the binding affinity and/or other biological properties of the precursor tri-specific antibody construct. For example, amino acid sequence variants of a linker sequence, or a binding domain, or a regulatory component(s) thereof may be prepared by introducing appropriate nucleotide changes into a polynucleotide that encodes the precursor tri-specific antibody construct polypeptides, or a domain thereof, or by peptide synthesis. Such modifications include, for example, deletions from, and/or insertions into and/or substitutions of, residues within the amino acid sequences of the precursor tri-specific antibody construct polypeptides. Any combination of deletion, insertion, and substitution may be made to arrive at the final precursor tri-specific antibody construct polypeptides, provided that the final construct possesses the desired characteristics, such as specific binding to a target antigen of interest by a first or second binding domain or both, or a third binding domain, or enhanced half-life by an HSA polypeptide comprised in a regulatory domain, or specific binding to a third binding domain by a regulatory domain comprising a CAP component, or protease cleavage by a protease cleavage domain(s) (linker). The amino acid changes also may alter post-translational processes of the precursor tri-specific antibody construct polypeptides, such as changing the number or position of glycosylation sites. Any of the variations and modifications described above for polypeptides as disclosed herein, may be included in precursor tri-specific antibody constructs presented herein.

The present disclosure provides variants of the precursor tri-specific antibody construct polypeptides disclosed herein. In certain embodiments, such variant precursor tri-specific antibody construct polypeptides comprise variant binding domains or fragments thereof, or antigen-binding fragments, or TAA binding fragments, or NK binding fragments, or CDRs of binding domains, bind to a target of interest at least about 50%, at least about 70%, and in certain embodiments, at least about 90% as well as a given reference or wild-type sequence, including any such sequences specifically set forth herein. In further embodiments, such variants bind to a target antigen with greater affinity the reference or wild-type sequence set forth herein, for example, that bind quantitatively at least about 105%, 106%, 107%, 108%, 109%, or 110% as well as a reference sequence specifically set forth herein. In certain embodiments, such variant precursor tri-specific antibody construct polypeptides comprise variant regulatory domains or fragments thereof, or HSA components, or CAP components or fragments thereof, wherein said variant has at least about 50%, at least about 70%, and in certain embodiments, at least about 90% of the activity of a reference or wild-type regulatory domain or component, including any such sequences specifically set forth herein.

In certain embodiments, the present disclosure provides variants of the precursor tri-specific antibody constructs or polypeptides thereof, disclosed herein where such variants comprise third binding domains that have been modified with regard to the disulfide bond between the VH and VL chains. As would be recognized by the skilled person, in certain embodiments the third binding domain, which in some embodiments comprises a Fab fragment, used in the precursor tri-specific antibody construct described herein may not comprise a disulfide bond. In this regard, the heavy and light chains may be engineered in such a way so as to stably interact without the need for disulfide bond. For example, in certain embodiments, the heavy or light chain can be engineered to remove a cysteine residue and wherein the heavy and light chains still stably interact and function as a binding domain e.g. a Fab fragment. In some embodiments, mutations are made to facilitate stable interaction between the heavy and light chains. For example, a “knobs into holes” engineering strategy can be used to facilitate dimerization between the heavy and light chains of a Fab second binding domain (see e.g., 1996 Protein Engineering, 9:617-621). Thus, also contemplated for use herein are variant amino acid sequences of the third binding domain (e.g., Fab fragments) designed for a particular purpose, for example, removal of a disulfide bond addition of tax for purification, etc.

In particular embodiments, a subject precursor tri-specific antibody construct polypeptide may have: an amino acid sequence that is at least 80% identical, at least 95% identical, at least 90%, at least 95% or at least 98% or 99% identical, to the VH and VL portions of the precursor tri-specific antibody construct polypeptides described herein.

Determination of the three-dimensional structures of representative polypeptides may be made through routine methodologies such that substitution, addition, deletion or insertion of one or more amino acids with selected natural or non-natural amino acids can be virtually modeled for purposes of determining whether a so derived structural variant retains the space-filling properties of presently disclosed species. See, for instance, Donate et al., 1994 Prot. Sci. 3:2378; Bradley et al., Science 309: 1868-1871 (2005); Schueler-Furman et al., Science 310:638 (2005); Dietz et al., Proc. Nat. Acad. Sci. USA 103:1244 (2006); Dodson et al., Nature 450:176 (2007); Qian et al., Nature 450:259 (2007); Raman et al. Science 327:1014-1018 (2010). Some additional non-limiting examples of computer algorithms that may be used for these and related embodiments, include VMD which is a molecular visualization program for displaying, animating, and analyzing large biomolecular systems using 3-D graphics and built-in scripting (see the website for the Theoretical and Computational Biophysics Group, University of Illinois at Urbana-Champagne, at ks.uiuc.edu/Research/vmd/). Many other computer programs are known in the art and available to the skilled person and which allow for determining atomic dimensions from space-filling models (van der Waals radii) of energy-minimized conformations; GRID, which seeks to determine regions of high affinity for different chemical groups, thereby enhancing binding, Monte Carlo searches, which calculate mathematical alignment, and CHARMM (Brooks et al. (1983) J. Comput. Chem. 4:187-217) and AMBER (Weiner et al (1981) J. Comput. Chem. 106: 765), which assess force field calculations, and analysis (see also, Eisenfield et al. (1991) Am. J. Physiol. 261:C376-386; Lybrand (1991) J. Pharm. Belg. 46:49-54; Froimowitz (1990) Biotechniques 8:640-644; Burbam et al. (1990) Proteins 7:99-111; Pedersen (1985) Environ. Health Perspect. 61:185-190; and Kini et al. (1991) J. Biomol. Struct. Dyn. 9:475-488). A variety of appropriate computational computer programs are also commercially available, such as from Schrodinger (Munich, Germany).

### Polynucleotides Encoding Precursor Bispecific Antibody Construct Components, Vectors, Host Cells, and Methods of Producing Precursor Bispecific Antibody Constructs

The present disclosure further provides in certain embodiments an isolated nucleic acid encoding the polypeptide precursor tri-specific antibody construct as described herein. Illustrative polynucleotides and fragments thereof, are provided in Table 3 below. Nucleic acids include DNA and RNA. These and related embodiments may include polynucleotides encoding the precursor tri-specific antibody construct as described herein. The term “isolated polynucleotide” as used herein shall mean a polynucleotide of genomic, cDNA, or synthetic origin or some combination thereof, which by virtue of its origin the isolated polynucleotide (1) is not associated with all or a portion of a polynucleotide in which the isolated polynucleotide is found in nature, (2) is linked to a polynucleotide to which it is not linked in nature, or (3) does not occur in nature as part of a larger sequence.

A skilled artisan would appreciate that the terms “polynucleotide” and “nucleic acid sequence” may in some embodiments be used interchangeably having all the same meanings and qualities.

In some embodiments, isolated nucleic acid sequences encode a polypeptide A and a polypeptide B of a precursor tri-specific antibody construct as disclosed herein throughout in detail. In some embodiments, an isolated nucleic acid sequences encodes polypeptide A of a precursor tri-specific antibody construct, as described above in detail. In some embodiments, an isolated nucleic acid sequences encodes polypeptide B of a precursor tri-specific antibody construct, as described above in detail. In some embodiments, polypeptides A and B form a heterodimer comprising a precursor construct as described in detail above..

TABLE 3

Nucleotide Sequences of Encoding Anti-CD3 VH, VL, HC, LC, Anti-EGFR, Regulatory components, and Combinations thereof (See also nucleotide sequences in Example 1 below

| SEQ ID No | Description |
|---|---|
| 155 | Anti-CD3 epsilon VH-CH1 |
| 159 | Anti-CD3epsilon VL-CL |
| 36 | Anti-EGFR VL |
| 38 | Anti-EGFR VH |
| 43 | Anti-EGFR VL-L-VH |
| 44 | Anti-EGFR VH-L-VL |
| 8 | HSA |
| 164 | CAP |
| 33 | MMP Cleavable Sequence |

The term “operably linked” encompasses components to which the term is applied are in a relationship that allows them to carry out their inherent functions under suitable conditions. For example, a transcription control sequence “operably linked” to a protein coding sequence is ligated thereto so that expression of the protein coding sequence is achieved under conditions compatible with the transcriptional activity of the control sequences.

The term “control sequence” as used herein encompasses polynucleotide sequences that can affect expression, processing or intracellular localization of coding sequences to which they are ligated or operably linked. The nature of such control sequences may depend upon the host organism. In particular embodiments, transcription control sequences for prokaryotes may include a promoter, ribosomal binding site, and transcription termination sequence. In other particular embodiments, transcription control sequences for eukaryotes may include promoters comprising one or a plurality of recognition sites for transcription factors, transcription enhancer sequences, transcription termination sequences and polyadenylation sequences. In certain embodiments, “control sequences” can include leader sequences and/or fusion partner sequences.

The term “polynucleotide” as used herein encompasses single-stranded or double-stranded nucleic acid polymers. In certain embodiments, the nucleotides comprising the polynucleotide can be ribonucleotides or deoxyribonucleotides or a modified form of either type of nucleotide. Said modifications include base modifications such as bromouridine, ribose modifications such as arabinoside and 2′,3′-dideoxyribose and internucleotide linkage modifications such as phosphorothioate, phosphorodithioate, phosphoroselenoate, phosphorodiselenoate, phosphoroanilothioate, phoshoraniladate and phosphoroamidate. The term “polynucleotide” specifically includes single and double stranded forms of DNA.

The term “naturally occurring nucleotides” includes deoxyribonucleotides and ribonucleotides. The term “modified nucleotides” includes nucleotides with modified or substituted sugar groups and the like. The term “oligonucleotide linkages” includes oligonucleotide linkages such as phosphorothioate, phosphorodithioate, phosphoroselenoate, phosphorodiselenoate, phosphoroanilothioate, phoshoraniladate, phosphoroamidate, and the like. See, e.g., LaPlanche et al., 1986, Nucl. Acids Res., 14:9081; Stec et al., 1984, J. Am. Chem. Soc., 106:6077; Stein et al., 1988, Nucl. Acids Res., 16:3209; Zon et al., 1991, AntiCancer Drug Design, 6:539; Zon et al., 1991, OLIGONUCLEOTIDES AND ANALOGUES: A PRACTICAL APPROACH, pp. 87-108 (F. Eckstein, Ed.), Oxford University Press, Oxford England; Stec et al., U.S. Pat. No. 5,151,510; Uhlmann and Peyman, 1990, Chemical Reviews, 90:543, the disclosures of which are hereby incorporated by reference for any purpose. An oligonucleotide can include a detectable label to enable detection of the oligonucleotide or hybridization thereof.

In other related embodiments, polynucleotide variants may have substantial identity to a polynucleotide sequence encoding a precursor tri-specific antibody construct, or domain thereof as described herein. For example, a polynucleotide may be a polynucleotide comprising at least 70% sequence identity, preferably at least 75%, 80%, 85%, 90%, 95%, 96%, 97%, 98%, or 99% or higher, sequence identity compared to a reference polynucleotide sequence such as a sequence encoding a precursor bispecific antibody construct or domain thereof described herein, using the methods described herein, (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like.

Typically, polynucleotide variants will contain one or more substitutions, additions, deletions and/or insertions, preferably such that the binding affinity of a binding domain, or binding affinity of a first or second or a third binding domain, or function of the precursor tri-specific antibody construct polypeptide encoded by the variant polynucleotide is not substantially diminished relative to the unmodified reference protein encoded by a polynucleotide sequence specifically set forth herein.

In certain other related embodiments, polynucleotide fragments may comprise or consist essentially of various lengths of contiguous stretches of sequence identical to or complementary to a sequence encoding a precursor tri-specific antibody construct polypeptide or domain thereof as described herein. For example, polynucleotides are provided that comprise or consist essentially of at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, 150, 200, 300, 400, 500 or 1000 or more contiguous nucleotides of a sequences the encodes a precursor tri-specific antibody construct polypeptide or domain thereof, such as a first binding domain or a second binding domain or a third binding domain or a first regulatory domain or a second regulatory domain, or components thereof, disclosed herein as well as all intermediate lengths there between. It will be readily understood that “intermediate lengths”, in this context, means any length between the quoted values, such as 50, 51, 52, 53, etc.; 100, 101, 102, 103, etc.; 150, 151, 152, 153, etc.; including all integers through 200-500; 500-1,000, and the like. A polynucleotide sequence as described here may be extended at one or both ends by additional nucleotides not found in the native sequence. This additional sequence may consist of 1, 2, 3, 4, 5, 6, 7, 8, 9, 10, 11, 12, 13, 14, 15, 16, 17, 18, 19, or 20 nucleotides at either end of a polynucleotide encoding a precursor tri-specific antibody construct polypeptide or domain or component part thereof described herein or at both ends of a polynucleotide encoding a precursor tri-specific antibody construct polypeptide or domain or component part thereof described herein.

In another embodiment, polynucleotides are provided that are capable of hybridizing under moderate to high stringency conditions to a polynucleotide sequence encoding precursor tri-specific antibody construct polypeptide or domain or component part thereof, such as a first binding domain or a second binding domain or a third bindig domain or a first regulatory domain or a second regulatory domain, or component parts thereof, as provided herein, or a fragment thereof, or a complementary sequence thereof. Hybridization techniques are well known in the art of molecular biology. For purposes of illustration, suitable moderately stringent conditions for testing the hybridization of a polynucleotide as provided herein with other polynucleotides include prewashing in a solution of 5XSSC, 0.5% SDS, 1.0 mM EDTA (pH 8.0); hybridizing at 50° C.-60° C., 5XSSC, overnight; followed by washing twice at 65°. C. for 20 minutes with each of 2X, 0.5X and 0.2XSSC containing 0.1% SDS. One skilled in the art will understand that the stringency of hybridization can be readily manipulated, such as by altering the salt content of the hybridization solution and/or the temperature at which the hybridization is performed. For example, in another embodiment, suitable highly stringent hybridization conditions include those described above, with the exception that the temperature of hybridization is increased, e.g., to 60° C.-65° C. or 65° C.-70° C.

In certain embodiments, the polynucleotides described above, e.g., polynucleotide variants, fragments and hybridizing sequences, encode a precursor tri-specific antibody construct polypeptide or domain thereof or component part thereof, such as a first or a second binding domain or both, e.g., a scFv that binds to a human EGFR, or a third binding domain, e.g., a Fab fragment that binds CD3 epsilon, or a regulatory domain comprising an HSA polypeptide that extends half-life, or a regulatory domain comprising a CAP component that specifically binds to a third binding domain. In other embodiments, such polynucleotides encode precursor tri-specific antibody construct polypeptides or domains or components thereof that bind to T or NK cells and/or a tumor associated antigen at least about 50%, at least about 70%, and in certain embodiments, at least about 90% as well as a precursor tri-specific antibody construct polypeptide sequence specifically set forth herein. In other embodiments, such polynucleotides encode precursor tri-specific antibody construct polypeptides or domains or components thereof that extend the half-life of the precursor construct at least about 50%, at least about 70%, and in certain embodiments, at least about 90% as well as a precursor tri-specific antibody construct polypeptide sequence specifically set forth herein. In other embodiments, such polynucleotides encode precursor tri-specific antibody construct polypeptides or domains or components thereof that specifically bind to the third binding site of the precursor construct at least about 50%, at least about 70%, and in certain embodiments, at least about 90% as well as a precursor tri-specific antibody construct polypeptide sequence specifically set forth herein. In further embodiments, such polynucleotides encode a precursor tri-specific antibody construct polypeptide or domain thereof, that, e.g., bind to CD3 and/or a tumor associated antigen with greater affinity than the precursor tri-specific antibody construct polypeptide, or domain thereof, set forth herein, for example, that bind quantitatively at least about 105%, 106%, 107%, 108%, 109%, or 110% as well as a precursor tri-specific antibody construct polypeptide or domain thereof sequence specifically set forth herein.

The polynucleotides described herein, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative polynucleotide segments with total lengths of about 10,000, about 5000, about 3000, about 2,000, about 1,000, about 500, about 200, about 100, about 50 base pairs in length, and the like, (including all intermediate lengths) are contemplated to be useful.

When comparing polynucleotide sequences, two sequences are said to be “identical” if the sequence of nucleotides in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins--Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., Unified Approach to Alignment and Phylogenes, pp. 626-645 (1990); Methods in Enzymology vol. 183, Academic Press, Inc., San Diego, Calif.; Higgins, D. G. and Sharp, P. M., CABIOS 5:151-153 (1989); Myers, E. W. and Muller W., CABIOS 4:11-17 (1988); Robinson, E. D., Comb. Theor 11:105 (1971); Santou, N. Nes, M., Mol. Biol. Evol. 4:406-425 (1987); Sneath, P. H. A. and Sokal, R. R., Numerical Taxonomythe Principles and Practice of Numerical Taxonomy, Freeman Press, San Francisco, Calif. (1973); Wilbur, W. J. and Lipman, D. J., Proc. Natl. Acad., Sci. USA 80:726-730 (1983).

Alternatively, optimal alignment of sequences for comparison may be conducted by the local identity algorithm of Smith and Waterman, Add. APL. Math 2:482 (1981), by the identity alignment algorithm of Needleman and Wunsch, J. Mol. Biol. 48:443 (1970), by the search for similarity methods of Pearson and Lipman, Proc. Natl. Acad. Sci. USA 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One example of algorithms that are suitable for determining percent sequence identity and sequence similarity are the BLAST and BLAST 2.0 algorithms, which are described in Altschul et al., Nucl. Acids Res. 25:3389-3402 (1977), and Altschul et al., J. Mol. Biol. 215:403-410 (1990), respectively. BLAST and BLAST 2.0 can be used, for example with the parameters described herein, to determine percent sequence identity among two or more the polynucleotides. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff, Proc. Natl. Acad. Sci. USA 89:10915 (1989)) alignments, (B) of 50, expectation (E) of 10, M=5, N=-4 and a comparison of both strands.

In certain embodiments, the “percentage of sequence identity” is determined by comparing two optimally aligned sequences over a window of comparison of at least 20 positions, wherein the portion of the polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases occurs in both sequences to yield the number of matched positions, dividing the number of matched positions by the total number of positions in the reference sequence (i.e., the window size) and multiplying the results by 100 to yield the percentage of sequence identity.

It will be appreciated by those of ordinary skill in the art that, as a result of the degeneracy of the genetic code, there are many nucleotide sequences that encode a precursor bispecific antibody construct as described herein. Some of these polynucleotides bear minimal sequence identity to the nucleotide sequence of the native or original polynucleotide sequence that encode precursor tri-specific antibody construct polypeptides or domains or components thereof, for example forming a precursor bispecific antibody construct that binds to CD3 and or a tumor associated antigen. Nonetheless, polynucleotides that vary due to differences in codon usage are expressly contemplated by the present disclosure. In certain embodiments, sequences that have been codon-optimized for mammalian expression are specifically contemplated.

Therefore, in another embodiment as disclosed herein, a mutagenesis approach, such as site-specific mutagenesis, may be employed for the preparation of variants and/or derivatives of the precursor tri-specific antibody construct polypeptides described herein. By this approach, specific modifications in a polypeptide sequence can be made through mutagenesis of the underlying polynucleotides that encode them. These techniques provide a straightforward approach to prepare and test sequence variants, for example, incorporating one or more of the foregoing considerations, by introducing one or more nucleotide sequence changes into the polynucleotide.

Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or otherwise change the properties of the polynucleotide itself, and/or alter the properties, activity, composition, stability, or primary sequence of the encoded polypeptide.

In certain embodiments, mutagenesis of the polynucleotide sequences that encode a precursor tri-specific antibody construct polypeptide or domain thereof or component part thereof, as disclosed herein, is contemplated in order to alter one or more properties of the encoded polypeptide/domain/component, such as the binding affinity of a first binding domain or a second binding domain or a third binding domain, or the function of a first or second regulatory domain or component thereof. The techniques of site-specific mutagenesis are well-known in the art and are widely used to create variants of both polypeptides and polynucleotides. For example, site-specific mutagenesis is often used to alter a specific portion of a DNA molecule. In such embodiments, a primer comprising typically about 14 to about 25 nucleotides or so in length is employed, with about 5 to about 10 residues on both sides of the junction of the sequence being altered.

As will be appreciated by those of skill in the art, site-specific mutagenesis techniques have often employed a phage vector that exists in both a single stranded and double stranded form. Typical vectors useful in site-directed mutagenesis include vectors such as the M13 phage. These phages are readily commercially-available and their use is generally well-known to those skilled in the art. Double-stranded plasmids are also routinely employed in site directed mutagenesis that eliminates the step of transferring the gene of interest from a plasmid to a phage.

In general, site-directed mutagenesis in accordance herewith is performed by first obtaining a single-stranded vector or melting apart of two strands of a double-stranded vector that includes within its sequence a DNA sequence that encodes the desired peptide. An oligonucleotide primer bearing the desired mutated sequence is prepared, generally synthetically. This primer is then annealed with the single-stranded vector and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement.

The preparation of sequence variants of the selected peptide-encoding DNA segments using site-directed mutagenesis provides a means of producing potentially useful species and is not meant to be limiting as there are other ways in which sequence variants of peptides and the DNA sequences encoding them may be obtained. For example, recombinant vectors encoding the desired peptide sequence may be treated with mutagenic agents, such as hydroxylamine, to obtain sequence variants. Specific details regarding these methods and protocols are found in the teachings of Maloy et al., 1994; Segal, 1976; Prokop and Bajpai, 1991; Kuby, 1994; and Maniatis et al., 1982, each incorporated herein by reference, for that purpose.

As used herein, the term “oligonucleotide directed mutagenesis procedure” encompasses template-dependent processes and vector-mediated propagation which result in an increase in the concentration of a specific nucleic acid molecule relative to its initial concentration, or in an increase in the concentration of a detectable signal, such as amplification. As used herein, the term “oligonucleotide directed mutagenesis procedure” encompasses a process that involves the template-dependent extension of a primer molecule. The term “template dependent process” encompasses nucleic acid synthesis of an RNA or a DNA molecule wherein the sequence of the newly synthesized strand of nucleic acid is dictated by the well-known rules of complementary base pairing (see, for example, Watson, 1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224, specifically incorporated herein by reference in its entirety.

In another approach for the production of polypeptide variants, recursive sequence recombination, as described in U.S. Pat. No. 5,837,458, may be employed. In this approach, iterative cycles of recombination and screening or selection are performed to “evolve” individual polynucleotide variants having, for example, increased binding affinity. Certain embodiments also provide constructs in the form of plasmids, vectors, transcription or expression cassettes which comprise at least one polynucleotide as described herein.

In certain embodiments, the isolated polynucleotide is inserted into a vector. The term “vector” as used herein encompasses a vehicle into which a polynucleotide encoding a protein may be covalently inserted so as to bring about the expression of that protein and/or the cloning of the polynucleotide. The isolated polynucleotide may be inserted into a vector using any suitable methods known in the art, for example, without limitation, the vector may be digested using appropriate restriction enzymes and then may be ligated with the isolated polynucleotide having matching restriction ends.

Examples of suitable vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40).

For expression of the polypeptide, the vector may be introduced into a host cell to allow expression of the polypeptide within the host cell. The expression vectors may contain a variety of elements for controlling expression, including without limitation, promoter sequences, transcription initiation sequences, enhancer sequences, selectable markers, and signal sequences. These elements may be selected as appropriate by a person of ordinary skill in the art. For example, the promoter sequences may be selected to promote the transcription of the polynucleotide in the vector. Suitable promoter sequences include, without limitation, T7 promoter, T3 promoter, SP6 promoter, beta-actin promoter, EFla promoter, CMV promoter, and SV40 promoter. Enhancer sequences may be selected to enhance the transcription of the polynucleotide. Selectable markers may be selected to allow selection of the host cells inserted with the vector from those not, for example, the selectable markers may be genes that confer antibiotic resistance. Signal sequences may be selected to allow the expressed polypeptide to be transported outside of the host cell.

A vector may also include materials to aid in its entry into the cell, including but not limited to a viral particle, a liposome, or a protein coating.

In some embodiments, an expression vector comprises an isolated nucleic acid sequence encoding a polypeptide of a precursor construct, or encoding a domain within a polypeptide of the precursor construct, or encoding a component part of a domain within a polypeptide of the precursor construct. Binding domains and the components thereof have been described in detail above.

In some embodiments, an expression vector comprises an isolated nucleic acid sequence encoding a polypeptide A. In some embodiments, an expression vector comprises an isolated nucleic acid sequence encoding a polypeptide B. In some embodiments, an expression vector comprises an isolated nucleic acid sequence encoding a part of a polypeptide A. In some embodiments, an expression vector comprises an isolated nucleic acid sequence encoding a part of a polypeptide B. In some embodiments, an expression vector comprises an isolated nucleic acid sequence encoding a first binding domain. In some embodiments, an expression vector comprises an isolated nucleic acid sequence encoding an scFv of a first binding domain. In some embodiments, an expression vector comprises an isolated nucleic acid sequence encoding an scFv of a second binding domain. In some embodiments, an expression vector comprises an isolated nucleic acid sequence encoding part of an scFv of a first binding domain. In some embodiments, an expression vector comprises an isolated nucleic acid sequence encoding part of an scFv of a second binding domain. In some embodiments, an expression vector comprises an isolated nucleic acid sequence encoding an EGFR binding domain. In some embodiments, an expression vector comprises an isolated nucleic acid sequence encoding an EGFR scFv binding domain. In some embodiments, an expression vector comprises an isolated nucleic acid sequence encoding a Natural Killer cell scFv binding domain. In some embodiments, an expression vector comprises an isolated nucleic acid sequence encoding a VH region of a CD3 epsilon binding domain. In some embodiments, an expression vector comprises an isolated nucleic acid sequence encoding a VL region of a CD3 epsilon binding domain. In some embodiments, an expression vector comprises an isolated nucleic acid sequence encoding a CAP regulatory domain. In some embodiments, an expression vector comprises an isolated nucleic acid sequence encoding an HSA regulatory domain. In some embodiments, an expression vector comprises an isolated nucleic acid sequence encoding a component part of a regulatory domain. In some embodiments, an expression vector comprises an isolated nucleic acid sequence encoding a CAP component of a regulatory domain. In some embodiments, an expression vector comprises an isolated nucleic acid sequence encoding an HSA component of a regulatory domain. In some embodiments, an expression vector comprises an isolated nucleic acid sequence encoding a CAP component of a regulatory domain, and a linker(s) including a protease cleavable linker. In some embodiments, an expression vector comprises an isolated nucleic acid sequence encoding a HSA component of a regulatory domain, and a linker(s) including a protease cleavable linker.

For cloning of the polynucleotide, the vector may be introduced into a host cell (an isolated host cell) to allow replication of the vector itself and thereby amplify the copies of the polynucleotide contained therein. The cloning vectors may contain sequence components generally include, without limitation, an origin of replication, promoter sequences, transcription initiation sequences, enhancer sequences, and selectable markers. These elements may be selected as appropriate by a person of ordinary skill in the art. For example, the origin of replication may be selected to promote autonomous replication of the vector in the host cell.

In certain embodiments, the present disclosure provides isolated host cells containing the vector provided herein. The host cells containing the vector may be useful in expression or cloning of the polynucleotide(s) contained in the vector.

Suitable host cells can include, without limitation, prokaryotic cells, fungal cells, yeast cells, or higher eukaryotic cells such as mammalian cells.

Suitable prokaryotic cells for this purpose include, without limitation, eubacteria, such as Gram-negative or Gram-positive organisms, for example, Enterobactehaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

The expression of antibodies and antigen-binding fragments in prokaryotic cells such as E. coli is well established in the art. For a review, see for example Pluckthun, A. Bio/Technology 9: 545-551 (1991). Expression in eukaryotic cells in culture is also available to those skilled in the art as an option for production of antibodies or antigen-binding fragments thereof, see recent reviews, for example Ref, M. E. (1993) Curr. Opinion Biotech. 4: 573-576; Trill J. J. et al. (1995) Curr. Opinion Biotech 6: 553-560.

Suitable fungal cells for this purpose include, without limitation, filamentous fungi and yeast. Illustrative examples of fungal cells include, Saccharomyces cerevisiae, common baker’s yeast, Schizosaccharomyces pombe, Kluyveromyces hosts such as, eg., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Higher eukaryotic cells, in particular, those derived from multicellular organisms can be used for expression of glycosylated polypeptide provided herein. Suitable higher eukaryotic cells include, without limitation, invertebrate cells and insect cells, and vertebrate cells. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruiffly), and Bombyx mori have been identified. A variety of viral strains for transfection are publicly available, e.g., the K-1 variant of Autographa californica NPV and the Bm-5 strain of Bombyx mori NPV, and such viruses may be used as the virus herein as described herein, particularly for transfection of Spodoptera frugiperda cells. Plant cell cultures of cotton, corn, potato, soybean, petunia, tomato, and tobacco can also be utilized as hosts. Examples of vertebrate cells include mammalian host cell lines such as monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse Sertoli cells (TM4, Mather, Biol. Reprod. 23:243-251 (1980)); monkey kidney cells (CV1 ATCC CCL 70); African green monkey kidney cells (VERO-76, ATCC CRK-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

The vector can be introduced to the host cell using any suitable methods known in the art, including, without limitation, DEAE-dextran mediated delivery, calcium phosphate precipitate method, cationic lipids mediated delivery, liposome mediated transfection, electroporation, microprojectile bombardment, receptor-mediated gene delivery, delivery mediated by polylysine, histone, chitosan, and peptides. Standard methods for transfection and transformation of cells for expression of a vector of interest are well known in the art.

In certain embodiments, the host cells comprise a first vector encoding a first polypeptide and a second vector encoding a second polypeptide. In certain embodiments, the first vector and the second vector may be the same or not the same. In certain embodiments, the first polypeptide and the second polypeptide may be the same or not the same.

In certain embodiments, the host cells comprise a first vector encoding a polypeptide A and a second vector encoding a polypeptide B. In certain embodiments, the first vector and the second vector may be the same or not the same. In certain embodiments, the polypeptide A and the polypeptide B may be encoded on the same vector.

In some embodiments, an isolated cell comprises an isolated nucleic acid sequence, as disclosed herein. In some embodiments, an isolated cell comprises two isolated nucleic acid sequences as disclosed herein, wherein one nucleic acid encodes polypeptide A and the other nucleic acid encodes polypeptide B. In some embodiments, an isolated cell comprises two expression vectors as disclosed herein, wherein one vector comprises a nucleic acid encoding polypeptide A and the other vector comprises a nucleic acid encoding polypeptide B.

In certain embodiments, the first vector and the second vector may or may not be introduced simultaneously. In certain embodiments, the first vector and the second vector may be introduced together into the host cell. In certain embodiments, the first vector may be introduced first into the host cell, and then the second vector may be introduced. In certain embodiments, the first vector may be introduced into the host cell which is then established into a stable cell line expressing the first polypeptide, and then the second vector may be introduced into the stable cell line.

In certain embodiments, the host cells comprise a vector encoding for a first polypeptide and a second polypeptide.

In certain embodiments, the present disclosure provides methods of expressing the polypeptide provided herein, comprising culturing the host cell containing the vector under conditions in which the inserted polynucleotide in the vector is expressed.

Suitable conditions for expression of the polynucleotide may include, without limitation, suitable medium, suitable density of host cells in the culture medium, presence of necessary nutrients, presence of supplemental factors, suitable temperatures and humidity, and absence of microorganism contaminants. A person with ordinary skill in the art can select the suitable conditions as appropriate for the purpose of the expression.

In some embodiments, a method of producing a precursor tri-specific antibody construct comprising (a) a first binding domain binding to a cell surface tumor associated antigen (TAA binding domain); (b) a second binding domain binding to a NK surface antigen; (c) a third binding domain binding to an extracellular epitope of a T cell or NK cell surface antigen; (d) a CAP regulatory domain; and (e) an HSA regulatory domain, comprises steps of culturing a cell or cells comprising a nucleic acid sequence encoding polypeptide A and polypeptide B of the precursor tri-specific antibody construct, wherein said precursor tri-specific antibody construct polypeptides are expressed and isolated, and wherein said isolated polypeptides A and B form a heterodimer. As disclosed herein in detail, the isolated nucleic acid sequences encoding polypeptides A and B may be comprised within vectors, wherein the same vector or different vectors are used. In some embodiments, each polypeptide may be expressed from a different host cell, wherein dimerization occurs following isolation or purification of the component polypeptides A and B. In some embodiments, polypeptides A and B may be expressed from a same host cell, wherein dimerization occurs in culture or following isolation or purification of the component polypeptides A and B.

In certain embodiments, the polypeptide expressed in the host cell can form a dimer and thus produce a precursor tri-specific antibody construct dimer, for example a heterodimer comprising a polypeptide A and a polypeptide B. In certain embodiments, where the host cells express a first polynucleotide and a second polynucleotide, the first polynucleotide (A) and the second polynucleotide (B) can form a polypeptide complex which is a heterodimer.

In certain embodiments, the polypeptide complex may be formed inside the host cell. For example, the heterodimer may be formed inside the host cell with the aid of relevant enzymes and/or cofactors. In certain embodiments, the polypeptide complex may be secreted out of the cell. In certain embodiments, the first polypeptide (A) and the second polypeptide (B) may be secreted out of the host cell and form a heterodimer outside of the host cell.

In certain embodiments, the first polypeptide and the second polypeptide may be separately expressed and allowed to dimerize under suitable conditions. For example, the first polypeptide (A) and the second polypeptide (B) may be combined in a suitable buffer and allow the first protein monomer (A) and the second protein monomer (B) to dimerize through appropriate interactions such as hydrophobic interactions. For another example, the first polypeptide (A) and the second polypeptide (B) may be combined in a suitable buffer containing an enzyme and/or a cofactor which can promote the dimerization of the first polypeptide (A) and the second polypeptide (B). For another example, the first polypeptide (A) and the second polypeptide (A) may be combined in a suitable vehicle and allow them to react with each other in the presence of a suitable reagent and/or catalyst.

In certain embodiments, the first polypeptide (A) and the second polypeptide (B) may be generated by DNA synthesis and PCR. In certain embodiments, the generated sequences may be subcloned into an expression vector. In certain embodiments, the generated sequences may be subcloned into two expression vectors. In certain embodiments, said expression vector is a plasmid. In certain embodiments, said plasmid is pTT5-based plasmid.

In certain embodiments, transient expression is performed by co-transfecting the expression vector encoding the first polypeptide (A) and the second polypeptide (B) or by transfecting an expression vector encoding both into a suitable cell. A skilled artisan would appreciate that there are a number of transfection methods and protocols that can be used for this purpose. In certain embodiments, transfection or co-transfection is executed using the PEI method. In certain embodiments, 1L of CHO cells at approximately 2.3×106/ml in a 3 L shake flask is used as the host. Transfection is initiated by adding a mixture of 2 mg of total DNA and 4 mg PEI in 100 ml OptiMEM medium (Invitrogen) to the cells and gentle mixing. Cells are then cultured in an incubator shaker at 120 rpm, 37° C., and 8% CO2, for 8-10 days. Feeding with peptone and glucose is carried out 24 h later and every 2-3 days thereafter depending on the cell density and viability. The cell culture is terminated on day 8-10 when cell viability reduces to <70%. The conditioned medium is then harvested for protein purification.

The expressed polypeptides (A) and (B) and/or the polypeptide complex can be collected using any suitable methods. The polypeptides (A) and (B) and/or the polypeptide complex can be expressed intracellularly, in the periplasmic space or be secreted outside of the cell into the medium. If the polypeptides (A) and (B) and/or the polypeptide complex is expressed intracellularly, the host cells containing the polypeptides (A) and (B) and/or the polypeptide complex may be lysed and polypeptide and/or the polypeptide complex may be isolated from the lysate by removing the unwanted debris by centrifugation or ultrafiltration. If the polypeptides (A) and (B) and/or the polypeptide complex is secreted into periplasmic space of E. coli, the cell paste may be thawed in the presence of agents such as sodium acetate (pH 3.5), EDTA, and phenylmethylsulfonylfluoride (PMSF) over about 30 min, and cell debris can be removed by centrifugation (Carter et al., BioTechnology 10:163-167 (1992)). If the polypeptides (A) and (B) and/or the polypeptide complex is secreted into the medium, the supernatant of the cell culture may be collected and concentrated using a commercially available protein concentration filter, for example, an Amincon or Millipore Pellicon ultrafiltration unit. A protease inhibitor and/or an antibiotic may be included in the collection and concentration steps to inhibit protein degradation and/or growth of contaminated microorganisms.

The expressed polypeptides (A) and (B) and/or the polypeptide complex can be further purified by a suitable method, such as without limitation, affinity chromatography, hydroxylapatite chromatography, size exclusion chromatography, gel electrophoresis, dialysis, ion exchange fractionation on an ion-exchange column, ethanol precipitation, reverse phase HPLC, chromatography on silica, chromatography on heparin sepharose, chromatography on an anion or cation exchange resin (such as a polyaspartic acid column), chromatofocusing, SDS-PAGE, and ammonium sulfate precipitation (see, for review, Bonner, P. L., Protein purification, published by Taylor & Francis, 2007; Janson, J. C., et al, Protein purification: principles, high resolution methods and applications, published by Wiley-VCH, 1998).

In certain embodiments, the polypeptides (A) and (B) and/or polypeptide dimer complexes can be purified by affinity chromatography. In certain embodiments, protein A chromatography or protein A/G (fusion protein of protein A and protein G) chromatography can be useful for purification of polypeptides and/or polypeptide complexes comprising a component derived from antibody CH2 domain and/or CH3 domain (Lindmark et al., J. Immunol. Meth. 62:1-13 (1983)); Zettlit, K. A., Antibody Engineering, Part V, 531-535, 2010). In certain embodiments, a precursor tri-specific antibody construct disclosed herein does not bind to protein A. In certain embodiments, protein G chromatography can be useful for purification of polypeptides and/or polypeptide complexes comprising IgGγ3 heavy chain (Guss et al., EMBO J. 5:1567 1575 (1986)). In certain embodiments, protein L chromatography can be useful for purification of polypeptides and/or polypeptide complexes comprising K light chain (Sudhir, P., Antigen engineering protocols, Chapter 26, published by Humana Press, 1995; Nilson, B. H. K. et al, J. Biol. Chem., 267, 2234-2239 (1992)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX resin (J. T. Baker, Phillipsburg, N.J.) is useful for purification.

Following any preliminary purification step(s), the mixture comprising the precursor tri-specific antibody construct and contaminants may be subjected to low pH hydrophobic interaction chromatography using an elution buffer at a pH between about 2.5-4.5, preferably performed at low salt concentrations (e.g., from about 0-0.25 M salt).

In certain embodiments, the polypeptides (A) and (B) and/or polypeptide dimer complexes can be purified by affinity chromatography and size exclusion chromatography (SEC). A skilled artisan would appreciate that there are a number of methods and protocols suitable for this purpose. In certain embodiments, protein purification by affinity chromatography and SEC is performed using an AKTA pure instrument (GE Lifesciences). In certain embodiments, affinity capture of the precursor bispecific antibody is achieved by passing the harvested supernatants over a column of CaptureSelect™ CH1-XL Affinity Matrix (Thermo Scientific). After washing column with PBS, the protein is eluted with 0.1 M Glycine, pH 2.5, and immediately neutralized with ⅙ volume of 1 M Tris-HCl, pH 8.0. The affinity purified protein is then concentrated to 5-10 mg/ml using Amicon 30 kD concentrator (Merck Millipore) and subjected to SEC purification on a Superdex200 column (GE Lifesciences) equilibrated with PBS. Protein fractions are then collected and analyzed using SDS-PAGE and HPLC-SEC.

### Methods of Use of Precursor Tri-specific Antibody Constructs

In some embodiments, described herein are compositions comprising the precursor tri-specific antibody construct as described herein and administration of such composition in a variety of therapeutic settings. In one embodiment, the precursor tri-specific antibody construct comprises three binding domains. In one embodiment, the third binding domain is a Fab, and the first and second binding domains are scFv. In one embodiment, the third binding domain binds to a T cell surface antigen, the first binding domain binds to a TAA and the second binding domain binds to a NK cell surface antigen. In another embodiment, the third binding domain binds to a NK cell surface antigen, the first binding domain binds to a TAA and the second binding domain binds to another NK cell surface antigen. In another embodiment, the third binding domain binds to a NK cell surface antigen, the first binding domain binds to a TAA and the second binding domain comprises a cytokine receptor engager (e.g. IL-15).

Administration of the precursor tri-specific antibody constructs described herein, in pure form or in an appropriate pharmaceutical composition, can be carried out via any of the accepted modes of administration of agents for serving similar utilities. The pharmaceutical compositions can be prepared by combining a precursor tri-specific antibody construct or a precursor tri-specific antibody construct-containing composition with an appropriate physiologically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. In addition, other pharmaceutically active ingredients (including other anti-cancer agents as described elsewhere herein) and/or suitable excipients such as salts, buffers and stabilizers may, but need not, be present within the composition. Administration may be achieved by a variety of different routes, including oral, parenteral, nasal, intravenous, intradermal, subcutaneous or topical. In some embodiments, modes of administration depend upon the nature of the condition to be treated or prevented. An amount that, following administration, reduces, inhibits, prevents or delays the progression and/or metastasis of a cancer is considered effective. A skilled artisan would appreciate that the term “physiologically acceptable carrier, diluent or excipient”, may in some embodiments be used interchangeably with the term “pharmaceutically acceptable carrier” having all the same means and qualities.

In some embodiments, a pharmaceutical composition described herein comprises a nucleotide sequence encoding a precursor tri-specific antibody construct. In some embodiments, a nucleotide sequence encoding a precursor construct disclosed herein, comprises a single linear nucleotide sequence. In some embodiments, a nucleotide sequence encoding a precursor construct disclosed herein, comprises two nucleotide sequences. In some embodiments, a nucleotide sequence encoding a precursor construct disclosed herein, comprises two nucleotide sequences present on the same vector. In some embodiments, a nucleotide sequence encoding a precursor construct disclosed herein, comprises two nucleotide sequences present on different vectors.

In some embodiments, the nucleotide sequence encodes polypeptide A and polypeptide B. In some embodiments, the same nucleotide sequence encodes polypeptide A and polypeptide B. In some embodiments, different nucleotide sequences encode polypeptide A and polypeptide B. In some embodiments, one nucleotide sequence encodes polypeptide A and another nucleotide sequence encodes polypeptide B. In some embodiments, one nucleotide sequence encodes polypeptide A and another nucleotide sequence encodes polypeptide B having a protease cleavage sequence between them, thus allowing polypeptide A and polypeptide B to hetero-dimerize, as described in Duperret EK et al., Cancer Res, Oct. 4 (doi: 10.1158/0008-5472.CAN-18-1429)In some embodiments, a method of treating, preventing, inhibiting the growth of, delaying disease progression, reducing the tumor load, or reducing the incidence of a cancer or a tumor in a subject, or any combination thereof, comprises a step of administering to a subject in need thereof a pharmaceutical composition comprising one of the precursor tri-specific antibody constructs disclosed herein, wherein the method treats, prevents, inhibits the growth of, delays the disease progression, reduces the tumor load, or reduces the incidence of the cancer or a tumor in said subject, or reduces the minimal residual disease, increases remission, increases remission duration, reduces tumor relapse rate, prevents metastasis of said tumor or said cancer, or reduces the rate of metastasis of said tumor or said cancer, or any combination thereof, compared with a subject not administered said pharmaceutical composition.

In some embodiments, a method of treating, preventing, inhibiting the growth of, delaying disease progression, reducing the tumor load, or reducing the incidence of a cancer or a tumor in a subject, or any combination thereof, comprises a step of administering to a subject in need thereof a pharmaceutical composition comprising a nucleotide sequence encoding one of the precursor bispecific antibody constructs disclosed herein, wherein the method treats, prevents, inhibits the growth of, delays the disease progression, reduces the tumor load, or reduces the incidence of the cancer or a tumor in said subject, or reduces the minimal residual disease, increases remission, increases remission duration, reduces tumor relapse rate, prevents metastasis of said tumor or said cancer, or reduces the rate of metastasis of said tumor or said cancer, or any combination thereof, compared with a subject not administered said pharmaceutical composition.

A skilled artisan would appreciate that the term “treating” and grammatical forms thereof, may in some embodiments encompass both therapeutic treatment and prophylactic or preventative measures with respect to a tumor or cancer as described herein, wherein the object is to prevent or lessen the targeted tumor or cancer as described herein. Thus, in some embodiments of methods disclosed herein, treating may include directly affecting or curing, suppressing, inhibiting, preventing, reducing the severity of, delaying the onset of, reducing symptoms associated with the disease, disorder or condition, or a combination thereof; for example, when said disease or disorder comprises a cancer or tumor. Thus, in some embodiments, “treating” encompasses preventing, delaying progression, inhibiting the growth of, delaying disease progression, reducing tumor load, reducing the incidence of, expediting remission, inducing remission, augmenting remission, speeding recovery, increasing efficacy of or decreasing resistance to alternative therapeutics, or a combination thereof. In some embodiments, “preventing” encompasses delaying the onset of symptoms, preventing relapse to a disease, decreasing the number or frequency of relapse episodes, increasing latency between symptomatic episodes, or a combination thereof. In some embodiments, “suppressing” or “inhibiting”, encompass reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

In some embodiments, the size of a cancer or tumor is reduced. In some embodiments, the growth rate of a cancer or tumor is reduced. In some embodiments, the size or the growth rate or a combination thereof, of a cancer or tumor is reduced. In some embodiments, the survival of the subject in need is increased. In some embodiments, the size or the growth rate or a combination thereof, of a cancer or tumor is reduced, or wherein the survival of the subject in need is increased or a combination thereof.

In some embodiments, the subject in need is a human subject. In some embodiments, the subject in need is a human child. In some embodiments, the subject in need is an adult human. In some embodiments, the subject in need is a human infant.

In certain embodiments, the amount administered is sufficient to result in tumor regression, as indicated by a statistically significant decrease in the amount of viable tumor, for example, at least a 50% decrease in tumor mass, or by altered (e.g., decreased with statistical significance) scan dimensions. In other embodiments, the amount administered is sufficient to result in clinically relevant reduction in disease symptoms as would be known to the skilled clinician.

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

The precursor tri-specific antibody construct-containing compositions may be administered alone or in combination with other known cancer treatments, such as radiation therapy, chemotherapy, transplantation, immunotherapy, hormone therapy, photodynamic therapy, etc. In some embodiments, compositions comprising nucleotide sequences encoding a precursor bispecific antibody construct, may be administered alone or in combination with other known cancer treatments, such as radiation therapy, chemotherapy, transplantation, immunotherapy, hormone therapy, photodynamic therapy, etc. The compositions may also be administered in combination with antibiotics.

Typical routes of administering these and related pharmaceutical compositions thus include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Pharmaceutical compositions according to certain embodiments as described herein, are formulated so as to allow the active ingredients contained therein to be bioavailable upon administration of the composition to a patient. Compositions that will be administered to a subject or patient may take the form of one or more dosage units, where for example, a tablet may be a single dosage unit, and a container of a herein described precursor tri-specific antibody construct in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of a precursor bispecific antibody construct of the present disclosure, for treatment of a disease or condition of interest in accordance with teachings herein.

A pharmaceutical composition may be in the form of a solid or liquid. In one embodiment, the pharmaceutically acceptable carrier(s) are particulate, so that the compositions are, for example, in tablet or powder form. The pharmaceutically acceptable carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like. Such a solid composition will typically contain one or more inert diluents or edible pharmaceutically acceptable carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the pharmaceutical composition is in the form of a capsule, for example, a gelatin capsule, it may contain, in addition to materials of the above type, a liquid pharmaceutically acceptable carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions, whether they be solutions, suspensions or other like form, may include one or more of the following adjuvants: sterile diluents such as water for injection, saline solution, preferably physiological saline, Ringer’s solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition intended for either parenteral or oral administration should contain an amount of a precursor tri-specific antibody construct as herein disclosed such that a suitable dosage will be obtained. Typically, this amount is at least 0.01% of the precursor bispecific antibody construct in the composition. When intended for oral administration, this amount may be varied to be between 0.1 and about 70% of the weight of the composition. Certain oral pharmaceutical compositions contain between about 4% and about 75% of the precursor bispecific antibody construct. In certain embodiments, pharmaceutical compositions and preparations according to the embodiments described herein, are prepared so that a parenteral dosage unit contains between 0.01 to 10% by weight of the precursor tri-specific antibody construct prior to dilution.

The pharmaceutical composition may be intended for topical administration, in which case the pharmaceutically acceptable carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. The pharmaceutical composition may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The pharmaceutical composition in solid or liquid form may include an agent that binds to the antibody as disclosed herein, and thereby assists in the delivery of the compound. Suitable agents that may act in this capacity include other monoclonal or polyclonal antibodies, one or more proteins or a liposome. The pharmaceutical composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi-phasic, or tri-phasic systems in order to deliver the active ingredient(s). Delivery of the aerosol includes the necessary container, activators, valves, subcontainers, and the like, which together may form a kit. One of ordinary skill in the art, without undue experimentation may determine preferred aerosols.

The pharmaceutical compositions may be prepared by methodology well known in the pharmaceutical art. For example, a pharmaceutical composition intended to be administered by injection can be prepared by combining a composition that comprises a precursor tri-specific antibody construct as described herein and optionally, one or more of salts, buffers and/or stabilizers, with sterile, distilled water so as to form a solution. A surfactant may be added to facilitate the formation of a homogeneous solution or suspension. Surfactants are compounds that non-covalently interact with the precursor bispecific antibody construct composition so as to facilitate dissolution or homogeneous suspension of the precursor bispecific antibody construct in the aqueous delivery system.

The compositions may be administered in a therapeutically effective amount, which will vary depending upon a variety of factors including the activity of the specific compound (e.g., precursor bispecific antibody construct) employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose is (for a 70 kg mammal) from about 0.001 mg/kg (i.e., 0.07 mg) to about 100 mg/kg (i.e., 7.0 g); preferably a therapeutically effective dose is (for a 70 kg mammal) from about 0.01 mg/kg (i.e., 0.7 mg) to about 50 mg/kg (i.e., 3.5 g); more preferably a therapeutically effective dose is (for a 70 kg mammal) from about 1 mg/kg (i.e., 70 mg) to about 25 mg/kg (i.e., 1.75 g).

Compositions comprising the precursor tri-specific antibody construct of the present disclosure or comprising a nucleotide sequence encoding the precursor tri-specific antibody construct may also be administered simultaneously with, prior to, or after administration of one or more other therapeutic agents. Such combination therapy may include administration of a single pharmaceutical dosage formulation which contains a compound as disclosed herein, and one or more additional active agents, as well as administration of compositions comprising precursor tri-specific antibody construct as disclosed herein, and each active agent in its own separate pharmaceutical dosage formulation. For example, a precursor tri-specific antibody construct or comprising a nucleotide sequence encoding the precursor tri-specific antibody construct, as described herein, and the other active agent can be administered to the patient together in a single oral dosage composition such as a tablet or capsule, or each agent administered in separate oral dosage formulations. Similarly, a precursor tri-specific antibody construct or comprising a nucleotide sequence encoding the precursor tri-specific antibody construct, as described herein, and the other active agent can be administered to the patient together in a single parenteral dosage composition such as in a saline solution or other physiologically acceptable solution, or each agent administered in separate parenteral dosage formulations. Where separate dosage formulations are used, the compositions comprising precursor tri-specific antibody construct or comprising a nucleotide sequence encoding the precursor tri-specific antibody construct, and one or more additional active agents can be administered at essentially the same time, i.e., concurrently, or at separately staggered times, i.e., sequentially and in any order; combination therapy is understood to include all these regimens.

Thus, in certain embodiments, also contemplated is the administration of precursor tri-specific antibody construct compositions of this disclosure or comprising a nucleotide sequence encoding the precursor tri-specific antibody construct, in combination with one or more other therapeutic agents. Such therapeutic agents may be accepted in the art as a standard treatment for a particular disease state as described herein, such as cancer, inflammatory disorders, allograft transplantation, type I diabetes, and multiple sclerosis. Exemplary therapeutic agents contemplated include cytokines, growth factors, steroids, NSAIDs, DMARDs, anti-inflammatories, chemotherapeutics, radiotherapeutics, or other active and ancillary agents.

In certain embodiments, the precursor tri-specific antibody construct or comprising a nucleotide sequence encoding the precursor tri-specific antibody construct, disclosed herein may be administered in conjunction with any number of chemotherapeutic agents. Examples of chemotherapeutic agents include alkylating agents such as thiotepa and cyclophosphamide (CYTOXAN.TM.); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK.RTM.; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL.RTM., Bristol-Myers Squibb Oncology, Princeton, N.J.) and doxetaxel (TAXOTERE.RTM., Rhne-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid derivatives such as Targretin.TM. (bexarotene), Panretin.TM. (alitretinoin); ONTAK.TM. (denileukin diftitox); esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

A variety of other therapeutic agents may be used in conjunction with the precursor tri-specific antibody construct described herein. In one embodiment, the precursor tri-specific antibody construct or comprising a nucleotide sequence encoding the precursor tri-specific antibody construct, is administered with an anti-inflammatory agent. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate.

The compositions comprising herein described precursor tri-specific antibody construct or comprising a nucleotide sequence encoding the precursor tri-specific antibody construct may be administered to an individual afflicted with a disease as described herein, including, but not limited to cancer and autoimmune and inflammatory diseases. For in vivo use for the treatment of human disease, the precursor tri-specific antibody construct or comprising a nucleotide sequence encoding the precursor tri-specific antibody construct described herein are generally incorporated into a pharmaceutical composition prior to administration. A pharmaceutical composition comprises one or more of the precursor tri-specific antibody construct or comprising a nucleotide sequence encoding the precursor tri-specific antibody construct described herein in combination with a pharmaceutically acceptable carrier or excipient as described elsewhere herein. To prepare a pharmaceutical composition, an effective amount of one or more of the precursor tri-specific antibody constructs or comprising a nucleotide sequence encoding the precursor tri-specific antibody construct is mixed with any pharmaceutically acceptable carrier(s) or excipient known to those skilled in the art to be suitable for the particular mode of administration.

A pharmaceutically acceptable carrier may be liquid, semi-liquid or solid. Solutions or suspensions used for parenteral, intradermal, subcutaneous or topical application may include, for example, a sterile diluent (such as water), saline solution, fixed oil, polyethylene glycol, glycerin, propylene glycol or other synthetic solvent; antimicrobial agents (such as benzyl alcohol and methyl parabens, phenols or cresols, mercurials, chlorobutanol, methyl and propyl p-hydroxybenzoic acid esters, thimerosal, benzalkonium chloride and benzethonium chloride); antioxidants (such as ascorbic acid and sodium bisulfite; methionine, sodium thiosulfate, platinum, catalase, citric acid, cysteine, thioglycerol, thioglycolic acid, thiosorbitol, butylated hydroxyanisol, butylated hydroxytoluene, and/or propyl gallate) and chelating agents (such as ethylenediaminetetraacetic acid (EDTA)); buffers (such as acetates, citrates and phosphates). If administered intravenously, suitable pharmaceutically acceptable carriers include physiological saline or phosphate buffered saline (PBS), and solutions containing thickening and solubilizing agents, such as glucose, polyethylene glycol, polypropylene glycol and mixtures thereof.

The compositions comprising precursor tri-specific antibody construct as described herein may be prepared with pharmaceutically acceptable carriers that protect the precursor tri-specific antibody construct against rapid elimination from the body, such as time release formulations or coatings. Such pharmaceutically acceptable carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

The present precursor tri-specific antibody construct are useful for the treatment of a variety of cancers or tumors. In some embodiments, the cancer or tumor comprises a solid tumor. In some embodiments, the cancer or tumor comprises a non-solid tumor. In some embodiments, the cancer or tumor comprises a metastasis of a cancer or tumor.

For example, some embodiments of a method for the treatment of a cancer are directed to cancers including, but not limited to, melanoma, non-Hodgkin’s lymphoma, Hodgkin’s disease, leukemia, plasmocytoma, sarcoma, glioma, thymoma, breast cancer, prostate cancer, colo-rectal cancer, kidney cancer, renal cell carcinoma, uterine cancer, pancreatic cancer, esophageal cancer, brain cancer, lung cancer, ovarian cancer, cervical cancer, testicular cancer, gastric cancer, esophageal cancer, multiple myeloma, hepatoma, acute lymphoblastic leukemia (ALL), acute myelogenous leukemia (AML), chronic myelogenous leukemia (CML), and chronic lymphocytic leukemia (CLL), or other cancers, by administering to a cancer patient a therapeutically effective amount of a herein disclosed precursor bispecific antibody construct or a nucleotide sequence encoding the precursor tri-specific antibody construct.

Solid tumors may be benign (not cancer), or malignant (cancer). Different types of solid tumors are named for the type of cells that form them. Examples of solid tumors for which treatment may be provided include sarcomas, carcinomas, and lymphomas. In some embodiments, solid tumors for which treatment may be provided include neoplasms (new growth of cells) or lesions (damage of anatomic structures or disturbance of physiological functions) formed by an abnormal growth of body tissue cells other than blood, bone marrow or lymphatic cells. In some embodiments, a solid tumor for which treatment may be provided consists of an abnormal mass of cells which may stem from different tissue types such as liver, colon, breast, or lung, and which initially grows in the organ of its cellular origin. However, such cancers may spread to other organs through metastatic tumor growth in advanced stages of the disease.

In some embodiments of a method for treatment of a cancer or tumor, the solid tumor or cancer comprises a sarcoma or a carcinoma, adrenocortical tumor (adenoma and carcinoma), a fibrosarcoma, a myxo-sarcoma, a liposarcoma, a chondrosarcoma, an osteogenic sarcoma, a chordoma, an angiosarcoma, an endothelio sarcoma, a lymphangiosarcoma, a lymphangioendothelio sarcoma, a synovioma, a mesothelioma, an Ewing’s tumor, a leiomyosarcoma, a rhabdomyosarcoma, a colon carcinoma, a pancreatic cancer or tumor, a breast cancer or tumor, an ovarian cancer or tumor, a prostate cancer or tumor, a squamous cell carcinoma, a squamous cell carcinoma of the lung, a basal cell carcinoma, an adenocarcinoma, a sweat gland carcinoma, a sebaceous gland carcinoma, a papillary carcinoma, a papillary adenocarcinomas, a cystadenocarcinoma, a medullary carcinoma, a bronchogenic carcinoma, a renal cell carcinoma, a hepatoma, a bile duct carcinoma, a choriocarcinoma, a seminoma, an embryonal carcinoma, a colorectal carcinoma, a desmoid tumor, a desmoplastic small round cell tumor, an endocrine tumor, a germ cell tumor, a hepatoblastoma, a hepatocellular carcinoma, a melanoma, a neuroblastoma, an osteosarcoma, a retinoblastoma, a rhabdomyosarcoma, a soft tissue sarcoma other than rhabdomyosarcoma, a Wilms, Tumor, a cervical cancer or tumor, a uterine cancer or tumor, a testicular cancer or tumor, a lung carcinoma, a small cell lung carcinoma, an anal cancer, a glioblastoma, an epithelial tumor of the head and neck, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodenroglioma, a schwannoma, a meningioma, a melanoma, a neuroblastoma, or a retinoblastoma.

In some embodiments of a method for treatment of a cancer or tumor, the tumor or cancer comprises a non-solid tumor, that is a non-solid cancer. In some embodiments, methods for treatment of a cancer or tumor may be for a diffuse cancer, wherein the cancer is widely spread; not localized or confined. In some embodiments, a diffuse cancer may comprise a non-solid tumor. Examples of diffuse cancers include leukemias. Leukemias comprise a cancer that starts in blood-forming tissue, such as the bone marrow, and causes large numbers of abnormal blood cells to be produced and enter the bloodstream.

In some embodiments of a method for treatment of a cancer or tumor, the diffuse cancer comprises a B-cell malignancy. In some embodiments, the diffuse cancer comprises leukemia. In some embodiments, the cancer is lymphoma. In some embodiments, the lymphoma is large B-cell lymphoma.

In some embodiments of a method for treatment of a cancer or tumor, the diffuse cancer or tumor comprises a hematological tumor. In some embodiments, hematological tumors are cancer types affecting blood, bone marrow, and lymph nodes. Hematological tumors may derive from either of the two major blood cell lineages: myeloid and lymphoid cell lines. The myeloid cell line normally produces granulocytes, erythrocytes, thrombocytes, macrophages, and masT-cells, whereas the lymphoid cell line produces B, T, NK and plasma cells. Lymphomas (e.g. Hodgkin’s Lymphoma), lymphocytic leukemias, and myeloma are derived from the lymphoid line, while acute and chronic myelogenous leukemia (AML, CML), myelodysplastic syndromes and myeloproliferative diseases are myeloid in origin.

In some embodiments of a method for treatment of a cancer or tumor, the non-solid (diffuse) cancer or tumor comprises a hematopoietic malignancy, a blood cell cancer, a leukemia, a myelodysplastic syndrome, a lymphoma, a multiple myeloma (a plasma cell myeloma), an acute lymphoblastic leukemia, an acute myelogenous leukemia, a chronic myelogenous leukemia, a Hodgkin lymphoma, a non-Hodgkin lymphoma, or plasma cell leukemia.

An amount that, following administration, inhibits, prevents reduces the incidence of, reduces the tumor load, or delays the growth, progression and/or metastasis of a cancer in a statistically significant manner (i.e., relative to an appropriate control as will be known to those skilled in the art) is considered effective.

Another embodiment provides a method for preventing metastasis of a cancer including, but not limited to a solid or non-solid tumor or cancer as disclosed above, by administering to a cancer patient a therapeutically effective amount of a herein disclosed precursor tri-specific antibody construct or a nucleotide sequence encoding the precursor bispecific antibody construct (e.g., an amount that, following administration, inhibits, prevents or delays metastasis of a cancer in a statistically significant manner, i.e., relative to an appropriate control as will be known to those skilled in the art).

Another embodiment provides a method for preventing a cancer including, but not limited to a solid or non-solid tumor or cancer as disclosed above, by administering to a cancer patient a therapeutically effective amount of a herein disclosed precursor tri-specific antibody construct or a nucleotide sequence encoding the precursor bispecific antibody construct.

Another embodiment provides a method for treating, inhibiting the progression of a tumor or cancer including but not limited to a solid or non-solid tumor or cancer as disclosed above, by administering to a patient afflicted by one or more of these diseases a therapeutically effective amount of a herein disclosed precursor bispecific antibody construct or a nucleotide sequence encoding the precursor tri-specific antibody construct.

In one embodiment, the present disclosure provides a method for directing T cell and/or NK cell activation, comprising administering to a patient in need thereof an effective amount of a precursor tri-specific antibody construct that comprises a CD3 binding domain or NK cell binding domain, as described herein, that is able to specifically binds NK cells, TCRα, TCRβ, CD3γ, CD3δ, CD3ε, or a combination thereof, and a TAA first binding domain that specifically binds a TAA target, for instance, a tumor-specific antigen (e.g., EGFR) or other antigens of choice at a site or cell where T-cell and/or NK cell activation is desired.

In one embodiment, the present disclosure provides a precursor tri-specific antibody construct, comprising: (i) a first binding domain that binds to a tumor associated antigen (TAA); (ii) a second binding domain that binds to a first natural killer (NK) cell surface antigen or a second binding domain comprising a cytokine receptor engager; (iii) a third binding domain that binds to a T cell surface antigen or a second NK cell surface antigen; and (iv) a regulatory domain, said regulatory domain comprising either (a) a first and a second sub-regulatory domain, said first sub-regulatory domain comprising a first protease cleavage domain and a half-life prolonging (HLP) domain, and said second sub-regulatory domain comprising a second protease cleavage domain and a CAP component that reduces the ability of the third binding domain to bind to its target antigen; or (b) a single regulatory domain comprising a protease cleavage domain, a half-life prolonging (HLP) domain, and a CAP component that reduces the ability of the third binding domain to bind to its target antigen.

In one embodiment, the second binding domain further comprises a third regulatory domain comprising a third protease cleavage domain and a CAP component that reduces the ability of the second binding domain to bind to said first NK cell surface antigen.

In one embodiment, the first binding domain and the second binding domain each comprises a single chain variable fragment (scFv), and the third binding domain comprises a Fab antigen binding fragment. In another embodiment, the first binding domain comprises a single chain variable fragment (scFv), the second binding domain comprises two scFv, and the third binding domain comprises a Fab antigen binding fragment.

In one embodiment, the first binding domain binds to a TAA, the second binding domain binds to a NK cell surface antigen, and the third binding domain binds to T cell surface antigen CD3. In one embodiment, the first binding domain binds to 5T4, and the second binding domain binds to NKG2A. In another embodiment, the first binding domain binds to 5T4, and the second binding domain binds to NKG2D. In another embodiment, the first binding domain binds to 5T4, and the second binding domain binds to CD16.

In one embodiment, the first binding domain that binds to 5T4 comprises three complementarity determining regions (CDRs) on a heavy chain (HCDR1, HCDR2, and HCDR3) and three CDRs on a light chain (LCDR1, LCDR2, and LCDR3), wherein
(i) the HCDR1, HCDR2, and HCDR3 comprises the amino acid sequences of SEQ ID NOs:476-478, and the LCDR1, LCDR2, and LCDR3 comprises the amino acid sequences of SEQ ID NOs:480-482; or
(ii) the HCDR1, HCDR2, and HCDR3 comprises the amino acid sequences of SEQ ID NOs:484-486, and the LCDR1, LCDR2, and LCDR3 comprises the amino acid sequences of SEQ ID NOs:488-490; or
(iii) the HCDR1, HCDR2, and HCDR3 comprises the amino acid sequences of SEQ ID NOs:492-494, and the LCDR1, LCDR2, and LCDR3 comprises the amino acid sequences of SEQ ID NOs:496-498; or
(iv) the HCDR1, HCDR2, and HCDR3 comprises the amino acid sequences of SEQ ID NOs:500-502, and the LCDR1, LCDR2, and LCDR3 comprises the amino acid sequences of SEQ ID NOs:504-506; or
(v) the HCDR1, HCDR2, and HCDR3 comprises the amino acid sequences of SEQ ID NOs:508-510, and the LCDR1, LCDR2, and LCDR3 comprises the amino acid sequences of SEQ ID NOs:512-514; or
(vi) the HCDR1, HCDR2, and HCDR3 comprises the amino acid sequences of SEQ ID NOs:516-518, and the LCDR1, LCDR2, and LCDR3 comprises the amino acid sequences of SEQ ID NOs:520-522; or
(vii) the HCDR1, HCDR2, and HCDR3 comprises the amino acid sequences of SEQ ID NOs:524-526, and the LCDR1, LCDR2, and LCDR3 comprises the amino acid sequences of SEQ ID NOs:528-530; or
(viii) the HCDR1, HCDR2, and HCDR3 comprises the amino acid sequences of SEQ ID NOs:532-534, and the LCDR1, LCDR2, and LCDR3 comprises the amino acid sequences of SEQ ID NOs:536-538; or
(ix) the HCDR1, HCDR2, and HCDR3 comprises the amino acid sequences of SEQ ID NOs:540-542, and the LCDR1, LCDR2, and LCDR3 comprises the amino acid sequences of SEQ ID NOs:544-546.

In one embodiment, the first binding domain that binds to 5T4 comprises a heavy chain variable region and a light chain variable region, said heavy chain variable region and light chain variable region comprise the amino acid sequences of SEQ ID NOs:475 and 479; SEQ ID NOs:483 and 487; SEQ ID NOs:491 and 495; SEQ ID NOs:499 and 503; SEQ ID NOs:507 and 511; SEQ ID NOs:515 and 519; SEQ ID NOs:523 and 527; or SEQ ID NOs:531 and 535; SEQ ID NOs:539 and 543; SEQ ID NOs:547-548; SEQ ID NOs:549-550; SEQ ID NOs:551-552; SEQ ID NOs:553-554; SEQ ID NOs:555-556; SEQ ID NOs:557-558; SEQ ID NOs:559-560; SEQ ID NOs:561-562; SEQ ID NOs:563-564; SEQ ID NOs:565-566; SEQ ID NOs:567-568; SEQ ID NOs:569-570; SEQ ID NOs:571-572; SEQ ID NOs:573-574; SEQ ID NOs:575-576; SEQ ID NOs:577-578; SEQ ID NOs:579-580; SEQ ID NOs:581-582; SEQ ID NOs:583-584; SEQ ID NOs:585-586; SEQ ID NOs:587-588; SEQ ID NOs:589-590; SEQ ID NOs:591-592; SEQ ID NOs:593-594; SEQ ID NOs:595-596; SEQ ID NOs:597-598; SEQ ID NOs:599-600; SEQ ID NOs:601-602; SEQ ID NOs:603-604; SEQ ID NOs:605-606; SEQ ID NOs:607-608; SEQ ID NOs:609-610; SEQ ID NOs:611-612; SEQ ID NOs:613-614; SEQ ID NOs:615-616; SEQ ID NOs:617-618; SEQ ID NOs:619-620; SEQ ID NOs:621-622; SEQ ID NOs:623-624; SEQ ID NOs:625-626; SEQ ID NOs:627-628; SEQ ID NOs:629-630; SEQ ID NOs:631-632; or SEQ ID NOs:633-634.

In one embodiment, for any precursor tri-specific antibody constructs disclosed above, the second binding domain that binds to NKG2D comprises three complementarity determining regions (CDRs) on a heavy chain (HCDR1, HCDR2, and HCDR3) and three CDRs on a light chain (LCDR1, LCDR2, and LCDR3), wherein
(i) the HCDR1, HCDR2, and HCDR3 comprises the amino acid sequences of SEQ ID NOs:646-648, and the LCDR1, LCDR2, and LCDR3 comprises the amino acid sequences of SEQ ID NOs:650-652; or
(ii) the HCDR1, HCDR2, and HCDR3 comprises the amino acid sequences of SEQ ID NOs:656-658, and the LCDR1, LCDR2, and LCDR3 comprises the amino acid sequences of SEQ ID NOs:660-662; or
(iii) the HCDR1, HCDR2, and HCDR3 comprises the amino acid sequences of SEQ ID NOs:664-666, and the LCDR1, LCDR2, and LCDR3 comprises the amino acid sequences of SEQ ID NOs:668-670; or
(iv) the HCDR1, HCDR2, and HCDR3 comprises the amino acid sequences of SEQ ID NOs:672-674, and the LCDR1, LCDR2, and LCDR3 comprises the amino acid sequences of SEQ ID NOs:676-678; or
(v) the HCDR1, HCDR2, and HCDR3 comprises the amino acid sequences of SEQ ID NOs:680-682, and the LCDR1, LCDR2, and LCDR3 comprises the amino acid sequences of SEQ ID NOs:684-686.

In one embodiment, the binding domain that binds to NKG2D comprises a heavy chain variable region and a light chain variable region, said heavy chain variable region and light chain variable region comprise the amino acid sequences of SEQ ID NOs:645 and 649; SEQ ID NOs:655 and 659; SEQ ID NOs:663 and 667; SEQ ID NOs:671 and 675; SEQ ID NOs:653 and 654; or SEQ ID NOs:679 and 683.

In one embodiment, for any precursor tri-specific antibody constructs disclosed above, the second binding domain that binds to NKG2A comprises three complementarity determining regions (CDRs) on a heavy chain (HCDR1, HCDR2, and HCDR3) and three CDRs on a light chain (LCDR1, LCDR2, and LCDR3), wherein the HCDR1, HCDR2, and HCDR3 comprises the amino acid sequences of SEQ ID NOs:636-638, and the LCDR1, LCDR2, and LCDR3 comprises the amino acid sequences of SEQ ID NOs:640-642. In one embodiment, the second binding domain that binds to NKG2A comprises a heavy chain variable region and a light chain variable region, said heavy chain variable region and light chain variable region comprise the amino acid sequences of SEQ ID NOs:635 and 639; or SEQ ID NOs:643 and 644.

In one embodiment, the tri-specific antibody derived from the ProTribody construct disclosed herein comprises polypeptide A and polypeptide B, said polypeptide A and polypeptide B comprise amino acid sequences having the sequences of SEQ ID NOs: 180 and 177 (Tribody IM1062).

In one embodiment, the precursor tri-specific antibody constructs disclosed herein comprise a binding region having 2 scFv connected in tandem. Such constructs comprise polypeptide A and polypeptide B, said polypeptide A and polypeptide B comprise amino acid sequences having the sequences of SEQ ID NOs: 248 and 177; SEQ ID NOs: 249 and 177; SEQ ID NOs: 248 and 392; SEQ ID NOs: 249 and 392; SEQ ID NOs: 250 and 399; or SEQ ID NOs: 251 and 399. In another embodiment, the tri-specific antibodies derived from the ProTribody constructs disclosed herein comprise a binding region having 2 scFv connected in tandem. Such constructs comprise polypeptide A and polypeptide B, having the sequences of SEQ ID NOs: 246 and 177, or SEQ ID NOs: 247 and 358.

In one embodiment, the precursor tri-specific antibody constructs disclosed herein comprise a first binding domain binding to a TAA, a second binding domain comprises a cytokine receptor engager, and a third binding domain binding to T cell surface antigen CD3. Examples of TAA include, but are not limited to, 5T4, ROR1, EGFR, FcγRI, FcγRIIa FcγRIIb FcγRIIIa FcγRIIIb, CD28, CD137, CTLA-4, FAS, fibroblast growth factor receptor 1 (FGFR1), FGFR2, FGFR3, FGFR4, glucocorticoid-induced TNFR-related (GITR) protein, lymphotoxin-beta receptor (LTβR), toll-like receptors (TLR), tumor necrosis factor-related apoptosis-inducing ligand-receptor 1 (TRAIL receptor 1), TRAIL receptor 2, prostate-specific membrane antigen (PSMA) protein, prostate stem cell antigen (PSCA) protein, tumor-associated protein carbonic anhydrase IX (CAIX), epidermal growth factor receptor 1 (EGFR1), EGFRvIII, human epidermal growth factor receptor 2 (Her2/neu; Erb2), ErbB3 (HER3), Folate receptor, ephrin receptors, PDGFRa, ErbB-2, CD20, CD22, CD30, CD33, CD40, CD37, CD38, CD70, CD74, CD56, CD40), CD80, CD86, CD2, p53, cMet (tyrosine-protein kinase Met) (hepatocyte growth factor receptor) (HGFR), MAGE-A1, MAGE-A2, MAGE-A3, MAGE-A4, MAGE-A6, MAGE-A10, MAGE-A12, BAGE, DAM-6, DAM -10, GAGE-1, GAGE-2, GAGE-8, GAGE-3, GAGE -4, GAGE-5, GAGE-6, GAGE-7B, NA88-A, NY-ESO-1, BRCA1, BRCA2, MART-1, MC1R, Gp100, PSA, PSM, Tyrosinase, Wilms’ tumor antigen (WT1), TRP-1, TRP-2, ART-4, CAMEL, Cyp-B, hTERT, hTRT, iCE, MUC1, MUC2, P-cadherin, Myostatin (GDF8), Cripto (TDGF1), MUC5AC, PRAME, P15, RU1, RU2, SART-1, SART-3, WT1, AFP, β-catenin/m, Caspase-8/m, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, ETV6/AML, LDLR/FUT, Pml/RARα, TEL/AML1, CD28, CD137, CanAg, Mesothelin, DR5, PD-1, PD1L, IGF-1R, CXCR4, Neuropilin 1, Glypicans, EphA2, CD138, B7-H3, B7-H4, gpA33, GPC3, SSTR2, or VEGF-R2

In one embodiment, examples of NK cell surface antigens recognized by the precursor tri-specific antibody constructs disclosed herein include, but are not limited to, NKG2A, NKG2D, CD16, NKp46, CD16a (FcγRIIIa), CD56, sMICA/B, ILT, SLAMF7, NKp44, NKp30, DNAM-1, NKG2C/CD94, KIR2/DL3, KIR2DL1, NKRP1, NKG2E/CD94, NKG2F/CD94, CD69, LLT1, ILT2, AICL, CD26, NKp80, KIR family receptors, or CD122/IL-2Rbeta.

In one embodiment, the precursor tri-specific antibody constructs disclosed herein comprise a cytokine receptor engager such as IL-15, IL-2, IL-12, TNF-alpha, IL-6, TGF-beta, IL-10, IL-8, IL-17, IL-21, INF, or VEGF.

In one embodiment, for any precursor tri-specific antibody constructs disclosed above, the third binding domain comprises a Fab region comprising a heavy chain polypeptide and a light chain polypeptide, said heavy chain polypeptide comprises a heavy chain variable region and a heavy chain constant region (VH-CH), said light chain polypeptide comprises a light chain variable region and a light chain constant region (VL-CL), wherein when said first binding domain is located C-terminally to said VL-CL region, said second binding domain is located C-terminally to said VH-CH region, or when said first binding domain is located C-terminally to said VH-CH region, said second binding domain is located C-terminally to said VL-CL region. In some embodiments for the precursor tri-specific antibody constructs disclosed above, a single regulatory domain comprising a protease cleavage domain, a half-life prolonging (HLP) domain, and a CAP component is located N-terminally to said VH region or to said VL region of said third binding domain.

In one embodiment, the present disclosure provides a pharmaceutical composition comprising a precursor tri-specific antibody construct disclosed herein, and a pharmaceutically acceptable carrier.

In one embodiment, the present disclosure provides a nucleic acid construct comprising one or more nucleic acid sequences, said sequences encoding a precursor tri-specific antibody construct disclosed herein. In one embodiment, the present disclosure provides an expression vector comprising such nucleic acid construct.

In one embodiment, the present disclosure provides a method of treating, preventing, or delaying disease progression, reducing tumor load, or reducing the incidence of a cancer or a tumor, or any combination thereof, in a subject in need of such treatment, comprising a step of administering to the subject a pharmaceutical composition comprising the precursor tri-specific antibody constructs disclosed herein. In one embodiment, the cancer or tumor comprises a solid tumor or non-solid tumor, or the cancer or tumor comprises a metastasis of a cancer or tumor.

While certain features of the precursor tribody constructs have been illustrated and described herein, many modifications, substitutions, changes, and equivalents will now occur to those of ordinary skill in the art. It is, therefore, to be understood that the appended claims are intended to cover all such modifications and changes as fall within the true spirit of these precursor constructs.

### EXAMPLE 1

### Expression and Purification of Tribody and ProTribody Antibody Constructs

Objective: To express and purify a cleaved precursor trispecific antibody, a non-cleaved precursor trispecific antibody, and a trispecific antibody construct.

Methods: Gene Synthesis And Plasmid Construction. The coding sequences for the heavy chain (HC) and light chain (LC) of the precursor bispecific antibody were generated by DNA synthesis and PCR, subsequently subcloned into pCDNA3.4-based plasmid (Invitrogen) for protein expression in mammalian cell system. Finally, the gene sequences in the expression vectors were confirmed by DNA sequencing.

Expression of Trispecific Antibody Construct. Transient expression of the Tribody/ProTribody antibodies was performed by co-transfection of paired HC and LC constructs (at 1:1 HC/LC ratio for the Tribody format or 2.5:1 HC/LC ratio for the ProTribody format) into CHO cells using PEI method. Briefly, 1 L of CHO cells at approximately 5.5×10⁶/ml in a 3 L shake flask was used as the host, Transfection was initiated by adding a mixture of 1 mg of total DNA and 4 mg PEI in 100 ml OptiMEM medium (Invitrogen) to the cells and gentle mixing. Cells were then cultured in an incubator shaker at 120 rpm, 37° C., and 8% CO2, for 8-10 days. Feeding with peptone and glucose was carried out 24 h later and every 2-3 days thereafter depending on the cell density and viability. The cell culture was terminated on day 8-10 when cell viability reduced to <80%. The conditioned medium was harvested for protein purification.

Purification of Trispecific Antibody Construct. Protein purification by affinity chromatography and SEC was performed using an AKTA pure instrument (GE Lifesciences). Affinity capture of the tribody was achieved by passing the harvested supernatants over a column of CaptureSelect™ CH1-XL Affinity Matrix (Thermo Scientific). After washing column with Buffer A (25 mM Tris, 150 mM NaCl, 5 mM EDTA, pH 7.5), the protein was eluted with Buffer B (50 mM Sodium citrate, 150 mM NaCl, pH 3.0), and immediately neutralized with ⅙ volume of Buffer D (1 M Aarginine, 400 mM Succinic acid, pH 9.0). The affinity purified protein was then concentrated to 5-10 mg/ml using Amicon 30 kD concentrator (Merck Millipore) and subjected to SEC purification on a Superdex200 column (GE Lifesciences) equilibrated with SEC Buffer: 200 mM Arginine, 137 mM Succinic acid, 0.05%Tween-80,150 mM NaCl, pH5.0. The target tribody fractions were collected, then added 5% trehalose (146 mM). The target tribody will be analysized using SDS-PAGE and HPLC-SEC.

SDS-PAGE Analysis of Trispecific Antibody Construct. SDS-PAGE analysis of tribody was carried out under reducing and non-reducing conditions in pre-cast polyacrylamide gels. Briefly, 2 ug tribody samples were mixed by NuPAGE™ LDS sample buffer (thermofisher-NP0008) with 70 mM DTT add or not. After incubating at 25° C. or 90° C. for 10 min, the samples and Unstained Protein Standards (BIO RAD-161-0363 were loaded onto the gels. Electrophoresis was carried out at a constant voltage of 120 V with 1× Tris-glycine-SDS running buffer. Following electrophoresis, gels were stained for overnight using Coomassie blue and de-stained with destaining solution (10% acetic acid, 40% methanol and 50% water). Destained gels were scanned with a Gel imaging system (Tanon-2500R).

SEC-HPLC Analysis of Precursor Trispecific Antibody Construct. Analytical SEC-HPLC was performed using Shimadzu LC-10 HPLC instrument (Shimadzu Corp.). 20µl sample on 1 mg/ml will be loaded to a Superdex 200 Increase 5/150 GL column (GE Lifesciences). The mobile phase was 2*PBS with a flow rate of 0.3 ml/min, 15 min.

LC-MS Analysis of Tribody Construct. The Tribody was separated with ACQUITY UPLC BEH200 Å, SEC column (Waters 1.7 µm, 4.6x 300 mm) at room temperature and detected by ESI-MS(Thermo, MS-B20-03). The mobile phase was 0.1% formic acid: acetonitrile (75:25, v/v) with a flow rate of 0.2 mL/min. Mass spectrometry was performed in the positive ion. Other parameters for mass spectrometry were: resolution of 17500, Scan range of 1000-5000 m/z, In-source CID of 60 eV, sheath gas flow rate of 30 L/min, capillary temperature of 350° C., Spray voltage of 2.5 KV.

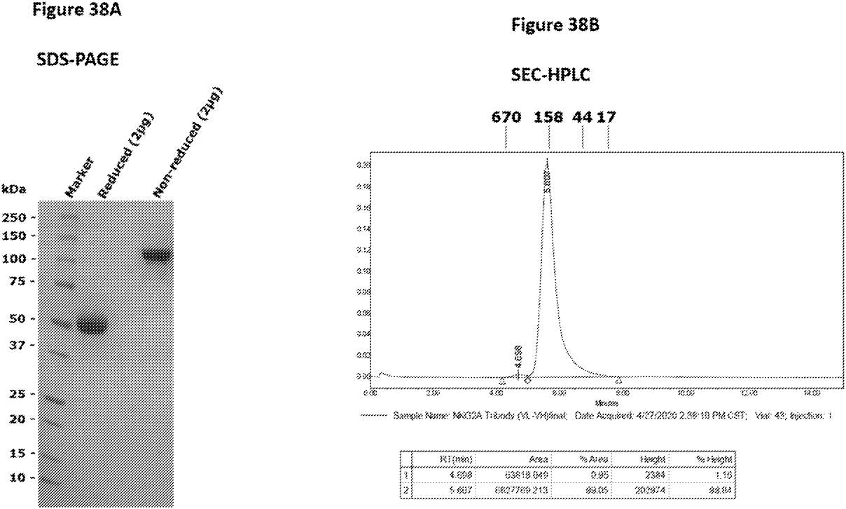

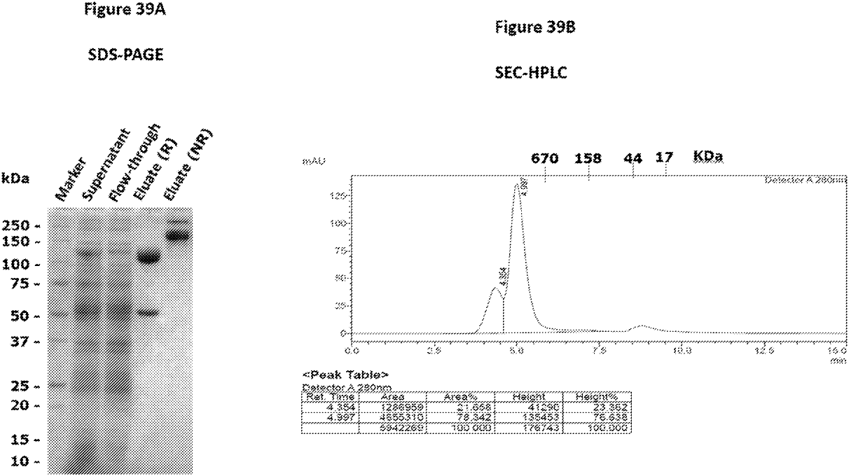

Results: The expressed HC and LC Tribody constructs associate to form a single molecule, as indicated by the single ~100 kDA band observed in the SDS-PAGE, and by a single major peak at retention time of ~5.6 min in SEC-HPLC (FIGS. 38A and 38B, respectively). The expressed HC and LC of the ProTribody cleaved/non-cleaved trispecific antibody construct produced a ~180 kDA band in SDS-PAGE, and a major peak at retention time of ~4.9 min in SEC-HPLC (FIGS. 39A and 39B, respectively). These results are in agreement with the expected retention time of the expected Mw based on mass calibration curve.

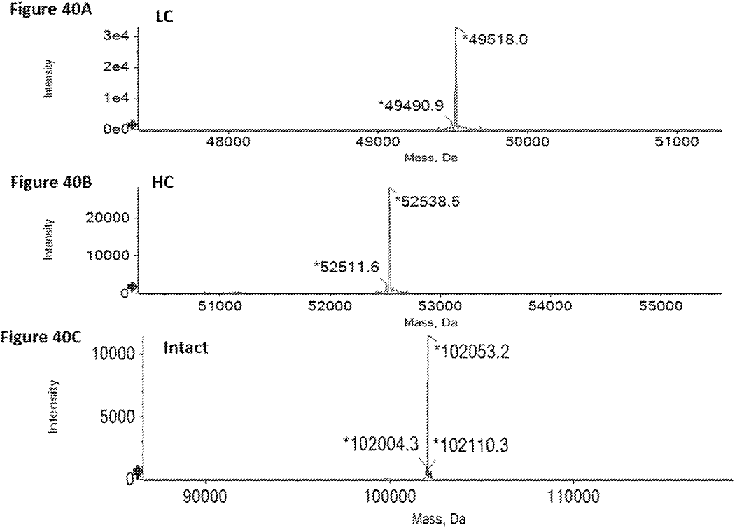

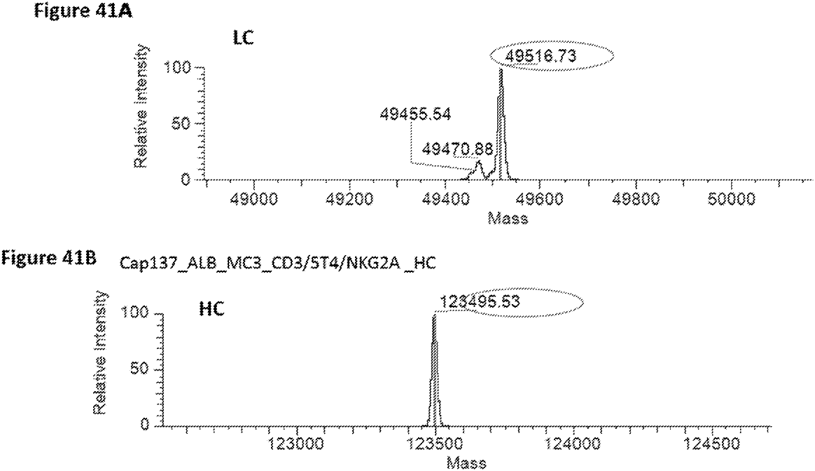

MS analysis of the Tribody constructs confirmed a 49.5 kDa pick for the LC, a 52 kD pick for the HC and 102 kDa for the intact protein (FIGS. 40A-C, respectively). MS analysis of the ProTribody constructs confirmed a 49.5 kDa pick for the LC and a 123.5 kD pick for the HC (FIGS. 41A-B, respectively).

Conclusion: a Trispecific Tribody and ProTribody format constructs can be successfully expressed and purified.

### EXAMPLE 2

### Protease Cleavage Assay of ProTribody Products

Objective: To validate in vitro the cleavage of the purified ProTribody formats by specific proteases.

Methods: Conversion of the ProTribody variants to the active activated formats was performed by recombinant Human proteases (R&D Systems). Briefly, The ProTribody with multiple cleavage sites MC2 and MC3 (MMP-9 and uPA/ MMP-9, Matriptase and uPA respectively) as well as the non-cleavable (NC) format were digested by different protease separately on R.T. overnight. The MMP-9 cleavage assay were carried out in 50 mM Tris,10 mM CaCl₂, 150 mM NaCl, 0.05%(w/v) Brij-35,pH7.5, and the mass ratio of MMP-9 to tribody is 100:1. For the uPA cleavage assay, the assay buffer is 50 mM Tris, 0.01%(v/v)Tween20,pH8.5, and the mass ratio of uPA to tribody is 100:1. The assay buffer for Matriptase cleavage assay is 50 mM Tris, 50 mM NaCl, 0.01%(v/v)Tween20, pH9.0 and the mass ratio of Matriptase to tribody is 1000:1. The cleavage products were detected by SDS-PAGE.

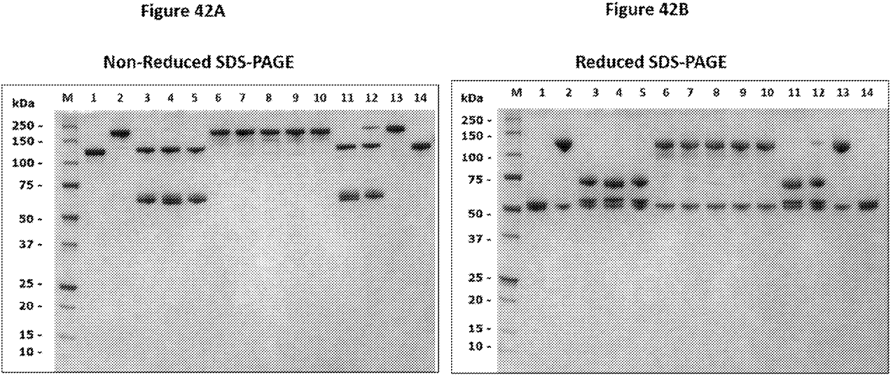

Results: The cleavable ProTribody variants (IM-1188, IM-1184) as well as the non-cleavable Protribody format (IM-1193) were treated with the respective proteases and 1ug was loaded on 4-20% SDS-PAGE. In non-reduced conditions (FIG. 42A), IM-1188 Matriptase treated, MMP-9 treated and uPA treated (FIG. 42A, lanes 3,4,5, respectively) revealed the cleaved formats at the expected bands size of ~130 kDa +50 kDa, while in the untreated (lane2 FIG. 42A) an expected ~180 kDa is observed. Similarly, IM-1184 MMP-9 treated and uPA treated (FIG. 42A, lanes 11,12, respectively) indicate for the cleaved formats at the expected bands size of ~130 kDa +50 kDa while in the untreated (lane13 FIG. 42A) an expected band of ~180 kDa is observed. IM-1193 untreated, Matriptase treated, MMP-9 treated and uPA treated (FIG. 42A, lanes 6,7,8,9, respectively) revealed an expected band of ~180 kDa, indicating for uncleavable product.

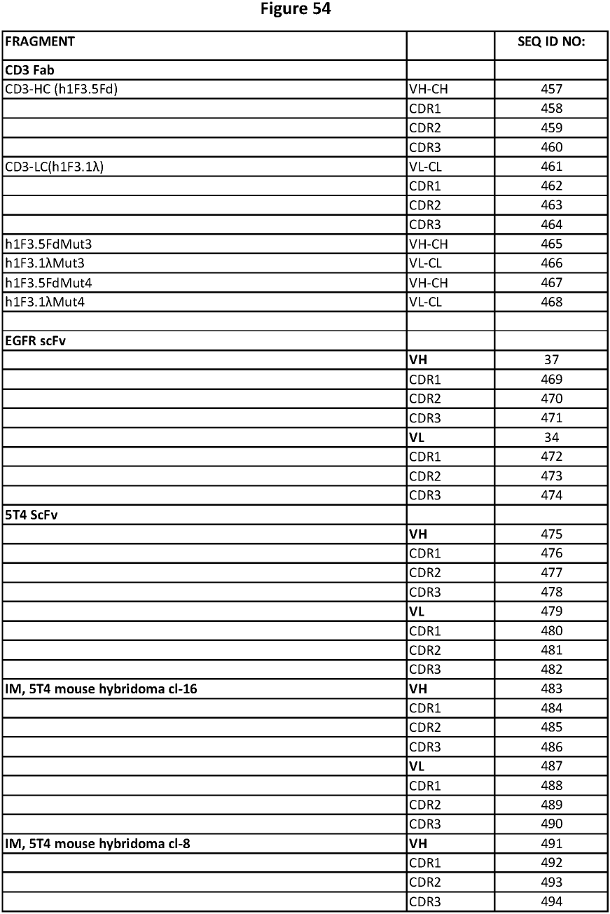

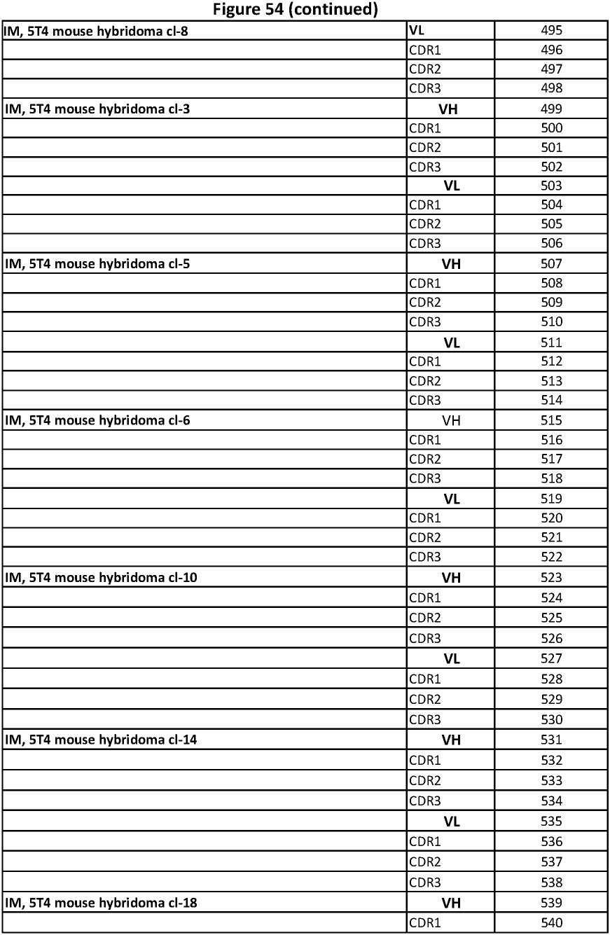

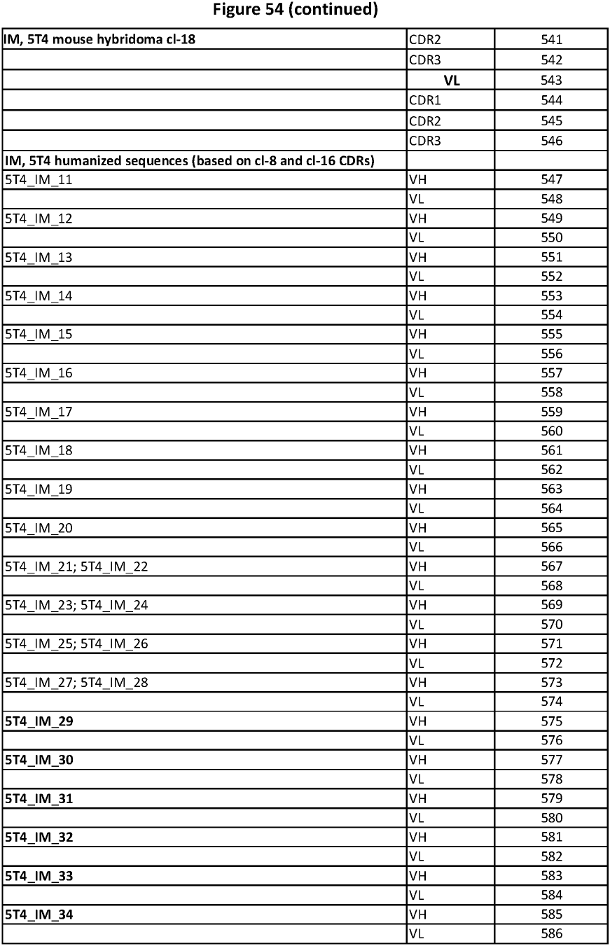

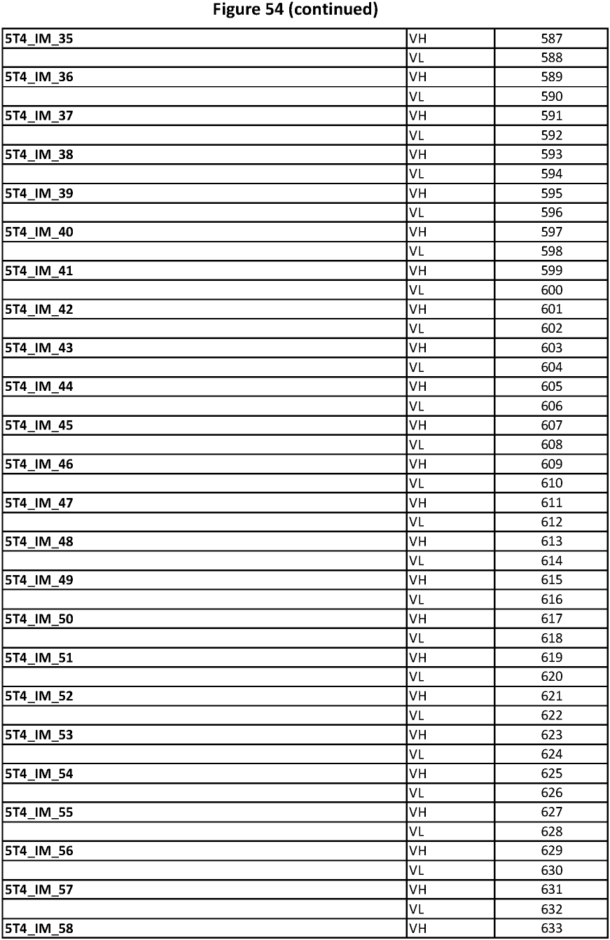

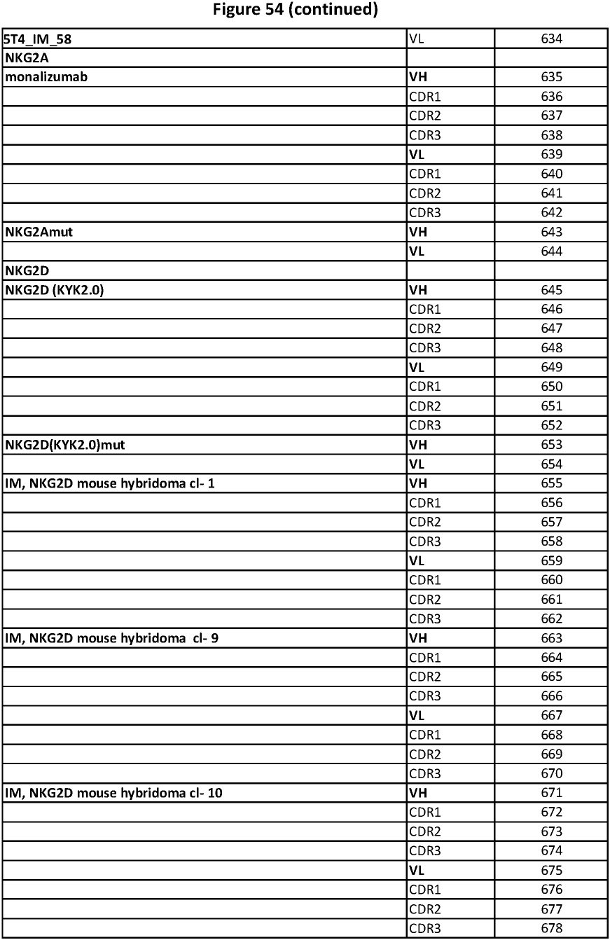

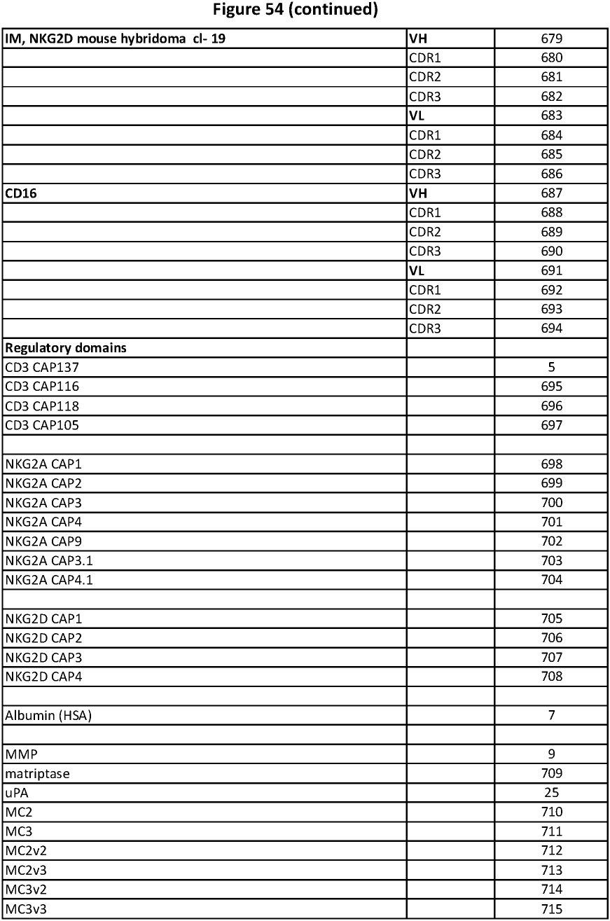

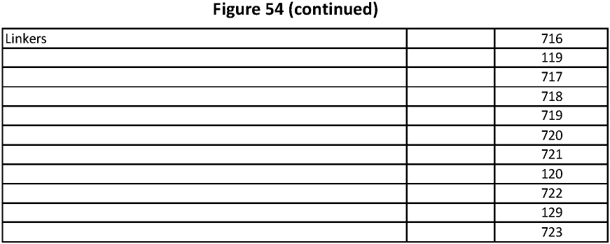

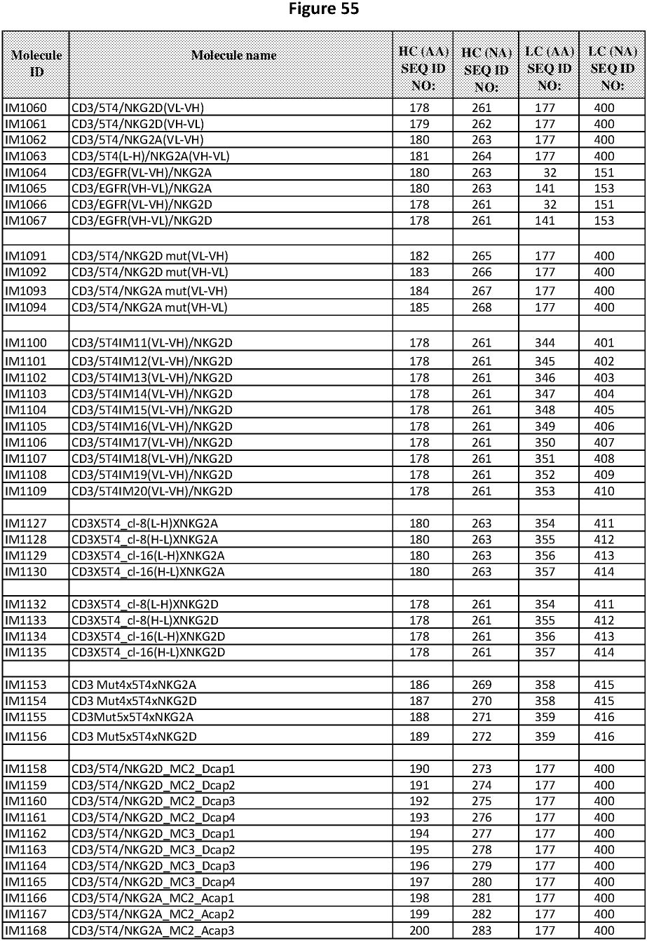

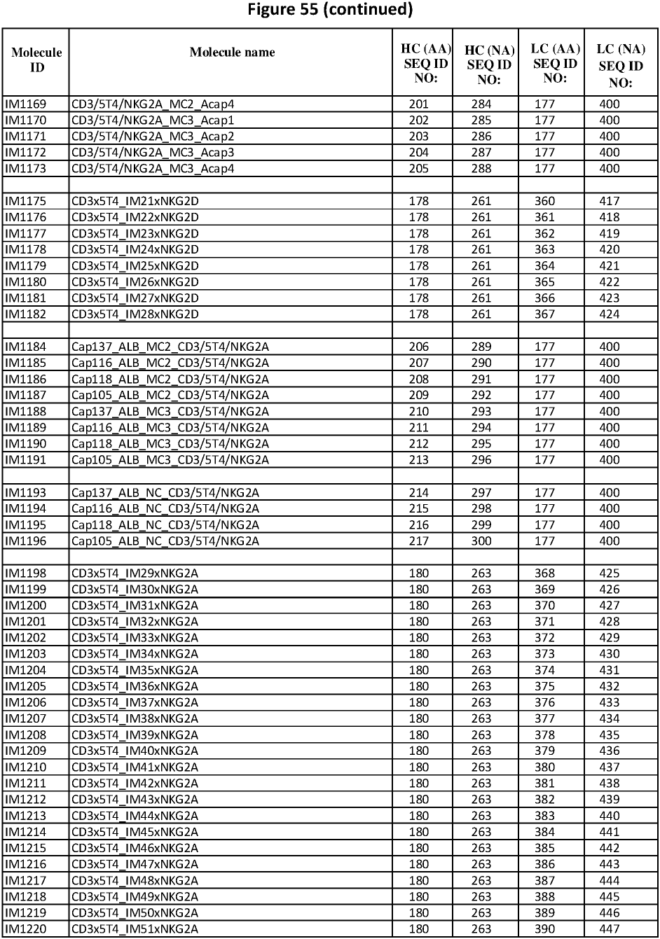

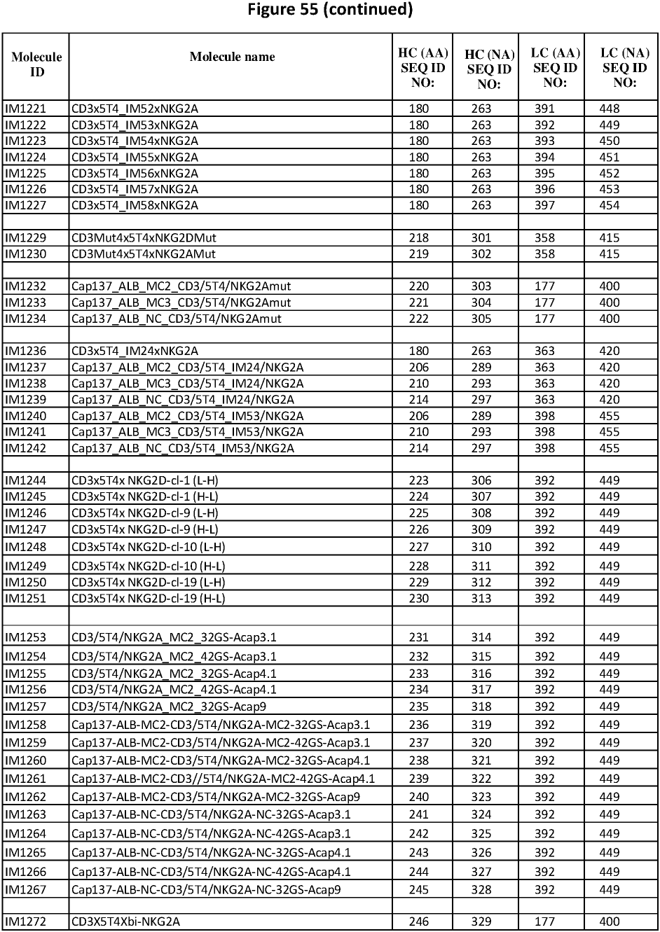

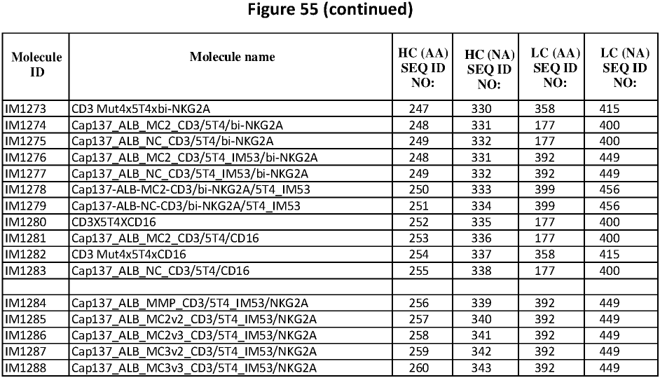

In reduced conditions (FIG. 42B) IM-1188 Matriptase treated, MMP-9 treated and uPA treated (FIG. 54 lanes 3,4,5, respectively) revealed the cleaved formats at the expected bands size of ~70 kDa+50+60 kDa, while in the untreated (lane2 FIG. 42B) expected bands of -50+130 kDa are observed. Similarly, IM-1184 MMP-9 treated and uPA treated (FIG. 42B, lanes 11,12, respectively) indicate for the cleaved formats at the expected bands size of ~70 kDa+50+60 kDa, while in the untreated (lane13 FIG. 42B) expected bands of ~50 kDa+130 kDa are observed. IM-1193 untreated, Matriptase treated, MMP-9 treated and uPA treated (FIG. 42B, lanes 6,7,8,9, respectively) revealed the expected bands of ~50 kDa+130 kDa.

Conclusion: Cleavable formats can be successfully cleaved in vitro by the respective proteases, and a non-cleavable format is not cleaved by the same proteases.

### EXAMPLE 3

### Binding of Tribody and ProTribody Antibody Constructs to Recombinant Proteins by ELISA

Objective: To study the binding efficacy of a variety of Tribody/ProTribody antibody constructs that binds by the TAA ScFv domain (5T4), by the Tcell engager domain (anti CD3ε Fab), and by the NK engager domain (anti NKG2A/ anti NKG2D) to 5T4, CD3ε and NKG2A recombinant proteins, respectively by ELISA. The various formats may be comprised of a CAP masking sequences, a cleavable linker, a non-cleavable linker, as well as a point-mutated engager sequences that are lack of binding activity to the specific engager and serve as negative controls for the Tribody/Protribody formats.

Methods: ELISA binding of Tribody/ProTribody antibody constructs to antigens: Dilute target protein (hCD3epsilon-His (cat # 10977-H08S, supplier Sino Biological); h5T4-His (cat# 19845-H08H, supplier Sino Biological); NKG2A-CD94-ECD-hFc, Lot:20200724002, provided by CP; or NKG2D-ECD-hFc, Lot: 20200413002, provided by CP) into PBS with final concentration of 0.01 µg/mL for hCD3epsilon-His, 0.3 µg/mL for h5T4-His, 1 µg/mL for NKG2A-CD94-ECD-hFc and 0.7 µg/mL for hNKG2D-ECD-hFc respectively and coat 100 µL/well on ELISA plate (cat: 9018, supplier Corning) respectively. Incubate O/N, 4° C. The plates were blocked with 250 µL 1% BSA in PBST for 1 hr at 37° C.Wash 4 times with PBST. All the washes are done using Biotek (Elx 405). All the Tribody set antibodies were diluted to 400 nM and make 4-fold serially dilutions (12 points, including 0 point). Add 100 µL/well diluted antibody constructs solution to plate, incubate for 1 hr at 37° C.Wash 4 times with PBST. Add 100 µL/well anti-human kappa light chain-HRP (1:10000), incubate for 0.5 hr at 37° C. Wash 4 times with PBST. 100 µL/well of TMB substrate was added and incubated at room temperature for 5 min. 100 µL/well of 1N HCl to terminate reaction. Plates were read using ELISA plate reader at 450 nm wavelength (instrunet SpectraMax M5e). Data Analysis was performed using Graphpad prism 5 software by using nonlinear regression (curve fit): log (agonist) vs. response, agonist is antibody concentration (nM) and response is OD value.

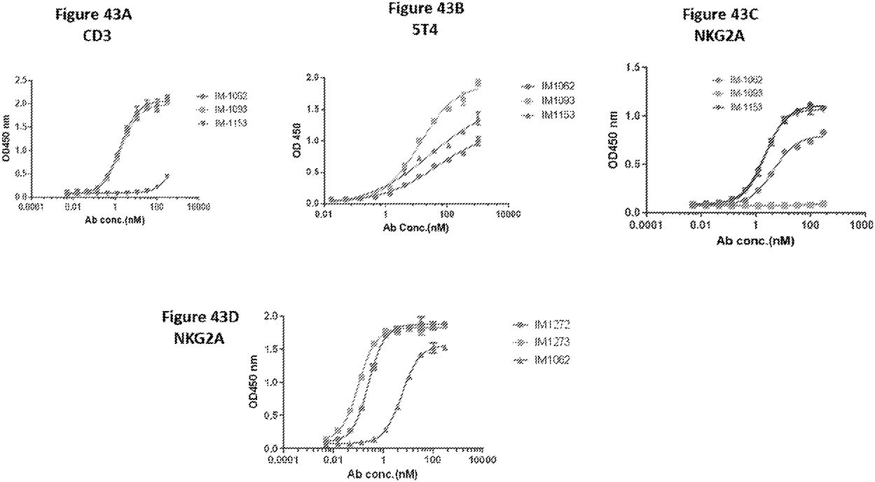

Results: The expressed trispecific constructs were analyzed for their binding to CD3ε, to 5T4 and to NKG2A/NKG2D. The binding EC50 to human CD3ε was of 1.69 nM for the IM-1062 (circles) and 1.84 nM for IM-1093 NKG2A mutant variant (squares), while no binding observed for IM-1153 CD3 mutant variant (triangles), as expected (FIG. 43A). The EC50 to human 5T4 was of 45 nM for the IM-1062 (circles), 12 nM for IM-1093 NKG2A mutant variant (squares), and 31 nM for IM-1153 CD3 mutant variant (triangles) (FIG. 43B). The EC50 to human NKG2A was of 4.4 nM for the IM-1062 (circles) and 2.1 nM for IM-1153 CD3 mutant variant (squares), while no binding observed for IM-1093 NKG2A mutant variant(triangles), as expected (FIG. 43C). The EC50 to human NKG2A tested with the bi-NKG2A scFv Tribodies variants that harbors two anti NKG2A scFv in tandem, was of 0.2 nM for the IM-1272 (circles) and 0.1 nM for IM-1273 CD3 mutant variant (squares), while 5.7 nM was observed for IM-1062 (triangles), suggesting ~60-fold improved binding affinities for the double-ScFv NKG2A arm. (FIG. 43D).

Conclusion: As shown in FIGS. 43A-D, the binding of the Tribody format was confirmed in each of the Tribody arms. The Tribody format that harbors point mutations in the NKG2A arm (IM-1093) lack binding to the NKG2A antigen as expected, while maintaining the binding of the other structural arms to the respective proteins (5T4 and CD3ε). Similar data is observed on the NKG2D mutant variant (IM-1091, data not shown). The Tribody format that harbors point mutations in the CD3ε CDR domain arm (IM-1153) lack binding to the CD3ε antigen protein as expected, while maintaining the binding of the other arms to the respective proteins (5T4 and NKG2A). Similar data is observed on additional version of CD3ε mutant variant where other mutations were introduced into the CDRs, which lacks biding (IM-1155, data not shown). In addition, the Tribody format that harbors two anti NKG2A scFv in tandem exhibited ~60-fold higher binding affinities to NKG2A than the single scFv.

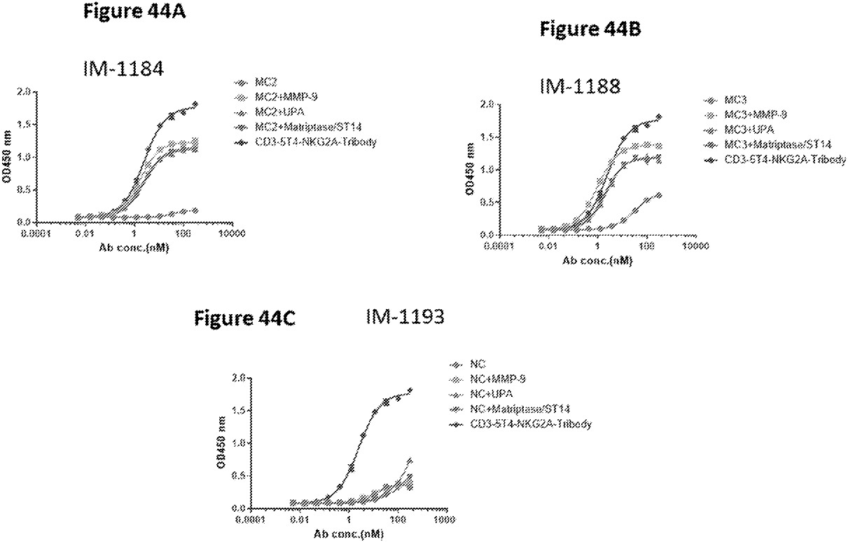

Results: EC50 of 1.7 nM for the MMP-9 cleaved IM-1184 construct (squares) and 2.2 nM for the uPA cleaved IM-1184 construct (triangles up) binding to recombinant hCD3ε protein were observed, while no binding of the un-cleaved IM-1184 construct (circles) as expected (FIG. 44A). EC50 of 0.9 nM for the MMP-9 cleaved IM-1188 construct (squares), 2.1 nM for the uPA cleaved IM-1188 construct (triangles up) and 1.9 nM for the Matriptase cleaved IM-1188 construct (triangles down) binding to recombinant hCD3ε protein were observed, while no binding of the un-cleaved IM-1188 construct (circles) as expected (FIG. 44B). Almost no binding was detected for the non-cleavable ProTribody format (IM-1193) as expected (FIG. 44C).

As shown in FIGS. 44A-C, binding to human CD3ε is influenced by the presence or absence of a CAP and serum half-life prolonging (HLP) moiety elements. Absence of a CAP in the Tribody construct (IM-1062) or in the cleaved ProTribody results in increased binding affinity compared to the either non-cleaved ProTribody constructs or the non-cleavable construct (IM-1193). The results demonstrate regulatory binding of ProTribody constructs (IM-1184, IM-1188 and IM-1193) to human CD3ε, wherein the incubation of cleavable ProTribody constructs (IM-1184 and IM-1188) with each of the proteases leads to increased affinity of the CAP-removed structure with the CD3ε antigen . A similar increase in affinity was not observed in the construct having a non-cleavable linker (ProTribody-NC, IM-1193) incubated with the same proteases.

The Tribody construct and the cleaved ProTribody constructs have a relatively similar binding affinity to CD3ε by ELISA, while un-cleaved ProTribody constructs as well as non-cleavable Protribody format in the presence of proteases were shown dramatically reduced binding to CD3e.

Conclusion: ProTriBody variants having the CAP-HLP masking fused lack binding to CD3e, while constructs lacking a CAP element showed increased binding. Binding affinity to CD3ε of ProTribody constructs could be regulated by incubation with proteases, wherein incubation of a cleavable ProTribody construct in the presence of protease result with a significant increase in binding affinity of the construct. Protease-cleaved ProTribody has restored CD3ε binding.

### EXAMPLE 4

### Binding of Trispecific Antibody Constructs to Cells

Objective: To study the binding efficacy of a variety of Tribody/ProTribody antibody constructs that binds by the TAA ScFv domain (5T4), by the Tcell engager domain (anti CD3ε Fab), and by the NK engager domain (anti NKG2A/anti NKG2D) to cells expressing membrane bound endogenous 5T4, CD3ε and NKG2A/NKG2D proteins, respectively, as well as ectopic expression in CHO cells over expressing 5T4, CD3ε and NKG2A/NKG2D proteins, respectively, by FACS. Specifically, to study the binding efficacy of Tribody (IM-1062, IM-1093 and IM-1153), to Jurkat T-cell line (CD3ε), NCI-H226 (5T4), NK92 cell line (NKG2A) and to CHO cells over expressing either 5T4 or NKG2A proteins. In addition to study the binding efficacy of the ProTribody (cleavable and non-cleavable formats (IM-1184/IM-1188 and IM-1193, respectively) to Jurkat Tcell line (CD3ε). The various formats may be comprised of a CAP masking sequences, a cleavable linker, a non-cleavable linker, as well as a point-mutated engager sequences that are lack of binding activity to the specific engager and serve as negative controls for the Tribody/Protribody formats.

Methods: FACS binding of Tribody/ProTribody antibody constructs to cells

Suspension cultured cells was harvested directly, and adherent cell were digested using TrypLE Express Enzyme (cat: 12604-013, supplier Life technologies). Centrifuge at 1000 rpm for 5 min and discard the supernatant. Cells are suspended at a concentration of 2×10⁶/mL in FACS buffer (2% FBS in PBS) and add 100 µL/well of cell suspension to the plate (cat #3799, supplier Corning). Centrifuging the plates at 2000 rpm for 5 min and discard the supernatant. Re-suspend the cells in 100 µL/well of Tribody set antibodies (400 nM start, 4-fold dilution, 8 point including 0 point) and incubate the plate for 60 min at 4° C. Centrifuge the plate at 2000 rpm, 4° C. for 5 min and discard supernatant. Then wash the cells 3 times with 170 µL FACS buffer. Re-suspend the cells at 100 µL/well with secondary antibody (goat anti-human Ig Fab-FITC, Cat # 2085-02, Southern biotech) with 1:400 dilution and incubate the plate for 30 min at 4° C. in dark. Centrifuge the plate at 2000 rpm, 4° C. for 5 min and discard supernatant. Then wash the cells 3 times with FACS buffer and analyze the sample with FACS verse. The fluorescence intensity of the staining was measured using flow cytometer (BD, FACSVerse). The geometric mean fluorescence intensity (GMFI; median fluorescence intensity (MFI)) of set antibodies staining was calculated (BD FACSuite software). Dose-response curves were generated and EC50s for thetrispecific variants binding were calculated using GraphPad Prism software.

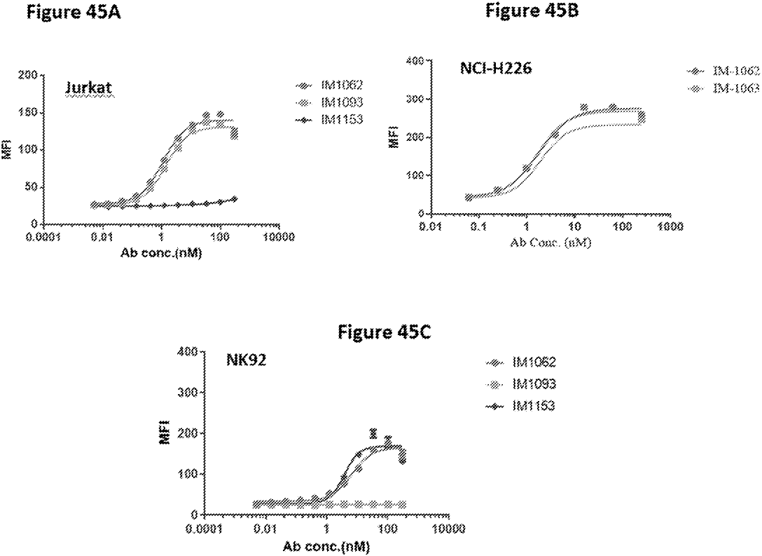

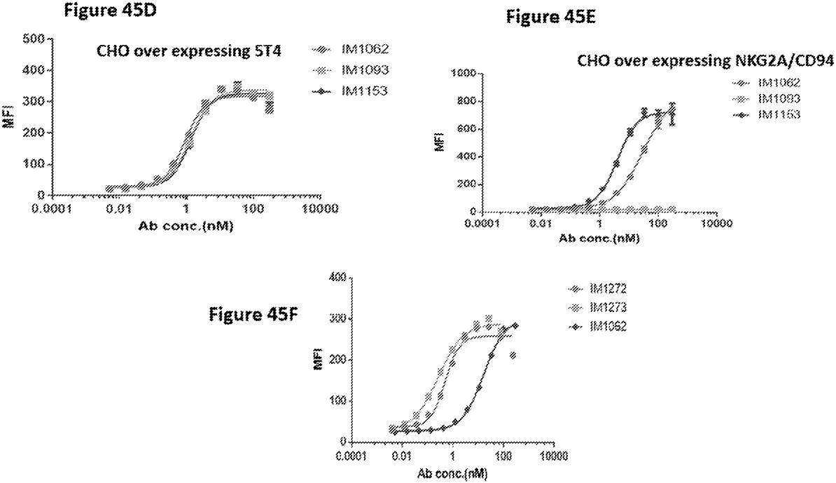

Results: The expressed trispecific constructs were analyzed for their binding to cells expressing endogenous CD3ε, 5T4 and NKG2A/NKG2D. The binding EC50 to Jurkat Tcells expressing endogenous hCD3ε was 1.1 nM for the IM-1062 (circles) and 1.3 nM for IM-1093 NKG2A mutant variant (squares), while no binding observed for IM-1153 CD3 mutant variant (rhombus), as expected (FIG. 45A). The EC50 to NCI-H226 cell line expressing endogenous 5T4 was of 1.78 nM for the IM-1062 (circles), and 1.64 nM for IM-1063 variant (squares) (FIG. 45B). The EC50 to NK92 cell line expressing endogenous NKG2A was 6.5 nM for the IM-1062 (circle) and 3.7 nM for IM-1153 CD3 mutant variant (rhombus), while no binding observed for IM-1093 NKG2A mutant variant (squares), as expected (FIG. 45C). The EC50 to CHO cell line over expressing ectopic 5T4 was of 0.9 nM for the IM-1062 (circles), and 1.3 nM for IM-1093 variant (squares) and 1.33 nM for the IM-1153 CD3 mutant variant (rhombus) (FIG. 45D). The EC50 to CHO cell line over expressing ectopic NKG2A was 29 nM for the IM-1062 (circle) and 3 nM for IM-1153 CD3 mutant variant (rhombus), while no binding observed for IM-1093 NKG2A mutant variant (squares), as expected (FIG. 45E). The EC50 to CHO cells over expressing human NKG2A tested with the bi-NKG2A scFv Tribody variants that harbor two anti NKG2A scFv in tandem, was of 0.4 nM for the IM-1272 (circles) and 0.2 nM for IM-1273 CD3 mutant variant (squares), while 16 nM was observed for IM-1062 (rhombus), suggesting ~80-fold improved binding affinities for the double-ScFv NKG2A arm. (FIG. 45F).

Conclusion: As shown in FIGS. 45A-F, the binding of the Tribody format to cell membrane bound form was confirmed in each of the Tribody arms. The Tribody format that harbors point mutations in the NKG2A arm (IM-1093) lack binding to the NKG2A-expressing cells as expected, while maintaining the binding of the other arms to the respective expressing receptors (5T4 and CD3ε). Similar data is observed on the NKG2D mutant variant (IM-1091, data not shown). The Tribody format that harbors point mutations in the CD3ε arm (IM-1153) lack binding to the CD3ε expressing cells as expected, while maintaining the binding of the other arms to the respective expressing receptors (5T4 and NKG2A). Similar data is observed on additional version of CD3ε mutant variant (IM-1155, data not shown). In addition, the Tribody format that harbors two scFv anti NKG2A in tandem exhibited higher binding affinities to cells expressing NKG2A than the single scFv.

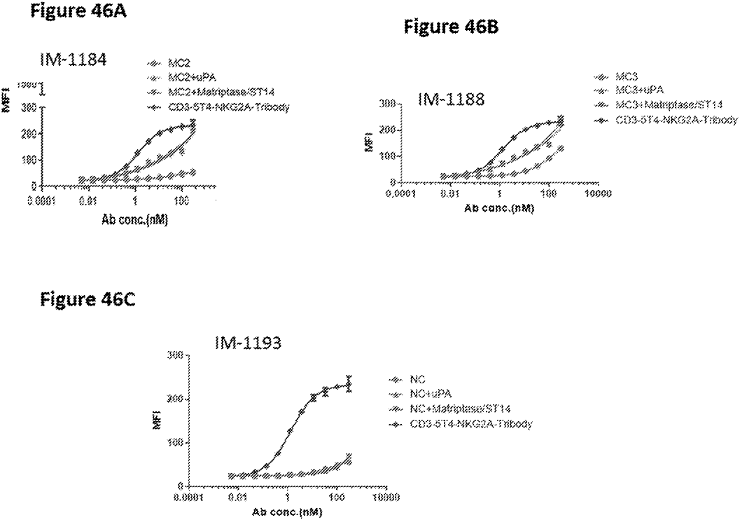

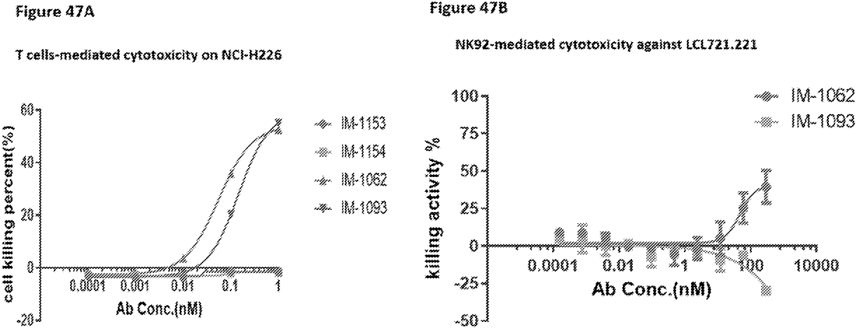

Results: Binding of the uPA cleaved IM-1184 construct (triangles up) to Jurkat cells was observed, with lower efficacy than the IM-1062 Tribody, while no binding of the un-cleaved IM-1184 construct (circles) as expected (FIG. 46A). Similarly, binding of the uPA cleaved IM-1188 construct (triangles up) and the Matriptase cleaved IM-1188 construct (triangles down) to Jurkat cells was observed, while lower binding of the un-cleaved IM-1188 construct (circles) as expected (FIG. 46B). Almost no binding is detected for the non-cleavable ProTribody format (IM-1193) as expected (FIG. 46C).

Conclusion: The ProTribody formats were analyzed for their ability to bind to tumor cells expressing CD3 epsilon (Jurkat T cells). The cleaved cleavable ProTribody formats had significantly increased affinity to Jurkat cells as compared to the non-cleaved ProTriboby formats. Constructs lacking a CAP-HLP element showed increased binding. Binding affinity to CD3ε of ProTribody constructs could be regulated by incubation with proteases, wherein incubation of a cleavable ProTribody construct in the presence of protease result with significant increased binding affinity of the construct. Protease-cleaved ProTribody has restored CD3ε binding.

### EXAMPLE 5

### In Vitro Functional Evaluation of Tribody and ProTribody Antibody Constructs

Objective: To evaluate in vitro, dose dependent T-cell/PBMCs mediated cytotoxicity of Tribody/ProTribody variants of colon, breast, lung cancer cells (HCT116, MDA-MB-231, NCI-H226 and A549, respectively).

Methods: Lactate Dehydrogenase (LDH) Cytotoxicity Assay: Tribody and ProTribody variants were analyzed for their potential to induce Tcells/PBMCs cell-mediated cytotoxicity in 5T4 expressing cancer cells. Briefly, Isolate T cell using EasySep Human T Cell Isolation Kit (STEMCELL, Cat: 17951). Adjust concentration of target cell to 2×10⁵/mL in assay buffer (blank RPMI 1640, Gibco, Cat-10491 plus 5%FBS), and add 50 µL to wells of a round-bottom 96-well plate (Cat-3799, Corning). Adjust concentration of effect cell (Isolated T cells or PBMCs from ALLCELLS) to 2E6/mL in assay buffer, and add 50 µL to wells with ET ratio of 10:1. Then add 100 µL/well of 2-fold diluted antibodies, mix sufficiently. Incubate at 37° C., 5% CO₂ for 24 hr. Centrifuge the plate at 300 g for 5 min and collect supernatant. LDH release would be tested by CytoTox 96 Non-radioactive cytotoxicity assay kit (Promega, G1780). Add 20 µL lysis solution (10*) to max well, mix sufficiently and incubate at 37° C. for 45 min. Then transfer 50 µL aliquots from all test and control wells to a fresh 96-well flat clear bottom plate (Cat-3599, Corning). Add 50 µL CytoTox reagent to each well. Protect plates from light and incubate for 30 min at room temperature. Finally add 50 µL Stop Solution to each well of the 96-well plate. Record the absorbance at 490 nm or 492 nm within 1 hr after adding the Stop Solution. The result of Calculation is %Cytotoxicity = (Experimental - E only - T only)/ (T Max - T only) ×100). The EC50 values were measured using GraphPad Prism software.

DELFIA Assay. LCL721.221 cells were induced by loading 1.0 mM peptide (GL Biochem, cat-217445) and incubate at 26° C. O/N. Label LCL721.221 cells with fluorescence enhancing ligand (DELFIA BATDA Reagent, Perkin Elmer, cat: AD0116), incubate at 37° C. for 20 min. Re-suspend the LCL721.221 pellet in RPMI 1640 plus 5% FBS and 1 mM peptide at a concentration of 1×105 cells/mL after 3 times of washing by PBS. NK92 cells was re-suspended at a concentration of 2×106 cells/mL in assay buffer (RPMI 1640 containing 5% FBS plus 10 ng/mL IL-2). Serially dilute antibody in assay buffer and add 50 µL (4*) diluted antibodies to assay plate (Cat-3599, Corning). Add 50 µL NK92 cell suspension to the plate and incubate with antibodies for 0.5 hr. And then add 100 µL labeled LCL721.221 cell suspension to the plate, mix sufficiently and incubate for 2-4 hrs at 37° C. Add 10 µL of Lysis Buffer to the maximum release well. Centrifuge the plate for 5 min at 500 g. Transfer 25 µL supernatant to a flat-bottom detection plate. Add 200 µL Europium Solution and shake the plate at 250 rpm for 15 min at room temperature. Measure fluorescence in a time-resolved fluorometer within 5 hrs.

PBMCs Mediated Cytotoxicity Assay Using Annexin V Apoptosis Marker. PBMCs were thawed and allowed to rest in growth medium for 18h in 37C prior to assay beginning. On the day of the assay A549 cells (Target) were trypsinized and stained with CFSE. CFSE-stained cells were seeded 50,000 cells per well in 96-well plate in triplicates. Then, 500,000 PBMCs were added to each well with target cells (E:T ratio of 10:1). Subsequently, test items 1062, 1093 and 1153 at 6 concentrations (10 nM, 1 nM, 0.1 nM, 0.01 nM, 0.001 nM and 0.0001 nM) wad added. PBS was used as vehicle control. The co-culture was incubated for 24 h at 37C 5% CO2. PBMCs from healthy donor was used. After the incubation period, conditioned media were collected and frozen. Annexin V staining was performed, and cells were analyzed by flow cytometry using CytoFLEX instrument.

PBMCs Mediated Cytotoxicity Assay Using Incucyte Live Imaging on GFP Labeled Cells. Abs diluents were prepared using 96 V plate. Seed MDA-MB-231-GFP to greiner-655090 plate,100 ul/well. Cells were counted before seeding. Add Ab to target cells, 20ul/well.Final Ab buffer concentration in total media is 2% only for the top dose. Seed effeft cells,100 ul/well(NK from isolation of fresh PBMC, NK:target cells=3:1),1 donor;NK media must be change to EMEM. Plate was analyzed in IncucyteS3 incubator every 2 hrs.

IFNy secretion. Supernatants analysis from the PBMC LDH cytotoxicity assay were analyzed for IFNg levels by ELISA. IFN-gamma was measured according to the protocol of Human INF gamma DuoSet ELISA kit (R&D, DY285). In brief, coat the Capture Antibody with the working concentration in PBS onto a 96-well microplate (Cat-9018, Corning), incubate overnight at RT. After the three times of wash, block plates with 300 µL of Reagent Diluent (1%BSA in PBS) for at least 1 hr at RT. After the three times of wash, add 100 µL sample with proper dilution or standards in Reagent Diluent, and incubate for 2 hrs at RT. After the three times of wash, add 100 µL diluted Detection Antibody, and incubate for 2 hrs at RT. After the three times of wash, add 100 µL of the working dilution of Streptavidin-HRP B, and incubate for 20 min at RT. After the three times of wash, add 100 µL of Substrate Solution, and incubate for 20 min at RT. Add 50 µL of Stop Solution. Read the OD450 using a microplate reader (Molecular Device, cat: Spectra Max M5e).

Receptor-Ligand Blocking (RBA) Assay. Competitive FACS based RBA: 1.3×105/well of CHOK1-NKG2A-CD94 cells were put into 96-well round-bottom polystyrene plates. After centrifuging the plates at 2000 rpm for 5 min, discard the supernatant. Re-suspend the cells with 50 µL diluted antibodies solution and incubate the plates at 4° C. for 0.5 hr, then add 50 µL/well HLA-E PE (1:2000) into plate, mix and incubate the plates at 4° C. for 2.5 hrs. Centrifuge the plate at 2000 rpm, 4° C. for 5 min and discard supernatant. Then wash the cells 3 times with FACS buffer and analyze the sample with FACS verse.

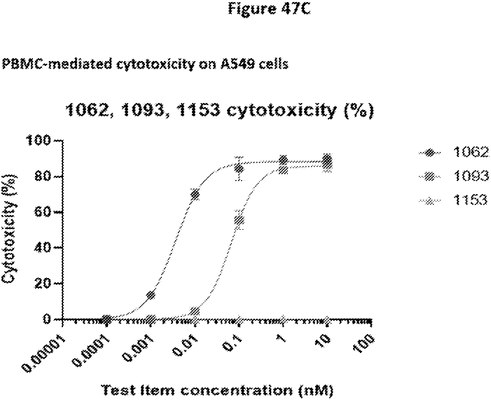

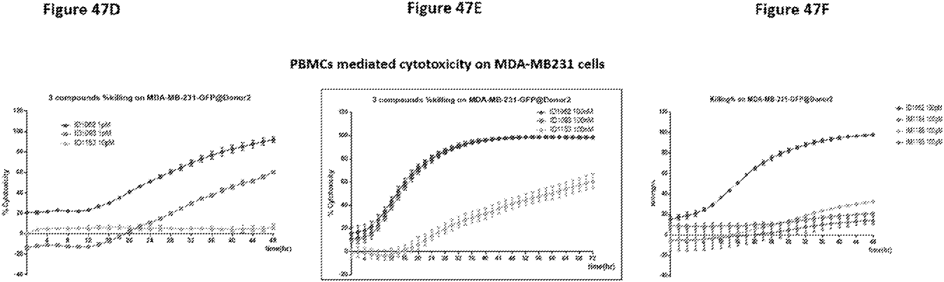

Results: Using lactate dehydrogenase assay, ~60% T cells mediated cytotoxicity was achieved with EC50 of 0.054 nM for IM-1062 Tribody (triangles up) and 0.144 nM for IM-1093 Tribody NKG2A mutated format (triangles down), while no cytotoxicity for the CD3-mutated Tribody variants IM-1153 and IM-1154 (circles and squares, respectively) in NCI-H226 lung cancer cells (FIG. 47A).

Using DELIFIA assay, ~40% NK mediated cytotoxicity was achieved at >100 nM concentration in the presence of IM-1062 Tribody (circles), while no activity of the NKG2A mutated form, IM-1093 (squares) in breast LCL721.221 cancer cells (FIG. 47B).

In PBMCs mediated cytotoxicity assay performed on CFSE labeled A549 cells using Annexin V, ~85% cell cytotoxicity was observed with EC50 of 0.0036 nM for IM-1062 Tribody (circles) and 0.0662 nM for IM-1093 Tribody NKG2A mutated variant (squares), while no cytotoxicity for the CD3mutated Tribody variant IM-1153 (triangles) in A549 lung cancer cells (FIG. 47C).

In PBMCs mediated cytotoxicity assay performed on GFP-MDA-MB231 cells, using Incucyte live cell imaging, maximum of ~70% killing was observed at 1pM following 36 hrs in the presence of for IM-1062 Tribody (circles) and ~30% killing for IM-1093 Tribody NKG2A mutated variant (squares), while no cytotoxicity for the CD3mutated Tribody variant, IM-1153 (triangles) (FIG. 47D, left). At higher Ab concentration of 100 nM, maximum of >85% killing was observed following 36 hrs in the presence of for IM-1062 Tribody (circles) and IM-1093 Tribody NKG2A mutated variant (squares), while at 100 nM, ~30% cytotoxicity with maximum of ~70% cytotoxicity over time for the CD3mutated Tribody variant IM-1153 (triangles) (FIG. 47D, middle). Similar performance was observed at 0.1 nM for IM-1062 and IM093 suggesting saturating conditions (data not shown). These findings were also observed when using HCT116-GFP expressing cells as the target cells (data not shown).

In PBMCs mediated cytotoxicity assay performed on GFP-MDA-MB231 cells, the ProTribody formats IM-1184, IM-1188 and IM-1193 exhibited ~15%, ~25% and ~10% cytotoxicity, respectively, following 40 hrs, as compared to the Tribody IM-1062 that exhibited ~90% cytotoxicity (FIG. 47D, right). No activity was observed at lower Abs concentrations of 10 pM, 1 pM and 0.1 pM (data not shown).

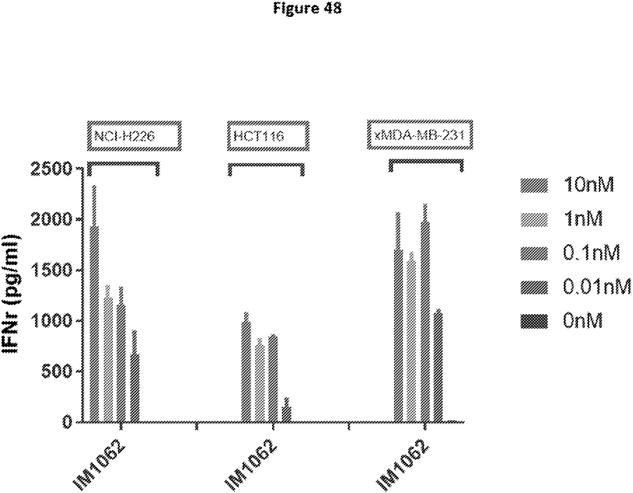

Supernatants from the PBMCs mediated cytotoxicity assay were analyzed for IFNg levels upon Tribody IM-1062 treatment. Tribody IM-1062 induce IFNg secretion in a dose dependent manner (10 nM,1 nM, 0.1 nM, 0.01 nM, 0 nM; left to right, respectively) in NCI-H226, HCT116, and MDA-MB-231 target cell lines co-cultured with PBMCs effector cells (FIG. 48). Similar pattern was also observed in the supernatants derived from the PBMCs mediated cytotoxicity assay using Annexin V apoptosis marker using A549 target cells (data not shown).

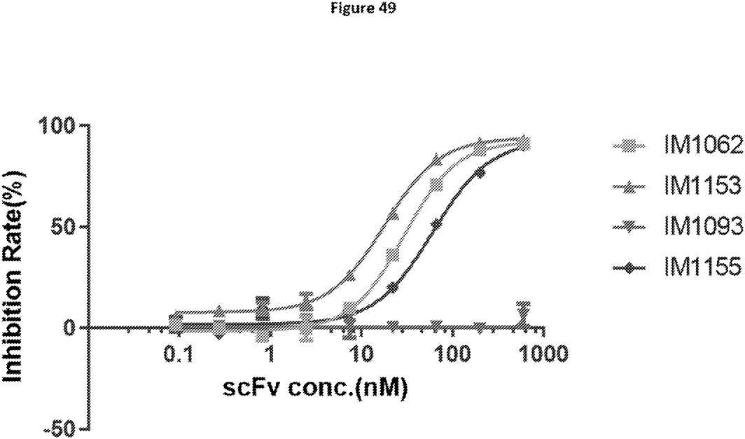

Various Tribody variants IM-1062, IM-1153 and IM-1155 (CD3 mutant variants) as well as IM-1093 (NKG2A mutant variant) were analyzed for their ability to block NKG2A-HLA interaction. The inhibition rate of IM-1062 (squares), IM-1153(triangles up), IM-1155 (rhombus) and IM-1093 (triangles down) measured as IC50 of 29 nM, 18 nM and 60 nM, respectively, while no inhibition activity was observed in the IM-1093 NKG2A mutated variant (FIG. 49).

Conclusion: In T cells and PBMCs mediated cell cytotoxicity assays, various cancer cell lines were undergoing cell killing in the presence of Tribody format IM-1062 and IM1093, with higher efficacy for IM-1062. IM-1062 also induced NK cell mediated cytotoxicity as shown in FIG. 47B. The ProTribody formats had significantly reduced activity. A pronounced difference in cell killing can be observed between Tribody and ProTribody due to the un-cleaved CD3 CAP masking. In addition, IM-1062 was also shown to induce dose dependent IFNg secretion as shown in FIG. 48, as well as blocking activity for the NKG2A-HLA-E interaction, as shown in FIG. 49.

### EXAMPLE 6

### In Vivo Efficacy in Xenograft NSG Mouse Model

Objective: To examine the inhibition of tumor growth induced by the Tribody in humanized mouse model.

Methods: In-vivo xenograft assay: NOD/SCID/IL2Rγnull (NSG) Mice (Charles River Laboratory) were used in accordance with a protocol reviewed and approved by the Institutional Animal Care and User Ethical Committee. Mice were housed in sterile conditions using high-efficiency particulate arrestance filtered micro-isolators and fed with irradiated food and acidified water. Xenograft tumors were generated by SC injection of 3×10e6 cancer target cells (in 200 µl of PBS) into 6-8 weeks old mice. When tumors of ~50 mm3 were formed, PBMC cells derived from two healthy donors peripheral blood were IV injected into tail vain at E:T ratio of 3:1. When tumors of ~80-120 mm³ were formed, different Tribodies were daily IP-injected (Intraperitoneal injection). After tumor inoculation, the animals were checked daily for morbidity and mortality. At the time of routine monitoring, the animals were checked for any effects of tumor growth and treatments on normal behavior such as mobility, food and water consumption, body weight gain/loss (body weights were measured twice weekly), eye/hair, matting and any other abnormal effect. The major endpoint was the tumor take rate and the tumor growth curve. Tumor sizes were measured twice weekly in two dimensions using a caliper, and the volume expressed in mm3 using the formula: V = 0.5 (a) × (b) 2 where a and b are the long and short diameters of the tumor, respectively. Body weights were measured twice weekly.

Specifically, mice were inoculated either with MDA-MB-231 cells, A549 cells or HCT116 cells. The study included three treatment arms, IM-1062, IM-1093 and control (PBS only). Each arm was comprised of 6 animals, where 3 mice were PBMCs injected from one healthy donor and 3 mice were PBMCs injected with second healthy donor at E:T ratios of 3:1, and dosed daily with 100ug/Kg/day.

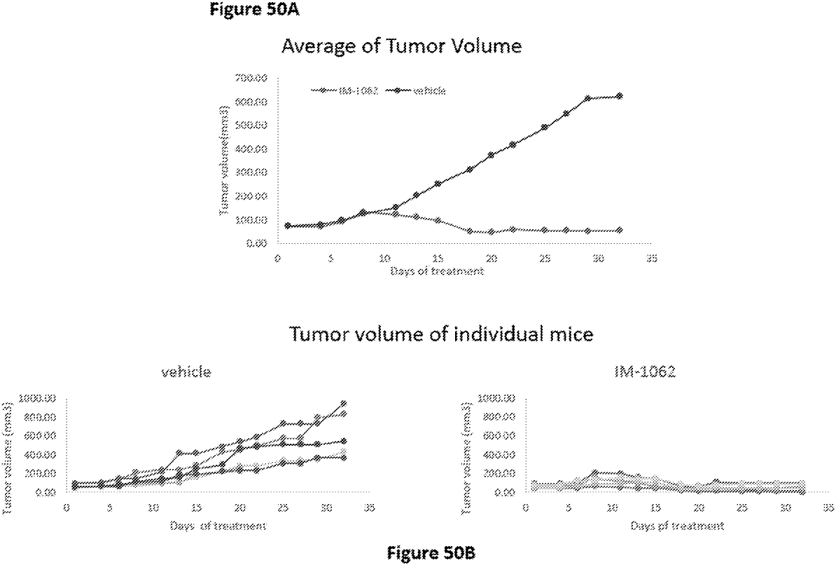

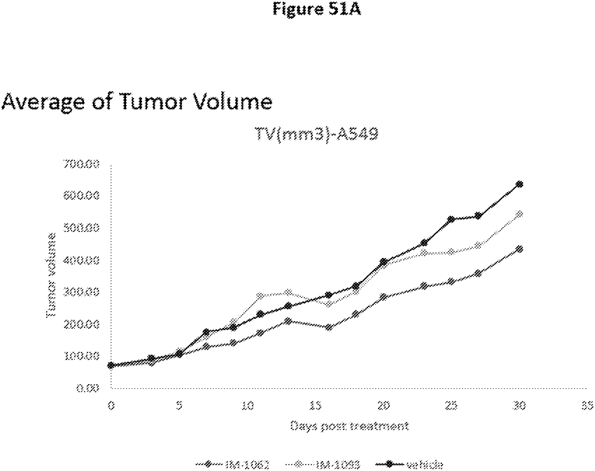

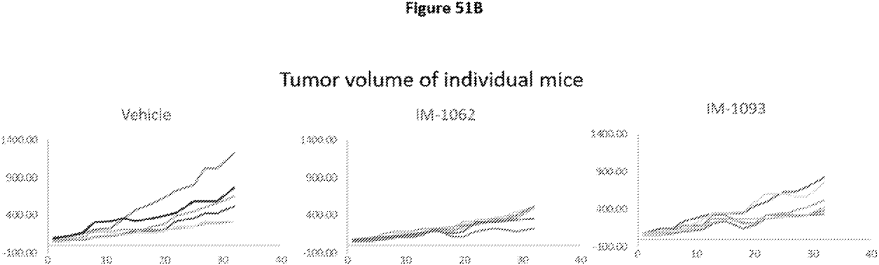

Results: FIG. 50A presents the tumor volume (mm3) for the mice treated with Tribody (IM-1062, light lower line) and the mice treated with PBS control (dark upper line). Similar observations were when treating with IM-1093, suggesting saturating conditions in this model (data not shown). FIG. 50B present tumor volume of individual mice treated with Tribody IM-1062 (left) and mice treated with PBS (right). Administration of Tribody reduced dramatically tumor size compared to the control samples and showed complete response.

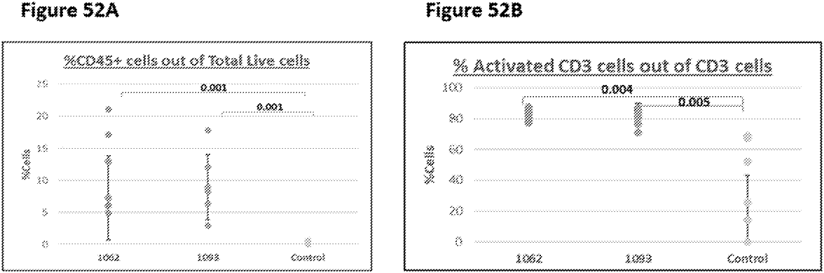

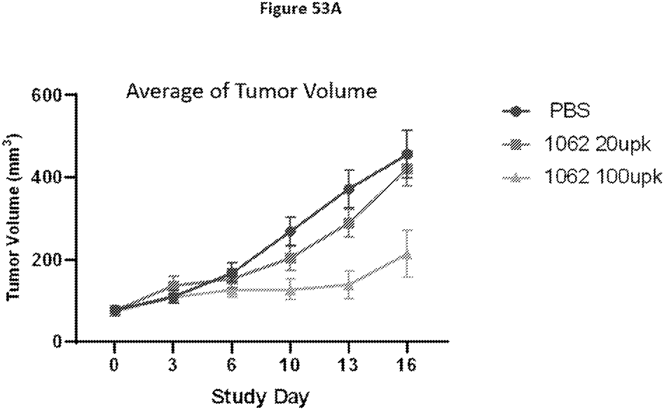

In additional study administrated with A549 cells (FIG. 51A), efficacy of ~45% tumor growth inhibition (TGI) was observed in the IM-1062 Tribody treated mice (dark grey line) versus the PBS treated mice (black line), while no significant activity for the NKG2A mutated Tribody IM-1093 (light grey line). FIG. 51B demonstrate tumor volume of individual mice of PBS treated mice (left), Tribody IM1062 treated mice (middle) and IM-1093 treated mice (left). Similar observation of 47% and 55% TGI for IM-1062 in HCT116 engrafted mice and NCI-H226 engrafted NCG mice, respectively (data not shown). Tumors were further dissected, and cells were dissociated for immune phenotyping. FIG. 52 presents % immune cells population in the tumor microenvironment (TME), with significantly higher %CD45 cells in the IM-1062 and IM-1093 Tribody treated groups versus PBS treated control group. Similarly, % activated T-cells is significantly higher in the IM-1062 and IM-1093 Tribody treated groups versus PBS treated control group (FIG. 52).

Conclusion: Efficacy of Tribody IM-1062 was consistently demonstrated in multiple in-vivo xenograft models with responses ranges from ~50% TGI up to complete response, with some studies indicating for lower efficacy for the NKG2A mutated IM-1093 Tribody. In addition, tumors were evaluated for % immune cell populations by FACS and the analysis indicated for massive recruitment and infiltration of immune cells into the TME in the Tribody treated groups.

### EXAMPLE 7

### In Vivo Efficacy of Tribody/ProTribody in Humanized Engrafted CD34+ NSG Mouse Expressing Human IL15

Objective: To examine the inhibition of tumor growth induced by the Tribody in HCT116 or MDA-MB-231engrafted hu-CD34 NSG-TM -IL15 mice expressing human IL15 for maintaining NK cells and other immune cell’s proliferation and population.

Methods: female hu-CD34 NSG™- SGM3 mice (Jax stock # 030890) mice from 4 different donors (150,165,173,174) were enrolled on this study.

HCT-116 cells were cultured as per ATCC protocol. HCT- 116 cells were suspended in PBS mixed 1:1 with GFR Matrigel to a final concentration of 50×10⁶ cells/ml. 100 ul per mouse were injected SC in the right flank of each mouse for a total of 5×10⁶ cells per mouse. Mice were randomized into treatment groups based on the tumor volume (50-150 mm³). 10 mice (10 animals/ 4 donors) in each group were inoculated with HCT116 cells. The study included IM-1062, IM-1093 and control (PBS only) and IP dosed daily with either 20ug/Kg/day or 100ug/Kg/day. Animals were monitored daily and twice a week measured for tumor volume and body weight.

In another study, MDA-MB231 cells were used. 6 mice (6 animals/ 2 donors) in each group were inoculated with MDA-MB-231 cells. The study included Tribody IM-1062, ProTribody IM-1184, ProTribody IM-1193 and control (PBS only) and IP dosed daily with either 20 ug/Kg/day or 100 ug/Kg/day for the Tribody, and either with 50 ug/Kg/day or 200 ug/Kg/day for the ProTribodies. Animals were monitored daily and twice a weekly measured for tumor volume and body weight.

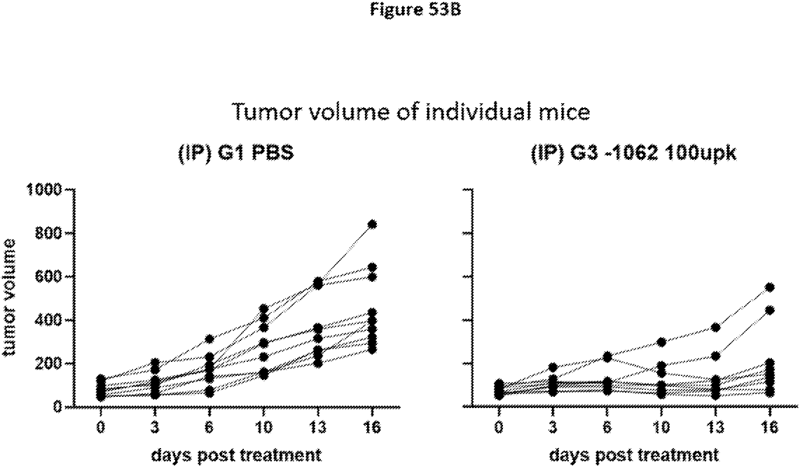

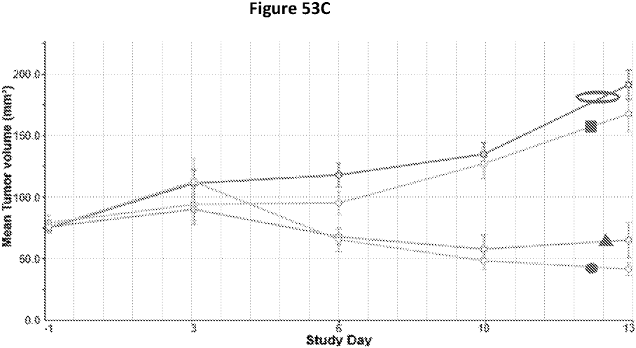

Results: FIG. 53A presents the tumor volume (mm3) for the mice treated with Tribody IM-1062 at 20 ug/Kg/day (squares), 100 ug/Kg/day (triangles) and the mice treated with PBS control (circles). Administration of Tribody reduced dramatically tumor size compared with the control samples and showed >80% TGI. Similar observations were when treating with IM-1093, suggesting saturating conditions in this model (data not shown). FIG. 53B present tumor volume of individual mice treated with Tribody PBS (left) and mice treated with IM-1062 (right).

FIG. 53C presents the tumor volume (mm3) for the mice treated with Tribody IM-1062 at 100 ug/Kg/day (line marked with triangles), or with ProTribody IM-1184 at 200 ug/Kg/day (line marked with circle), or with non-cleavable ProTribody IM-1193 at 200 ug/Kg/day (line marked with square) or with PBS vehicle control (line marked with ellipse). Administration of Tribody reduced dramatically tumor size compared with the vehicle control samples. Similar observations were when treating with ProTribody IM-1184 showing dramatic tumor volume reduction as compared to the control group (vehicle). IM-1193 non-cleavable control ProTribody showed very little tumor growth inhibition as expected, indicating the masking of the CD3 CAP that blocks the binding to CD3 and hence lack of protease linker in this molecule prevents prominent activity in the TME.

In additional study administrated with HCT116 cells to NSG CD34+ engrafted mice boosted with IL15 plasmid, where mice dosed with 20 ug/Kg/day, significant efficacy of ~85% tumor growth inhibition (TGI) was observed in the IM-1062 Tribody treated mice versus the PBS treated mice, while no significant activity for the NKG2A mutated Tribody IM-1093 (data not shown). Tumors were further dissected, and cells were dissociated for immune phenotyping with significantly higher #CD45 and CD3 cells in the IM-1062 and IM-1093 Tribody treated groups versus s PBS treated control group (data not shown).

Conclusions: Efficacy of Tribody IM-1062 was consistently demonstrated in CD34+ engrafted mouse models with responses ranges from ~80%% TGI up to complete response . Efficacy of the ProTribody IM-1184 was demonstrated as well with complete response indicating that the active form was generated in the TME upon protease cleavage, while the non-cleavable ProTribody control format of IM-1193 showed low activity due to lack of cap removal at the TME.

What is claimed is:

1-26. (canceled)

27. A precursor tri-specific antibody construct, comprising:
(a) a first binding domain that binds to a tumor associated antigen (TAA);
(b) a second binding domain that binds to a first natural killer (NK) cell surface antigen or a second binding domain comprising a cytokine receptor engager;
(c) a third binding domain that binds to a T cell surface antigen or a second NK cell surface antigen; and
(d) a regulatory domain comprising a protease cleavage domain, a half-life prolonging (HLP) domain, and a CAP component that reduces the ability of the third binding domain to bind to its target antigen.

28. The precursor tri-specific antibody construct of claim 27, wherein said second binding domain binds to the natural killer (NK) cell surface antigen NKG2A.

29. The precursor tri-specific antibody construct of claim 27, wherein the first binding domain and the second binding domain each comprises a single chain variable fragment (scFv), and the third binding domain comprises a Fab antigen binding fragment.

30. The precursor tri-specific antibody construct of claim 27, wherein the first binding domain binds to a TAA selected from EGFR, ROR1, PSMA, and 5T4, the second binding domain binds to the NK cell surface antigen NKG2A, and the third binding domain binds to T cell surface antigen CD3.

31. The precursor tri-specific antibody construct of claim 30, wherein the first binding domain binds to 5T4.

32. The precursor tri-specific antibody construct of claim 27, wherein the first binding domain comprises a scFv that binds to 5T4, the second binding domain is a scFv that binds to NKG2A, and the third binding domain is a Fab that binds to CD3.

33. The precursor tri-specific antibody construct of claim 31, wherein the first binding domain that binds to 5T4 comprises a variable heavy (VH) chain sequence set forth in SEQ ID NO: 623 and a variable light (VL) chain set forth in SEQ ID NO: 624; or comprises three complementarity determining regions (CDRs) on a heavy chain (HCDR1, HCDR2, and HCDR3) and three CDRs on a light chain (LCDR1, LCDR2, and LCDR3), wherein
(i) the HCDR1, HCDR2, and HCDR3 comprise the amino acid sequences of SEQ ID NOs:476-478, and the LCDR1, LCDR2, and LCDR3 comprise the amino acid sequences of SEQ ID NOs:480-482; or
(ii) the HCDR1, HCDR2, and HCDR3 comprise the amino acid sequences of SEQ ID NOs:484-486, and the LCDR1, LCDR2, and LCDR3 comprise the amino acid sequences of SEQ ID NOs:488-490; or
(iii) the HCDR1, HCDR2, and HCDR3 comprise the amino acid sequences of SEQ ID NOs:492-494, and the LCDR1, LCDR2, and LCDR3 comprise the amino acid sequences of SEQ ID NOs:496-498; or
(iv) the HCDR1, HCDR2, and HCDR3 comprise the amino acid sequences of SEQ ID NOs:500-502, and the LCDR1, LCDR2, and LCDR3 comprise the amino acid sequences of SEQ ID NOs:504-506; or
(v) the HCDR1, HCDR2, and HCDR3 comprise the amino acid sequences of SEQ ID NOs:508-510, and the LCDR1, LCDR2, and LCDR3 comprise the amino acid sequences of SEQ ID NOs:512-514; or
(vi) the HCDR1, HCDR2, and HCDR3 comprise the amino acid sequences of SEQ ID NOs:516-518, and the LCDR1, LCDR2, and LCDR3 comprise the amino acid sequences of SEQ ID NOs:520-522; or
(vii) the HCDR1, HCDR2, and HCDR3 comprise the amino acid sequences of SEQ ID NOs:524-526, and the LCDR1, LCDR2, and LCDR3 comprise the amino acid sequences of SEQ ID NOs:528-530; or
(viii) the HCDR1, HCDR2, and HCDR3 comprise the amino acid sequences of SEQ ID NOs:532-534, and the LCDR1, LCDR2, and LCDR3 comprise the amino acid sequences of SEQ ID NOs:536-538; or
(ix) the HCDR1, HCDR2, and HCDR3 comprise the amino acid sequences of SEQ ID NOs:540-542, and the LCDR1, LCDR2, and LCDR3 comprise the amino acid sequences of SEQ ID NOs:544-546.

34. The precursor tri-specific antibody construct of claim 28, wherein the second binding domain, that binds to NKG2A, comprises a VH chain sequence set forth in SEQ ID NO: 635 and a VL chain sequence set forth in SEQ ID NO: 639; or comprises three complementarity determining regions (CDRs) on a heavy chain (HCDR1, HCDR2, and HCDR3) and three CDRs on a light chain (LCDR1, LCDR2, and LCDR3), wherein the HCDR1, HCDR2, and HCDR3 comprise the amino acid sequences of SEQ ID NOs:636-638, and the LCDR1, LCDR2, and LCDR3 comprise the amino acid sequences of SEQ ID NOs:640-642.

35. The precursor tri-specific antibody construct of claim 27, wherein said antibody construct comprises polypeptide A and polypeptide B, said polypeptide A and polypeptide B comprise amino acid sequences having the sequences of SEQ ID NOs: 206 and 398.

36. The precursor tri-specific antibody construct of claim 35, wherein said antibody construct is IM1240.

37. A tri-specific antibody derived from the precursor tri-specific antibody construct of claim 27.

38. The tri-specific antibody of claim 37 comprising:
(e) a first binding domain that binds to a tumor associated antigen (TAA);
(f) a second binding domain that binds to the natural killer (NK) cell surface antigen NKG2A; and
(g) a third binding domain that binds to a T cell surface antigen or a second NK cell surface antigen.

39. The tri-specific antibody of claim 38, wherein the first binding domain is a scFv that binds to 5T4, the second binding domain is a scFv that binds to NKG2A, and the third binding domain is a Fab that binds to CD3.

40. The tri-specific antibody of claim 39, wherein said tri-specific antibody comprises polypeptide A and polypeptide B, said polypeptide A and polypeptide B comprise amino acid sequences having the sequences SEQ ID NOs: 180 and 177; or wherein said polypeptide A and polypeptide B comprise amino acid sequences having the sequences SEQ ID NOs: 180 and 392.

41. The tri-specific antibody of claim 40, wherein said tri-specific antibody is IM1222.

42. The precursor tri-specific antibody construct of claim 27, wherein said third binding domain comprises a Fab region comprising a heavy chain polypeptide and a light chain polypeptide, said heavy chain polypeptide comprises a heavy chain variable region and a heavy chain constant region (VH-CH), said light chain polypeptide comprises a light chain variable region and a light chain constant region (VL-CL), wherein
when said first binding domain is located C-terminally to said VL-CL region, said second binding domain is located C-terminally to said VH-CH region, or
when said first binding domain is located C-terminally to said VH-CH region, said second binding domain is located C-terminally to said VL-CL region.

43. The precursor tri-specific antibody construct of claim 27, wherein a single regulatory domain comprising a protease cleavage domain, a half-life prolonging (HLP) domain, and a CAP component is located N-terminally to said VH region or to said VL region of said third binding domain.

44. A pharmaceutical composition, comprising a precursor tri-specific antibody construct of claim 27, and a pharmaceutically acceptable carrier.

45. A pharmaceutical composition comprising the tri-specific antibody of claim 38.

46. A nucleic acid construct comprising one or more nucleic acid sequences, said sequences encoding a precursor tri-specific antibody construct of claim 27.

47. An expression vector comprising the nucleic acid construct of claim 46.

48. A nucleic acid construct comprising one or more nucleic acid sequences, said sequences encoding a tri-specific antibody of claim 38.

49. A method of treating, preventing, or delaying disease progression, reducing tumor load, or reducing the incidence of a cancer or a tumor, or any combination thereof, in a subject in need of such treatment, comprising a step of administering to the subject a pharmaceutical composition of claim 44.

50. The method of claim 49, wherein the cancer or tumor comprises a solid tumor or non-solid tumor, or the cancer or tumor comprises a metastasis of a cancer or tumor.

51. The method of claim 50, wherein the cancer or tumor expresses the TAA to which the first binding domain binds.

52. The method of claim 51, wherein the TAA is 5T4.
